(19) United States
(12) Patent Application Publication
(10) Pub. No.: US 2016/0053318 A1
(43) Pub. Date: Feb. 25, 2016

(54) MARKERS OF ENDOTHELIAL PROGENITOR CELLS AND USES THEREOF

(71) Applicant: Medvet Science Pty Ltd., Underdale (AU)

(72) Inventors: CLAUDINE SHARON BONDER, Adelaide (AU); Angel Francisco Lopez, Adelaide (AU); Gert Hoy Talbo, Bundoora (AU)

(73) Assignee: Medvet Science Pty Ltd., Underdale (AU)

(21) Appl. No.: 14/845,525

(22) Filed: Sep. 4, 2015

Related U.S. Application Data
(63) Continuation of application No. 13/882,806, filed on May 1, 2013, now abandoned.
(60) Provisional application No. 61/410,674, filed on Nov. 5, 2010.

(51) Int. Cl. C12Q 1/68; A61K 49/00; A61K 35/44; G01N 33/569

(52) U.S. Cl. CPC C12Q 1/6881; G01N 33/56966; A61K 49/005; A61K 35/44; G01N 2333/70596

(57) ABSTRACT

The present invention provides markers of endothelial progenitor cells (EPCs) and use of those markers and reagents that bind thereto to detect EPC cells or diagnose, prognose, treat or prevent EPC-associated conditions.

### RELATED APPLICATION DATA

This application is a continuation of U.S. application Ser. No. 13/882,806 filed May 1, 2013, which is a National Phase application of International Application No. PCT/AU2011/001415 filed Nov. 7, 2011, which claims priority to U.S. Provisional Patent Application No. 61/410,674 filed Nov. 5, 2010, each of which is incorporated herein by reference in its entirety.

### FIELD

The present invention relates to nucleic acid or protein markers of endothelial progenitor cells (EPCs) and uses thereof.

### SEQUENCE LISTING

A Sequence Listing of nucleotide and amino acid sequences referenced in this application as “SEQ ID NO: 1-340” is submitted in computer readable form along with this application. The computer readable form of the Sequence Listing is hereby incorporated by reference into this application.

### BACKGROUND

Various disorders are associated with insufficient neovascularization, e.g., ischemia or aberrant angiogenesis/vasculogenesis, e.g., cancer. In this regard, the skilled artisan will be aware that neovascularization encompasses angiogenesis and vasculogenesis. Angiogenesis is the growth of new blood vessels from pre-existing vessels. Angiogenesis can take two forms, i.e., sprouting angiogenesis is the formation of new vessels toward an angiogenic signal, and intussusceptive angiogenesis is the process by which a blood vessel is split into two new vessels. In contrast, vasculogenesis is the de novo formation of blood vessels by tissue resident endothelial progenitor cells (EPCs). EPCs are considered to play a role in both angiogenesis and vasculogenesis.

Various types of tissue resident or circulating blood cells can be induced to display endothelial characteristics and are referred to as EPCs. Two of the more commonly studied forms of EPCs are monocytic EPCs and hemangioblastic EPCs.

Monocytic EPCs are found in peripheral blood mononuclear cells (PBMCs) and in culture are capable of forming colonies of endothelial-like cells that augment neovascularization in animal models (Asahara et al., 1997). Monocytic EPCs can be obtained from blood and are potent secretors of angiogenic factors, indicating a role in promoting angiogenesis and endothelial repair through paracrine stimulation of resident endothelium (Rehman et al., 2007). Following culture of a mixed population of EPCs, monocytic EPCs give rise to “early outgrowth EPCs”, which possess only transient proliferation potential in vitro, cannot be passaged, express the monocytic marker CD14 and display overlap between endothelial and macrophage functions, e.g., phagocytosis, antithrombogenic activity and production of vasoactive substances (Krenning et al., 2009).

Hemangioblastic EPCs circulate in peripheral blood and are also detectable in bone marrow. These cells are also mobilized from bone marrow under conditions of hypoxia, e.g., during ischemia, or in response to hematopoietic stem cell mobilization, e.g., using granulocyte colony stimulating factor (G-CSF) (Kawamoto and Losordo, 2008; Liu et al., 2008). These cells undergo clonal expansion and give rise to “late outgrowth EPCs”. These cells are positive for CD34 (Krenning et al., 2009).

While monocytic EPCs and hemangioblastic EPCs arise from distinct lineages and show functional differences in vitro, both forms contribute to in vivo neovascularization in several disease models (Krenning et al., 2009). In this regard, EPCs have been shown to integrate into newly forming blood vessels (Asahara et al., 1997). In particular, injury or hypoxia induces production of factors such as vascular endothelial growth factor (VEGF) and/or monocyte chemotactic protein-1 (MCP-1), which result in break-down of extracellular matrix between endothelial cells in existing blood vessels facilitating extravasation of EPCs (particularly, monocytic EPCs). These EPCs secrete various proteases including matrix metalloproteases, matrix metalloelastases and elastases, which further degrade the endothelial extracellular matrix. The EPCs also form a network of tunnels that link to existing blood vessels. Hemangioblastic EPCs are recruited to and line the lumen of these tunnels. Both monocytic and hemangioblastic EPCs secrete high levels of pro-angiogenic cytokines, and the presence of both forms of EPCs results in a synergistic increase in these compounds. These cytokines are considered to cause differentiation of EPCs into mature endothelium and to recruit mature endothelial cells to form blood vessels (Krenning et al., 2009).

### EPCs and Autoimmune/Inflammatory/Rheumatic Diseases and Connective Tissue Disorders

EPC numbers and/or function have been shown to be aberrant in subjects suffering from a variety of disorders, such as cardiovascular disease, rheumatoid arthritis, psoriatic arthritis, systemic lupus erythematosus (SLE), systemic sclerosis and ANCA-associated vasculitis.

Subjects suffering from cardiovascular disease have reduced levels of hemangioblastic EPCs, and this reduction is associated with higher systolic blood pressure, higher low density lipoprotein (LDL) cholesterol levels, metabolic syndrome and coronary artery disease. Monocytic EPCs derived from subjects suffering from cardiovascular disease have a reduced capacity for outgrowth in vitro, which is associated with type I and type II diabetes, hypertension and renal insufficiency. Prospective data also shows an association between lower levels of hemangioblastic and monocytic EPCs with increased rates of cardiovascular disease (Westerweel and Verhaar, 2009).

Subjects suffering from rheumatoid arthritis have reduced levels of hemangioblastic and monocytic EPCs (Egan et al., 2008). Levels of hemangioblastic EPCs also show an inverse correlation with rheumatoid arthritis disease severity score, erythrocyte sedimentation rate and rheumatoid factor levels (Egan et al., 2008; Grisar et al., 2005). Monocytic EPCs from subjects suffering from rheumatoid arthritis also show reduced migratory response to VEGF, and serum from rheumatoid arthritis patients has been shown to inhibit migration of EPCs isolated from healthy controls (Herbrig et al., 2006).

Patients with SLE that show no overt or subclinical vascular disease or suffering from active SLE have reduced numbers of circulating hemangioblastic EPCs (Westerweel et al., 2007; Denny et al., 2007). The ability of monocytic EPCs from SLE patients to secrete pro-angiogenic factors and to form colonies when cultured in vitro have also been shown to be inhibited (Westerweel and Verhaar, 2009).

In systemic sclerosis, levels of hemangioblastic EPCs show a biomodal pattern with numbers increasing during the first five or so years after disease onset and then reducing to levels below those of healthy controls (Westerweel and Verhaar, 2009). Monocytic EPCs have also been found to be reduced in systemic sclerosis patients (Zhu et al., 2008).

EPC dysfunction has also been described in diabetes (Tepper et al., 2002). For example, hyperglycemia associated with diabetes has been shown to directly reduce EPC numbers (Ding and Triggle, 2005; Kang et al., 2009). Furthermore, a mouse model of diabetes was shown to have suppressed levels of EPC mobilization in response to ischemia (Kang et al., 2009).

As is apparent from the foregoing, various individual studies have found aberrant levels of EPCs in various disease states. However, many of these studies use different assays in an attempt to quantify EPC numbers, including detecting EPCs using antibodies against CD34 and/or VEGF receptor 2 (VEGFR2/KDR), neither of which is specific for EPCs. Furthermore, some researchers pre-culture mononuclear cells before surface marker analysis, which may affect EPC quantification. Other detection methods involve culturing isolated cells to form colony forming units (CFU) and/or double staining cultured cells with acetylated-LDL and Ulex europaeus I lectin. Both of these methods involve multiple steps and are difficult to reproduce between laboratories (Avouac et al., 2008). Accordingly, comparing data obtained from different laboratories is difficult. These difficulties have also hampered production of standardized assays for detecting, isolating or quantifying EPCs. It follows that there is a need in the art for methods that facilitate detection and/or quantification of EPCs in samples from subjects.

Studies using therapeutics of rheumatic disease have also shown that EPC numbers return to normal levels or close to normal levels following treatment, indicating that modulation of EPC numbers may also provide therapeutic benefit in these diseases (Avouac et al., 2008).

### EPCs and Vascular/Tissue Regeneration

EPC levels have been shown to increase at sites of ischemia, such as following a stroke or during ischemia following a transplant. Moreover, the number of circulating EPCs has been shown to be significantly higher in patients suffering from acute ischemic stroke than in at-risk control subjects, and the magnitude of this difference is directly related to positive clinical outcome (Yip et al., 2008). Sobrino et al. (2007) have also shown that the magnitude of EPC population size increase is associated with positive outcome three months after a stroke and reduced infarct growth and neurological impairment at days 7 and 90. Accordingly, methods that facilitate rapid determination of EPC numbers in a sample will permit prognosis of subjects suffering from ischemia and determination of suitable therapeutic options.

Animal studies have also shown that administration of EPC containing populations of cells can improve outcome after an ischemic event. For example, administration of CD34+ cells accelerated neovascularization in a cerebral ischemic zone 48 hours after stroke, increased neurogenesis and improved functional indexes in a mouse model (Taguchi et al., 2004). Bone marrow-derived cells and peripheral blood cells have also been shown to improve neurological function in mouse and rat models of cerebral ischemia (Zhang et al., 2002 and Ukai et al., 2007).

In preclinical studies, EPC-containing cell populations were found to contribute directly to blood vessel formation as well as significantly increase vascular density (angiogenesis) from endogenous endothelial cells. These data demonstrate that the administered cells promote neovascularization by endogenous tissue, e.g., by secretion of angiogenic factors (Young et al., 2007).

CD34⁺ bone marrow-derived cells (which contain EPCs) have also been shown to improve ventricular ejection fraction, reduce infarct size and improve myocardial perfusion in human phase I and II clinical trials (Krenning et al., 2009).

Blood-derived angioblasts have also been shown to improve blood-flow in a mouse model of diabetes, thereby reducing the risk of diabetic wounds (Schatterman et al., 2000).

A disadvantage of all of the foregoing studies is that mixed populations of cells are administered to subjects. For example, administration of unselected bone marrow cells from an autologous source leads to an increased risk of graft-versus-host disease. Furthermore, administration of relatively uncharacterized mixed cell populations is undesirable from a human clinical perspective.

Another application of EPCs is in the construction of endothelial-coated vascular grafts. In this regard, the poor patency rate of bypass grafts has been largely attributed to thrombosis caused by delayed endothelialization of their lumen (Young et al., 2007). Autologous, vessel-derived endothelial cells have been used to seed these grafts. However, insufficient numbers of cells has limited the clinical utility of this approach (Young et al., 2007). A separate approach taken by Rotmans et al. (2006) was to coat vascular grafts with anti-CD34 antibodies to capture EPCs in circulation. This approach resulted in complete coverage of the grafts within three days of implantation. However, the authors observed a hyperplastic response, which they believe may have occurred because the anti-CD34 antibodies were not specific for EPCs and additionally captured CD34⁺ non-endothelial cells which induced restenosis.

Increasing neovascularization using EPC-based treatments is also likely to provide therapeutic benefits in treatment of wounds, bone defects and hypertension and for improving tissue grafting. For example, increasing neovascularization results in increased delivery of oxygen, nutrients and components of the inflammatory response to regions requiring those factors.

### EPCs and Infection

EPC levels have also been shown to increase in subjects suffering from sepsis. For example, Becchi et al., (2008) found increased levels of circulating EPCs in subjects suffering from sepsis and that the number of EPCs detected is correlated with disease severity. Raffat et al., (2007) also found increased levels of circulating EPCs in subjects suffering from sepsis and that the number of EPCs detected is inversely correlated with survival.

### EPCs and Unregulated Angiogenesis

Unregulated or excessive angiogenesis is observed in a number of conditions, such as psoriasis, nephropathy, cancer and retinopathy (Gupta and Zhang, 2005).

In the case of cancer, increased levels of EPCs have been observed in subjects suffering from multiple myeloma (Zhang et al., 2005). Furthermore, Shaked et al. (2005) studied numerous mouse tumor models (transplanted versus spontaneous, solid versus leukemic, syngeneic Lewis lung carcinoma LL/2, nerythrolukemic, orthotopic human breast cancer MDA-MB-231 and human lymphoma) and showed a strong correlation between tumor growth and EPC numbers. The authors were also able to effectively define optimal anti-angiogenic therapy dosage based on EPC monitoring. These data indicate that methods and/or reagents which facilitate rapid and/or simple detection and/or quantification of EPCs will also facilitate diagnosis and/or prognosis of cancer and/or prediction of suitable therapy.

Progression of tumor growth and/or metastasis is/are angiogenesis dependent. For example, Folkman et al. (1971) showed that tumors cannot grow between 1 mm or 2 mm without new blood vessels. Some data indicate that marrow-derived endothelial progenitor cells can be mobilized and incorporated into new blood vessels (Rusinova et al., 2003).

Inhibitors of angiogenesis have also shown efficacy in the treatment of cancers as is exemplified by Bevacizumab (Avastin®, Genentech/Roche), a humanized antibody against VEGF (Zondor et al., 2004). Some advantages of angiogenesis-based treatments are:
A single vessel provides nutrition for thousands of tumor cells and has to be damaged at only one point to block blood flow;
Endothelial cells and endothelial progenitor cells are normal diploid cells that are unlikely to acquire genetic mutations that render them drug resistant; and
Blood flow, a surrogate marker for biological activity of a drug, is measurable in the clinic (Gupta and Zhang, 2005).

EPCs from subjects suffering from macular degeneration have also been shown to expand more rapidly than those from normal subjects. Anti-VEGF therapeutics, such as bevacizumab and ranibuzumab (Lucentis®) have also been shown to be useful for treating macular degeneration.

As discussed above, the markers currently used for EPCs are not sufficiently specific for those cells. Accordingly, drugs targeting those markers are not sufficiently specific to kill or inhibit EPCs for the treatment of conditions associated with uncontrolled angiogenesis, e.g., cancer. Moreover, drugs targeting such markers may target non-endothelial cell types, potentially leading to detrimental side-effects.

It will be apparent from the foregoing discussion that depletion of EPCs provides an attractive means for treating various conditions, e.g., cancer. However, as discussed above, insufficient markers that permit removal of EPCs has hampered therapeutic strategies targeting these cells. Accordingly, there is a need in the art for new markers, for example cell surface markers of EPCs that permit detection, isolation, removal or destruction of EPCs, e.g., for therapeutic and/or prophylactic purposes.

### SUMMARY

The inventors have produced EPCs by overexpressing the enzyme sphingosine kinase-1 (SK-1) in human umbilical cord vein endothelial cells (HUVECs) (Bonder et al., (2009). SK-1 is expressed at high levels and is responsible, at least in part, for maintaining an endothelial progenitor cell (EPC) phenotype, i.e., preventing the cells from differentiating into mature endothelial cells. Using these cells as a model for EPCs generally, the inventors identified proteins, such as cell surface proteins, upregulated in EPCs compared to other cells, such as endothelial cells.

The inventors have also isolated non-adherent CD133 expressing EPCs from umbilical cord blood and identified cell surface biomarkers that are expressed at increased levels on these cells compared to other cells, such as endothelial cells. The inventors have identified these markers using nucleic acid-based and proteomic-based approaches.

The inventors have also shown that a marker of EPCs (DSG2) is also expressed on vascular cells in vivo. DSG2 is also expressed on some melanoma cells, and the inventors have shown that by inhibiting DSG2 they can reduce tube formation when endothelial cells and melanoma cells are co-cultured.

Accordingly, an example of the present invention provides a method for detecting an EPC, the method comprising determining the level of expression of a nucleic acid or protein set forth in Table 1, or a nucleic acid or protein having at least about 70% identity thereto, in, on or secreted from a cell, wherein an increased level of expression of a nucleic acid or protein set forth in Table 1 or a nucleic acid or protein having at least about 70% identity thereto compared to another cell type is indicative of an EPC.

In one example, the nucleic acid or protein is expressed in, on or secreted from EPCs at a level at least 1.5 fold greater than in, on or secreted by human umbilical cord vascular endothelial cells (HUVECs), for example at a level at least 2 fold greater than in, on or secreted by HUVECs, such as at a level at least 3 or 4 or 5 fold greater than in, on or secreted by HUVECs.

For example, the nucleic acid or protein is expressed in, on or secreted by non-adherent CD133⁺ EPCs at a level at least 1.5 fold greater than in, on or secreted by HUVECs, for example, at a level at least 2 fold greater than in, on or secreted by HUVECs, such as at a level at least 3 or 4 or 5 fold greater than in, on or secreted by HUVECs.

For example, the nucleic acid or protein is expressed in, on or secreted from EPCs at a level at least 1.5 fold greater than in, on or secreted by HUVECs and the nucleic acid comprises a sequence set forth in any one of SEQ ID NOs: 15, 1, 17, 9, 13, 3, 5, 7, 11, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 237, 239, 241, 243, 245, 247, 249, 251, 253, 255, 257, 259, 265, 267, 269, 271, 273, 275, 277, 279, 281, 305, 307, 309, 311, 313, 315, 317, 319, 321, 323, 325 or 327 or a nucleic acid having at least about 70% identity thereto, or the protein comprises a sequence set forth in any one of SEQ ID NOs: 16, 2, 18, 10, 14, 4, 6, 8, 12, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 238, 240, 242, 244, 246, 248, 250, 252, 254, 256, 258, 260, 266, 268, 270, 272, 274, 276, 278, 280, 282, 306, 308, 310, 312, 314, 316, 318, 320, 322, 324, 326 or 328 or a protein having at least about 70% identity thereto.

For example, the nucleic acid or protein is expressed in, on or secreted by non-adherent CD133⁺ EPCs at a level at least 1.5 fold greater than in, on or secreted by HUVECs and the nucleic acid comprises a sequence set forth in any one of SEQ ID NOs: 15, 1, 17, 9, 13, 3, 5, 7, 11, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163 or 327 or a nucleic acid having at least about 70% identity thereto, or the protein comprises a sequence set forth in any one of SEQ ID NOs: 16, 2, 18, 10, 14, 4, 6, 8, 12, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164 or 328 or a protein having at least about 70% identity thereto.

For example, the nucleic acid or protein is expressed in, on or secreted from EPCs at a level at least 2 fold greater than in, on or secreted by HUVECs and the nucleic acid comprises a sequence set forth in any one of SEQ ID NOs: 15, 1, 17, 9, 13, 3, 5, 7, 19, 21, 23, 27, 29, 31, 33, 37, 39, 43, 45, 47, 49, 51, 53, 55, 57, 59, 61, 63, 65, 67, 69, 71, 73, 75, 77, 79, 81, 83, 85, 87, 93, 95, 97, 99, 101, 103, 105, 111, 113, 115, 117, 119, 121, 123, 125, 131, 133, 135, 137, 139, 141, 143, 145, 155, 159, 161, 163, 237, 239, 241, 243, 245, 247, 249, 251, 265, 305, 307, 309, 311 or 327 or a nucleic acid having at least about 70% identity thereto, or the protein comprises a sequence set forth in any one of SEQ ID NOs: 16, 2, 18, 10, 14, 4, 6, 8, 20, 22, 24, 28, 30, 32, 34, 38, 40, 44, 46, 48, 50, 52, 54, 56, 58, 60, 62, 64, 66, 68, 70, 72, 74, 76, 78, 80, 82, 84, 86, 88, 94, 96, 98, 100, 102, 104, 106, 112, 114, 116, 118, 120, 122, 124, 126, 132, 134, 136, 138, 140, 142, 144, 146, 156, 160, 162, 164, 238, 240, 242, 244, 246, 248, 250, 252, 266, 306, 308, 310, 312 or 328 or a protein having at least about 70% identity thereto.

For example, the nucleic acid or protein is expressed in, on or secreted from non-adherent CD133⁺ EPCs at a level at least 2 fold greater than in, on or secreted from HUVECs and the nucleic acid comprises a sequence set forth in any one of SEQ ID NOs: 15, 1, 17, 9, 13, 3, 5, 7, 19, 21, 23, 27, 29, 31, 33, 37, 39, 43, 45, 47, 49, 51, 53, 55, 57, 59, 61, 63, 65, 67, 69, 71, 73, 75, 77, 79, 81, 83, 85, 87, 93, 95, 97, 99, 101, 103, 105, 111, 113, 115, 117, 119, 121, 123, 125, 131, 133, 135, 137, 139, 141, 143, 145, 155, 159, 161, 163 or 327 or a nucleic acid having at least about 70% identity thereto, or the protein comprises a sequence set forth in any one of SEQ ID NOs: 16, 2, 18, 10, 14, 4, 6, 8, 20, 22, 24, 28, 30, 32, 34, 38, 40, 46, 48, 50, 52, 56, 58, 60, 62, 64, 66, 68, 70, 72, 74, 76, 78, 80, 82, 100, 104, 112, 114, 120, 122, 124, 126, 132, 134, 136, 138, 140, 162, 164, 238, 306 or 328 or a protein having at least about 70% identity thereto.

For example, the nucleic acid or protein is expressed in, on or secreted from EPCs at a level at least 3 fold greater than in, on or secreted by HUVECs and the nucleic acid comprises a sequence set forth in any one of SEQ ID NOs: 15, 1, 17, 9, 13, 3, 5, 7, 19, 21, 23, 27, 29, 31, 33, 37, 39, 43, 45, 47, 49, 51, 53, 55, 57, 59, 61, 63, 65, 67, 69, 71, 73, 75, 77, 79, 81, 83, 85, 87, 93, 95, 97, 99, 101, 103, 105, 111, 113, 115, 117, 119, 121, 123, 125, 131, 133, 135, 137, 139, 141, 143, 145, 155, 159, 161, 163 or 327 or a nucleic acid having at least about 70% identity thereto, or the protein comprises a sequence set forth in any one of SEQ ID NOs: 16, 2, 18, 10, 14, 4, 6, 8, 20, 22, 24, 28, 30, 32, 34, 38, 40, 44, 46, 48, 50, 52, 54, 56, 58, 60, 62, 64, 66, 68, 70, 72, 74, 76, 78, 80, 82, 84, 86, 88, 94, 96, 98, 100, 102, 104, 106, 112, 114, 116, 118, 120, 122, 124, 126, 132, 134, 136, 138, 140, 142, 144, 146, 156, 160, 162, 164 or 328 or a protein having at least about 70% identity thereto.

For example, the nucleic acid or protein is expressed in, on or secreted from non-adherent CD133⁺ EPCs at a level at least 3 fold greater than in, on or secreted from HUVECs and the nucleic acid comprises a sequence set forth in any one of SEQ ID NOs: 15, 1, 17, 9, 13, 3, 7, 19, 21, 23, 27, 29, 31, 33, 37, 39, 45, 47, 49, 51, 55, 57, 59, 61, 63, 65, 67, 69, 71, 73, 75, 77, 79, 81, 99, 103, 111, 113, 119, 121, 123, 125, 131, 133, 135, 137, 139, 161, 163, 237, 305 or 327 or a nucleic acid having at least about 70% identity thereto, or the protein comprises a sequence set forth in any one of SEQ ID NOs: 16, 2, 18, 10, 14, 4, 8, 18, 20, 22, 24, 28, 30, 32, 34, 38, 40, 46, 48, 50, 52, 56, 58, 60, 62, 64, 66, 68, 70, 72, 74, 76, 78, 80, 82, 100, 104, 112, 114, 120, 122, 124, 126, 132, 134, 136, 138, 140, 162, 164, 238, 306 or 328 or a protein having at least about 70% identity thereto.

For example, the nucleic acid or protein is expressed in, on or secreted from non-adherent CD133⁺ EPCs at a level at least 4 fold greater than in, on or secreted from HUVECs and the nucleic acid comprises a sequence set forth in any one of SEQ ID NOs: 15, 1, 17, 13, 7, 19, 21, 27, 29, 37, 39, 45, 47, 55, 57, 59, 61, 63, 65, 67, 69, 71, 73, 75, 77, 79, 99, 103, 111, 121, 123, 125, 131, 133, 135, 161, 163 or 327 or a nucleic acid having at least about 70% identity thereto, or the protein comprises a sequence set forth in any one of SEQ ID NOs: 16, 2, 18, 14, 8, 20, 22, 28, 30, 40, 46, 48, 56, 58, 60, 62, 64, 66, 68, 104, 122, 124, 126, 132, 134, 162, 164 or 328 or a protein having at least about 70% identity thereto.

For example, the nucleic acid or protein is expressed in, on or secreted from non-adherent CD133⁺ EPCs at a level at least 5 fold greater than in, on or secreted from HUVECs and the nucleic acid comprises a sequence set forth in any one of SEQ ID NOs: 15, 1, 7, 19, 27, 29, 39, 45, 47, 55, 57, 59, 61, 63, 65, 67, 103, 121, 123, 125, 131, 133, 161, 163 or 327 or a nucleic acid having at least about 70% identity thereto, or the protein comprises a sequence set forth in any one of SEQ ID NOs: 16, 2, 8, 28, 32, 36, 38, 46, 48, 50, 52, 54, 56, 58, 102, 104, 122, 124, 126, 132, 134, 162, 164 or 328 or a protein having at least about 70% identity thereto.

For example, the nucleic acid or protein is expressed in, on or secreted from non-adherent CD133⁺ EPCs at a level at least 6 fold greater than in, on or secreted from HUVECs and the nucleic acid comprises a sequence set forth in any one of SEQ ID NOs: 15, 1, 7, 19, 39, 45, 47, 55, 57, 59, 61, 63, 121, 123, 125, 133, 161, 163 or 327 or a nucleic acid having at least about 70% identity thereto, or the protein comprises a sequence set forth in any one of SEQ ID NOs: 16, 2, 8, 20, 40, 46, 48, 56, 58, 60, 62, 64, 122, 124, 126, 134, 162, 164 or 328 or a protein having at least about 70% identity thereto.

In one example, the method comprises determining the level of expression of a nucleic acid comprising the sequence of SEQ ID NO: 1, 3, 5, 7, 9, 11, 13, 15 or 17 or a nucleic acid having at least about 70% identity thereto, or comprising determining the level of expression of the protein encoded by the nucleic acid, the protein comprising the sequence of SEQ ID NO: 2, 4, 6, 8, 10, 12, 14, 16 or 18 or a protein having at least about 70% identity thereto.

In one example, the level of expression of the nucleic acid is assessed using a microarray.

In one example, a protein or nucleic acid has one or more (e.g., has all) of the following characteristics:
Is expressed on EPCs and has low, or undetectable expression on endothelial cells;
A protein is expressed on the cell surface; and
A protein contains a transmembrane domain.

In one example, the protein is selected from the group consisting of DSG2, EMB, EMR2, NKG7, ADCY7, SLC39A8, TM7SF3, NCSTN, SIRPB1, INSRR, PKD2L1, DPP6, LRRC33, SLC1A5 or the nucleic acid encodes one of the foregoing proteins.

In one example, the method comprises determining the level of expression of a nucleic acid comprising the sequence of SEQ ID NO: 15, 1, 17, 337, 9, 13, 3, 5, 177, 331, 233, 227, 193, 339 or 225 or a nucleic acid having at least about 70% identity thereto, or comprising determining the level of expression of the protein encoded by the nucleic acid, the protein comprising the sequence of SEQ ID NO: 16, 2, 18, 338, 10, 14, 4, 6, 178, 332, 234, 228, 194, 340 or 226 or a protein having at least about 70% identity thereto.

In one example, the method comprises determining the level of expression of a nucleic acid comprising the sequence of SEQ ID NO: 15 or a nucleic acid having at least about 70% identity thereto, or comprising determining the level of expression of the protein encoded by the nucleic acid, the protein comprising the sequence of SEQ ID NO: 16 or a protein having at least about 70% identity thereto.

In one example, the method comprises determining the level of expression of a nucleic acid comprising the sequence of SEQ ID NO: 17 or a nucleic acid having at least about 70% identity thereto, or comprising determining the level of expression of the protein encoded by the nucleic acid, the protein comprising the sequence of SEQ ID NO: 18 or a protein having at least about 70% identity thereto.

In one example, the method comprises determining the level of expression of a nucleic acid comprising the sequence of SEQ ID NO: 1 or a nucleic acid having at least about 70% identity thereto, or comprising determining the level of expression of the protein encoded by the nucleic acid, the protein comprising the sequence of SEQ ID NO: 2 or a protein having at least about 70% identity thereto.

Another example of the present disclosure provides a method for detecting an EPC comprising determining the level of expression of a protein that is a cell adhesion molecule or a nucleic acid encoding the protein as set forth in Table 2, or a nucleic acid or protein having at least about 70% identity thereto, in, on or secreted from a cell, wherein an increased level of expression of a nucleic acid or protein set forth in Table 2 or a nucleic acid or protein having at least about 70% identity thereto compared to another cell type is indicative of an EPC.

In one example, a method of the disclosure comprises determining the level of expression of a protein that is an immunoglobulin, cell adhesion protein comprising the sequence of SEQ ID NO: 2, 24 or 26 or a protein having at least about 70% identity thereto, or comprising determining the level of expression of a nucleic acid that encodes the protein, the nucleic acid comprising the sequence of SEQ ID NO: 1, 23 or 25 or a nucleic acid having at least about 70% identity thereto.

A further example of the present disclosure provides a method for detecting an EPC comprising determining the level of expression of a transporter protein or a nucleic acid encoding the protein as set forth in Table 3, or a nucleic acid or protein having at least about 70% identity thereto, in, on or secreted from a cell, wherein an increased level of expression of a nucleic acid or protein set forth in Table 3 or a nucleic acid or protein having at least about 70% identity thereto compared to another cell type is indicative of an EPC.

Another example of the disclosure provides a method for detecting an EPC comprising determining the level of expression of a growth factor protein or a nucleic acid encoding the protein as set forth in Table 4, or a nucleic acid or protein having at least about 70% identity thereto, in, on or secreted from a cell, wherein an increased level of expression of a nucleic acid or protein set forth in Table 4 or a nucleic acid or protein having at least about 70% identity thereto compared to another cell type is indicative of an EPC.

A further example of the disclosure provides a method for detecting an EPC comprising determining the level of expression of a receptor protein or a nucleic acid encoding the protein as set forth in Table 5, or a nucleic acid or protein having at least about 70% identity thereto, in, on or secreted from a cell, wherein an increased level of expression of a nucleic acid or protein set forth in Table 5 or a nucleic acid or protein having at least about 70% identity thereto compared to another cell type is indicative of an EPC.

A still further example of the disclosure provides a method for detecting an EPC comprising determining the level of expression of an enzyme protein or a nucleic acid encoding the protein as set forth in Table 6, or a nucleic acid or protein having at least about 70% identity thereto, in, on or secreted from a cell, wherein an increased level of expression of a nucleic acid or protein set forth in Table 6 or a nucleic acid or protein having at least about 70% identity thereto compared to another cell type is indicative of an EPC.

In one example, a protein subject of any method of the present disclosure is a cell surface protein in, or secreted from an EPC.

In one example, the level of expression of the nucleic acid or protein is increased in/on an EPC compared to the level of expression of the nucleic acid or protein in/on an endothelial cell other than an EPC and, for example, in or on a vascular endothelial cell. In one example, the cell other than an EPC is an endothelial cell expressing CD34.

In one example, the level of expression of a protein set forth in any one of Tables 1-6, or a protein having at least about 70% identity thereto, in, on or secreted from the cell is determined. For example, the level of the protein is determined by contacting the cell with a compound that binds to said protein for a time and under conditions sufficient for a compound-protein complex to form and detecting the level of said complex, wherein the level of said complex is indicative of the level of said protein on said cell. In this respect, any compound that binds specifically to the protein is suitable for performance of a method of the disclosure.

Exemplary compounds include antibodies and polypeptides comprising an antigen binding domain of an antibody.

In one example, the method additionally comprises detecting a cell that expresses CD34 (for example, expressing a high level of CD34) and/or VEGFR2/KDR and/or CD133 and/or CD31. Alternatively, or in addition, the method additionally comprises removing cells or selecting against cells expressing CD144 (for example, high levels of CD144) and/or von Willebrand Factor (vWF) and/or endothelial nitric oxide synthase (eNOS) and/or Tie2.

In one example, the method is performed using a sample from a subject, e.g., a blood sample or fraction thereof (e.g., plasma or serum or buffy coat fraction or peripheral blood mononuclear cell fraction) or bone marrow or a fraction thereof or umbilical cord blood or a fraction thereof. Exemplary blood samples include samples from subjects treated to mobilize stem cells from bone marrow, e.g., with granulocyte colony stimulating factor. Alternatively, the method is performed using one or more isolated cells or a lysate or extract thereof.

In one example, the method is performed in vitro or ex vivo.

Another example of the present disclosure provides a method for isolating an EPC, the method comprising detecting an EPC by performing the method of the disclosure to detect an EPC and isolating the detected EPC.

Another example of the present disclosure provides a method for isolating a population of cells enriched for EPCs, the method comprising contacting a population of cells comprising EPCs with a compound that binds to a protein set forth in Table 1 or a protein having at least about 70% identity thereto for a time and under conditions sufficient for said compound to bind to a cell and isolating cells to which the compound is bound.

For example, the protein is expressed in, on or secreted from EPCs at a level at least 1.5 fold greater than in, on or secreted by human umbilical cord vascular endothelial cells (HUVECs), for example at a level at least 2 fold greater than in, on or secreted by HUVECs, such as at a level at least 3 or 4 or 5 fold greater than in, on or secreted by HUVECs.

For example, the protein is expressed in, on or secreted by non-adherent CD133⁺ EPCs at a level at least 1.5 fold greater than in, on or secreted by HUVECs, for example at a level at least 2 fold greater than in, on or secreted by HUVECs, such as at a level at least 3 or 4 or 5 fold greater than in, on or secreted by HUVECs.

For example, the protein is expressed in, on or secreted from EPCs at a level at least 1.5 fold greater than in, on or secreted by HUVECs and the protein comprises a sequence set forth in any one of SEQ ID NOs: 16, 2, 18, 10, 14, 4, 6, 8, 12, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 238, 240, 242, 244, 246, 248, 250, 252, 254, 256, 258, 260, 266, 268, 270, 272, 274, 276, 278, 280, 282, 306, 308, 310, 312, 314, 316, 318, 320, 322, 324, 326 or 328 or a protein having at least about 70% identity thereto.

For example, the protein is expressed in, on or secreted by non-adherent CD133⁺ EPCs at a level at least 1.5 fold greater than in, on or secreted by HUVECs and the protein comprises a sequence set forth in any one of SEQ ID NOs: 16, 2, 18, 10, 14, 4, 6, 8, 12, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164 or 328 or a protein having at least about 70% identity thereto.

For example, the protein is expressed in, on or secreted from EPCs at a level at least 2 fold greater than in, on or secreted by HUVECs and the protein comprises a sequence set forth in any one of SEQ ID NOs: 16, 2, 18, 10, 14, 4, 6, 8, 20, 22, 24, 28, 30, 32, 34, 38, 40, 44, 46, 48, 50, 52, 54, 56, 58, 60, 62, 64, 66, 68, 70, 72, 74, 76, 78, 80, 82, 84, 86, 88, 94, 96, 98, 100, 102, 104, 106, 112, 114, 116, 118, 120, 122, 124, 126, 132, 134, 136, 138, 140, 142, 144, 146, 156, 160, 162, 164, 238, 240, 242, 244, 246, 248, 250, 252, 266, 306, 308, 310, 312 or 328 or a protein having at least about 70% identity thereto.

For example, the protein is expressed in, on or secreted from non-adherent CD133⁺ EPCs at a level at least 2 fold greater than in, on or secreted from HUVECs and the protein comprises a sequence set forth in any one of SEQ ID NOs: 16, 2, 18, 10, 14, 4, 6, 8, 20, 22, 24, 28, 30, 32, 34, 38, 40, 44, 46, 48, 50, 52, 54, 56, 58, 60, 62, 64, 66, 68, 70, 72, 74, 76, 78, 80, 82, 84, 86, 88, 94, 96, 98, 100, 102, 104, 106, 112, 114, 116, 118, 120, 122, 124, 126, 132, 134, 136, 138, 140, 142, 144, 146, 156, 160, 162, 164, 238, 240, 242, 244, 246, 248, 250, 252, 266, 306, 308, 310, 312 or 328 or a protein having at least about 70% identity thereto.

For example, the protein is expressed in, on or secreted from EPCs at a level at least 3 fold greater than in, on or secreted by HUVECs and the protein comprises a sequence set forth in any one of SEQ ID NOs: 16, 2, 18, 10, 14, 4, 6, 8, 20, 22, 24, 28, 30, 32, 34, 38, 40, 46, 48, 50, 52, 56, 58, 60, 62, 64, 66, 68, 70, 72, 74, 76, 78, 80, 82, 100, 104, 112, 114, 120, 122, 124, 126, 132, 134, 136, 138, 140, 162, 164, 238, 306 or 328 or a protein having at least about 70% identity thereto.

For example, the protein is expressed in, on or secreted from non-adherent CD133⁺ EPCs at a level at least 3 fold greater than in, on or secreted from HUVECs and the protein comprises a sequence set forth in any one of SEQ ID NOs: 16, 2, 18, 10, 14, 4, 6, 8, 20, 22, 24, 28, 30, 32, 34, 38, 40, 44, 46, 48, 50, 52, 54, 56, 58, 60, 62, 64, 66, 68, 70, 72, 74, 76, 78, 80, 82, 84, 86, 88, 94, 96, 98, 100, 102, 104, 106, 112, 114, 116, 118, 120, 122, 124, 126, 132, 134, 136, 138, 140, 142, 144, 146, 156, 160, 162, 164 or 328 or a protein having at least about 70% identity thereto.

For example, the protein is expressed in, on or secreted from non-adherent CD133⁺ EPCs at a level at least 4 fold greater than in, on or secreted from HUVECs and the protein comprises a sequence set forth in any one of SEQ ID NOs: 16, 2, 18, 14, 8, 20, 22, 28, 30, 40, 46, 48, 56, 58, 60, 62, 64, 66, 68, 104, 122, 124, 126, 132, 134, 162, 164 or 328 or a protein having at least about 70% identity thereto.

For example, the protein is expressed in, on or secreted from non-adherent CD133⁺ EPCs at a level at least 5 fold greater than in, on or secreted from HUVECs and the protein comprises a sequence set forth in any one of SEQ ID NOs: 16, 2, 8, 28, 32, 36, 38, 46, 48, 50, 52, 54, 56, 58, 102, 104, 122, 124, 126, 132, 134, 162, 164 or 328 or a protein having at least about 70% identity thereto.

For example, the protein is expressed in, on or secreted from non-adherent CD133⁺ EPCs at a level at least 6 fold greater than in, on or secreted from HUVECs and the protein comprises a sequence set forth in any one of SEQ ID NOs: 16, 2, 8, 20, 40, 46, 48, 56, 58, 60, 62, 64, 122, 124, 126, 134, 162, 164 or 328 or a protein having at least about 70% identity thereto.

In one example, the level of expression is determined using a microarray.

In one example, a protein has one or more (e.g., has all) of the following characteristics:
Is expressed on EPCs and has low, or undetectable expression on endothelial cells;
A protein is expressed on the cell surface; and
A protein contains a transmembrane domain.

In one example, the protein is selected from the group consisting of DSG2, EMB, EMR2, NKG7, ADCY7, SLC39A8, TM7SF3, NCSTN, SIRPB1, INSRR, PKD2L1, DPP6, LRRC33 or SLC1A5.

In one example, the compound binds to a protein comprising the sequence of SEQ ID NO: 16, 2, 18, 338, 10, 14, 4, 6, 178, 332, 234, 228, 194, 340 or 226 or a protein having at least about 70% identity thereto.

In one example, compound binds to a protein comprising the sequence of SEQ ID NO: 16 or a protein having at least about 70% identity thereto.

In one example, the compound binds to protein comprising the sequence of SEQ ID NO: 18 or a protein having at least about 70% identity thereto.

In one example, the compound binds to a protein comprising the sequence of SEQ ID NO: 2 or a protein having at least about 70% identity thereto.

In one example, compound binds to a protein comprising the sequence of SEQ ID NO: 2, 4, 6, 8, 10, 12, 14, 16 or 18 or a protein having at least about 70% identity thereto.

In another example, the compound binds to a protein selected from the group consisting of a protein that is a cell adhesion protein as set forth in Table 2, a transporter protein as set forth in Table 3, a growth factor as set forth in Table 4, a receptor as set forth in Table 5 and an enzyme as set forth in Table 6.

In a further example, the protein is an immunoglobulin, cell adhesion protein comprising the sequence of SEQ ID NO: 2, 24 or 26 or a protein having at least about 70% identity thereto.

For example, the method comprises isolating cells to which the compound binds to an increased level compared to other cells in the population.

In an example, the compound that binds to the protein is an antibody or a polypeptide comprising an antigen binding domain of an antibody.

The skilled artisan will be aware of suitable methods for isolating cells making use of compounds that bind to proteins, such as fluorescence-activated cell sorting (FACS) or magnetic cell separation cell techniques, e.g., MACS or techniques using Dynabeads™.

In one example, the enriched population is isolated from a sample from a subject, e.g., as discussed herein in more detail. Accordingly, the present disclosure also encompasses a method additionally comprising providing or obtaining a sample from a subject. Such a sample may have been isolated previously from a subject, e.g., the method is performed in vitro or ex vivo. The population of cells can also be an isolated population of cells, e.g., produced using tissue culture techniques.

In one example, the method additionally comprises culturing the isolated cells, e.g., to increase the number of EPCs or to expand the EPCs. In one example, the EPCs express a nucleic acid or protein as set out in Table 1 after culturing, e.g., after a time sufficient for the cells to expand to a level sufficient or compatible for administration to a subject, such as at least about 3 days or 5 days or 7 days.

In another example, the method comprises determining the activity of an EPC, e.g., by performing a method known in the art and/or described herein, such as by determining the ability of the cells to form CFU and/or to take up acetylated-LDL and/or binding of Ulex europaeus lectin.

In an example, the method additionally comprises formulating the isolated EPCs with a pharmaceutically acceptable carrier to thereby produce a pharmaceutical composition.

In a further example, the method additionally comprises immobilizing the isolated EPCs and/or cells isolated therefrom on a solid or semi-solid matrix.

The present disclosure additionally provides a composition comprising a population of cells enriched for EPCs, wherein the EPCs are population is isolated by performing a method according to the present disclosure.

The present disclosure also provides a composition comprising a population of cells enriched for EPCs expressing one or more nucleic acids or proteins set forth in Table 1.

For example, the population is enriched for EPCs expressing a nucleic acid or protein at a level at least 1.5 fold greater than human umbilical cord vascular endothelial cells (HUVECs), for example at a level at least 2 fold greater than HUVECs, such as at a level at least 3 or 4 or 5 fold greater than HUVECs.

For example, the population is enriched for EPCs expressing a nucleic acid or protein expressed by non-adherent CD133⁺ EPCs at a level at least 1.5 fold greater than HUVECs, for example at a level at least 2 fold greater than HUVECs, such as at a level at least 3 or 4 or 5 fold greater than HUVECs.

For example, the population is enriched for EPCs expressing a nucleic acid or protein at a level at least 1.5 fold greater HUVECs and the nucleic acid comprises a sequence set forth in any one of SEQ ID NOs: 15, 1, 17, 9, 13, 3, 5, 7, 11, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 237, 239, 241, 243, 245, 247, 249, 251, 253, 255, 257, 259, 265, 267, 269, 271, 273, 275, 277, 279, 281, 305, 307, 309, 311, 313, 315, 317, 319, 321, 323, 325 or 327 or a nucleic acid having at least about 70% identity thereto, or the protein comprises a sequence set forth in any one of SEQ ID NOs: 16, 2, 18, 10, 14, 4, 6, 8, 12, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 238, 240, 242, 244, 246, 248, 250, 252, 254, 256, 258, 260, 266, 268, 270, 272, 274, 276, 278, 280, 282, 306, 308, 310, 312, 314, 316, 318, 320, 322, 324, 326 or 328 or a protein having at least about 70% identity thereto.

For example, the population is enriched for EPCs expressing a nucleic acid or protein expressed by non-adherent CD133⁺ EPCs at a level at least 1.5 fold greater than HUVECs and the nucleic acid comprises a sequence set forth in any one of SEQ ID NOs: 15, 1, 17, 9, 13, 3, 5, 7, 11, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163 or 327 or a nucleic acid having at least about 70% identity thereto, or the protein comprises a sequence set forth in any one of SEQ ID NOs: 16, 2, 18, 10, 14, 4, 6, 8, 12, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164 or 328 or a protein having at least about 70% identity thereto.

For example, the population is enriched for EPCs expressing a nucleic acid or protein at a level at least 2 fold greater HUVECs and the nucleic acid comprises a sequence set forth in any one of SEQ ID NOs: 15, 1, 17, 9, 13, 3, 5, 7, 19, 21, 23, 27, 29, 31, 33, 37, 39, 43, 45, 47, 49, 51, 53, 55, 57, 59, 61, 63, 65, 67, 69, 71, 73, 75, 77, 79, 81, 83, 85, 87, 93, 95, 97, 99, 101, 103, 105, 111, 113, 115, 117, 119, 121, 123, 125, 131, 133, 135, 137, 139, 141, 143, 145, 155, 159, 161, 163, 237, 239, 241, 243, 245, 247, 249, 251, 265, 305, 307, 309, 311 or 327 or a nucleic acid having at least about 70% identity thereto, or the protein comprises a sequence set forth in any one of SEQ ID NOs: 16, 2, 18, 10, 14, 4, 6, 8, 20, 22, 24, 28, 30, 32, 34, 38, 40, 44, 46, 48, 50, 52, 54, 56, 58, 60, 62, 64, 66, 68, 70, 72, 74, 76, 78, 80, 82, 84, 86, 88, 94, 96, 98, 100, 102, 104, 106, 112, 114, 116, 118, 120, 122, 124, 126, 132, 134, 136, 138, 140, 142, 144, 146, 156, 160, 162, 164, 238, 240, 242, 244, 246, 248, 250, 252, 266, 306, 308, 310, 312 or 328 or a protein having at least about 70% identity thereto.

For example, the population is enriched for EPCs expressing a nucleic acid or protein expressed by non-adherent CD133⁺ EPCs at a level at least 2 fold greater than HUVECs and the nucleic acid comprises a sequence set forth in any one of SEQ ID NOs: 15, 1, 17, 9, 13, 3, 5, 7, 11, 19, 21, 23, 27, 29, 31, 33, 37, 39, 43, 45, 47, 49, 51, 53, 55, 57, 59, 61, 63, 65, 67, 69, 71, 73, 75, 77, 79, 81, 83, 85, 87, 93, 95, 97, 99, 101, 103, 105, 111, 113, 115, 117, 119, 121, 123, 125, 131, 133, 135, 137, 139, 141, 143, 145, 155, 159, 161, 163 or 327 or a nucleic acid having at least about 70% identity thereto, or the protein comprises a sequence set forth in any one of SEQ ID NOs: 16, 2, 18, 10, 14, 4, 6, 8, 20, 22, 24, 28, 30, 32, 34, 38, 40, 46, 48, 50, 52, 56, 58, 60, 62, 64, 66, 68, 70, 72, 74, 76, 78, 80, 82, 100, 104, 112, 114, 120, 122, 124, 126, 132, 134, 136, 138, 140, 162, 164, 238, 306 or 328 or a protein having at least about 70% identity thereto.

For example, the population is enriched for EPCs expressing a nucleic acid or protein at a level at least 3 fold greater HUVECs and the nucleic acid comprises a sequence set forth in any one of SEQ ID NOs: 15, 1, 17, 9, 13, 3, 5, 7, 19, 21, 23, 27, 29, 31, 33, 37, 39, 43, 45, 47, 49, 51, 53, 55, 57, 59, 61, 63, 65, 67, 69, 71, 73, 75, 77, 79, 81, 83, 85, 87, 93, 95, 97, 99, 101, 103, 105, 111, 113, 115, 117, 119, 121, 123, 125, 131, 133, 135, 137, 139, 141, 143, 145, 155, 159, 161, 163 or 327 or a nucleic acid having at least about 70% identity thereto, or the protein comprises a sequence set forth in any one of SEQ ID NOs: 16, 2, 18, 10, 14, 4, 6, 8, 20, 22, 24, 28, 30, 32, 34, 38, 40, 44, 46, 48, 50, 52, 54, 56, 58, 60, 62, 64, 66, 68, 70, 72, 74, 76, 78, 80, 82, 84, 86, 88, 94, 96, 98, 100, 102, 104, 106, 112, 114, 116, 118, 120, 122, 124, 126, 132, 134, 136, 138, 140, 142, 144, 146, 156, 160, 162, 164 or 328 or a protein having at least about 70% identity thereto.

For example, the population is enriched for EPCs expressing a nucleic acid or protein expressed by non-adherent CD133⁺ EPCs at a level at least 3 fold greater than HUVECs and the nucleic acid comprises a sequence set forth in any one of SEQ ID NOs: 15, 1, 17, 9, 13, 3, 7, 19, 21, 23, 27, 29, 31, 33, 37, 39, 45, 47, 49, 51, 55, 57, 59, 61, 63, 65, 67, 69, 71, 73, 75, 77, 79, 81, 99, 103, 111, 113, 119, 121, 123, 125, 131, 133, 135, 137, 139, 161, 163, 237, 305 or 327 or a nucleic acid having at least about 70% identity thereto, or the protein comprises a sequence set forth in any one of SEQ ID NOs: 16, 2, 18, 10, 14, 4, 8, 18, 20, 22, 24, 28, 30, 32, 34, 38, 40, 46, 48, 50, 52, 56, 58, 60, 62, 64, 66, 68, 70, 72, 74, 76, 78, 80, 82, 100, 104, 112, 114, 120, 122, 124, 126, 132, 134, 136, 138, 140, 162, 164, 238, 306 or 328 or a protein having at least about 70% identity thereto.

For example, the population is enriched for EPCs expressing a nucleic acid or protein expressed by non-adherent CD133⁺ EPCs at a level at least 4 fold greater than HUVECs and the nucleic acid comprises a sequence set forth in any one of SEQ ID NOs: 15, 1, 17, 13, 7, 19, 21, 27, 29, 37, 39, 45, 47, 55, 57, 59, 61, 63, 65, 67, 69, 71, 73, 75, 77, 79, 99, 103, 111, 121, 123, 125, 131, 133, 135, 161, 163 or 327 or a nucleic acid having at least about 70% identity thereto, or the protein comprises a sequence set forth in any one of SEQ ID NOs: 16, 2, 18, 14, 8, 20, 22, 28, 30, 40, 46, 48, 56, 58, 60, 62, 64, 66, 68, 104, 122, 124, 126, 132, 134, 162, 164 or 328 or a protein having at least about 70% identity thereto.

For example, the population is enriched for EPCs expressing a nucleic acid or protein expressed by non-adherent CD133⁺ EPCs at a level at least 5 fold greater than HUVECs and the nucleic acid comprises a sequence set forth in any one of SEQ ID NOs: 15, 1, 7, 19, 27, 29, 39, 45, 47, 55, 57, 59, 61, 63, 65, 67, 103, 121, 123, 125, 131, 133, 161, 163 or 327 or a nucleic acid having at least about 70% identity thereto, or the protein comprises a sequence set forth in any one of SEQ ID NOs: 16, 2, 8, 28, 32, 36, 38, 46, 48, 50, 52, 54, 56, 58, 102, 104, 122, 124, 126, 132, 134, 162, 164 or 328 or a protein having at least about 70% identity thereto.

For example, the population is enriched for EPCs expressing a nucleic acid or protein expressed by non-adherent CD133⁺ EPCs at a level at least 6 fold greater than HUVECs and the nucleic acid comprises a sequence set forth in any one of SEQ ID NOs: 15, 1, 7, 19, 39, 45, 47, 55, 57, 59, 61, 63, 121, 123, 125, 133, 161, 163 or 327 or a nucleic acid having at least about 70% identity thereto, or the protein comprises a sequence set forth in any one of SEQ ID NOs: 16, 2, 8, 20, 40, 46, 48, 56, 58, 60, 62, 64, 122, 124, 126, 134, 162, 164 or 328 or a protein having at least about 70% identity thereto.

In one example, the level of expression is determined using a microarray.

In one example, a protein or nucleic acid has one or more (e.g., has all) of the following characteristics:
Is expressed on EPCs and has low, or undetectable expression on endothelial cells;
A protein is expressed on the cell surface; and
A protein contains a transmembrane domain.

In one example, the protein is selected from the group consisting of DSG2, EMB, EMR2, NKG7, ADCY7, SLC39A8, TM7SF3, NCSTN, SIRPB1, INSRR, PKD2L1, DPP6, LRRC33 or SLC1A5 or the nucleic acid encodes one of the foregoing proteins.

In one example, the population is enriched for EPCs expressing a nucleic acid comprising the sequence of SEQ ID NO: 15, 1, 17, 337, 9, 13, 3, 5, 177, 331, 233, 227, 193, 339 or 225 or a nucleic acid having at least about 70% identity thereto, or a protein comprising the sequence of SEQ ID NO: 16, 2, 18, 338, 10, 14, 4, 6, 178, 332, 234, 228, 194, 340 or 226 or a protein having at least about 70% identity thereto.

In one example, the population is enriched for EPCs expressing a nucleic acid comprising the sequence of SEQ ID NO: 15 or a nucleic acid having at least about 70% identity thereto, or comprising determining the level of expression of the protein encoded by the nucleic acid, the protein comprising the sequence of SEQ ID NO: 16 or a protein having at least about 70% identity thereto.

In one example, the population is enriched for EPCs expressing a nucleic acid comprising the sequence of SEQ ID NO: 17 or a nucleic acid having at least about 70% identity thereto, or comprising determining the level of expression of the protein encoded by the nucleic acid, the protein comprising the sequence of SEQ ID NO: 18 or a protein having at least about 70% identity thereto.

In one example, the population is enriched for EPCs expressing a nucleic acid comprising the sequence of SEQ ID NO: 1 or a nucleic acid having at least about 70% identity thereto, or a protein encoded by the nucleic acid, the protein comprising the sequence of SEQ ID NO: 2 or a protein having at least about 70% identity thereto.

In one example, the population is enriched for EPCs expressing a protein comprising the sequence of SEQ ID NO: 2, 4, 6, 8, 10, 12, 14, 16 or 18 or a protein having at least about 70% identity thereto or a nucleic acid comprising the sequence of SEQ ID NO: 1, 3, 5, 7, 9, 11, 13, 15 or 17 or a nucleic acid having at least about 70% identity thereto.

In another example, the population is enriched for EPCs expressing a protein selected from the group consisting of a protein that is a cell adhesion protein as set forth in Table 2, a transporter protein as set forth in Table 3, a growth factor as set forth in Table 4, a receptor as set forth in Table 5 and an enzyme as set forth in Table 6 or a nucleic acid encoding any of the foregoing proteins.

In a further example, the population is enriched for EPCs expressing a protein that is an immunoglobulin, cell adhesion protein comprising the sequence of SEQ ID NO: 2, 24 or 26 or a protein having at least about 70% identity thereto, or the nucleic acid encodes the immunoglobulin, cell adhesion protein and comprises the sequence of SEQ ID NO: 1, 23 or 25 or a nucleic acid having at least about 70% identity thereto.

In one example, the EPCs express one or more proteins selected from the group consisting of CD133, CD117, CD34 and CD31.

In one example, the present disclosure provides a population of cells enriched for EPCs expressing DSG2 and one or more proteins selected from the group consisting of CD133, CD117, CD34 and CD31. In one example, the present disclosure provides a population of cells enriched for EPCs expressing DSG2, CD133, and CD117. In one example, the present disclosure provides a population of cells enriched for EPCs expressing DSG2, CD133, CD117, CD34 and CD31.

The skilled artisan will appreciate that a method for identifying EPCs in a sample from a subject is useful for diagnosing or prognosing a condition associated with EPCs, e.g., by assessing the number and/or activity of EPCs in the sample. Such assessment can be made using standard techniques, e.g., FACS, MACS, immunohistochemistry or immunofluorescence or activity assays described above. Accordingly, an example of the present disclosure provides a method for diagnosing and/or prognosing an EPC-associated condition in a subject, comprising performing a method of the disclosure to detect an EPC in a sample from a subject and/or performing a method of the disclosure to determine the activity of an EPC in a sample from a subject wherein detection of EPC(s) and/or EPC activity or failure to detect EPCs and/or EPC activity is diagnostic or prognostic of the EPC-associated condition.

In one example, the method comprises:
(i) determining or estimating the number of EPCs in the sample or determining or estimating EPC activity in the sample;
(ii) comparing the number of EPCs or EPC activity at (i) to the number of EPCs or EPC activity in a sample from a normal and/or healthy subject;
   wherein an increased or decreased number of EPCs or increased or decreased EPC activity at (i) compared to the number or activity of EPCs in a sample from the normal and/or healthy subject is diagnostic or prognostic of the EPC-associated condition.

In one example, the subject is receiving treatment for the condition and wherein:
(a) a similar number of EPCs or EPC activity at (i) compared to the number or activity of EPCs in a sample from a normal and/or healthy subject indicates that the subject is responding to treatment for the EPC-associated condition;
(b) an increased or decreased number of EPCs or EPC activity at (i) compared to the number or activity of EPCs in a sample from a normal and/or healthy subject indicates that the subject is not responding to treatment for the EPC-associated condition;
(c) an increased or decreased number or activity of EPCs compared to the number or activity of EPCs in a sample from the subject prior to treatment indicates that the subject is responding to treatment for the EPC-associated condition; or
(d) a similar number or activity of EPCs at (i) compared to the number or activity of EPCs in a sample from the subject prior to treatment indicates that the subject is not responding to treatment for the EPC-associated condition.

In one example, the method comprises contacting a sample with a compound that binds to a protein set forth in Table 1 for a time and under conditions sufficient for the compound to bind to a cell expressing the protein and determining the number of cells to which the compound has bound. For example, the compound is labeled with a detectable marker to facilitate detection. Exemplary compounds include antibodies and polypeptides comprising an antigen binding domain of an antibody.

The skilled artisan will also appreciate that the provision of markers of EPCs provides the basis for methods for diagnosing and/or prognosing an EPC-associated condition without necessarily assessing the number of cells in a sample, e.g., by detecting the level of the marker(s) in a sample, e.g., using an immunoassay. Accordingly, the present disclosure additionally provides a method for diagnosing and/or prognosing an EPC-associated condition in a subject, the method comprising:
(i) detecting the level of a nucleic acid or protein set forth in Table 1 or a nucleic acid or protein having at least about 70% identity thereto in a sample from a subject;
(ii) comparing the level at (i) to the level of the nucleic acid or protein in a normal and/or healthy subject,
   wherein an increased level of the nucleic acid or protein at (i) compared to the level in the normal and/or healthy subject is diagnostic or prognostic of the EPC-associated condition.

For example, the method comprises detecting the level of a protein set forth in Table 1.

In one example, the subject is receiving treatment for said condition and wherein
(a) a similar level of the nucleic acid or protein at (i) compared to the level of the nucleic acid or protein in a sample from a normal and/or healthy subject indicates that the subject is responding to treatment for the EPC-associated condition;
(b) an increased or decreased level of the nucleic acid or protein at (i) compared to the level of the nucleic acid or protein in a sample from a normal and/or healthy subject indicates that the subject is not responding to treatment for the EPC-associated condition;
(c) an increased or decreased level of the nucleic acid or protein compared to the level of the nucleic acid or protein in a sample from the subject prior to treatment indicates that the subject is responding to treatment for the EPC-associated condition; or
(iv) a similar level of the nucleic acid or protein at (i) compared to the level of the nucleic acid or protein in a sample from the subject prior to treatment indicates that the subject is not responding to treatment for the EPC-associated condition.

In one example, the method comprises contacting a sample with a compound that binds to a protein set forth in Table 1 for a time and under conditions sufficient for a compound-protein complex to form and determining the level of the complex. For example, the compound is labeled with a detectable marker to facilitate detection.

For example, the nucleic acid or protein is expressed in, on or secreted from EPCs at a level at least 1.5 fold greater than in, on or secreted by human umbilical cord vascular endothelial cells (HUVECs), for example at a level at least 2 fold greater than in, on or secreted by HUVECs, such as at a level at least 3 or 4 or 5 fold greater than in, on or secreted by HUVECs.

For example, the nucleic acid or protein is expressed in, on or secreted by non-adherent CD133⁺ EPCs at a level at least 1.5 fold greater than in, on or secreted by HUVECs, for example, at a level at least 2 fold greater than in, on or secreted by HUVECs, such as at a level at least 3 or 4 or 5 fold greater than in, on or secreted by HUVECs.

For example, the nucleic acid or protein is expressed in, on or secreted from EPCs at a level at least 1.5 fold greater than in, on or secreted by HUVECs and the nucleic acid comprises a sequence set forth in any one of SEQ ID NOs: 15, 1, 17, 9, 13, 3, 5, 7, 11, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 237, 239, 241, 243, 245, 247, 249, 251, 253, 255, 257, 259, 265, 267, 269, 271, 273, 275, 277, 279, 281, 305, 307, 309, 311, 313, 315, 317, 319, 321, 323, 325 or 327 or a nucleic acid having at least about 70% identity thereto, or the protein comprises a sequence set forth in any one of SEQ ID NOs: 16, 2, 18, 10, 14, 4, 6, 8, 12, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 238, 240, 242, 244, 246, 248, 250, 252, 254, 256, 258, 260, 266, 268, 270, 272, 274, 276, 278, 280, 282, 306, 308, 310, 312, 314, 316, 318, 320, 322, 324, 326 or 328 or a protein having at least about 70% identity thereto.

For example, the nucleic acid or protein is expressed in, on or secreted by non-adherent CD133⁺ EPCs at a level at least 1.5 fold greater than in, on or secreted by HUVECs and the nucleic acid comprises a sequence set forth in any one of SEQ ID NOs: 15, 1, 17, 9, 13, 3, 5, 7, 11, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163 or 327 or a nucleic acid having at least about 70% identity thereto, or the protein comprises a sequence set forth in any one of SEQ ID NOs: 16, 2, 18, 10, 14, 4, 6, 8, 12, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164 or 328 or a protein having at least about 70% identity thereto.

For example, the nucleic acid or protein is expressed in, on or secreted from EPCs at a level at least 2 fold greater than in, on or secreted by HUVECs and the nucleic acid comprises a sequence set forth in any one of SEQ ID NOs: 15, 1, 17, 9, 13, 3, 5, 7, 19, 21, 23, 27, 29, 31, 33, 37, 39, 43, 45, 47, 49, 51, 53, 55, 57, 59, 61, 63, 65, 67, 69, 71, 73, 75, 77, 79, 81, 83, 85, 87, 93, 95, 97, 99, 101, 103, 105, 111, 113, 115, 117, 119, 121, 123, 125, 131, 133, 135, 137, 139, 141, 143, 145, 155, 159, 161, 163, 237, 239, 241, 243, 245, 247, 249, 251, 265, 305, 307, 309, 311 or 327 or a nucleic acid having at least about 70% identity thereto, or the protein comprises a sequence set forth in any one of SEQ ID NOs: 16, 2, 18, 10, 14, 4, 6, 8, 20, 22, 24, 28, 30, 32, 34, 38, 40, 44, 46, 48, 50, 52, 54, 56, 58, 60, 62, 64, 66, 68, 70, 72, 74, 76, 78, 80, 82, 84, 86, 88, 94, 96, 98, 100, 102, 104, 106, 112, 114, 116, 118, 120, 122, 124, 126, 132, 134, 136, 138, 140, 142, 144, 146, 156, 160, 162, 164, 238, 240, 242, 244, 246, 248, 250, 252, 266, 306, 308, 310, 312 or 328 or a protein having at least about 70% identity thereto.

For example, the nucleic acid or protein is expressed in, on or secreted from non-adherent CD133⁺ EPCs at a level at least 2 fold greater than in, on or secreted from HUVECs and the nucleic acid comprises a sequence set forth in any one of SEQ ID NOs: 15, 1, 17, 9, 13, 3, 5, 7, 11, 19, 21, 23, 27, 29, 31, 33, 37, 39, 43, 45, 47, 49, 51, 53, 55, 57, 59, 61, 63, 65, 67, 69, 71, 73, 75, 77, 79, 81, 83, 85, 87, 93, 95, 97, 99, 101, 103, 105, 111, 113, 115, 117, 119, 121, 123, 125, 131, 133, 135, 137, 139, 141, 143, 145, 155, 159, 161, 163 or 327 or a nucleic acid having at least about 70% identity thereto, or the protein comprises a sequence set forth in any one of SEQ ID NOs: 16, 2, 18, 10, 14, 4, 6, 8, 20, 22, 24, 28, 30, 32, 34, 38, 40, 46, 48, 50, 52, 56, 58, 60, 62, 64, 66, 68, 70, 72, 74, 76, 78, 80, 82, 100, 104, 112, 114, 120, 122, 124, 126, 132, 134, 136, 138, 140, 162, 164, 238, 306 or 328 or a protein having at least about 70% identity thereto.

For example, the nucleic acid or protein is expressed in, on or secreted from EPCs at a level at least 3 fold greater than in, on or secreted by HUVECs and the nucleic acid comprises a sequence set forth in any one of SEQ ID NOs: 15, 1, 17, 9, 13, 3, 5, 7, 19, 21, 23, 27, 29, 31, 33, 37, 39, 43, 45, 47, 49, 51, 53, 55, 57, 59, 61, 63, 65, 67, 69, 71, 73, 75, 77, 79, 81, 83, 85, 87, 93, 95, 97, 99, 101, 103, 105, 111, 113, 115, 117, 119, 121, 123, 125, 131, 133, 135, 137, 139, 141, 143, 145, 155, 159, 161, 163 or 327 or a nucleic acid having at least about 70% identity thereto, or the protein comprises a sequence set forth in any one of SEQ ID NOs: 16, 2, 18, 10, 14, 4, 6, 8, 20, 22, 24, 28, 30, 32, 34, 38, 40, 44, 46, 48, 50, 52, 54, 56, 58, 60, 62, 64, 66, 68, 70, 72, 74, 76, 78, 80, 82, 84, 86, 88, 94, 96, 98, 100, 102, 104, 106, 112, 114, 116, 118, 120, 122, 124, 126, 132, 134, 136, 138, 140, 142, 144, 146, 156, 160, 162, 164 or 328 or a protein having at least about 70% identity thereto.

For example, the nucleic acid or protein is expressed in, on or secreted from non-adherent CD133⁺ EPCs at a level at least 3 fold greater than in, on or secreted from HUVECs and the nucleic acid comprises a sequence set forth in any one of SEQ ID NOs: 15, 1, 17, 9, 13, 3, 7, 19, 21, 23, 27, 29, 31, 33, 37, 39, 45, 47, 49, 51, 55, 57, 59, 61, 63, 65, 67, 69, 71, 73, 75, 77, 79, 81, 99, 103, 111, 113, 119, 121, 123, 125, 131, 133, 135, 137, 139, 161, 163, 237, 305 or 327 or a nucleic acid having at least about 70% identity thereto, or the protein comprises a sequence set forth in any one of SEQ ID NOs: 16, 2, 18, 10, 14, 4, 8, 18, 20, 22, 24, 28, 30, 32, 34, 38, 40, 46, 48, 50, 52, 56, 58, 60, 62, 64, 66, 68, 70, 72, 74, 76, 78, 80, 82, 100, 104, 112, 114, 120, 122, 124, 126, 132, 134, 136, 138, 140, 162, 164, 238, 306 or 328 or a protein having at least about 70% identity thereto.

For example, the nucleic acid or protein is expressed in, on or secreted from non-adherent CD133⁺ EPCs at a level at least 4 fold greater than in, on or secreted from HUVECs and the nucleic acid comprises a sequence set forth in any one of SEQ ID NOs: 15, 1, 17, 13, 7, 19, 21, 27, 29, 37, 39, 45, 47, 55, 57, 59, 61, 63, 65, 67, 69, 71, 73, 75, 77, 79, 99, 103, 111, 121, 123, 125, 131, 133, 135, 161, 163 or 327 or a nucleic acid having at least about 70% identity thereto, or the protein comprises a sequence set forth in any one of SEQ ID NOs: 16, 2, 18, 14, 8, 20, 22, 28, 30, 40, 46, 48, 56, 58, 60, 62, 64, 66, 68, 104, 122, 124, 126, 132, 134, 162, 164 or 328 or a protein having at least about 70% identity thereto.

For example, the nucleic acid or protein is expressed in, on or secreted from non-adherent CD133⁺ EPCs at a level at least 5 fold greater than in, on or secreted from HUVECs and the nucleic acid comprises a sequence set forth in any one of SEQ ID NOs: 15, 1, 7, 19, 27, 29, 39, 45, 47, 55, 57, 59, 61, 63, 65, 67, 103, 121, 123, 125, 131, 133, 161, 163 or 327 or a nucleic acid having at least about 70% identity thereto, or the protein comprises a sequence set forth in any one of SEQ ID NOs: 16, 2, 8, 28, 32, 36, 38, 46, 48, 50, 52, 54, 56, 58, 102, 104, 122, 124, 126, 132, 134, 162, 164 or 328 or a protein having at least about 70% identity thereto.

For example, the nucleic acid or protein is expressed in, on or secreted from non-adherent CD133⁺ EPCs at a level at least 6 fold greater than in, on or secreted from HUVECs and the nucleic acid comprises a sequence set forth in any one of SEQ ID NOs: 15, 1, 7, 19, 39, 45, 47, 55, 57, 59, 61, 63, 121, 123, 125, 133, 161, 163 or 327 or a nucleic acid having at least about 70% identity thereto, or the protein comprises a sequence set forth in any one of SEQ ID NOs: 16, 2, 8, 20, 40, 46, 48, 56, 58, 60, 62, 64, 122, 124, 126, 134, 162, 164 or 328 or a protein having at least about 70% identity thereto.

In one example, the level of expression is determined using a microarray.

In one example, a protein or nucleic acid has one or more (e.g., has all) of the following characteristics:
Is expressed on EPCs and has low, or undetectable expression on endothelial cells;
A protein is expressed on the cell surface; and
A protein contains a transmembrane domain.

In one example, the protein is selected from the group consisting of DSG2, EMB, EMR2, NKG7, ADCY7, SLC39A8, TM7SF3, NCSTN, SIRPB1, INSRR, PKD2L1, DPP6, LRRC33 or SLC1A5 or the nucleic acid encodes one of the foregoing proteins.

In one example, the nucleic acid comprises the sequence of SEQ ID NO: 15, 1, 17, 337, 9, 13, 3, 5, 177, 331, 233, 227, 193, 339 or 225 or a sequence having at least about 70% identity thereto, or the protein comprises the sequence of SEQ ID NO: 16, 2, 18, 338, 10, 14, 4, 6, 178, 332, 234, 228, 194, 340 or 226 or a sequence having at least about 70% identity thereto.

In one example, the nucleic acid comprises the sequence of SEQ ID NO: 15 or a sequence having at least about 70% identity thereto, or the protein comprises the sequence of SEQ ID NO: 16 or a sequence having at least about 70% identity thereto.

In one example, the nucleic acid comprises the sequence of SEQ ID NO: 17 or a sequence having at least about 70% identity thereto, or the protein comprises the sequence of SEQ ID NO: 18 or a sequence having at least about 70% identity thereto.

In one example, the nucleic acid comprises the sequence of SEQ ID NO: 1 or a sequence having at least about 70% identity thereto, or the protein comprises the sequence of SEQ ID NO: 2 or a sequence having at least about 70% identity thereto.

In one example of a diagnostic or prognostic method described herein, the protein comprises the sequence of SEQ ID NO: 2, 4, 6, 8, 10, 12, 14, 16 or 18 or a protein having at least about 70% identity thereto or the nucleic acid comprises the sequence of SEQ ID NO: 1, 3, 5, 7, 9, 11, 13, 15 or 17 or a nucleic acid having at least about 70% identity thereto.

In another example of a diagnostic or prognostic method described herein, the protein is selected from the group consisting of a protein that is a cell adhesion protein as set forth in Table 2, a transporter protein as set forth in Table 3, a growth factor as set forth in Table 4, a receptor as set forth in Table 5 and an enzyme as set forth in Table 6 or wherein the nucleic acid encodes any of the foregoing proteins.

In a further example of a diagnostic or prognostic method described herein, the protein is an immunoglobulin, cell adhesion protein comprising the sequence of SEQ ID NO: 2, 24 or 26 or a protein having at least about 70% identity thereto, or the nucleic acid encodes the immunoglobulin, cell adhesion protein and comprises the sequence of SEQ ID NO: 1, 23 or 25 or a nucleic acid having at least about 70% identity thereto.

The identification of cell surface markers of EPCs also provides the basis for in vivo methods for detecting EPCs or diagnosing/prognosing conditions (e.g., imaging methods). Accordingly, the disclosure also provides a method for localising and/or detecting and/or diagnosing and/or prognosing an EPC-associated condition in a subject, the method comprising:

(i) administering to a subject a compound that binds specifically to a compound that binds to a protein set forth in Table 1 such that the compound binds to the protein, if present; and
(ii) detecting the compound bound to the protein in vivo, wherein detection of the bound compound localises and/or detects and/or diagnoses and/or prognoses the EPC-associated condition.

In one example, the compound is conjugated to a detectable label and the method comprises detecting the label to detect the compound bound to the protein.

For example, the protein is expressed in, on or secreted from EPCs at a level at least 1.5 fold greater than in, on or secreted by human umbilical cord vascular endothelial cells (HUVECs), for example, at a level at least 2 fold greater than in, on or secreted by HUVECs, such as at a level at least 3 or 4 or 5 fold greater than in, on or secreted by HUVECs.

For example, the protein is expressed in, on or secreted by non-adherent CD133⁺ EPCs at a level at least 1.5 fold greater than in, on or secreted by HUVECs, for example, at a level at least 2 fold greater than in, on or secreted by HUVECs, more such as at a level at least 3 or 4 or 5 fold greater than in, on or secreted by HUVECs.

For example, the protein is expressed in, on or secreted from EPCs at a level at least 1.5 fold greater than in, on or secreted by HUVECs and the protein comprises a sequence set forth in any one of SEQ ID NOs: 16, 2, 18, 10, 14, 4, 6, 8, 12, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 238, 240, 242, 244, 246, 248, 250, 252, 254, 256, 258, 260, 266, 268, 270, 272, 274, 276, 278, 280, 282, 306, 308, 310, 312, 314, 316, 318, 320, 322, 324, 326 or 328 or a protein having at least about 70% identity thereto.

For example, the protein is expressed in, on or secreted by non-adherent CD133⁺ EPCs at a level at least 1.5 fold greater than in, on or secreted by HUVECs and the protein comprises a sequence set forth in any one of SEQ ID NOs: 16, 2, 18, 10, 14, 4, 6, 8, 12, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164 or 328 or a protein having at least about 70% identity thereto.

For example, the protein is expressed in, on or secreted from EPCs at a level at least 2 fold greater than in, on or secreted by HUVECs and the protein comprises a sequence set forth in any one of SEQ ID NOs: 16, 2, 18, 10, 14, 4, 6, 8, 20, 22, 24, 28, 30, 32, 34, 38, 40, 44, 46, 48, 50, 52, 54, 56, 58, 60, 62, 64, 66, 68, 70, 72, 74, 76, 78, 80, 82, 84, 86, 88, 94, 96, 98, 100, 102, 104, 106, 112, 114, 116, 118, 120, 122, 124, 126, 132, 134, 136, 138, 140, 142, 144, 146, 156, 160, 162, 164, 238, 240, 242, 244, 246, 248, 250, 252, 266, 306, 308, 310, 312 or 328 or a protein having at least about 70% identity thereto.

For example, the protein is expressed in, on or secreted from non-adherent CD133⁺ EPCs at a level at least 2 fold greater than in, on or secreted from HUVECs and the protein comprises a sequence set forth in any one of SEQ ID NOs: 16, 2, 18, 10, 14, 4, 6, 8, 20, 22, 24, 28, 30, 32, 34, 38, 40, 46, 48, 50, 52, 56, 58, 60, 62, 64, 66, 68, 70, 72, 74, 76, 78, 80, 82, 100, 104, 112, 114, 120, 122, 124, 126, 132, 134, 136, 138, 140, 162, 164, 238, 306 or 328 or a protein having at least about 70% identity thereto.

For example, the protein is expressed in, on or secreted from EPCs at a level at least 3 fold greater than in, on or secreted by HUVECs and the protein comprises a sequence set forth in any one of SEQ ID NOs: 16, 2, 18, 10, 14, 4, 6, 8, 20, 22, 24, 28, 30, 32, 34, 38, 40, 44, 46, 48, 50, 52, 54, 56, 58, 60, 62, 64, 66, 68, 70, 72, 74, 76, 78, 80, 82, 84, 86, 88, 94, 96, 98, 100, 102, 104, 106, 112, 114, 116, 118, 120, 122, 124, 126, 132, 134, 136, 138, 140, 142, 144, 146, 156, 160, 162, 164 or 328 or a protein having at least about 70% identity thereto.

For example, the protein is expressed in, on or secreted from non-adherent CD133⁺ EPCs at a level at least 3 fold greater than in, on or secreted from HUVECs and the protein comprises a sequence set forth in any one of SEQ ID NOs: 16, 2, 18, 10, 14, 4, 8, 18, 20, 22, 24, 28, 30, 32, 34, 38, 40, 46, 48, 50, 52, 56, 58, 60, 62, 64, 66, 68, 70, 72, 74, 76, 78, 80, 82, 100, 104, 112, 114, 120, 122, 124, 126, 132, 134, 136, 138, 140, 162, 164, 238, 306 or 328 or a protein having at least about 70% identity thereto.

For example, the protein is expressed in, on or secreted from non-adherent CD133⁺ EPCs at a level at least 4 fold greater than in, on or secreted from HUVECs and the protein comprises a sequence set forth in any one of SEQ ID NOs: 16, 2, 18, 14, 8, 20, 22, 28, 30, 40, 46, 48, 56, 58, 60, 62, 64, 66, 68, 104, 122, 124, 126, 132, 134, 162, 164 or 328 or a protein having at least about 70% identity thereto.

For example, the protein is expressed in, on or secreted from non-adherent CD133⁺ EPCs at a level at least 5 fold greater than in, on or secreted from HUVECs and the protein comprises a sequence set forth in any one of SEQ ID NOs: 16, 2, 8, 28, 32, 36, 38, 46, 48, 50, 52, 54, 56, 58, 102, 104, 122, 124, 126, 132, 134, 162, 164 or 328 or a protein having at least about 70% identity thereto.

For example, the protein is expressed in, on or secreted from non-adherent CD133⁺ EPCs at a level at least 6 fold greater than in, on or secreted from HUVECs and the protein comprises a sequence set forth in any one of SEQ ID NOs: 16, 2, 8, 20, 40, 46, 48, 56, 58, 60, 62, 64, 122, 124, 126, 134, 162, 164 or 328 or a protein having at least about 70% identity thereto.

In one example, the level of expression is determined using a microarray.

In one example, a protein or nucleic acid has one or more (e.g., has all) of the following characteristics:
Is expressed on EPCs and has low, or undetectable expression on endothelial cells;
A protein is expressed on the cell surface; and
A protein contains a transmembrane domain.

In one example, the protein is selected from the group consisting of DSG2, EMB, EMR2, NKG7, ADCY7, SLC39A8, TM7SF3, NCSTN, SIRPB1, INSRR, PKD2L1, DPP6, LRRC33 or SLC1A5.

In one example, the nucleic acid comprises the sequence of SEQ ID NO: 15, 1, 17, 337, 9, 13, 3, 5, 177, 331, 233, 227, 193, 339 or 225 or a sequence having at least about 70% identity thereto, or the protein comprises the sequence of SEQ ID NO: 16, 2, 18, 338, 10, 14, 4, 6, 178, 332, 234, 228, 194, 340 or 226 or a sequence having at least about 70% identity thereto.

In one example, the nucleic acid comprises the sequence of SEQ ID NO: 15 or a sequence having at least about 70% identity thereto, or the protein comprises the sequence of SEQ ID NO: 16 or a sequence having at least about 70% identity thereto.

In one example, the nucleic acid comprises the sequence of SEQ ID NO: 17 or a sequence having at least about 70% identity thereto, or the protein comprises the sequence of SEQ ID NO: 18 or a sequence having at least about 70% identity thereto.

In one example, the nucleic acid comprises the sequence of SEQ ID NO: 1 or a sequence having at least about 70% identity thereto, or the protein comprises the sequence of SEQ ID NO: 2 or a sequence having at least about 70% identity thereto.

In one example, the protein comprises the sequence of SEQ ID NO: 2, 4, 6, 8, 10, 12, 14, 16 or 18 or a protein having at least about 70% identity thereto.

In another example, the protein is selected from the group consisting of a protein that is a cell adhesion protein as set forth in Table 2, a transporter protein as set forth in Table 3, a growth factor as set forth in Table 4, a receptor as set forth in Table 5 and an enzyme as set forth in Table 6.

In a further example, the protein is an immunoglobulin, cell adhesion protein comprising the sequence of SEQ ID NO: 2, 24 or 26 or a protein having at least about 70% identity thereto.

Exemplary compounds include antibodies or proteins comprising an antigen binding domain of an antibody.

In one example, the EPC-associated condition is a cardiovascular disease and/or cancer and/or preeclampsia and/or hepatitis and/or sepsis and/or an autoimmune disease and/or an inflammatory disease and/or ischemia and/or a condition caused by or associated with excessive neovascularization. Exemplary conditions associated with excessive neovascularization include psoriasis, nephropathy, cancer neovascularization or retinopathy

Exemplary EPC-associated conditions for diagnosis/prognosis using a method as described herein according to any example of the disclosure include the following:
A cardiovascular disease (including coronary artery disease or dysfunctional bicuspid aortic valve) or cerebrovascular disease which is diagnosed/prognosed by detecting reduced levels of EPCs or a reduced level of a nucleic acid or protein set forth in Table 1 in a sample from a subject;
An autoimmune disease, e.g., rheumatoid arthritis, SLE, diabetes (e.g., type 1 diabetes) or systemic sclerosis, e.g., more than five years after onset which is/are diagnosed/prognosed by detecting reduced levels of EPCs or a reduced level of a nucleic acid or protein set forth in Table 1 in a sample from a subject;
Ischemia, e.g., stroke, which is diagnosed/prognosed by detecting reduced levels of EPCs or an increased level of a nucleic acid or protein set forth in Table 1 in a sample from a subject.
Sepsis, which is diagnosed by detecting reduced levels of EPCs or a reduced level of a protein set forth in Table 1 in a sample from a subject.
A condition associated with excessive neovascularization, e.g., psoriasis, nephropathy, cancer neovascularization, cancer or retinopathy, which is/are diagnosed/prognosed by detecting increased levels of EPCs or an increased level of a nucleic acid or protein set forth in Table 1 in a sample from a subject.

In one example, a diagnostic method described herein predicts likelihood that a subject will suffer from a condition. For example, a reduced number of EPCs (e.g., detected by performing a method as described herein according to any example) is indicative of a subject likely to suffer from a cardiovascular disease (including coronary artery disease or dysfunctional bicuspid aortic valve) or cerebrovascular disease or an autoimmune disease, e.g., rheumatoid arthritis, SLE or systemic sclerosis or ischemia, e.g., a stroke, or sepsis. In another example, an increased number of EPCs indicates a risk of cancer.

The skilled artisan will appreciate that methods described herein for isolating an EPC also provide the basis for increasing EPC numbers in a subject, e.g., by adoptive transfer or cell therapy. Increasing EPCs numbers is useful for, for example, treating or preventing a condition associated with reduced EPC numbers and/or inducing neovascularization, e.g., to improve grafting or wound healing or reduce the effects of ischemia and/or to reduce hypertension and/or to improve healing of bone defects. Accordingly, another example of the present disclosure provides a method of treating or preventing a condition associated with reduced EPCs or activity, treating or preventing a condition associated with insufficient neovascularization and/or improving grafting and/or improving wound healing in a subject, said method comprising:
(i) isolating a population enriched for EPCs by performing a method of the disclosure; and
(ii) administering the cells at (i) to the subject.

In another example, the disclosure provides a method of treating or preventing a condition associated with reduced EPC numbers or activity, treating or preventing a condition associated with insufficient neovascularization and/or improving grafting and/or improving wound healing in a subject, the method comprising administering a composition comprising a population of cells enriched for EPCs of the disclosure.

In the situation of a graft, e.g., a blood vessel graft, the cells can be administered immobilized on a solid support or semi-solid support, e.g., in the form of a vascular graft.

In one example, the subject suffers from or is at risk of developing a condition associated with reduced EPC numbers and/or activity and/or a condition associated with insufficient neovascularization and/or requires a graft or has undergone grafting and/or requires improved wound healing.

Exemplary conditions to be treated by administering populations of cells enriched for EPCs include cardiovascular disease, cerebrovascular disease, hypertension, chronic kidney disease, vessel occlusion, ischemia (including stroke), an autoimmune disease, or sepsis.

In one example, the condition is coronary artery disease or dysfunctional bicuspid aortic valve.

In one example, the condition is stroke.

In one example, a method for treating or preventing a condition comprises additionally administering another cell or another therapeutic compound to a subject. For example, to treat a subject suffering from diabetes (e.g., type 1 diabetes) a population enriched for EPCs according to the present disclosure are administered to a subject, e.g., in combination with pancreatic islet cells.

For example, the cells are from the subject to be treated, i.e., an autologous transplant, or from a related subject of the same or unrelated species (e.g., a HLA matched subject or xenograft), i.e., an allogeneic or xenogeneic transplant.

For example, an effective amount, e.g., a therapeutically or prophylactically effective amount of cells is administered to the subject.

The present disclosure also provides a method of treating or preventing a condition associated with reduced EPC numbers or activity and/or treating or preventing a condition associated with insufficient neovascularization and/or improving grafting and/or improving wound healing in a subject, said method comprising administering to a subject in need thereof a solid support or a semi-solid support having immobilized thereon a compound that binds to a protein set forth in Table 1 for a time and under conditions for the compound to bind to EPCs from the subject, and for example, induces vascularization.

In one example, the condition associated with reduced EPC numbers or activity is a cardiovascular disease and/or an autoimmune disease and/or an inflammatory disease.

For example, the protein is expressed in, on or secreted from EPCs at a level at least 1.5 fold greater than in, on or secreted by human umbilical cord vascular endothelial cells (HUVECs), for example, at a level at least 2 fold greater than in, on or secreted by HUVECs, such as at a level at least 3 or 4 or 5 fold greater than in, on or secreted by HUVECs.

For example, the protein is expressed in, on or secreted by non-adherent CD133⁺ EPCs at a level at least 1.5 fold greater than in, on or secreted by HUVECs, for example at a level at least 2 fold greater than in, on or secreted by HUVECs, such as at a level at least 3 or 4 or 5 fold greater than in, on or secreted by HUVECs.

For example, the protein is expressed in, on or secreted from EPCs at a level at least 1.5 fold greater than in, on or secreted by HUVECs and the protein comprises a sequence set forth in any one of SEQ ID NOs: 16, 2, 18, 10, 14, 4, 6, 8, 12, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 238, 240, 242, 244, 246, 248, 250, 252, 254, 256, 258, 260, 266, 268, 270, 272, 274, 276, 278, 280, 282, 306, 308, 310, 312, 314, 316, 318, 320, 322, 324, 326 or 328 or a protein having at least about 70% identity thereto.

For example, the protein is expressed in, on or secreted by non-adherent CD133⁺ EPCs at a level at least 1.5 fold greater than in, on or secreted by HUVECs and the protein comprises a sequence set forth in any one of SEQ ID NOs: 16, 2, 18, 10, 14, 4, 6, 8, 12, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164 or 328 or a protein having at least about 70% identity thereto.

For example, the protein is expressed in, on or secreted from EPCs at a level at least 2 fold greater than in, on or secreted by HUVECs and the protein comprises a sequence set forth in any one of SEQ ID NOs: 16, 2, 18, 10, 14, 4, 6, 8, 20, 22, 24, 28, 30, 32, 34, 38, 40, 44, 46, 48, 50, 52, 54, 56, 58, 60, 62, 64, 66, 68, 70, 72, 74, 76, 78, 80, 82, 84, 86, 88, 94, 96, 98, 100, 102, 104, 106, 112, 114, 116, 118, 120, 122, 124, 126, 132, 134, 136, 138, 140, 142, 144, 146, 156, 160, 162, 164, 238, 240, 242, 244, 246, 248, 250, 252, 266, 306, 308, 310, 312 or 328 or a protein having at least about 70% identity thereto.

For example, the protein is expressed in, on or secreted from non-adherent CD133⁺ EPCs at a level at least 2 fold greater than in, on or secreted from HUVECs and the protein comprises a sequence set forth in any one of SEQ ID NOs: 16, 2, 18, 10, 14, 4, 6, 8, 20, 22, 24, 28, 30, 32, 34, 38, 40, 46, 48, 50, 52, 56, 58, 60, 62, 64, 66, 68, 70, 72, 74, 76, 78, 80, 82, 100, 104, 112, 114, 120, 122, 124, 126, 132, 134, 136, 138, 140, 162, 164, 238, 306 or 328 or a protein having at least about 70% identity thereto.

For example, the protein is expressed in, on or secreted from EPCs at a level at least 3 fold greater than in, on or secreted by HUVECs and the protein comprises a sequence set forth in any one of SEQ ID NOs: 16, 2, 18, 10, 14, 4, 6, 8, 20, 22, 24, 28, 30, 32, 34, 38, 40, 44, 46, 48, 50, 52, 54, 56, 58, 60, 62, 64, 66, 68, 70, 72, 74, 76, 78, 80, 82, 84, 86, 88, 94, 96, 98, 100, 102, 104, 106, 112, 114, 116, 118, 120, 122, 124, 126, 132, 134, 136, 138, 140, 142, 144, 146, 156, 160, 162, 164 or 328 or a protein having at least about 70% identity thereto.

For example, the protein is expressed in, on or secreted from non-adherent CD133⁺ EPCs at a level at least 3 fold greater than in, on or secreted from HUVECs and the protein comprises a sequence set forth in any one of SEQ ID NOs: 16, 2, 18, 10, 14, 4, 8, 18, 20, 22, 24, 28, 30, 32, 34, 38, 40, 46, 48, 50, 52, 56, 58, 60, 62, 64, 66, 68, 70, 72, 74, 76, 78, 80, 82, 100, 104, 112, 114, 120, 122, 124, 126, 132, 134, 136, 138, 140, 162, 164, 238, 306 or 328 or a protein having at least about 70% identity thereto.

For example, the protein is expressed in, on or secreted from non-adherent CD133⁺ EPCs at a level at least 4 fold greater than in, on or secreted from HUVECs and the protein comprises a sequence set forth in any one of SEQ ID NOs: 16, 2, 18, 14, 8, 20, 22, 28, 30, 40, 46, 48, 56, 58, 60, 62, 64, 66, 68, 104, 122, 124, 126, 132, 134, 162, 164 or 328 or a protein having at least about 70% identity thereto.

For example, the protein is expressed in, on or secreted from non-adherent CD133⁺ EPCs at a level at least 5 fold greater than in, on or secreted from HUVECs and the protein comprises a sequence set forth in any one of SEQ ID NOs: 16, 2, 8, 28, 32, 36, 38, 46, 48, 50, 52, 54, 56, 58, 102, 104, 122, 124, 126, 132, 134, 162, 164 or 328 or a protein having at least about 70% identity thereto.

For example, the protein is expressed in, on or secreted from non-adherent CD133⁺ EPCs at a level at least 6 fold greater than in, on or secreted from HUVECs and the protein comprises a sequence set forth in any one of SEQ ID NOs: 16, 2, 8, 20, 40, 46, 48, 56, 58, 60, 62, 64, 122, 124, 126, 134, 162, 164 or 328 or a protein having at least about 70% identity thereto.

In one example, a protein has one or more (e.g., has all) of the following characteristics:
Is expressed on EPCs and has low, or undetectable expression on endothelial cells;
A protein is expressed on the cell surface; and
A protein contains a transmembrane domain.

In one example, the protein is selected from the group consisting of DSG2, EMB, EMR2, NKG7, ADCY7, SLC39A8, TM7SF3, NCSTN, SIRPB1, INSRR, PKD2L1, DPP6, LRRC33 or SLC1A5.

In one example, the population of cells is enriched for EPCs expressing a protein comprising the sequence of SEQ ID NO: 16, 2, 18, 338, 10, 14, 4, 6, 178, 332, 234, 228, 194, 340 or 226 or a sequence having at least about 70% identity thereto.

In one example, the population of cells is enriched for EPCs expressing a protein comprising the sequence of SEQ ID NO: 16 or a sequence having at least about 70% identity thereto.

In one example, the population of cells is enriched for EPCs expressing a protein comprising the sequence of SEQ ID NO: 18 or a sequence having at least about 70% identity thereto.

In one example, the population of cells is enriched for EPCs expressing a protein comprising the sequence of SEQ ID NO: 2 or a sequence having at least about 70% identity thereto.

In one example, the population of cells administered to the subject are enriched for EPCs expressing a protein comprising the sequence of SEQ ID NO: 2, 4, 6, 8, 10, 12, 14, 16 or 18 or a protein having at least about 70% identity thereto or a nucleic acid comprising the sequence of SEQ ID NO: 1, 3, 5, 7, 9, 11, 13, 15 or 17 or a nucleic acid having at least about 70% identity thereto.

In one example, the population of cells administered to the subject are enriched for EPCs expressing a protein selected from the group consisting of a protein that is a cell adhesion protein as set forth in Table 2, a transporter protein as set forth in Table 3, a growth factor as set forth in Table 4, a receptor as set forth in Table 5 and an enzyme as set forth in Table 6 or expressing a nucleic acid encodes any of the foregoing proteins.

In one example, the population of cells administered to the subject are enriched for EPCs expressing an immunoglobulin, cell adhesion protein comprising the sequence of SEQ ID NO: 2, 24 or 26 or a protein having at least about 70% identity thereto, or a nucleic acid encoding the immunoglobulin, cell adhesion protein and comprises the sequence of SEQ ID NO: 1, 23 or 25 or a nucleic acid having at least about 70% identity thereto.

The identification of cell surface proteins preferentially expressed by EPCs also provides the means for modulating the number of those cells in a subject, e.g., to reduce or prevent neovascularization or to induce or enhance neovascularisation. Accordingly, another example of the present disclosure provides a method of modulating neovascularization and/or EPC numbers or activity in a subject, the method comprising administering to a subject in need thereof a compound that modulates expression and/or activity of a protein or nucleic acid set forth in Table 1, and/or administering a compound that binds to a protein set forth in Table 1 and modulates EPC activity and/or induces EPC death and/or EPC proliferation.

A further example of the disclosure provides a method for modulating neovascularization, the method comprising administering to a subject in need thereof a compound that modulates expression and/or activity of a protein or nucleic acid set forth in Table 1, and/or administering a compound that binds to a protein set forth in Table 1 and modulates EPC activity and/or induces EPC death and/or EPC proliferation.

Another example of the present disclosure provides a method of treating or preventing a condition associated with excessive neovascularization and/or excessive EPC numbers or activity in a subject, the method comprising administering to a subject in need thereof a compound that reduces expression and/or activity of a protein or nucleic acid set forth in Table 1, and/or administering a compound that binds to a protein set forth in Table 1 and reduces EPC activity and/or induces EPC death and/or suppresses EPC proliferation.

A further example of the disclosure provides a method for reducing or preventing neovascularization, the method comprising administering to a subject in need thereof a compound that reduces expression and/or activity of a protein or nucleic acid set forth in Table 1, and/or administering a compound that binds to a protein set forth in Table 1 and reduces EPC activity and/or induces EPC death and/or suppresses EPC proliferation.

A further example of the present disclosure provides a method of treating or preventing a condition associated with insufficient neovascularization and/or insufficient EPC numbers or activity in a subject, the method comprising administering to a subject in need thereof a compound that reduces expression and/or activity of a protein or nucleic acid set forth in Table 1, and/or administering a compound that binds to a protein set forth in Table 1 and induces or enhances EPC activity and/or suppresses EPC death and/or induces or enhances EPC proliferation.

A further example of the disclosure provides a method for inducing or enhancing neovascularization, the method comprising administering to a subject in need thereof a compound that reduces expression and/or activity of a protein or nucleic acid set forth in Table 1, and/or administering a compound that binds to a protein set forth in Table 1 and induces or enhances EPC activity and/or suppresses EPC death and/or induces or enhances EPC proliferation.

Another example of the present disclosure provides a method of treating or preventing a condition associated with excessive neovascularization and/or excessive EPC numbers or activity in a subject, the method comprising administering to a subject in need thereof a compound that induces or enhances expression and/or activity of a protein or nucleic acid set forth in Table 1, and/or administering a compound that binds to a protein set forth in Table 1 and reduces EPC activity and/or induces EPC death and/or suppresses EPC proliferation.

A further example of the disclosure provides a method for reducing or preventing neovascularization, the method comprising administering to a subject in need thereof a compound that induces or enhances expression and/or activity of a protein or nucleic acid set forth in Table 1, and/or administering a compound that binds to a protein set forth in Table 1 and reduces EPC activity and/or induces EPC death and/or suppresses EPC proliferation.

A further example of the present disclosure provides a method of treating or preventing a condition associated with insufficient neovascularization and/or insufficient EPC numbers or activity in a subject, the method comprising administering to a subject in need thereof a compound that induces or enhances expression and/or activity of a protein or nucleic acid set forth in Table 1, and/or administering a compound that binds to a protein set forth in Table 1 and induces or enhances EPC activity and/or suppresses EPC death and/or induces or enhances EPC proliferation.

A further example of the disclosure provides a method for inducing or enhancing neovascularization, the method comprising administering to a subject in need thereof a compound that induces or enhances expression and/or activity of a protein or nucleic acid set forth in Table 1, and/or administering a compound that binds to a protein set forth in Table 1 and induces or enhances EPC activity and/or suppresses EPC death and/or induces or enhances EPC proliferation.

For example, the method comprises administering a compound that binds to a protein set forth in Table 1 and modulates EPC activity and/or modulates EPC death for a time and under conditions sufficient to modulate EPC numbers and/or activity and/or neovascularization in the subject or in a tissue or organ thereof. Exemplary compounds include antibodies and/or proteins comprising an antigen binding domain of an antibody, including, conjugates of said antibodies or proteins comprising a toxic compound to thereby kill an EPC.

In one example, the condition is an autoimmune condition and/or sepsis and/or nephropathy and/or cancer and/or cancer neovascularization and/or retinopathy.

In one example, the condition is cancer. For example, the cancer is melanoma. In this regard, the inventors have demonstrated that a marker of EPCs (e.g., DSG2) is also expressed by some melanoma cells, thus providing the basis for a dual mechanism therapeutic, e.g., directly targeting melanoma cells and by reducing or preventing neovascularisation.

In one example, the condition is cancer metastasis, i.e., the present disclosure provides a method for reducing or preventing cancer metastasis. Such a method can involve performing a method described herein according to any example to treat cancer and administering an additional anti-cancer agent or treating the subject with radiation therapy.

For example, the nucleic acid or protein is expressed in, on or secreted from EPCs at a level at least 1.5 fold greater than in, on or secreted by human umbilical cord vascular endothelial cells (HUVECs), for example, at a level at least 2 fold greater than in, on or secreted by HUVECs, such as at a level at least 3 or 4 or 5 fold greater than in, on or secreted by HUVECs.

For example, the nucleic acid or protein is expressed in, on or secreted by non-adherent CD133⁺ EPCs at a level at least 1.5 fold greater than in, on or secreted by HUVECs, for example, at a level at least 2 fold greater than in, on or secreted by HUVECs, such as at a level at least 3 or 4 or 5 fold greater than in, on or secreted by HUVECs.

For example, the nucleic acid or protein is expressed in, on or secreted from EPCs at a level at least 1.5 fold greater than in, on or secreted by HUVECs and the nucleic acid comprises a sequence set forth in any one of SEQ ID NOs: 15, 1, 17, 9, 13, 3, 5, 7, 11, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 237, 239, 241, 243, 245, 247, 249, 251, 253, 255, 257, 259, 265, 267, 269, 271, 273, 275, 277, 279, 281, 305, 307, 309, 311, 313, 315, 317, 319, 321, 323, 325 or 327 or a nucleic acid having at least about 70% identity thereto, or the protein comprises a sequence set forth in any one of SEQ ID NOs: 16, 2, 18, 10, 14, 4, 6, 8, 12, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 238, 240, 242, 244, 246, 248, 250, 252, 254, 256, 258, 260, 266, 268, 270, 272, 274, 276, 278, 280, 282, 306, 308, 310, 312, 314, 316, 318, 320, 322, 324, 326 or 328 or a protein having at least about 70% identity thereto.

For example, the nucleic acid or protein is expressed in, on or secreted by non-adherent CD133⁺ EPCs at a level at least 1.5 fold greater than in, on or secreted by HUVECs and the nucleic acid comprises a sequence set forth in any one of SEQ ID NOs: 15, 1, 17, 9, 13, 3, 5, 7, 11, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163 or 327 or a nucleic acid having at least about 70% identity thereto, or the protein comprises a sequence set forth in any one of SEQ ID NOs: 16, 2, 18, 10, 14, 4, 6, 8, 12, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164 or 328 or a protein having at least about 70% identity thereto.

For example, the nucleic acid or protein is expressed in, on or secreted from EPCs at a level at least 2 fold greater than in, on or secreted by HUVECs and the nucleic acid comprises a sequence set forth in any one of SEQ ID NOs: 15, 1, 17, 9, 13, 3, 5, 7, 19, 21, 23, 27, 29, 31, 33, 37, 39, 43, 45, 47, 49, 51, 53, 55, 57, 59, 61, 63, 65, 67, 69, 71, 73, 75, 77, 79, 81, 83, 85, 87, 93, 95, 97, 99, 101, 103, 105, 111, 113, 115, 117, 119, 121, 123, 125, 131, 133, 135, 137, 139, 141, 143, 145, 155, 159, 161, 163, 237, 239, 241, 243, 245, 247, 249, 251, 265, 305, 307, 309, 311 or 327 or a nucleic acid having at least about 70% identity thereto, or the protein comprises a sequence set forth in any one of SEQ ID NOs: 16, 2, 18, 10, 14, 4, 6, 8, 20, 22, 24, 28, 30, 32, 34, 38, 40, 44, 46, 48, 50, 52, 54, 56, 58, 60, 62, 64, 66, 68, 70, 72, 74, 76, 78, 80, 82, 84, 86, 88, 94, 96, 98, 100, 102, 104, 106, 112, 114, 116, 118, 120, 122, 124, 126, 132, 134, 136, 138, 140, 142, 144, 146, 156, 160, 162, 164, 238, 240, 242, 244, 246, 248, 250, 252, 266, 306, 308, 310, 312 or 328 or a protein having at least about 70% identity thereto.

For example, the nucleic acid or protein is expressed in, on or secreted from non-adherent CD133⁺ EPCs at a level at least 2 fold greater than in, on or secreted from HUVECs and the nucleic acid comprises a sequence set forth in any one of SEQ ID NOs: 15, 1, 17, 9, 13, 3, 5, 7, 11, 19, 21, 23, 27, 29, 31, 33, 37, 39, 43, 45, 47, 49, 51, 53, 55, 57, 59, 61, 63, 65, 67, 69, 71, 73, 75, 77, 79, 81, 83, 85, 87, 93, 95, 97, 99, 101, 103, 105, 111, 113, 115, 117, 119, 121, 123, 125, 131, 133, 135, 137, 139, 141, 143, 145, 155, 159, 161, 163 or 327 or a nucleic acid having at least about 70% identity thereto, or the protein comprises a sequence set forth in any one of SEQ ID NOs: 16, 2, 18, 10, 14, 4, 6, 8, 20, 22, 24, 28, 30, 32, 34, 38, 40, 46, 48, 50, 52, 56, 58, 60, 62, 64, 66, 68, 70, 72, 74, 76, 78, 80, 82, 100, 104, 112, 114, 120, 122, 124, 126, 132, 134, 136, 138, 140, 162, 164, 238, 306 or 328 or a protein having at least about 70% identity thereto.

For example, the nucleic acid or protein is expressed in, on or secreted from EPCs at a level at least 3 fold greater than in, on or secreted by HUVECs and the nucleic acid comprises a sequence set forth in any one of SEQ ID NOs: 15, 1, 17, 9, 13, 3, 5, 7, 19, 21, 23, 27, 29, 31, 33, 37, 39, 43, 45, 47, 49, 51, 53, 55, 57, 59, 61, 63, 65, 67, 69, 71, 73, 75, 77, 79, 81, 83, 85, 87, 93, 95, 97, 99, 101, 103, 105, 111, 113, 115, 117, 119, 121, 123, 125, 131, 133, 135, 137, 139, 141, 143, 145, 155, 159, 161, 163 or 327 or a nucleic acid having at least about 70% identity thereto, or the protein comprises a sequence set forth in any one of SEQ ID NOs: 16, 2, 18, 10, 14, 4, 6, 8, 20, 22, 24, 28, 30, 32, 34, 38, 40, 44, 46, 48, 50, 52, 54, 56, 58, 60, 62, 64, 66, 68, 70, 72, 74, 76, 78, 80, 82, 84, 86, 88, 94, 96, 98, 100, 102, 104, 106, 112, 114, 116, 118, 120, 122, 124, 126, 132, 134, 136, 138, 140, 142, 144, 146, 156, 160, 162, 164 or 328 or a protein having at least about 70% identity thereto.

For example, the nucleic acid or protein is expressed in, on or secreted from non-adherent CD133⁺ EPCs at a level at least 3 fold greater than in, on or secreted from HUVECs and the nucleic acid comprises a sequence set forth in any one of SEQ ID NOs: 15, 1, 17, 9, 13, 3, 7, 19, 21, 23, 27, 29, 31, 33, 37, 39, 45, 47, 49, 51, 55, 57, 59, 61, 63, 65, 67, 69, 71, 73, 75, 77, 79, 81, 99, 103, 111, 113, 119, 121, 123, 125, 131, 133, 135, 137, 139, 161, 163, 237, 305 or 327 or a nucleic acid having at least about 70% identity thereto, or the protein comprises a sequence set forth in any one of SEQ ID NOs: 16, 2, 18, 10, 14, 4, 8, 18, 20, 22, 24, 28, 30, 32, 34, 38, 40, 46, 48, 50, 52, 56, 58, 60, 62, 64, 66, 68, 70, 72, 74, 76, 78, 80, 82, 100, 104, 112, 114, 120, 122, 124, 126, 132, 134, 136, 138, 140, 162, 164, 238, 306 or 328 or a protein having at least about 70% identity thereto.

For example, the nucleic acid or protein is expressed in, on or secreted from non-adherent CD133⁺ EPCs at a level at least 4 fold greater than in, on or secreted from HUVECs and the nucleic acid comprises a sequence set forth in any one of SEQ ID NOs: 15, 1, 17, 13, 7, 19, 21, 27, 29, 37, 39, 45, 47, 55, 57, 59, 61, 63, 65, 67, 69, 71, 73, 75, 77, 79, 99, 103, 111, 121, 123, 125, 131, 133, 135, 161, 163 or 327 or a nucleic acid having at least about 70% identity thereto, or the protein comprises a sequence set forth in any one of SEQ ID NOs: 16, 2, 18, 14, 8, 20, 22, 28, 30, 40, 46, 48, 56, 58, 60, 62, 64, 66, 68, 104, 122, 124, 126, 132, 134, 162, 164 or 328 or a protein having at least about 70% identity thereto.

For example, the nucleic acid or protein is expressed in, on or secreted from non-adherent CD133⁺ EPCs at a level at least 5 fold greater than in, on or secreted from HUVECs and the nucleic acid comprises a sequence set forth in any one of SEQ ID NOs: 15, 1, 7, 19, 27, 29, 39, 45, 47, 55, 57, 59, 61, 63, 65, 67, 103, 121, 123, 125, 131, 133, 161, 163 or 327 or a nucleic acid having at least about 70% identity thereto, or the protein comprises a sequence set forth in any one of SEQ ID NOs: 16, 2, 8, 28, 32, 36, 38, 46, 48, 50, 52, 54, 56, 58, 102, 104, 122, 124, 126, 132, 134, 162, 164 or 328 or a protein having at least about 70% identity thereto.

For example, the nucleic acid or protein is expressed in, on or secreted from non-adherent CD133⁺ EPCs at a level at least 6 fold greater than in, on or secreted from HUVECs and the nucleic acid comprises a sequence set forth in any one of SEQ ID NOs: 15, 1, 7, 19, 39, 45, 47, 55, 57, 59, 61, 63, 121, 123, 125, 133, 161, 163 or 327 or a nucleic acid having at least about 70% identity thereto, or the protein comprises a sequence set forth in any one of SEQ ID NOs: 16, 2, 8, 20, 40, 46, 48, 56, 58, 60, 62, 64, 122, 124, 126, 134, 162, 164 or 328 or a protein having at least about 70% identity thereto.

In one example, the level of expression is determined using a microarray.

In one example, a protein or nucleic acid has one or more (e.g., has all) of the following characteristics:
Is expressed on EPCs and has low, or undetectable expression on endothelial cells;
A protein is expressed on the cell surface; and
A protein contains a transmembrane domain.

In one example, the protein is selected from the group consisting of DSG2, EMB, EMR2, NKG7, ADCY7, SLC39A8, TM7SF3, NCSTN, SIRPB1, INSRR, PKD2L1, DPP6, LRRC33 or SLC1A5 or the nucleic acid encodes one of the foregoing proteins.

In one example, the nucleic acid comprises the sequence of SEQ ID NO: 15, 1, 17, 337, 9, 13, 3, 5, 177, 331, 233, 227, 193, 339 or 225 or a sequence having at least about 70% identity thereto, or the protein comprises the sequence of SEQ ID NO: 16, 2, 18, 338, 10, 14, 4, 6, 178, 332, 234, 228, 194, 340 or 226 or a sequence having at least about 70% identity thereto.

In one example, the nucleic acid comprises the sequence of SEQ ID NO: 15 or a sequence having at least about 70% identity thereto, or the protein comprises the sequence of SEQ ID NO: 16 or a sequence having at least about 70% identity thereto.

In one example, the nucleic acid comprises the sequence of SEQ ID NO: 17 or a sequence having at least about 70% identity thereto, or the protein comprises the sequence of SEQ ID NO: 18 or a sequence having at least about 70% identity thereto.

In one example, the nucleic acid comprises the sequence of SEQ ID NO: 1 or a sequence having at least about 70% identity thereto, or the protein comprises the sequence of SEQ ID NO: 2 or a sequence having at least about 70% identity thereto.

In one example, the subject suffers from a cancer, and reduction in EPC numbers and/or activity in the subject reduces neovascularization in the cancer.

In one example, the protein comprises the sequence of SEQ ID NO: 2, 4, 6, 8, 10, 12, 14, 16 or 18 or a protein having at least about 70% identity thereto or the nucleic acid comprises the sequence of SEQ ID NO: 1, 3, 5, 7, 9, 11, 13, 15 or 17 or a nucleic acid having at least about 70% identity thereto.

In another example, the protein is selected from the group consisting of a protein that is a cell adhesion protein as set forth in Table 2, a transporter protein as set forth in Table 3, a growth factor as set forth in Table 4, a receptor as set forth in Table 5 and an enzyme as set forth in Table 6 or wherein the nucleic acid encodes any of the foregoing proteins.

In a further example, the protein is an immunoglobulin, cell adhesion protein comprising the sequence of SEQ ID NO: 2, 24 or 26 or a protein having at least about 70% identity thereto, or the nucleic acid encodes the immunoglobulin, cell adhesion protein and comprises the sequence of SEQ ID NO: 1, 23 or 25 or a nucleic acid having at least about 70% identity thereto.

Exemplary compounds include antibodies or polypeptides comprising antigen binding domains of antibodies. For example, the antibody or protein reduces EPC function and/or induces EPC death. In one example, the antibody or protein additionally comprises a toxic compound to thereby induce EPC death.

The present disclosure additionally provides an isolated antibody or polypeptide that binds specifically to a protein set forth in Table 1 or an immunogenic fragment or epitope thereof, or a polypeptide comprising antigen binding domain of an antibody that binds specifically to a protein set forth in Table 1 or an immunogenic fragment or epitope thereof when used in a method of the disclosure and/or packaged in an article of manufacture with instructions for use in a method of the disclosure.

The present disclosure also provides for use of an isolated antibody or polypeptide that binds specifically to a protein set forth in Table 1 or an immunogenic fragment or epitope thereof, or a polypeptide comprising antigen binding domain of an antibody that binds specifically to a protein set forth in Table 1 or an immunogenic fragment or epitope thereof in the manufacture of a medicament for treating, diagnosing or preventing an EPC-associated condition.

The present disclosure also provides an isolated antibody or polypeptide that binds specifically to a protein set forth in Table 1 or an immunogenic fragment or epitope thereof, or a polypeptide comprising antigen binding domain of an antibody that binds specifically to a protein set forth in Table 1 or an immunogenic fragment or epitope thereof for use in treating, diagnosing or preventing an EPC-associated condition.

The present disclosure additionally provides an isolated antibody or polypeptide, which binds specifically to a protein comprising the sequence of SEQ ID NO 2, 4, 6, 8, 10, 12, 14, 16 or 18 or a protein having at least about 70% identity thereto or an immunogenic fragment or epitope thereof, or a polypeptide comprising antigen binding domain of an antibody that binds specifically to a protein comprising the sequence of SEQ ID NO: 2, 4, 6, 8, 10, 12, 14, 16 or 18 or a protein having at least about 70% identity thereto.

Exemplary antibodies are chimeric antibodies, humanized antibodies or human antibodies.

Another example of the present disclosure provides a pharmaceutical composition comprising an antibody and/or polypeptide of the present disclosure and a pharmaceutically acceptable carrier or excipient. For example, the composition comprises an effective amount of the antibody or polypeptide.

Antibodies or proteins as described herein according to any example of the disclosure can be used in any method described herein requiring a compound that binds a protein.

Another example of the present disclosure provides for the use of an antibody and/or polypeptide of the present disclosure in medicine or in the manufacture of a medicament for administration to a subject in need thereof.

Another example of the present disclosure provides a nucleic acid encoding an antibody or polypeptide of the present disclosure. Such a nucleic acid may be included in an expression vector, e.g., in operable connection with a promoter.

Another example of the present disclosure provides a cell expressing an antibody or polypeptide of the present disclosure, e.g., a hybridoma or a transfectoma.

The present disclosure also provides a solid matrix or semi-solid matrix having immobilized thereon a compound (e.g., antibody or polypeptide comprising an antigen binding domain of an antibody that specifically binds to a protein set forth in Table 1) or a population of cells enriched for EPCs as described herein.

Another example of the present disclosure provides a method for identifying or isolating a compound that modulates EPC function, said method comprising identifying or isolating a compound that reduces expression and/or activity of a nucleic acid or protein set forth in Table 1 in an EPC.

Another example of the present disclosure provides a method for identifying or isolating a compound that binds an EPC, said method comprising identifying or isolating a compound that binds to a protein set forth in Table 1.

For example, the method additionally comprises determining a compound that enhances or reduces EPC activity and/or that induces EPC death, to thereby identify or isolate a compound that modulates EPC function.

For example, the nucleic acid or protein is expressed in, on or secreted from EPCs at a level at least 1.5 fold greater than in, on or secreted by human umbilical cord vascular endothelial cells (HUVECs), for example at a level at least 2 fold greater than in, on or secreted by HUVECs, such as at a level at least 3 or 4 or 5 fold greater than in, on or secreted by HUVECs.

For example, the nucleic acid or protein is expressed in, on or secreted by non-adherent CD133⁺ EPCs at a level at least 1.5 fold greater than in, on or secreted by HUVECs, for example at a level at least 2 fold greater than in, on or secreted by HUVECs, such as at a level at least 3 or 4 or 5 fold greater than in, on or secreted by HUVECs.

For example, the nucleic acid or protein is expressed in, on or secreted from EPCs at a level at least 1.5 fold greater than in, on or secreted by HUVECs and the nucleic acid comprises a sequence set forth in any one of SEQ ID NOs: 15, 1, 17, 9, 13, 3, 5, 7, 11, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 237, 239, 241, 243, 245, 247, 249, 251, 253, 255, 257, 259, 265, 267, 269, 271, 273, 275, 277, 279, 281, 305, 307, 309, 311, 313, 315, 317, 319, 321, 323, 325 or 327 or a nucleic acid having at least about 70% identity thereto, or the protein comprises a sequence set forth in any one of SEQ ID NOs: 16, 2, 18, 10, 14, 4, 6, 8, 12, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 238, 240, 242, 244, 246, 248, 250, 252, 254, 256, 258, 260, 266, 268, 270, 272, 274, 276, 278, 280, 282, 306, 308, 310, 312, 314, 316, 318, 320, 322, 324, 326 or 328 or a protein having at least about 70% identity thereto.

For example, the nucleic acid or protein is expressed in, on or secreted by non-adherent CD133⁺ EPCs at a level at least 1.5 fold greater than in, on or secreted by HUVECs and the nucleic acid comprises a sequence set forth in any one of SEQ ID NOs: 15, 1, 17, 9, 13, 3, 5, 7, 11, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163 or 327 or a nucleic acid having at least about 70% identity thereto, or the protein comprises a sequence set forth in any one of SEQ ID NOs: 16, 2, 18, 10, 14, 4, 6, 8, 12, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164 or 328 or a protein having at least about 70% identity thereto.

For example, the nucleic acid or protein is expressed in, on or secreted from EPCs at a level at least 2 fold greater than in, on or secreted by HUVECs and the nucleic acid comprises a sequence set forth in any one of SEQ ID NOs: 15, 1, 17, 9, 13, 3, 5, 7, 19, 21, 23, 27, 29, 31, 33, 37, 39, 43, 45, 47, 49, 51, 53, 55, 57, 59, 61, 63, 65, 67, 69, 71, 73, 75, 77, 79, 81, 83, 85, 87, 93, 95, 97, 99, 101, 103, 105, 111, 113, 115, 117, 119, 121, 123, 125, 131, 133, 135, 137, 139, 141, 143, 145, 155, 159, 161, 163, 237, 239, 241, 243, 245, 247, 249, 251, 265, 305, 307, 309, 311 or 327 or a nucleic acid having at least about 70% identity thereto, or the protein comprises a sequence set forth in any one of SEQ ID NOs: 16, 2, 18, 10, 14, 4, 6, 8, 20, 22, 24, 28, 30, 32, 34, 38, 40, 44, 46, 48, 50, 52, 54, 56, 58, 60, 62, 64, 66, 68, 70, 72, 74, 76, 78, 80, 82, 84, 86, 88, 94, 96, 98, 100, 102, 104, 106, 112, 114, 116, 118, 120, 122, 124, 126, 132, 134, 136, 138, 140, 142, 144, 146, 156, 160, 162, 164, 238, 240, 242, 244, 246, 248, 250, 252, 266, 306, 308, 310, 312 or 328 or a protein having at least about 70% identity thereto.

For example, the nucleic acid or protein is expressed in, on or secreted from non-adherent CD133⁺ EPCs at a level at least 2 fold greater than in, on or secreted from HUVECs and the nucleic acid comprises a sequence set forth in any one of SEQ ID NOs: 15, 1, 17, 9, 13, 3, 5, 7, 11, 19, 21, 23, 27, 29, 31, 33, 37, 39, 43, 45, 47, 49, 51, 53, 55, 57, 59, 61, 63, 65, 67, 69, 71, 73, 75, 77, 79, 81, 83, 85, 87, 93, 95, 97, 99, 101, 103, 105, 111, 113, 115, 117, 119, 121, 123, 125, 131, 133, 135, 137, 139, 141, 143, 145, 155, 159, 161, 163 or 327 or a nucleic acid having at least about 70% identity thereto, or the protein comprises a sequence set forth in any one of SEQ ID NOs: 16, 2, 18, 10, 14, 4, 6, 8, 20, 22, 24, 28, 30, 32, 34, 38, 40, 46, 48, 50, 52, 56, 58, 60, 62, 64, 66, 68, 70, 72, 74, 76, 78, 80, 82, 100, 104, 112, 114, 120, 122, 124, 126, 132, 134, 136, 138, 140, 162, 164, 238, 306 or 328 or a protein having at least about 70% identity thereto.

For example, the nucleic acid or protein is expressed in, on or secreted from EPCs at a level at least 3 fold greater than in, on or secreted by HUVECs and the nucleic acid comprises a sequence set forth in any one of SEQ ID NOs: 15, 1, 17, 9, 13, 3, 5, 7, 19, 21, 23, 27, 29, 31, 33, 37, 39, 43, 45, 47, 49, 51, 53, 55, 57, 59, 61, 63, 65, 67, 69, 71, 73, 75, 77, 79, 81, 83, 85, 87, 93, 95, 97, 99, 101, 103, 105, 111, 113, 115, 117, 119, 121, 123, 125, 131, 133, 135, 137, 139, 141, 143, 145, 155, 159, 161, 163 or 327 or a nucleic acid having at least about 70% identity thereto, or the protein comprises a sequence set forth in any one of SEQ ID NOs: 16, 2, 18, 10, 14, 4, 6, 8, 20, 22, 24, 28, 30, 32, 34, 38, 40, 44, 46, 48, 50, 52, 54, 56, 58, 60, 62, 64, 66, 68, 70, 72, 74, 76, 78, 80, 82, 84, 86, 88, 94, 96, 98, 100, 102, 104, 106, 112, 114, 116, 118, 120, 122, 124, 126, 132, 134, 136, 138, 140, 142, 144, 146, 156, 160, 162, 164 or 328 or a protein having at least about 70% identity thereto.

For example, the nucleic acid or protein is expressed in, on or secreted from non-adherent CD133⁺ EPCs at a level at least 3 fold greater than in, on or secreted from HUVECs and the nucleic acid comprises a sequence set forth in any one of SEQ ID NOs: 15, 1, 17, 9, 13, 3, 7, 19, 21, 23, 27, 29, 31, 33, 37, 39, 45, 47, 49, 51, 55, 57, 59, 61, 63, 65, 67, 69, 71, 73, 75, 77, 79, 81, 99, 103, 111, 113, 119, 121, 123, 125, 131, 133, 135, 137, 139, 161, 163, 237, 305 or 327 or a nucleic acid having at least about 70% identity thereto, or the protein comprises a sequence set forth in any one of SEQ ID NOs: 16, 2, 18, 10, 14, 4, 8, 18, 20, 22, 24, 28, 30, 32, 34, 38, 40, 46, 48, 50, 52, 56, 58, 60, 62, 64, 66, 68, 70, 72, 74, 76, 78, 80, 82, 100, 104, 112, 114, 120, 122, 124, 126, 132, 134, 136, 138, 140, 162, 164, 238, 306 or 328 or a protein having at least about 70% identity thereto.

For example, the nucleic acid or protein is expressed in, on or secreted from non-adherent CD133⁺ EPCs at a level at least 4 fold greater than in, on or secreted from HUVECs and the nucleic acid comprises a sequence set forth in any one of SEQ ID NOs: 15, 1, 17, 13, 7, 19, 21, 27, 29, 37, 39, 45, 47, 55, 57, 59, 61, 63, 65, 67, 69, 71, 73, 75, 77, 79, 99, 103, 111, 121, 123, 125, 131, 133, 135, 161, 163 or 327 or a nucleic acid having at least about 70% identity thereto, or the protein comprises a sequence set forth in any one of SEQ ID NOs: 16, 2, 18, 14, 8, 20, 22, 28, 30, 40, 46, 48, 56, 58, 60, 62, 64, 66, 68, 104, 122, 124, 126, 132, 134, 162, 164 or 328 or a protein having at least about 70% identity thereto.

For example, the nucleic acid or protein is expressed in, on or secreted from non-adherent CD133⁺ EPCs at a level at least 5 fold greater than in, on or secreted from HUVECs and the nucleic acid comprises a sequence set forth in any one of SEQ ID NOs: 15, 1, 7, 19, 27, 29, 39, 45, 47, 55, 57, 59, 61, 63, 65, 67, 103, 121, 123, 125, 131, 133, 161, 163 or 327 or a nucleic acid having at least about 70% identity thereto, or the protein comprises a sequence set forth in any one of SEQ ID NOs: 16, 2, 8, 28, 32, 36, 38, 46, 48, 50, 52, 54, 56, 58, 102, 104, 122, 124, 126, 132, 134, 162, 164 or 328 or a protein having at least about 70% identity thereto.

For example, the nucleic acid or protein is expressed in, on or secreted from non-adherent CD133⁺ EPCs at a level at least 6 fold greater than in, on or secreted from HUVECs and the nucleic acid comprises a sequence set forth in any one of SEQ ID NOs: 15, 1, 7, 19, 39, 45, 47, 55, 57, 59, 61, 63, 121, 123, 125, 133, 161, 163 or 327 or a nucleic acid having at least about 70% identity thereto, or the protein comprises a sequence set forth in any one of SEQ ID NOs: 16, 2, 8, 20, 40, 46, 48, 56, 58, 60, 62, 64, 122, 124, 126, 134, 162, 164 or 328 or a protein having at least about 70% identity thereto.

In one example, the level of expression is determined using a microarray.

In one example, a protein or nucleic acid has one or more (e.g., has all) of the following characteristics:
Is expressed on EPCs and has low, or undetectable expression on endothelial cells;
A protein is expressed on the cell surface; and
A protein contains a transmembrane domain.

In one example, the protein is selected from the group consisting of DSG2, EMB, EMR2, NKG7, ADCY7, SLC39A8, TM7SF3, NCSTN, SIRPB1, INSRR, PKD2L1, DPP6, LRRC33 or SLC1A5 or the nucleic acid encodes one of the foregoing proteins.

In one example, the nucleic acid comprises the sequence of SEQ ID NO: 15, 1, 17, 337, 9, 13, 3, 5, 177, 331, 233, 227, 193, 339 or 225 or a sequence having at least about 70% identity thereto, or the protein comprises the sequence of SEQ ID NO: 16, 2, 18, 338, 10, 14, 4, 6, 178, 332, 234, 228, 194, 340 or 226 or a sequence having at least about 70% identity thereto.

In one example, the nucleic acid comprises the sequence of SEQ ID NO: 15 or a sequence having at least about 70% identity thereto, or the protein comprises the sequence of SEQ ID NO: 16 or a sequence having at least about 70% identity thereto.

In one example, the nucleic acid comprises the sequence of SEQ ID NO: 17 or a sequence having at least about 70% identity thereto, or the protein comprises the sequence of SEQ ID NO: 18 or a sequence having at least about 70% identity thereto.

In one example, the nucleic acid comprises the sequence of SEQ ID NO: 1 or a sequence having at least about 70% identity thereto, or the protein comprises the sequence of SEQ ID NO: 2 or a sequence having at least about 70% identity thereto.

In one example, the protein comprises the sequence of SEQ ID NO: 2, 4, 6, 8, 10, 12, 14, 16 or 18 or a protein having at least about 70% identity thereto or the nucleic acid comprises the sequence of SEQ ID NO: 1, 3, 5, 7, 9, 11, 13, 15 or 17 or a nucleic acid having at least about 70% identity thereto.

In another example, the protein is selected from the group consisting of a protein that is a cell adhesion protein as set forth in Table 2, a transporter protein as set forth in Table 3, a growth factor as set forth in Table 4, a receptor as set forth in Table 5 and an enzyme as set forth in Table 6 or wherein the nucleic acid encodes any of the foregoing proteins.

In a further example, the protein is an immunoglobulin, cell adhesion protein comprising the sequence of SEQ ID NO: 2, 24 or 26 or a protein having at least about 70% identity thereto, or the nucleic acid encodes the immunoglobulin, cell adhesion protein and comprises the sequence of SEQ ID NO: 1, 23 or 25 or a nucleic acid having at least about 70% identity thereto.

Examples of the present disclosure also encompasses classes of proteins or nucleic acids expressed in, on or secreted by non-adherent CD133⁺ EPCs at a level at least 7 fold or 8 fold or 9 fold or 14 fold or 18 fold greater than in, on or secreted by HUVECs. The skilled artisan will be capable of determining such classes of proteins or nucleic acids and/or proteins based on the disclosure herein, e.g., in Tables 7 to 9, ad those disclosures shall be taken to provide explicit support for such classes of nucleic acids and/or proteins.

### BRIEF DESCRIPTION OF THE DRAWINGS

FIG. 1 is a graphical representation showing hierarchical clustering for gene expression for CD133+ cells freshly isolated from human umbilical cord blood mononuclear cells and cultured for 4 days in complete culture medium (EPCs) or endothelial cells isolated from human umbilical cords and cultured to 2 passages or less in complete culture medium (HUVEC). The overall transcriptional profiles of the EPCs are more similar to each other than to the profile for typical HUVEC. The heat map depicts gene expression.

FIG. 2A is a series of graphical representations showing expression of EMR2 on EPCs (left panel) and HUVECs (right panel). The dashed line indicates the level of binding of isotype control antibody, and the solid line indicates the level of binding of anti-EMR2 antibody (clone 2A1, targeting the stalk region of EMR2 only). The bar represents cells binding less than 1% of isotype control antibody.

FIG. 2B is a series of graphical representations showing expression of EMR2 on U937 myeloid cells (left panel) and Jurkat T cells (right panel). The dashed line indicates the level of binding of isotype control antibody, and the solid line indicates the level of binding of anti-EMR2 antibody. The bar represents cells binding less than 1% of isotype control antibody.

FIG. 3A is a series of graphical representations showing expression of DSG2 on EPCs (left panel) and HUVECs (right panel). The dashed line indicates the level of binding of isotype control antibody, and the solid line indicates the level of binding of anti-DSG2 antibody. The bar represents cells binding less than 1% of isotype control antibody.

FIG. 3B is a series of graphical representations showing expression of CD133 and CD117 on freshly isolated human peripheral blood mononuclear cells (PBMNCs) (left panel) and DSG2 expression on CD133⁺ CD117⁺ double positive PBMNCs (right panel). The dashed line indicates the level of binding of isotype control antibody, and the solid line indicates the level of binding of anti-DSG2 antibody.

FIG. 4A is a series of graphical representations showing that when an anti-DSG2 monoclonal antibody is used to pull down DSG2 expressing cells from freshly isolated umbilical cord blood (UCB) it enriches for cells that are CD34⁺ and CD31⁺, progenitor and vascular markers, respectively.

FIG. 4B is a series of graphical representations showing that when an anti-CD133 monoclonal antibody is used to pull down CD133 expressing cells from freshly isolated peripheral blood it also enriches for cells that are CD34+ and CD31+ but that two populations appear to be isolated.

FIGS. 5A, 5B and 5C are a series of graphical representations showing that when an anti-DSG2 monoclonal antibody is used to pull down DSG2 expressing cells from freshly isolated human umbilical cord blood (UCB) and then cultured for 4 days in EC supportive media (EGM-2+supplements) it enriches for cells that are (FIG. 5A) DSG2⁺ and CD133^{dim} (FIG. 5B) CD34⁺ and CD45^{dim} and (FIG. 5C) VEGFR2⁺ and CD31⁺.

FIGS. 6A and 6B are a series of graphical representations showing expression of DSG2 on C32 melanoma cells (FIG. 6A) and MM200 melanoma cells (FIG. 6B). The dashed line indicates the level of binding of isotype control antibody, and the solid line indicates the level of binding of anti-DSG2 antibody.

FIGS. 7A and 7B include a series of representations, with FIG. 7A showing HUVEC (labelled with DiI-acetylated low density lipoprotein and C32 or MM200 melanoma cells (labelled with CFSE-DA) co-cultured in the 3-dimensional matrix Matrigel® and the formation of tube-like structures from 7 h post seeding. FIG. 7B shows that, from one experiment with triplicate samples, quantification of the number of tubes formed per field of view at 12 hours suggests an increase in tube number when the DSG2⁺ C32 cells are co-cultured with HUVEC in Matrigel® (right graph). Co-culture of MM200 melanoma cells with HUVEC does not increase tube numbers above HUVEC alone.

FIGS. 8A and 8B contain a series of graphical representations showing results of a representative experiment in which DSG2 is knocked down in C32 cells. FIG. 8A shows changes in expression of DSG2 as detected by qPCR in the presence of various siRNAs (as indicated). FIG. 8B shows expression of DSG2 as detected by flow cytometry in the presence of various siRNAs (as indicated; mean+sd). This result has been repeatable in 3 separate experiments.

FIGS. 9A and 9B comprise a series of representations showing results of knockdown of DSG2 expression. FIG. 9A shows representative images showing HUVEC (labelled with DiI-acetylated low density lipoprotein) and C32 melanoma cells without or with knockdown of DSG2 by siRNA (unlabelled) co-cultured in the 3-dimensional matrix Matrigel® and the formation of tube-like structures at 12 h post seeding. FIG. 9B shows that, from one experiment with triplicate samples, quantification of the number of tubes formed per field of view at 12 hours suggests a decrease increase in tube number when the C32 cells have DSG2 knockdown and co-cultured with HUVEC in Matrigel (right graph).

FIG. 10 includes copies of two photomicrographs showing representative images of DSG2 expression on the vasculature of paraffin embedded human tissue (cells expressing DSG2 are indicated by arrows). The DSG2 of the ovary vasculature is stained with DAB and sections counter stained with hematoxylin for nuclei with an enlarged image depicted in the right panel.

FIG. 11 is a graphical representation showing expression of DSG2 on freshly isolated mouse bone marrow cells. The dashed lines indicate the level of autofluorescence of the cells as well as the binding of the secondary antibody alone, and the solid line indicates the level of binding of anti-DSG2 antibody. The bar represents cells binding less than 1% of secondary alone control.

FIG. 12 is a representative image of DSG2 expression in on the melanoma cells in a spontaneous mouse model (Tyr⁻Cre⁺:Braf^{V600E/+};Pten^{del/del}) of melanoma. The DSG2 of the mouse tissue paraffin embedded section is stained with an alkaline phosphatase/red chromagen system. Sections were counter stained with hematoxylin for nuclei with the secondary antibody alone depicted in the left panel.

FIGS. 13A and 13B include a series of graphical representations showing characterization expanded the expansion CD133⁺ isolated cells from human umbilical cord blood. FIG. 13A shows the fold expansion of CD133⁺ isolated cells from human umbilical cord blood in StemSpan media (Stem Cell Technologies) in BD tissue culture plates at a density ˜7.5×10⁵ cells/ml. The data represent the mean+/−sem from 5 independent donor experiments. FIG. 13B shows expression of DSG2 on EPCs expanded for 7 days in culture. The left line indicates the level of binding of isotype control antibody (iso), and the right line indicates the level of binding of anti-DSG2 antibody (as indicated). FIG. 13C shows expression of EMR2 on EPCs expanded for 7 days in culture. The left line indicates the level of binding of isotype control antibody (iso), and the right line indicates the level of binding of anti-EMR2 antibody (as indicated).

### KEY TO SEQUENCE LISTING

SEQ ID NO: 1 is a nucleotide sequence of a Homo sapiens embigin homolog;

SEQ ID NO: 2 is an amino acid sequence of a Homo sapiens embigin homolog;

SEQ ID NO: 3 is a nucleotide sequence of a Homo sapiens solute carrier family 39 (zinc transporter), member 8;

SEQ ID NO: 4 is an amino acid sequence of a Homo sapiens solute carrier family 39 (zinc transporter), member 8;

SEQ ID NO: 5 is a nucleotide sequence of a Homo sapiens transmembrane 7 superfamily member 3;

SEQ ID NO: 6 is an amino acid sequence of a Homo sapiens transmembrane 7 superfamily member 3;

SEQ ID NO: 7 is a nucleotide sequence of a Homo sapiens plexin C1;

SEQ ID NO: 8 is an amino acid sequence of a Homo sapiens plexin C1; SEQ ID NO: 9 is a nucleotide sequence of a Homo sapiens natural killer cell group 7 sequence;

SEQ ID NO: 10 is an amino acid sequence of a Homo sapiens natural killer cell group 7 sequence;

SEQ ID NO: 11 is a nucleotide sequence of a Homo sapiens olfactory receptor, family 52, subfamily B, member 6;

SEQ ID NO: 12 is an amino acid sequence of a Homo sapiens olfactory receptor, family 52, subfamily B, member 6;

SEQ ID NO: 13 is a nucleotide sequence of a Homo sapiens adenylate cyclase 7;

SEQ ID NO: 14 is an amino acid sequence of a Homo sapiens adenylate cyclase 7;

SEQ ID NO: 15 is a nucleotide sequence of a Homo sapiens desmoglein 2;

SEQ ID NO: 16 is an amino acid sequence of a Homo sapiens desmoglein 2;

SEQ ID NO: 17 is a nucleotide sequence of a Homo sapiens egf-like module containing, mucin-like, hormone receptor-like 2;

SEQ ID NO: 18 is an amino acid sequence of a Homo sapiens egf-like module containing, mucin-like, hormone receptor-like 2;

SEQ ID NO: 19 is a nucleotide sequence of a Homo sapiens solute carrier family 15 (H+/peptide transporter), member 2;

SEQ ID NO: 20 is an amino acid sequence of a Homo sapiens solute carrier family 15 (H+/peptide transporter), member 2;

SEQ ID NO: 21 is a nucleotide sequence of a Homo sapiens solute carrier family 16, member 6 (monocarboxylic acid transporter 7);

SEQ ID NO: 22 is an amino acid sequence of a Homo sapiens solute carrier family 16, member 6 (monocarboxylic acid transporter 7);

SEQ ID NO: 23 is a nucleotide sequence of a Homo sapiens sialic acid binding Ig-like lectin 10;

SEQ ID NO: 24 is an amino acid sequence of a Homo sapiens sialic acid binding Ig-like lectin 10;

SEQ ID NO: 25 is a nucleotide sequence of a Homo sapiens sialic acid binding Ig-like lectin 6;

SEQ ID NO: 26 is an amino acid sequence of a Homo sapiens sialic acid binding Ig-like lectin 6;

SEQ ID NO: 27 is a nucleotide sequence of a Homo sapiens amphiregulin;

SEQ ID NO: 28 is an amino acid sequence of a Homo sapiens amphiregulin;

SEQ ID NO: 29 is a nucleotide sequence of a Homo sapiens integral membrane protein 2A;

SEQ ID NO: 30 is an amino acid sequence of a Homo sapiens integral membrane protein 2A;

SEQ ID NO: 31 is a nucleotide sequence of a Homo sapiens glycoprotein M6B;

SEQ ID NO: 32 is an amino acid sequence of a Homo sapiens glycoprotein M6B;

SEQ ID NO: 33 is a nucleotide sequence of a Homo sapiens cannabinoid receptor 2 (macrophage);

SEQ ID NO: 34 is an amino acid sequence of a Homo sapiens cannabinoid receptor 2 (macrophage);

SEQ ID NO: 35 is a nucleotide sequence of a Homo sapiens protease, serine, 21 (testisin);

SEQ ID NO: 36 is an amino acid sequence of a Homo sapiens protease, serine, 21 (testisin);

SEQ ID NO: 37 is a nucleotide sequence of a Homo sapiens neuregulin 4;

SEQ ID NO: 38 is an amino acid sequence of a Homo sapiens neuregulin 4;

SEQ ID NO: 39 is a nucleotide sequence of a Homo sapiens epithelial mitogen homolog (mouse);

SEQ ID NO: 40 is an amino acid sequence of a Homo sapiens epithelial mitogen homolog (mouse);

SEQ ID NO: 41 is a nucleotide sequence of a Homo sapiens rhomboid domain containing 1;

SEQ ID NO: 42 is an amino acid sequence of a Homo sapiens rhomboid domain containing 1;

SEQ ID NO: 43 is a nucleotide sequence of a Homo sapiens ATP-binding cassette, sub-family C (CFTR/MRP), member 4;

SEQ ID NO: 44 is an amino acid sequence of a Homo sapiens ATP-binding cassette, sub-family C (CFTR/MRP), member 4;

SEQ ID NO: 45 is a nucleotide sequence of a Homo sapiens sortilin-related receptor, L(DLR class) A repeats-containing;

SEQ ID NO: 46 is an amino acid sequence of a Homo sapiens sortilin-related receptor, L(DLR class) A repeats-containing;

SEQ ID NO: 47 is a nucleotide sequence of a Homo sapiens solute carrier family 8 (sodium/calcium exchanger), member 1;

SEQ ID NO: 48 is an amino acid sequence of a Homo sapiens solute carrier family 8 (sodium/calcium exchanger), member 1;

SEQ ID NO: 49 is a nucleotide sequence of a Homo sapiens solute carrier family 22 (organic cation/carnitine transporter), member 16;

SEQ ID NO: 50 is an amino acid sequence of a Homo sapiens solute carrier family 22 (organic cation/carnitine transporter), member 16;

SEQ ID NO: 51 is a nucleotide sequence of a Homo sapiens solute carrier family 24 (sodium/potassium/calcium exchanger), member 3;

SEQ ID NO: 52 is an amino acid sequence of a Homo sapiens solute carrier family 24 (sodium/potassium/calcium exchanger), member 3;

SEQ ID NO: 53 is a nucleotide sequence of a Homo sapiens solute carrier family 2 (facilitated glucose/fructose transporter), member 5;

SEQ ID NO: 54 is an amino acid sequence of a Homo sapiens solute carrier family 2 (facilitated glucose/fructose transporter), member 5;

SEQ ID NO: 55 is a nucleotide sequence of a Homo sapiens NCK-associated protein 1-like;

SEQ ID NO: 56 is an amino acid sequence of a Homo sapiens NCK-associated protein 1-like;

SEQ ID NO: 57 is a nucleotide sequence of a Homo sapiens ecotropic viral integration site 2B;

SEQ ID NO: 58 is an amino acid sequence of a Homo sapiens ecotropic viral integration site 2B;

SEQ ID NO: 59 is a nucleotide sequence of a Homo sapiens potassium voltage-gated channel;

SEQ ID NO: 60 is an amino acid sequence of a Homo sapiens potassium voltage-gated channel;

SEQ ID NO: 61 is a nucleotide sequence of a Homo sapiens purinergic receptor P2Y, G-protein coupled, 14;

SEQ ID NO: 62 is an amino acid sequence of a Homo sapiens purinergic receptor P2Y, G-protein coupled, 14;

SEQ ID NO: 63 is a nucleotide sequence of a Homo sapiens 5-hydroxytryptamine (serotonin) receptor 1F;

SEQ ID NO: 64 is an amino acid sequence of a Homo sapiens 5-hydroxytryptamine (serotonin) receptor 1F;

SEQ ID NO: 65 is a nucleotide sequence of a Homo sapiens T cell receptor associated transmembrane adaptor 1;

SEQ ID NO: 66 is an amino acid sequence of a Homo sapiens T cell receptor associated transmembrane adaptor 1;

SEQ ID NO: 67 is a nucleotide sequence of a Homo sapiens G protein-coupled receptor 183;

SEQ ID NO: 68 is an amino acid sequence of a Homo sapiens G protein-coupled receptor 183;

SEQ ID NO: 69 is a nucleotide sequence of a Homo sapiens olfactory receptor, family 13, subfamily D, member 1;

SEQ ID NO: 70 is an amino acid sequence of a Homo sapiens olfactory receptor, family 13, subfamily D, member 1;

SEQ ID NO: 71 is a nucleotide sequence of a Homo sapiens V-set and immunoglobulin domain containing 4;

SEQ ID NO: 72 is an amino acid sequence of a Homo sapiens V-set and immunoglobulin domain containing 4;

SEQ ID NO: 73 is a nucleotide sequence of a Homo sapiens taste receptor, type 2, member 4;

SEQ ID NO: 74 is an amino acid sequence of a Homo sapiens taste receptor, type 2, member 4;

SEQ ID NO: 75 is a nucleotide sequence of a Homo sapiens G protein-coupled receptor 18;

SEQ ID NO: 76 is an amino acid sequence of a Homo sapiens G protein-coupled receptor 18;

SEQ ID NO: 77 is a nucleotide sequence of a Homo sapiens taste receptor, type 2, member 3;

SEQ ID NO: 78 is an amino acid sequence of a Homo sapiens taste receptor, type 2, member 3;

SEQ ID NO: 79 is a nucleotide sequence of a Homo sapiens major histocompatibility complex, class I-related;

SEQ ID NO: 80 is an amino acid sequence of a Homo sapiens major histocompatibility complex, class I-related;

SEQ ID NO: 81 is a nucleotide sequence of a Homo sapiens G protein-coupled receptor 34;

SEQ ID NO: 82 is an amino acid sequence of a Homo sapiens G protein-coupled receptor 34;

SEQ ID NO: 83 is a nucleotide sequence of a Homo sapiens potassium voltage-gated channel, shaker-related subfamily, beta member 2;

SEQ ID NO: 84 is an amino acid sequence of a Homo sapiens potassium voltage-gated channel, shaker-related subfamily, beta member 2;

SEQ ID NO: 85 is a nucleotide sequence of a Homo sapiens potassium voltage-gated channel, Isk-related family, member 3;

SEQ ID NO: 86 is an amino acid sequence of a Homo sapiens potassium voltage-gated channel, Isk-related family, member 3;

SEQ ID NO: 87 is a nucleotide sequence of a Homo sapiens linker for activation of T cells family, member 2;

SEQ ID NO: 88 is an amino acid sequence of a Homo sapiens linker for activation of T cells family, member 2;

SEQ ID NO: 89 is a nucleotide sequence of a Homo sapiens megalencephalic leukoencephalopathy with subcortical cysts 1;

SEQ ID NO: 90 is an amino acid sequence of a Homo sapiens megalencephalic leukoencephalopathy with subcortical cysts 1;

SEQ ID NO: 91 is a nucleotide sequence of a Homo sapiens ectonucleotide pyrophosphatase/phosphodiesterase 5 (putative function);

SEQ ID NO: 92 is an amino acid sequence of a Homo sapiens ectonucleotide pyrophosphatase/phosphodiesterase 5 (putative function);

SEQ ID NO: 93 is a nucleotide sequence of a Homo sapiens feline leukemia virus subgroup C cellular receptor 1;

SEQ ID NO: 94 is an amino acid sequence of a Homo sapiens feline leukemia virus subgroup C cellular receptor 1;

SEQ ID NO: 95 is a nucleotide sequence of a Homo sapiens G protein-coupled receptor 65;

SEQ ID NO: 96 is an amino acid sequence of a Homo sapiens G protein-coupled receptor 65;

SEQ ID NO: 97 is a nucleotide sequence of a Homo sapiens opsin 3;

SEQ ID NO: 98 is an amino acid sequence of a Homo sapiens opsin 3;

SEQ ID NO: 99 is a nucleotide sequence of a Homo sapiens taste receptor, type 2, member 13;

SEQ ID NO: 100 is an amino acid sequence of a Homo sapiens taste receptor, type 2, member 13;

SEQ ID NO: 101 is a nucleotide sequence of a Homo sapiens claudin 20;

SEQ ID NO: 102 is an amino acid sequence of a Homo sapiens claudin 20;

SEQ ID NO: 103 is a nucleotide sequence of a Homo sapiens solute carrier family 1 (glial high affinity glutamate transporter), member 3;

SEQ ID NO: 104 is an amino acid sequence of a Homo sapiens solute carrier family 1 (glial high affinity glutamate transporter), member 3;

SEQ ID NO: 105 is a nucleotide sequence of a Homo sapiens solute carrier family 1 (glutamate/neutral amino acid transporter), member 4;

SEQ ID NO: 106 is an amino acid sequence of a Homo sapiens solute carrier family 1 (glutamate/neutral amino acid transporter), member 4;

SEQ ID NO: 107 is a nucleotide sequence of a Homo sapiens claudin 10;

SEQ ID NO: 108 is an amino acid sequence of a Homo sapiens claudin 10;

SEQ ID NO: 109 is a nucleotide sequence of a Homo sapiens ADAM metallopeptidase with thrombospondin type 1 motif, 2;

SEQ ID NO: 110 is an amino acid sequence of a Homo sapiens ADAM metallopeptidase with thrombospondin type 1 motif, 2;

SEQ ID NO: 111 is a nucleotide sequence of a Homo sapiens thromboxane A synthase 1 (platelet);

SEQ ID NO: 112 is an amino acid sequence of a Homo sapiens thromboxane A synthase 1 (platelet);

SEQ ID NO: 113 is a nucleotide sequence of a Homo sapiens lysosomal protein transmembrane 5;

SEQ ID NO: 114 is an amino acid sequence of a Homo sapiens lysosomal protein transmembrane 5;

SEQ ID NO: 115 is a nucleotide sequence of a Homo sapiens vesicle-associated membrane protein 8 (endobrevin);

SEQ ID NO: 116 is an amino acid sequence of a Homo sapiens vesicle-associated membrane protein 8 (endobrevin);

SEQ ID NO: 117 is a nucleotide sequence of a Homo sapiens A kinase (PRKA) anchor protein 7;

SEQ ID NO: 118 is an amino acid sequence of a Homo sapiens A kinase (PRKA) anchor protein 7;

SEQ ID NO: 119 is a nucleotide sequence of a Homo sapiens sema domain, immunoglobulin domain (Ig), short basic domain, secreted, (semaphorin) 3C;

SEQ ID NO: 120 is an amino acid sequence of a Homo sapiens sema domain, immunoglobulin domain (Ig), short basic domain, secreted, (semaphorin) 3C;

SEQ ID NO: 121 is a nucleotide sequence of a Homo sapiens solute carrier family 38, member 1;

SEQ ID NO: 122 is an amino acid sequence of a Homo sapiens solute carrier family 38, member 1;

SEQ ID NO: 123 is a nucleotide sequence of a Homo sapiens CD302 molecule;

SEQ ID NO: 124 is an amino acid sequence of a Homo sapiens CD302 molecule;

SEQ ID NO: 125 is a nucleotide sequence of a Homo sapiens phospholipase B domain containing 1;

SEQ ID NO: 126 is an amino acid sequence of a Homo sapiens phospholipase B domain containing 1;

SEQ ID NO: 127 is a nucleotide sequence of a Homo sapiens lysyl oxidase-like 3;

SEQ ID NO: 128 is an amino acid sequence of a Homo sapiens lysyl oxidase-like 3;

SEQ ID NO: 129 is a nucleotide sequence of a Homo sapiens family with sequence similarity 46, member C;

SEQ ID NO: 130 is an amino acid sequence of a Homo sapiens family with sequence similarity 46, member C;

SEQ ID NO: 131 is a nucleotide sequence of a Homo sapiens microfibrillar-associated protein 4;

SEQ ID NO: 132 is an amino acid sequence of a Homo sapiens microfibrillar-associated protein 4;

SEQ ID NO: 133 is a nucleotide sequence of a Homo sapiens IQ motif containing B1;

SEQ ID NO: 134 is an amino acid sequence of a Homo sapiens IQ motif containing B1;

SEQ ID NO: 135 is a nucleotide sequence of a Homo sapiens fibrillin 2;

SEQ ID NO: 136 is an amino acid sequence of a Homo sapiens fibrillin 2;

SEQ ID NO: 137 is a nucleotide sequence of a Homo sapiens osteoglycin;

SEQ ID NO: 138 is an amino acid sequence of a Homo sapiens osteoglycin;

SEQ ID NO: 139 is a nucleotide sequence of a Homo sapiens osteomodulin;

SEQ ID NO: 140 is an amino acid sequence of a Homo sapiens osteomodulin;

SEQ ID NO: 141 is a nucleotide sequence of a Homo sapiens asporin;

SEQ ID NO: 142 is an amino acid sequence of a Homo sapiens asporin;

SEQ ID NO: 143 is a nucleotide sequence of a Homo sapiens pregnancy-zone protein;

SEQ ID NO: 144 is an amino acid sequence of a Homo sapiens pregnancy-zone protein;

SEQ ID NO: 145 is a nucleotide sequence of a Homo sapiens hereditary sensory neuropathy, type II (WNK1);

SEQ ID NO: 146 is an amino acid sequence of a Homo sapiens hereditary sensory neuropathy, type II (WNK1);

SEQ ID NO: 147 is a nucleotide sequence of a Homo sapiens serpin peptidase inhibitor, clade I (pancpin), member 2;

SEQ ID NO: 148 is an amino acid sequence of a Homo sapiens serpin peptidase inhibitor, clade I (pancpin), member 2;

SEQ ID NO: 149 is a nucleotide sequence of a Homo sapiens extracellular matrix protein 2, female organ and adipocyte specific;

SEQ ID NO: 150 is an amino acid sequence of a Homo sapiens extracellular matrix protein 2, female organ and adipocyte specific;

SEQ ID NO: 151 is a nucleotide sequence of a Homo sapiens ER lipid raft associated 1;

SEQ ID NO: 152 is an amino acid sequence of a Homo sapiens ER lipid raft associated 1;

SEQ ID NO: 153 is a nucleotide sequence of a Homo sapiens cadherin, EGF LAG seven-pass G-type receptor 2 (flamingo homolog, Drosophila);

SEQ ID NO: 154 is an amino acid sequence of a Homo sapiens cadherin, EGF LAG seven-pass G-type receptor 2 (flamingo homolog, Drosophila);

SEQ ID NO: 155 is a nucleotide sequence of a Homo sapiens neuroplastin;

SEQ ID NO: 156 is an amino acid sequence of a Homo sapiens neuroplastin;

SEQ ID NO: 157 is a nucleotide sequence of a Homo sapiens chromosome 20 open reading frame 3;

SEQ ID NO: 158 is an amino acid sequence of a Homo sapiens chromosome 20 open reading frame 3;

SEQ ID NO: 159 is a nucleotide sequence of a Homo sapiens gamma-aminobutyric acid (GABA) A receptor, alpha 3;

SEQ ID NO: 160 is an amino acid sequence of a Homo sapiens gamma-aminobutyric acid (GABA) A receptor, alpha 3;

SEQ ID NO: 161 is a nucleotide sequence of a Homo sapiens desmoglein 3 (pemphigus vulgaris antigen);

SEQ ID NO: 162 is an amino acid sequence of a Homo sapiens desmoglein 3 (pemphigus vulgaris antigen);

SEQ ID NO: 163 is a nucleotide sequence of a Homo sapiens plexin B2;

SEQ ID NO: 164 is an amino acid sequence of a Homo sapiens plexin B2;

SEQ ID NO: 165 is a nucleotide sequence of a Homo sapiens ORAI calcium release-activated calcium modulator 1;

SEQ ID NO: 166 is an amino acid sequence of a Homo sapiens ORAI calcium release-activated calcium modulator 1;

SEQ ID NO: 167 is a nucleotide sequence of a Homo sapiens Dystroglycan;

SEQ ID NO: 168 is an amino acid sequence of a Homo sapiens Dystroglycan;

SEQ ID NO: 169 is a nucleotide sequence of a Homo sapiens Transmembrane protein C14orf176;

SEQ ID NO: 170 is an amino acid sequence of a Homo sapiens Transmembrane protein C14orf176;

SEQ ID NO: 171 is a nucleotide sequence of a Homo sapiens Myelin protein zero-like protein 1;

SEQ ID NO: 172 is an amino acid sequence of a Homo sapiens Myelin protein zero-like protein 1;

SEQ ID NO: 173 is a nucleotide sequence of a Homo sapiens Claudin-17;

SEQ ID NO: 174 is an amino acid sequence of a Homo sapiens Claudin-17;

SEQ ID NO: 175 is a nucleotide sequence of a Homo sapiens Probable G-protein coupled receptor 125;

SEQ ID NO: 176 is an amino acid sequence of a Homo sapiens Probable G-protein coupled receptor 125;

SEQ ID NO: 177 is a nucleotide sequence of a Homo sapiens Nicastrin;

SEQ ID NO: 178 is an amino acid sequence of a Homo sapiens Nicastrin;

SEQ ID NO: 179 is a nucleotide sequence of a Homo sapiens Uroplakin-1a;

SEQ ID NO: 180 is an amino acid sequence of a Homo sapiens Uroplakin-1a;

SEQ ID NO: 181 is a nucleotide sequence of a Homo sapiens Teneurin-3;

SEQ ID NO: 182 is an amino acid sequence of a Homo sapiens Teneurin-3;

SEQ ID NO: 183 is a nucleotide sequence of a Homo sapiens Netrin receptor DCC;

SEQ ID NO: 184 is an amino acid sequence of a Homo sapiens Netrin receptor DCC;

SEQ ID NO: 185 is a nucleotide sequence of a Homo sapiens Uncharacterized protein KIAA0090;

SEQ ID NO: 186 is an amino acid sequence of a Homo sapiens Uncharacterized protein KIAA0090;

SEQ ID NO: 187 is a nucleotide sequence of a Homo sapiens Amiloride-sensitive cation channel 4;

SEQ ID NO: 188 is an amino acid sequence of a Homo sapiens Amiloride-sensitive cation channel 4;

SEQ ID NO: 189 is a nucleotide sequence of a Homo sapiens Voltage-dependent L-type calcium channel subunit alpha-1D;

SEQ ID NO: 190 is an amino acid sequence of a Homo sapiens Voltage-dependent L-type calcium channel subunit alpha-1D;

SEQ ID NO: 191 is a nucleotide sequence of a Homo sapiens Chondroitin sulfate proteoglycan 4;

SEQ ID NO: 192 is an amino acid sequence of a Homo sapiens Chondroitin sulfate proteoglycan 4;

SEQ ID NO: 193 is a nucleotide sequence of a Homo sapiens Dipeptidyl aminopeptidase-like protein 6;

SEQ ID NO: 194 is an amino acid sequence of a Homo sapiens Dipeptidyl aminopeptidase-like protein 6;

SEQ ID NO: 195 is a nucleotide sequence of a Homo sapiens Protocadherin Fat 2;

SEQ ID NO: 196 is an amino acid sequence of a Homo sapiens Protocadherin Fat 2;

SEQ ID NO: 197 is a nucleotide sequence of a Homo sapiens Low-density lipoprotein receptor-related protein 12;

SEQ ID NO: 198 is an amino acid sequence of a Homo sapiens Low-density lipoprotein receptor-related protein 12;

SEQ ID NO: 199 is a nucleotide sequence of a Homo sapiens Neuropeptide Y receptor type 2;

SEQ ID NO: 200 is an amino acid sequence of a Homo sapiens Neuropeptide Y receptor type 2;

SEQ ID NO: 201 is a nucleotide sequence of a Homo sapiens Olfactory receptor 11H4;

SEQ ID NO: 202 is an amino acid sequence of a Homo sapiens Olfactory receptor 11H4;

SEQ ID NO: 203 is a nucleotide sequence of a Homo sapiens Protocadherin alpha-4;

SEQ ID NO: 204 is an amino acid sequence of a Homo sapiens Protocadherin alpha-4;

SEQ ID NO: 205 is a nucleotide sequence of a Homo sapiens Protocadherin alpha-C1;

SEQ ID NO: 206 is an amino acid sequence of a Homo sapiens Protocadherin alpha-C1;

SEQ ID NO: 207 is a nucleotide sequence of a Homo sapiens Rhomboid domain-containing protein 2;

SEQ ID NO: 208 is an amino acid sequence of a Homo sapiens Rhomboid domain-containing protein 2;

SEQ ID NO: 209 is a nucleotide sequence of a Homo sapiens Sodium channel protein type 5 subunit alpha;

SEQ ID NO: 210 is an amino acid sequence of a Homo sapiens Sodium channel protein type 5 subunit alpha;

SEQ ID NO: 211 is a nucleotide sequence of a Homo sapiens Serine incorporator 5;

SEQ ID NO: 212 is an amino acid sequence of a Homo sapiens Serine incorporator 5;

SEQ ID NO: 213 is a nucleotide sequence of a Homo sapiens Solute carrier family 12 member 1;

SEQ ID NO: 214 is an amino acid sequence of a Homo sapiens Solute carrier family 12 member 1;

SEQ ID NO: 215 is a nucleotide sequence of a Homo sapiens Proton-coupled folate transporter;

SEQ ID NO: 216 is an amino acid sequence of a Homo sapiens Proton-coupled folate transporter;

SEQ ID NO: 217 is a nucleotide sequence of a Homo sapiens Solute carrier organic anion transporter family member 1B1;

SEQ ID NO: 218 is an amino acid sequence of a Homo sapiens Solute carrier organic anion transporter family member 1B1;

SEQ ID NO: 219 is a nucleotide sequence of a Homo sapiens Anoctamin-2;

SEQ ID NO: 220 is an amino acid sequence of a Homo sapiens Anoctamin-2;

SEQ ID NO: 221 is a nucleotide sequence of a Homo sapiens ATP-binding cassette sub-family A member 12;

SEQ ID NO: 222 is an amino acid sequence of a Homo sapiens ATP-binding cassette sub-family A member 12;

SEQ ID NO: 223 is a nucleotide sequence of a Homo sapiens Carboxypeptidase M;

SEQ ID NO: 224 is an amino acid sequence of a Homo sapiens Carboxypeptidase M;

SEQ ID NO: 225 is a nucleotide sequence of a Homo sapiens Neutral amino acid transporter B(0);

SEQ ID NO: 226 is an amino acid sequence of a Homo sapiens Neutral amino acid transporter B(0);

SEQ ID NO: 227 is a nucleotide sequence of a Homo sapiens Polycystic kidney disease 2-like 1 protein;

SEQ ID NO: 228 is an amino acid sequence of a Homo sapiens Polycystic kidney disease 2-like 1 protein;

SEQ ID NO: 229 is a nucleotide sequence of a Homo sapiens Probable phospholipid-transporting ATPase VA;

SEQ ID NO: 230 is an amino acid sequence of a Homo sapiens Probable phospholipid-transporting ATPase VA;

SEQ ID NO: 231 is a nucleotide sequence of a Homo sapiens Acetylcholine receptor subunit gamma;

SEQ ID NO: 232 is an amino acid sequence of a Homo sapiens Acetylcholine receptor subunit gamma;

SEQ ID NO: 233 is a nucleotide sequence of a Homo sapiens Insulin receptor-related protein;

SEQ ID NO: 234 is an amino acid sequence of a Homo sapiens Insulin receptor-related protein;

SEQ ID NO: 235 is a nucleotide sequence of a Homo sapiens Voltage-dependent N-type calcium channel subunit alpha-1B;

SEQ ID NO: 236 is an amino acid sequence of a Homo sapiens Voltage-dependent N-type calcium channel subunit alpha-1B;

SEQ ID NO: 237 is a nucleotide sequence of a Homo sapiens sperm associated antigen IIB;

SEQ ID NO: 238 is an amino acid sequence of a Homo sapiens sperm associated antigen II;

SEQ ID NO: 239 is a nucleotide sequence of a Homo sapiens Fraser Syndrome 1;

SEQ ID NO: 240 is an amino acid sequence of a Homo sapiens Fraser Syndrome 1;

SEQ ID NO: 241 is a nucleotide sequence of a Homo sapiens immunoglobulin-like domain containing receptor 1;

SEQ ID NO: 242 is an amino acid sequence of a Homo sapiens immunoglobulin-like domain containing receptor 1;

SEQ ID NO: 243 is a nucleotide sequence of a Homo sapiens EPB41L1—erythrocyte membrane protein band 4.1 like 1;

SEQ ID NO: 244 is an amino acid sequence of a Homo sapiens EPB41L1—erythrocyte membrane protein band 4.1 like 1;

SEQ ID NO: 245 is a nucleotide sequence of a Homo sapiens B melanoma antigen;

SEQ ID NO: 246 is an amino acid sequence of a Homo sapiens B melanoma antigen;

SEQ ID NO: 247 is a nucleotide sequence of a Homo sapiens glutamate receptor, ionotropic, AMPA2;

SEQ ID NO: 248 is an amino acid sequence of a Homo sapiens glutamate receptor, ionotropic, AMPA2;

SEQ ID NO: 249 is a nucleotide sequence of a Homo sapiens synaptotagmin XV;

SEQ ID NO: 250 is an amino acid sequence of a Homo sapiens synaptotagmin XV;

SEQ ID NO: 251 is a nucleotide sequence of a Homo sapiens NFASC—neurofascin homolog (chicken);

SEQ ID NO: 252 is an amino acid sequence of a Homo sapiens NFASC—neurofascin homolog (chicken);

SEQ ID NO: 253 is a nucleotide sequence of a Homo sapiens EST (IMAGE:2110090);

SEQ ID NO: 254 is an amino acid sequence of a Homo sapiens EST (IMAGE:2110090);

SEQ ID NO: 255 is a nucleotide sequence of a Homo sapiens solute carrier family 30, member 10;

SEQ ID NO: 256 is an amino acid sequence of a Homo sapiens solute carrier family 30, member 10;

SEQ ID NO: 257 is a nucleotide sequence of a Homo sapiens UNC-93 homologue A (C. elegans);

SEQ ID NO: 258 is an amino acid sequence of a Homo sapiens UNC-93 homologue A (C. elegans);

SEQ ID NO: 259 is a nucleotide sequence of a Homo sapiens Olfactory receptor, family 1, subfamily C, member 1;

SEQ ID NO: 260 is an amino acid sequence of a Homo sapiens Olfactory receptor, family 1, subfamily C, member 1;

SEQ ID NO: 261 is a nucleotide sequence of a Homo sapiens transmembrane and tetratricopeptide repeat containing 4;

SEQ ID NO: 262 is an amino acid sequence of a Homo sapiens transmembrane and tetratricopeptide repeat containing 4;

SEQ ID NO: 263 is a nucleotide sequence of a Homo sapiens chloride channel 4;

SEQ ID NO: 264 is an amino acid sequence of a Homo sapiens chloride channel 4;

SEQ ID NO: 265 is a nucleotide sequence of a Homo sapiens olfactory receptor, family 12, subfamily D, member 3;

SEQ ID NO: 266 is an amino acid sequence of a Homo sapiens olfactory receptor, family 12, subfamily D, member 3;

SEQ ID NO: 267 is a nucleotide sequence of a Homo sapiens Butyrophilin-like protein 8 precursor;

SEQ ID NO: 268 is an amino acid sequence of a Homo sapiens Butyrophilin-like protein 8 precursor;

SEQ ID NO: 269 is a nucleotide sequence of a Homo sapiens solute carrier, family 7 member 14;

SEQ ID NO: 270 is an amino acid sequence of a Homo sapiens solute carrier, family 7 member 14;

SEQ ID NO: 271 is a nucleotide sequence of a Homo sapiens olfactory receptor, family 7 subfamily D member 4;

SEQ ID NO: 272 is an amino acid sequence of a Homo sapiens olfactory receptor, family 7 subfamily D member 4;

SEQ ID NO: 273 is a nucleotide sequence of a Homo sapiens mucin 12, cell surface associated;

SEQ ID NO: 274 is an amino acid sequence of a Homo sapiens mucin 12, cell surface associated;

SEQ ID NO: 275 is a nucleotide sequence of a Homo sapiens T-cell receptor gamma chain C region PT-gamma-1/2;

SEQ ID NO: 276 is an amino acid sequence of a Homo sapiens T-cell receptor gamma chain C region PT-gamma-1/2;

SEQ ID NO: 277 is a nucleotide sequence of a Homo sapiens DEFb109—Defensin beta 109;

SEQ ID NO: 278 is an amino acid sequence of a Homo sapiens DEFb109—Defensin beta 109;

SEQ ID NO: 279 is a nucleotide sequence of a Homo sapiens Kv channel interacting protein 1 (variant 1);

SEQ ID NO: 280 is an amino acid sequence of a Homo sapiens Kv channel interacting protein 1 (variant 1);

SEQ ID NO: 281 is a nucleotide sequence of a Homo sapiens solute carrier family 45, member 4;

SEQ ID NO: 282 is an amino acid sequence of a Homo sapiens solute carrier family 45, member 4;

SEQ ID NO: 283 is a nucleotide sequence of a Homo sapiens ectonucleotide pyrophosphatase/phosphodiesterase 6;

SEQ ID NO: 284 is an amino acid sequence of a Homo sapiens ectonucleotide pyrophosphatase/phosphodiesterase 6;

SEQ ID NO: 285 is a nucleotide sequence of a Homo sapiens protocadherin beta 8;

SEQ ID NO: 286 is an amino acid sequence of a Homo sapiens protocadherin beta 8;

SEQ ID NO: 287 is a nucleotide sequence of a Homo sapiens olfactory receptor, family 2, sub family T, member 3;

SEQ ID NO: 288 is an amino acid sequence of a Homo sapiens olfactory receptor, family 2, sub family T, member 3;

SEQ ID NO: 289 is a nucleotide sequence of a Homo sapiens olfactory receptor family 5, subfamily M, member 10;

SEQ ID NO: 290 is an amino acid sequence of a Homo sapiens olfactory receptor family 5, subfamily M, member 10;

SEQ ID NO: 291 is a nucleotide sequence of a Homo sapiens olfactory receptor family 4, subfamily S, member 1;

SEQ ID NO: 292 is an amino acid sequence of a Homo sapiens olfactory receptor family 4, subfamily S, member 1;

SEQ ID NO: 293 is a nucleotide sequence of a Homo sapiens G protein-coupled receptor 83;

SEQ ID NO: 294 is an amino acid sequence of a Homo sapiens G protein-coupled receptor 83;

SEQ ID NO: 295 is a nucleotide sequence of a Homo sapiens taste receptor, type 2, member 19;

SEQ ID NO: 296 is an amino acid sequence of a Homo sapiens taste receptor, type 2, member 19;

SEQ ID NO: 297 is a nucleotide sequence of a Homo sapiens Kallmann syndrome 1 sequence;

SEQ ID NO: 298 is an amino acid sequence of a Homo sapiens Kallmann syndrome 1 sequence;

SEQ ID NO: 299 is a nucleotide sequence of a Homo sapiens solute carrier organic anion transporter family, member 1B3;

SEQ ID NO: 300 is an amino acid sequence of a Homo sapiens solute carrier organic anion transporter family, member 1B3;

SEQ ID NO: 301 is a nucleotide sequence of a Homo sapiens Gene and two pseudogenes for 7 transmembrane receptor (rhodopsin family) (olfactory receptor like) proteins and a 60S acidic ribosomal protein P2 (RPLP2) pseudogene;

SEQ ID NO: 302 is an amino acid sequence of a Homo sapiens Gene and two pseudogenes for 7 transmembrane receptor (rhodopsin family) (olfactory receptor like) proteins and a 60S acidic ribosomal protein P2 (RPLP2) pseudogene;

SEQ ID NO: 303 is a nucleotide sequence of a Homo sapiens major histocompatability complex, class II, DQ beta 1;

SEQ ID NO: 304 is an amino acid sequence of a Homo sapiens major histocompatability complex, class II, DQ beta 1;

SEQ ID NO: 305 is a nucleotide sequence of a Homo sapiens CD166 (ALCAM) activated leukocyte cell adhesion molecule;

SEQ ID NO: 306 is an amino acid sequence of a Homo sapiens CD166 (ALCAM) activated leukocyte cell adhesion molecule;

SEQ ID NO: 307 is a nucleotide sequence of a Homo sapiens IL-20Rbeta—Interleukin 20 receptor beta;

SEQ ID NO: 308 is an amino acid sequence of a Homo sapiens IL-20Rbeta—Interleukin 20 receptor beta;

SEQ ID NO: 309 is a nucleotide sequence of a Homo sapiens podoplanin-differentiation factor; O-glycosylated;

SEQ ID NO: 310 is an amino acid sequence of a Homo sapiens podoplanin-differentiation factor; O-glycosylated;

SEQ ID NO: 311 is a nucleotide sequence of a Homo sapiens cholinergic receptor, muscarinic 3;

SEQ ID NO: 312 is an amino acid sequence of a Homo sapiens cholinergic receptor, muscarinic 3;

SEQ ID NO: 313 is a nucleotide sequence of a Homo sapiens intergrin, beta 1 (fibronectin receptor, beta polypeptide, antigen CD29 includes MDF2, MSK12);

SEQ ID NO: 314 is an amino acid sequence of a Homo sapiens intergrin, beta 1 (fibronectin receptor, beta polypeptide, antigen CD29 includes MDF2, MSK12);

SEQ ID NO: 315 is a nucleotide sequence of a Homo sapiens sialic acid binding Ig-like lectin 8, CD329;

SEQ ID NO: 316 is an amino acid sequence of a Homo sapiens sialic acid binding Ig-like lectin 8, CD329;

SEQ ID NO: 317 is a nucleotide sequence of a Homo sapiens RAS-related protein RAP1A;

SEQ ID NO: 318 is an amino acid sequence of a Homo sapiens RAS-related protein RAP1A;

SEQ ID NO: 319 is a nucleotide sequence of a Homo sapiens Plexin A2;

SEQ ID NO: 320 is an amino acid sequence of a Homo sapiens Plexin A2;

SEQ ID NO: 321 is a nucleotide sequence of a Homo sapiens CD158b (KIR2DL3) killer cell immunoglobulin-like receptor, 2 domains, ligand 3;

SEQ ID NO: 322 is an amino acid sequence of a Homo sapiens CD158b (KIR2DL3) killer cell immunoglobulin-like receptor, 2 domains, ligand 3;

SEQ ID NO: 323 is a nucleotide sequence of a Homo sapiens CD314, killer cell lectin-like receptor, subfamily K, member 1;

SEQ ID NO: 324 is an amino acid sequence of a Homo sapiens CD314, killer cell lectin-like receptor, subfamily K, member 1;

SEQ ID NO: 325 is a nucleotide sequence of a Homo sapiens chemokine (C-X3-C) receptor 1, CCRL1;

SEQ ID NO: 326 is an amino acid sequence of a Homo sapiens chemokine (C-X3-C) receptor 1, CCRL1;

SEQ ID NO: 327 is a nucleotide sequence of a Homo sapiens G protein-coupled receptor 174; and

SEQ ID NO: 328 is an amino acid sequence of a Homo sapiens G protein-coupled receptor 174.

SEQ ID NO: 329 is a nucleotide sequence encoding a Homo sapiens Disintegrin and metalloproteinase domain-containing protein 10 (ADAM10).

SEQ ID NO: 330 is an amino acid sequence of a Homo sapiens Disintegrin and metalloproteinase domain-containing protein 10 (ADAM10).

SEQ ID NO: 331 is a nucleotide sequence encoding a Homo sapiens signal-regulatory protein beta 1 (SIRPB1).

SEQ ID NO: 332 is an amino acid sequence of a Homo sapiens signal-regulatory protein beta 1 (SIRPB1).

SEQ ID NO: 333 is a nucleotide sequence encoding a Homo sapiens GM-CSF receptor subunit alpha precursor (CSF2RA).

SEQ ID NO: 334 is an amino acid sequence of a Homo sapiens GM-CSF receptor subunit alpha precursor (CSF2RA).

SEQ ID NO: 335 is a nucleotide sequence encoding a Homo sapiens Ecotropic viral integration 5 (EVI5).

SEQ ID NO: 336 is an amino acid sequence of a Homo sapiens Ecotropic viral integration 5 (EVI5).

SEQ ID NO: 337 is a nucleotide sequence encoding a Homo sapiens lysyl oxidase-like 4 (LOXL4).

SEQ ID NO: 338 is an amino acid sequence of a Homo sapiens lysyl oxidase-like 4 (LOXL4).

SEQ ID NO: 339 is a nucleotide sequence encoding a Homo sapiens Leucine rich containing 33 (LRRC33).

SEQ ID NO: 340 is an amino acid sequence of a Homo sapiens Leucine rich containing 33 (LRRC33).

### DETAILED DESCRIPTION

### Selected Definitions

As used herein, the term “endothelial progenitor cell” or “EPC” shall be understood to mean a cell of the endothelial lineage capable of differentiating into a mature endothelial cell, for example a blood vessel endothelial cell. This term does not include embryonic stem cells or induced pluripotent cells (which are capable of differentiating into endothelium). Exemplary EPCs are monocytic EPCs or hemangioblastic EPCs. Exemplary EPCs express at least sphingosine kinase 1 (SK-1). Alternatively or in addition, EPCs express at least CD34 or at least CD14. Alternatively, or in addition, the EPCs express at least CD133. EPCs may also express CD45 and/or CD31 and/or VEGFR2. Alternatively, or in addition, an EPC does not express significant or above background levels of CD144 and/or vWF and/or eNOS and/or Tie2. Alternatively or in addition, EPCs produce pro-angiogenic factors, e.g., hepatocyte growth factor and/or insulin-like growth factor-1 and/or basic fibroblast growth factor and/or VEGF. In one example, the EPCs do not adhere to tissue culture plastic-ware, optionally plastic-ware coated with extracellular matrix or a component thereof (e.g., fibronectin).). Therefore, the EPCs used in the present disclosure are, for example, non-adherent EPCs. In one example, the EPCs are isolated from 4-7 day cultured non-adherent CD133 expressing mononuclear cells or are contained within a population of 4-7 day cultured non-adherent CD133 expressing mononuclear cells.

The term “endothelium” or “endothelial cell” shall be understood to mean a tissue or cell that lines tissues of the circulatory system. Endothelium is a form of epithelium, in particular, squamous epithelium.

The term “EPC-associated condition” shall be taken to encompass any disease or disorder or state in which modulation of EPC numbers and/or activity may provide a beneficial effect and/or characterized by excessive or insufficient EPC numbers and/or activity. Exemplary conditions are described herein and are to be taken to apply mutatis mutandis to those examples of the disclosure relating to diagnosis/prognosis/treatment/prophylaxis of an EPC-associated condition. In one example, an EPC-associated condition is characterized by insufficient EPC numbers and/or activity. Exemplary conditions include cardiovascular disease, autoimmune conditions (e.g., rheumatoid arthritis, psoriatic arthritis, systemic lupus erythematosus (SLE) and systemic sclerosis), antineutrophil cytoplasmic antibodies (ANCA)-associated vasculitis, ischemia (including ischemia resulting from a transplant) and testicular necrosis. In another example, the condition is associated with excessive EPC numbers and/or activity (including excessive neovascularization). Exemplary conditions include cancer (including solid tumors, leukemias, lymphoma, melanoma, glioma, breast cancer, colonic cancer, gastric cancer, esophageal cancer, renal cell cancer, ovarian cancer, cervical cancer, carcinoid cancer, testicular cancer, prostate cancer, head and neck cancer and hepatocellular carcinoma), cancer metastasis, cancer neovascularization, autoimmune disease (including psoriasis), nephropathy, retinopathy, preeclampsia hepatitis, sepsis and macular degeneration.

As used herein, the term “EPC activity” will be understood to encompass any function that is characteristic of an EPC and includes any one or more of the following:
Uptake of diacetylated LDL (Dil-Ac-LDL);
Binding of Ulex europaeus I lectin;
Labeling with antibodies that bind to CD34, CD133 and VEGF-R2;
Ability to form tubes in vitro;
Migration towards angiogenic factors (such as VEGF) in vitro or in vivo;
Secretion of angiogenic factors (such as VEGF, hepatocyte growth factor, granulocyte-colony stimulating factor, Macrophage migration inhibitory factor interleukin 8);
Ability to induce neovascularization in vivo; and
Ability to form colony forming units (CFUs).

Assays for determining EPC activity are known in the art and/or described in more detail herein.

Based on the foregoing, the skilled artisan will be aware that a compound or method that inhibits the activity of an EPC can inhibit any activity discussed above. Such inhibition can be by way of modulating a biological activity in an EPC to thereby inhibit the activity or by killing (including lysing) an EPC.

As used herein, the term “endothelial cell other than an EPC” or “non-EPC” includes mature endothelial cells, such as cells expressing CD144 and/or vWF and/or eNOS and/or Tie2.

Reference herein to a “fold change” in expression or “X fold greater expression” shall be understood to mean the ratio of the level of expression of one cell type compared to another cell type. Fold change in expression is calculated using standard methods in the art. For example, to determine the fold increase in expression of a nucleic acid or protein in an EPC compared to a HUVEC, the level of expression in an EPC is determined and the level of expression in a HUVEC is determined and the ratio between those values is calculated. Numerous methods for determining expression levels of nucleic acids and/or proteins are known in the art. Non-limiting examples of such methods are described herein and are to be taken to apply mutatis mutandis to the determination of fold change in expression of a protein or nucleic acid.

As used herein, the term “enriched” or “enrich” in the context of a cell population shall be taken to encompass a population of cells comprising EPCs, including a population in which the number or percentage of EPCs is greater than the number or percentage in a naturally occurring cell population. For example, a population enriched in EPCs is made up of at least about 0.02% of said cells, or at least about 0.05% of said cells or at least about 0.1% of said cells or at least about 0.2% of said cells or at least about 0.5% of said cells or at least about 0.5% of said cells or at least about 0.8% of said cells or at least about 1% of said cells or at least about 2% of said cells or at least about 3% of said cells or at least about 4% of said cells or at least about 5% of said cells or at least about 10% of said cells or at least about 15% of said cells or at least about 20% of said cells or at least about 25% of said cells or at least about 30% of said cells or at least about 40% of said cells or at least about 50% of said cells or at least about 60% of said cells or at least about 70% of said cells or at least about 80% of said cells or at least about 85% of said cells or at least about 90% of said cells or at least about 95% of said cells or at least about 97% of said cells or at least about 98% of said cells or at least about 99% of said cells.

As used herein, the terms “preventing”, “prevent” or “prevention” include administering a therapeutically effective amount of an inhibitor(s) and/or agent(s) described herein sufficient to stop or hinder the development of at least one symptom of a specified disease or condition.

The term “sample” shall be understood to mean a tissue or fluid from a subject, e.g., a blood sample (including blood for a subject treated to mobilize bone marrow stem cells or that from umbilical cord) or fraction thereof (e.g., an umbilical cord fraction, plasma or serum or buffy coat fraction or peripheral blood mononuclear cell fraction) or bone marrow or a part thereof. Accordingly, the present disclosure also encompasses a method additionally comprising providing or obtaining a sample from a subject. Such a sample may have been isolated previously from a subject, e.g., the method is performed in vitro or ex vivo.

As used herein, the term “specifically binds” shall be taken to mean a compound reacts or associates more frequently, more rapidly, with greater duration and/or with greater affinity with a particular cell or substance than it does with alternative cells or substances. For example, a compound that specifically binds to a target protein is a compound that binds that protein or an epitope or immunogenic fragment thereof with greater affinity, avidity, more readily, and/or with greater duration than it binds to unrelated protein and/or epitopes or immunogenic fragments thereof. It is also understood by reading this definition that, for example, a compound that specifically binds to a first target may or may not specifically bind to a second target. As such, “specific binding” does not necessarily require exclusive binding or non-detectable binding of another molecule, this is encompassed by the term “selective binding”. Generally, but not necessarily, reference to binding means specific binding.

As used herein, the term “subject” shall be taken to mean any subject comprising EPCs, for example a mammal. Exemplary subjects include but are not limited to human, primate, livestock (e.g. sheep, cow, horse, donkey, pig), companion animals (e.g. dogs, cats), laboratory test animals (e.g. mice, rabbits, rats, guinea pigs, hamsters), captive wild animal (e.g. fox, deer). For example, the mammal is a human or primate. In one example, the mammal is a human.

As used herein, the terms “treating”, “treat” or “treatment” include administering a therapeutically effective amount of a compound described herein sufficient to reduce or eliminate at least one symptom of a specified disease or condition.

### General

The term “and/or”, e.g., “X and/or Y” shall be understood to mean either “X and Y” or “X or Y” and shall be taken to provide explicit support for both meanings or for either meaning.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

Those skilled in the art will appreciate that the disclosure described herein is susceptible to variations and modifications other than those specifically described. It is to be understood that the disclosure includes all such variations and modifications. The disclosure also includes all of the steps, features, compositions and compounds referred to or indicated in this specification, individually or collectively, and any and all combinations or any two or more of said steps or features.

The present disclosure is not to be limited in scope by the specific examples described herein, which are intended for the purpose of exemplification only. Functionally-equivalent products, compositions and methods are clearly within the scope of the disclosure, as described herein.

Any example of the disclosure herein shall be taken to apply mutatis mutandis to any other example of the disclosure unless specifically stated otherwise.

Unless specifically defined otherwise, all technical and scientific terms used herein shall be taken to have the same meaning as commonly understood by one of ordinary skill in the art (for example, in cell culture, molecular genetics, immunology, immunohistochemistry, protein chemistry, and biochemistry).

Unless otherwise indicated, the recombinant protein, cell culture, and immunological techniques utilized in the present disclosure are standard procedures, well known to those skilled in the art. Such techniques are described and explained throughout the literature in sources such as, J. Perbal, A Practical Guide to Molecular Cloning, John Wiley and Sons (1984), J. Sambrook et al., Molecular Cloning: A Laboratory Manual, Cold Spring Harbor Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al., (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbor Laboratory, (1988), and J. E. Coligan et al., (editors) Current Protocols in Immunology, John Wiley & Sons (including all updates until present).

### EPC Markers and Encoding Nucleic Acids

Exemplary EPC protein markers and nucleic acids encoding same are discussed herein and/or set forth in any one or more of Tables 1 to 6. In this respect, the present disclosure encompasses nucleic acids or proteins having a sequence at least about 70% identical to a nucleic acid or protein recited in any one or more of Tables 1 to 6. The EPC protein markers may be a cell surface protein located on the plasma membrane, or a protein secreted into extracellular space and/or located in the cytoplasm of an EPC cell.

TABLE 1
Proteins and nucleic acids encoding same that are upregulated in, on or
secreted from EPCs.

| | | | | Exemplary | Exemplary |
|---|---|---|---|---|---|
| | | Reference | | Nucleotide | Amino Acid |
| Gene ID | Entrez Gene Name | Sequence(s) | Category | SEQ ID NO | SEQ ID NO |
| DSG2 | desmoglein 2 | NM_001943 | cell adhesion | 15 | 16 |
| EMB | embigin homolog | NM_198449; | cell adhesion | 1 | 2 |
| | | NR_003955 | | | |
| EMR2 | egf-like module containing, | NM_013447; | receptor | 17 | 18 |
| | mucin-like, hormone | NM_152916; | | | |
| | receptor-like 2 | NM_152919; | | | |
| | | NM_152917; | | | |
| | | NM_152920; | | | |
| | | NM_152921; | | | |
| | | NM_152918 | | | |
| LOXL4 | lysyl oxidase-like 4 | NM_032211 | enzyme | 337 | 338 |
| NKG7 | natural killer cell group 7 | NM_005601 | other | 9 | 10 |
| | sequence | | | | |
| ADCY7 | adenylate cyclase 7 | NM_001114 | enzyme | 13 | 14 |
| SLC39A8 | solute carrier family 39 | NM_022154; | transport | 3 | 4 |
| | (zinc transporter), member 8 | NM_001135148; | | | |
| | | NM_001135147; | | | |
| | | NM_001135146 | | | |
| TM7SF3 | transmembrane 7 | NM_016551 | other | 5 | 6 |
| | superfamily member 3 | | | | |
| NCSTN | Nicastrin | Q92542; Q5T207; | enzyme | 177 | 178 |
| | | Q86VV5 | | | |
| SIRPB1 | signal-regulatory protein | NM_006065 | receptor | 331 | 332 |
| | beta 1 | | | | |
| INSRR | insulin receptor-related | P14616; O60724; | enzyme | 233 | 234 |
| | protein | Q5VZS3 | | | |
| PKD2L1 | polycystic kidney disease 2- | Q9P0L9; O75972; | transport | 227 | 228 |
| | like 1 protein | Q5W039; | | | |
| | | Q9UP35; | | | |
| | | Q9UPA2 | | | |
| DPP6 | dipeptidyl aminopeptidase- | P42658 | enzyme | 193 | 194 |
| | like protein 6 | | | | |
| LRRC33 | Leucine rich containing 33 | NM_198565 | other | 339 | 340 |
| | | Q86YC3 | | | |
| SLC1A5 | Neutral amino acid | Q15758; | transport | 225 | 226 |
| | transporter B(0) | A8K9H5; | | | |
| | | D0EYG6; | | | |
| | | O95720; | | | |
| | | Q96RL9; | | | |
| | | Q9BWQ3; | | | |
| | | Q9UNP2 | | | |
| ADAM10 | Disintegrin and | NM_001110 | enzyme | 329 | 330 |
| | metalloproteinase domain- | | | | |
| | containing protein 10 | | | | |
| CSF2RA | GM-CSF receptor subunit | NM_006140 | receptor | 333 | 334 |
| | alpha precursor | NM_172245 | | | |
| | | NM_172246 | | | |
| | | NM_172247 | | | |
| | | NM_172249 | | | |
| | | NM_001161529 | | | |
| | | NM_001161530 | | | |
| | | NM_001161531 | | | |
| | | NR_027760 | | | |
| | | NM_001161532 | | | |
| EVI5 | Ecotropic viral integration 5 | NM_005665 | other | 335 | 336 |
| | | Q59FE7 | | | |
| EVI2B | esotropic viral integration | NM_001003927 | other | 57 | 58 |
| | site 2B | | | | |
| SORL1(LRP9) | sortilin-related receptor, | NM_003105 | transport | 45 | 46 |
| | L(DLR class) A repeats- | | | | |
| | containing | | | | |
| CNR2 | cannabinoid receptor 2 | NM_001841 | receptor | 33 | 34 |
| | (macrophage) | | | | |
| NCKAP1L | NCK-associated protein 1- | NM_005337; | other | 55 | 56 |
| | like | NM_001184976 | | | |
| SIGLEC10 | sialic acid binding Ig-like | NM_033130; | cell adhesion | 23 | 24 |
| | lectin 10 | NM_001171156; | | | |
| | | NM_001171157; | | | |
| | | NM_001171158; | | | |
| | | NM_001171159; | | | |
| | | NM_001171160; | | | |
| | | NM_001171161 | | | |
| SIGLEC6 | sialic acid binding Ig-like | NM_001245; | cell adhesion | 25 | 26 |
| | lectin 6 | NM_198845; | | | |
| | | NM_198846; | | | |
| | | NM_001177547; | | | |
| | | NM_001177548; | | | |
| | | NM_001177549 | | | |
| SLC15A2 | solute carrier family 15 | NM_021082; | transport | 19 | 20 |
| | (H+/peptide transporter), | NM_001145998 | | | |
| | member 2 | | | | |
| SLC22A16 | solute carrier family 22 | NM_033125 | transport | 49 | 50 |
| | (organic cation/carnitine | | | | |
| | transporter), member 16 | | | | |
| SLC24A3 | solute carrier family 24 | NM_020689 | transport | 51 | 52 |
| | (sodium/potassium/calcium | | | | |
| | exchanger), member 3 | | | | |
| SLC2A5 | solute carrier family 2 | NM_003039; | transport | 53 | 54 |
| | (facilitated glucose/fructose | NM_001135585 | | | |
| | transporter), member 5 | | | | |
| SLC1A3 | solute carrier family 1 (glial | NM_004172; | transport | 103 | 104 |
| | high affinity glutamate | NM_001166695; | | | |
| | transporter), member 3 | NM_001166696 | | | |
| PLXNC1 | plexin C1 | NM_005761 | cell adhesion | 7 | 8 |
| OR52B6 | olfactory receptor, family | NM_001005162 | receptor | 11 | 12 |
| | 52, subfamily B, member 6 | | | | |
| ABCC4 | ATP-binding cassette, sub- | NM_005845\|NM_001105515 | transport | 43 | 44 |
| | family C (CFTR/MRP), | | | | |
| | member 4 | | | | |
| SLC16A6 | solute carrier family 16, | NM_004694; | transport | 21 | 22 |
| | member 6 (monocarboxylic | NM_001174166 | | | |
| | acid transporter 7) | | | | |
| AREG | amphiregulin | NM_001657 | growth factor | 27 | 28 |
| ITM2A | integral membrane protein | NM_004867 | other | 29 | 30 |
| | 2A | | | | |
| GPM6B | glycoprotein M6B | NM_001001995; | other | 31 | 32 |
| | | NM_001001996; | | | |
| | | NM_005278; | | | |
| | | NM_001001994 | | | |
| PRSS21 | protease, serine, 21 | NM_006799; | enzyme | 35 | 36 |
| | (testisin) | NM_144956; | | | |
| | | NM_144957 | | | |
| NRG4 | neuregulin 4 | NM_138573 | growth factor | 37 | 38 |
| EPGN | epithelial mitogen homolog | NM_001013442 | growth factor | 39 | 40 |
| | (mouse) | | | | |
| RHBDD1 | rhomboid domain | NM_032276; | enzyme | 41 | 42 |
| | containing 1 | NM_001167608 | | | |
| SLC8A1 | solute carrier family 8 | NM_021097; NM_001112800; | transport | 47 | 48 |
| | (sodium/calcium exchanger), | NM_001112801; | | | |
| | member 1 | NM_001112802 | | | |
| KCNQ5 | potassium voltage-gated | NM_019842; | transport | 59 | 60 |
| | channel | NM_001160130; | | | |
| | | NM_001160132; | | | |
| | | NM_001160133; | | | |
| | | NM_001160134 | | | |
| P2RY14 | purinergic receptor P2Y, G- | NM_014879; | receptor | 61 | 62 |
| | protein coupled, 14 | Q15391; | | | |
| | | BC034989; | | | |
| | | Q15391; | | | |
| | | NM_001081455 | | | |
| HTR1F | 5-hydroxytryptamine | NM_000866; | receptor | 63 | 64 |
| | (serotonin) receptor 1F | Q4QRI9; | | | |
| | | BC069125; | | | |
| | | P30939; | | | |
| | | BC069125; | | | |
| | | Q4QRI9 | | | |
| TRAT1 | T cell receptor associated | NM_016388; | other | 65 | 66 |
| | transmembrane adaptor 1 | Q6PIZ9; | | | |
| | | BC025713; | | | |
| | | Q6PIZ9 | | | |
| GPR183 | G protein-coupled receptor | NM_004951 | receptor | 67 | 68 |
| | 183 | | | | |
| OR13D1 | olfactory receptor, family | NM_001004484 | receptor | 69 | 70 |
| | 13, subfamily D, member 1 | | | | |
| VSIG4 | V-set and immunoglobulin | NM_007268; | other | 71 | 72 |
| | domain containing 4 | NM_001100431; | | | |
| | | NM_001184830, | | | |
| | | NM_001184831 | | | |
| TAS2R4 | taste receptor, type 2, | NM_016944 | receptor | 73 | 74 |
| | member 4 | | | | |
| GPR18 | G protein-coupled receptor | NM_005292; | receptor | 75 | 76 |
| | 18 | NM_001098200 | | | |
| TAS2R3 | taste receptor, type 2, | NM_016943 | receptor | 77 | 78 |
| | member 3 | | | | |
| MR1 | major histocompatibility | NM_001531; | receptor | 79 | 80 |
| | complex, class I-related | Q95460; U22963; | | | |
| | | Q53GM1; | | | |
| | | NM_001531; | | | |
| | | Q53GM1; | | | |
| | | U22963; Q95460; | | | |
| | | NM_001194999, | | | |
| | | NM_001195000, | | | |
| | | NM_001195035 | | | |
| GPR34 | G protein-coupled receptor | NM_001097579 | receptor | 81 | 82 |
| | 34 | NM_005300 | | | |
| KCNAB2 | potassium voltage-gated | NM_003636 | transport | 83 | 84 |
| | channel, shaker-related | NM_172130 | | | |
| | subfamily, beta member 2 | | | | |
| KCNE3 | potassium voltage-gated | NM_005472 | transport | 85 | 86 |
| | channel, Isk-related family, | | | | |
| | member 3 | | | | |
| LAT2 | linker for activation of T | NM_032464 | other | 87 | 88 |
| | cells family, member 2 | NM_032463 | | | |
| | | NM_014146 | | | |
| MLC1 | megalencephalic | NM_015166 | transport | 89 | 90 |
| | leukoencephalopathy with | NM_139202 | | | |
| | subcortical cysts 1 | | | | |
| ENPP5 | ectonucleotide | NM_021572 | enzyme | 91 | 92 |
| | pyrophosphatase/phosphodiesterase | | | | |
| | 5 (putative function) | | | | |
| FLVCR1 | feline leukemia virus | NM_014053 | transport | 93 | 94 |
| | subgroup C cellular receptor 1 | | | | |
| GPR65 | G protein-coupled receptor | NM_003608 | receptor | 95 | 96 |
| | 65 | | | | |
| OPN3 | opsin 3 | NM_014322; | receptor | 97 | 98 |
| | | NM_001821 | | | |
| TAS2R13 | taste receptor, type 2, | NM_023920 | receptor | 99 | 100 |
| | member 13 | | | | |
| CLDN20 | claudin 20 | NM_001001346 | cell adhesion | 101 | 102 |
| SLC1A4 | solute carrier family 1 | NM_003038; | transport | 105 | 106 |
| | (glutamate/neutral amino | NM_001193493 | | | |
| | acid transporter), member 4 | | | | |
| CLDN10 | claudin 10 | NM_182848; | cell adhesion | 107 | 108 |
| | | NM_006984 | | | |
| ADAMTS2 | ADAM metallopeptidase | NM_014244; | enzyme | 109 | 110 |
| | with thrombospondin type 1 | NM_021599 | | | |
| | motif, 2 | | | | |
| TBXAS1 | thromboxane A synthase 1 | NM_001061; | enzyme | 111 | 112 |
| | (platelet) | NM_030984; | | | |
| | | NM_001130966, | | | |
| | | NM_001166253, | | | |
| | | NM_001166254 | | | |
| LAPTM5 | lysosomal protein | NM_006762 | transport | 113 | 114 |
| | transmembrane 5 | | | | |
| VAMP8 | vesicle-associated | NM_003761 | transport | 115 | 116 |
| | membrane protein 8 | | | | |
| | (endobrevin) | | | | |
| AKAP7 | A kinase (PRKA) anchor | NM_016377; | transport | 117 | 118 |
| | protein 7 | NM_138633; | | | |
| | | NM_004842 | | | |
| SEMA3C | sema domain, | NM_006379 | receptor | 119 | 120 |
| | immunoglobulin domain | | | | |
| | (Ig), short basic domain, | | | | |
| | secreted, (semaphorin) 3C | | | | |
| SLC38A1 | solute carrier family 38, | NM_001077484; | transport | 121 | 122 |
| | member 1 | Q9H2H9; | | | |
| | | NM_030674; | | | |
| | | Q9H2H9 | | | |
| CD302 | CD302 molecule | NM_014880 | receptor | 123 | 124 |
| PLBD1 | phospholipase B domain | NM_024829 | enzyme | 125 | 126 |
| | containing 1 | | | | |
| LOXL3 | lysyl oxidase-like 3 | NM_032603 | enzyme | 127 | 128 |
| FAM46C | family with sequence | NM_017709 | other | 129 | 130 |
| (includes | similarity 46, member C | | | | |
| EG: 54855) | | | | | |
| MFAP4 | microfibrillar-associated | NM_002404 | cell adhesion | 131 | 132 |
| | protein 4 | | | | |
| IQCB1 | IQ motif containing B1 | NM_001023570; | other | 133 | 134 |
| | | NM_001023571 | | | |
| FBN2 | fibrillin 2 | NM_001999 | structural | 135 | 136 |
| (includes | | | | | |
| EG: 2201) | | | | | |
| OGN | osteoglycin | NM_033014; | growth factor | 137 | 138 |
| | | NM_014057 | | | |
| OMD | osteomodulin | NM_005014 | cell adhesion | 139 | 140 |
| ASPN | asporin | NM_017680; | other | 141 | 142 |
| | | NM_001193335 | | | |
| PZP | pregnancy-zone protein | NM_002864 | other | 143 | 144 |
| HSN2 | hereditary sensory | NM_213655; | enzyme | 145 | 146 |
| | neuropathy, type II (WNK1) | NM_014823; | | | |
| | | NM_018979; | | | |
| | | NM_001184985 | | | |
| SERPINI2 | serpin peptidase inhibitor, | NM_006217 | other | 147 | 148 |
| | clade I (pancpin), member 2 | | | | |
| ECM2 | extracellular matrix protein | NM_001393 | cell adhesion | 149 | 150 |
| | 2, female organ and | | | | |
| | adipocyte specific | | | | |
| ERLIN1 | ER lipid raft associated 1 | NM_006459; | other | 151 | 152 |
| | | NM_001100626 | | | |
| CELR2_HUMAN | cadherin, EGF LAG seven- | Q9HCU4; | receptor | 153 | 154 |
| | pass G-type receptor 2 | Q5T2Y7; Q92566 | | | |
| | (flamingo homolog, | | | | |
| | Drosophila) | | | | |
| NPTN_HUMAN | neuroplastin | Q9Y639; | cell adhesion | 155 | 156 |
| | | B7Z4D3; | | | |
| | | B7ZLL2; | | | |
| | | Q17R52; Q59EJ9; | | | |
| | | Q6NVX7; | | | |
| | | Q9Y640 | | | |
| APMAP_HUMAN | chromosome 20 open | Q9HDC9; | enzyme | 157 | 158 |
| | reading frame 3 | A8K514; | | | |
| | | B4DXG1; | | | |
| | | Q6UVZ8; | | | |
| | | Q9GZS8; | | | |
| | | Q9NUB2 | | | |
| GBRA3_HUMAN | gamma-aminobutyric acid | P34903; Q8TAF9 | transport | 159 | 160 |
| | (GABA) A receptor, alpha 3 | | | | |
| DSG3_HUMAN | desmoglein 3 (pemphigus | P32926; A8K2V2 | cell adhesion | 161 | 162 |
| | vulgaris antigen) | | | | |
| PLXB2_HUMAN | plexin B2 | O15031; | receptor | 163 | 164 |
| | | A6QRH0; | | | |
| | | Q7KZU3; | | | |
| | | Q9BSU7 | | | |
| CRCM1_HUMAN | ORAI calcium release- | Q96D31; | transport | 165 | 166 |
| | activated calcium modulator 1 | Q3MHV3; | | | |
| | | Q6DHX2; | | | |
| | | Q96BP7; Q96K71 | | | |
| DAG1 | Dystroglycan | Q14118; | receptor | 167 | 168 |
| | | A8K6M7; | | | |
| | | Q969J9 | | | |
| C14ORF176 | Transmembrane protein | P0C7T8 | other | 169 | 170 |
| | C14orf176 | | | | |
| MPZL1 | Myelin protein zero-like | O95297; | receptor | 171 | 172 |
| | protein 1 | B2REB9; | | | |
| | | Q5R332; | | | |
| | | Q8IX11; | | | |
| | | Q9BWZ3; | | | |
| | | Q9NYK4; | | | |
| | | Q9UL20 | | | |
| CLDN17 | Claudin-17 | P56750; | cell adhesion | 173 | 174 |
| | | Q3MJB5; | | | |
| | | Q6UY37 | | | |
| GPR125 | Probable G-protein coupled | Q8IWK6; | receptor | 175 | 176 |
| | receptor 125 | Q6UXK9; | | | |
| | | Q86SQ5; | | | |
| | | Q8TC55 | | | |
| UPK1A | Uroplakin-1a | O00322; | other | 179 | 180 |
| | | Q3KNU5; | | | |
| | | Q3KNU6 | | | |
| ODZ3 | Teneurin-3 | Q9P273; | receptor | 181 | 182 |
| | | Q5XUL9; | | | |
| | | Q96SY2; | | | |
| | | Q9NV77; | | | |
| | | Q9NVW1; | | | |
| | | Q9NZJ2 | | | |
| DCC | Netrin receptor DCC | P43146 | receptor | 183 | 184 |
| KIAA0090 | Uncharacterized protein | Q8N766; | other | 185 | 186 |
| | KIAA0090 | A8K6F3; | | | |
| | | Q14700; | | | |
| | | Q5TG62; | | | |
| | | Q63HL0; | | | |
| | | Q63HL3; | | | |
| | | Q8NBH8 | | | |
| ACCN4 | Amiloride-sensitive cation | Q96FT7; | transport | 187 | 188 |
| | channel 4 | Q53SB7; | | | |
| | | Q6GMS1; | | | |
| | | Q6PIN9; | | | |
| | | Q9NQA4 | | | |
| CACNA1D | Voltage-dependent L-type | Q01668; Q13916; | transport | 189 | 190 |
| | calcium channel subunit | Q13931; | | | |
| | alpha-1D | Q9UDC3 | | | |
| CSPG4 | Chondroitin sulfate | Q6UVK1; | other | 191 | 192 |
| | proteoglycan 4 | D3DW77; | | | |
| | | Q92675 | | | |
| FAT2 | Protocadherin Fat 2 | Q9NYQ8; | cell adhesion | 195 | 196 |
| | | O75091; Q9NSR7 | | | |
| LRP12 | Low-density lipoprotein | Q9Y561; A8K137 | receptor | 197 | 198 |
| | receptor-related protein 12 | | | | |
| NPY2R | Neuropeptide Y receptor | P49146; Q13281; | receptor | 199 | 200 |
| | type 2 | Q13457; | | | |
| | | Q4W5G7; | | | |
| | | Q6AZZ6; | | | |
| | | Q9UE67 | | | |
| OR11H4 | Olfactory receptor 11H4 | Q8NGC9; | receptor | 201 | 202 |
| | | B2RNQ4; Q6IF07 | | | |
| PCDHA4 | Protocadherin alpha-4 | Q9UN74; | cell adhesion | 203 | 204 |
| | | O75285; Q2M253 | | | |
| PCDHAC1 | Protocadherin alpha-C1 | Q9H158; | cell adhesion | 205 | 206 |
| | | Q9Y5F5; Q9Y5I5 | | | |
| RHBDD2 | Rhomboid domain- | Q6NTF9; | other | 207 | 208 |
| | containing protein 2 | Q7L534; | | | |
| | | Q9H5W6; | | | |
| | | Q9UDT2 | | | |
| SCN5A | Sodium channel protein type | Q14524; | transport | 209 | 210 |
| | 5 subunit alpha | A5H1P8; | | | |
| | | A6N922; | | | |
| | | A6N923; | | | |
| | | B2RTU0; | | | |
| | | Q75RX9; | | | |
| | | Q75RY0; | | | |
| | | Q86UR3; | | | |
| | | Q8IZC9; Q96J69 | | | |
| SERINC5 | Serine incorporator 5 | Q86VE9; | transport | 211 | 212 |
| | | Q495A4; Q495A6 | | | |
| SLC12A1 | Solute carrier family 12 | Q13621; A8JYA2 | transport | 213 | 214 |
| | member 1 | | | | |
| SLC46A1 | Proton-coupled folate | Q96NT5; | transport | 215 | 216 |
| | transporter | Q1HE20; | | | |
| | | Q86T92; | | | |
| | | Q8TEG3; | | | |
| | | Q96FL0 | | | |
| SLCO1B1 | Solute carrier organic anion | Q9Y6L6; | transport | 217 | 218 |
| | transporter family member | B2R7G2; | | | |
| | 1B1 | Q9NQ37; | | | |
| | | Q9UBF3; | | | |
| | | Q9UH89 | | | |
| ANO2 | Anoctamin-2 | Q9NQ90; | transport | 219 | 220 |
| | | C4N787; Q9H847 | | | |
| ABCA12 | ATP-binding cassette sub- | Q86UK0; | transport | 221 | 222 |
| | family A member 12 | Q53QE2; | | | |
| | | Q53S55; | | | |
| | | Q8IZW6; | | | |
| | | Q96JT3; | | | |
| | | Q9Y4M5 | | | |
| CPM | Carboxypeptidase M | P14384; B2R800; | enzyme | 223 | 224 |
| | | Q9H2K9 | | | |
| ATP10A | Probable phospholipid | O60312; Q969I4 | transport | 229 | 230 |
| | transporting ATPase VA | | | | |
| CHRNG | Acetylcholine receptor | P07510; | receptor | 231 | 232 |
| | subunit gamma | B3KWM8; | | | |
| | | Q53RG2 | | | |
| CACNA1B | Voltage-dependent N-type | Q00975; | transport | 235 | 236 |
| | calcium channel subunit | B1AQK5 | | | |
| | alpha-1B | | | | |
| SPAG11B | sperm associated antigen | NM_016512 | other | 237 | 238 |
| | 11B | | | | |
| FRAS1 | Fraser Syndrome 1 | NM_025074; | other | 239 | 240 |
| | | NM_001166133 | | | |
| ILDR1 | immunoglobulin-like | NM_175924 | receptor | 241 | 242 |
| | domain containing receptor 1 | | | | |
| EPB41L1 | EPB41L1—erythrocyte | NM_012156; | structural | 243 | 244 |
| | membrane protein band 4.1 | NM_177996 | | | |
| | like 1 | | | | |
| BAGE | B melanoma antigen | NM_001187 | other | 245 | 246 |
| AMPA2 | glutamate receptor, | NM_000826; | transport | 247 | 248 |
| | ionotropic, AMPA2 | NM_001083619, | | | |
| | | NM_001083620 | | | |
| SYT15 | synaptotagmin XV | NM_031912, | transport | 249 | 250 |
| | | NM_181519 | | | |
| NFASC | NFASC—neurofascin | NM_015090; | cell adhesion | 251 | 252 |
| | homolog (chicken) | NM_001005388, | | | |
| | | NM_001160331, | | | |
| | | NM_001160332, | | | |
| | | NM_001160333, | | | |
| | | NM_001005389 | | | |
| NLGN1 | EST (IMAGE: 2110090) | AI401535 | enzyme | 253 | 254 |
| SLC30A10 | solute carrier family 30, | NM_001004433 | transport | 255 | 256 |
| | member 10 | | | | |
| UNC93A | UNC-93 homologue A | NM_018974; | other | 257 | 258 |
| | (C. elegans) | NM_001143947 | | | |
| OR1C1 | Olfactory receptor, family 1, | NM_012353 | receptor | 259 | 260 |
| | subfamily C, member 1 | | | | |
| TMTC4 | transmembrane and | NM_001079669; | other | 261 | 262 |
| | tetratricopeptide repeat | NM_032813 | | | |
| | containing 4 | | | | |
| CLCN4 | chloride channel 4 | NM_001830 | transport | 263 | 264 |
| OR12D3 | olfactory receptor, family | NM_030959 | receptor | 265 | 266 |
| | 12, subfamily D, member 3 | | | | |
| BTNL8 | Butyrophilin-like protein 8 | NM_024850; | other | 267 | 268 |
| | precursor | NM_001040462, | | | |
| | | NM_001159707, | | | |
| | | NM_001159708, | | | |
| | | NM_001159709, | | | |
| | | NM_001159710 | | | |
| SLC7A14 | solute carrier, family 7 | NM_020949 | transport | 269 | 270 |
| | member 14 | | | | |
| OR7D4 | olfactory receptor, family 7 | NM_001005191.1 | receptor | 271 | 272 |
| | subfamily D member 4 | | | | |
| MUC12 | mucin 12, cell surface | AF147791 | other | 273 | 274 |
| | associated | | | | |
| TRGC2 | T-cell receptor gamma chain | BC039116 | receptor | 275 | 276 |
| | C region PT-gamma-1/2 | | | | |
| DEF109P1B | DEFb109—Defensin beta | NR_003668 | other | 277 | 278 |
| | 109 | | | | |
| KCNIP1 | Kv channel interacting | NM_001034837 | transport | 279 | 280 |
| | protein 1 (variant 1) | | | | |
| SLC45A4 | solute carrier family 45, | BC033223 | transport | 281 | 282 |
| | member 4 | | | | |
| ENPP6 | ectonucleotide | NM_153343 | enzyme | 283 | 284 |
| | pyrophosphatase/ | | | | |
| | phosphodiesterase 6 | | | | |
| PCDHB8 | protocadherin beta 8 | NM_019120 | cell adhesion | 285 | 286 |
| OR2T3 | olfactory receptor, family 2, | NM_001005495 | other | 287 | 288 |
| | sub family T, member 3 | | | | |
| OR5M10 | olfactory receptor family 5, | NM_001004741 | receptor | 289 | 290 |
| | subfamily M, member 10 | | | | |
| OR4S1 | olfactory receptor family 4, | NM_001004725 | receptor | 291 | 292 |
| | subfamily S, member 1 | | | | |
| GPR83 | G protein-coupled receptor | NM_016540 | receptor | 293 | 294 |
| | 83 | | | | |
| TAS2R19 | taste receptor, type 2, | NM_176888 | other | 295 | 296 |
| | member 19 | | | | |
| KAL1 | Kallmann syndrome 1 | NM_000216 | other | 297 | 298 |
| | sequence | | | | |
| SLCO1B3 | solute carrier organic anion | NM_019844 | transport | 299 | 300 |
| | transporter family, member | | | | |
| | 1B3 | | | | |
| | Gene and two pseudogenes | AL133267 | receptor | 301 | 302 |
| | for 7 transmembrane | | | | |
| | receptor (rhodopsin family) | | | | |
| | (olfactory receptor like) | | | | |
| | proteins and a 60S acidic | | | | |
| | ribosomal protein P2 | | | | |
| | (RPLP2) pseudogene | | | | |
| HLA_DQB1 | major histocompatability | M60028 | receptor | 303 | 304 |
| | complex, class II, DQ beta 1 | | | | |
| ALCAM | CD166 (ALCAM) activated | NM_001627 | cell adhesion | 305 | 306 |
| | leukocyte cell adhesion | | | | |
| | molecule | | | | |
| IL20RB | IL-20Rbeta—Interleukin 20 | NM_144717 | receptor | 307 | 308 |
| | receptor beta | | | | |
| PDPN | podoplanin-differentiation | NM_001006624; | transport | 309 | 310 |
| | factor; O-glycosylated | NM_006474, | | | |
| | | NM_198389, | | | |
| | | NM_001006625 | | | |
| CHRM3 | cholinergic receptor, | NM_000740 | receptor | 311 | 312 |
| | muscarinic 3 | | | | |
| ITGB1 | intergrin, beta 1 (fibronectin | NM_002211; | receptor | 313 | 314 |
| | receptor, beta polypeptide, | NM_133376, | | | |
| | antigen CD29 includes | NM_033668 | | | |
| | MDF2, MSK12) | | | | |
| SIGLEC8 | sialic acid binding Ig-like | NM_014442 | receptor | 315 | 316 |
| | lectin 8, CD329 | | | | |
| RAP1A | RAS-related protein RAP1A | NM_001010935; | enzyme | 317 | 318 |
| | | NM_002884 | | | |
| PLXNA2 | Plexin A2 | NM_025179 | receptor | 319 | 320 |
| KIR2DL3 | CD158b (KIR2DL3) killer | NM_014511 | receptor | 321 | 322 |
| | cell immunoglobulin-like | | | | |
| | receptor, 2 domains, ligand 3 | | | | |
| KLRK1 | CD314, killer cell lectin-like | NM_007360.2 | receptor | 323 | 324 |
| | receptor, subfamily K, | | | | |
| | member 1 | | | | |
| CX3CR1 | chemokine (C—X3—C) | NM_001337; | receptor | 325 | 326 |
| | receptor 1, CCRL1 | NM_001171171, | | | |
| | | NM_001171172, | | | |
| | | NM_001171174 | | | |
| GPR174 | G protein-coupled receptor | NM_032553 | receptor | 327 | 328 |
| | 174 | | | | |

* All accession numbers are based on databases as at 5 Oct. 2010.

In one example, a protein or nucleic acid falls within a class set out in any of Tables 2-6.

TABLE 2
Cell adhesion proteins and nucleic acids encoding same
that are upregulated in, on or secreted from EPCs.

| | | | | Exemplary | Exemplary |
|---|---|---|---|---|---|
| | | Reference | | Nucleotide | Amino Acid |
| Gene ID | Entrez Gene Name | Sequence(s) | Category | SEQ ID NO | SEQ ID NO |
| DSG2 | desmoglein 2 | NM_001943 | cell adhesion | 15 | 16 |
| EMB | embigin homolog | NM_198449; | cell adhesion | 1 | 2 |
| | | NR_003955 | | | |
| SIGLEC10 | sialic acid binding Ig-like | NM_033130; | cell adhesion | 23 | 24 |
| | lectin 10 | NM_001171156; | | | |
| | | NM_001171157; | | | |
| | | NM_001171158; | | | |
| | | NM_001171159; | | | |
| | | NM_001171160; | | | |
| | | NM_001171161 | | | |
| SIGLEC6 | sialic acid binding Ig-like | NM_001245; | cell adhesion | 25 | 26 |
| | lectin 6 | NM_198845; | | | |
| | | NM_198846; | | | |
| | | NM_001177547; | | | |
| | | NM_001177548; | | | |
| | | NM_001177549 | | | |
| PLXNC1 | plexin C1 | NM_005761 | cell adhesion | 7 | 8 |
| CLDN20 | claudin 20 | NM_001001346 | cell adhesion | 101 | 102 |
| CLDN10 | claudin 10 | NM_182848; | cell adhesion | 107 | 108 |
| | | NM_006984 | | | |
| MFAP4 | microfibrillar-associated | NM_002404 | cell adhesion | 131 | 132 |
| | protein 4 | | | | |
| OMD | osteomodulin | NM_005014 | cell adhesion | 139 | 140 |
| ECM2 | extracellular matrix protein | NM_001393 | cell adhesion | 149 | 150 |
| | 2, female organ and | | | | |
| | adipocyte specific | | | | |
| NPTN_HUMAN | neuroplastin | Q9Y639; | cell adhesion | 155 | 156 |
| | | B7Z4D3; | | | |
| | | B7ZLL2; | | | |
| | | Q17R52; Q59EJ9; | | | |
| | | Q6NVX7; | | | |
| | | Q9Y640 | | | |
| DSG3_HUMAN | desmoglein 3 (pemphigus | P32926; A8K2V2 | cell adhesion | 161 | 162 |
| | vulgaris antigen) | | | | |
| CLDN17 | Claudin-17 | P56750; | cell adhesion | 173 | 174 |
| | | Q3MJB5; | | | |
| | | Q6UY37 | | | |
| FAT2 | Protocadherin Fat 2 | Q9NYQ8; | cell adhesion | 195 | 196 |
| | | O75091; Q9NSR7 | | | |
| PCDHA4 | Protocadherin alpha-4 | Q9UN74; | cell adhesion | 203 | 204 |
| | | O75285; Q2M253 | | | |
| PCDHAC1 | Protocadherin alpha-C1 | Q9H158; | cell adhesion | 205 | 206 |
| | | Q9Y5F5; Q9Y5I5 | | | |
| NFASC | NFASC—neurofascin | NM_015090; | cell adhesion | 251 | 252 |
| | homolog (chicken) | NM_001005388, | | | |
| | | NM_001160331, | | | |
| | | NM_001160332, | | | |
| | | NM_001160333, | | | |
| | | NM_001005389 | | | |
| PCDHB8 | protocadherin beta 8 | NM_019120 | cell adhesion | 285 | 286 |
| ALCAM | CD166 (ALCAM) activated | NM_001627 | cell adhesion | 305 | 306 |
| | leukocyte cell adhesion | | | | |
| | molecule | | | | |

In one example, the protein is a cadherin (e.g., a desmoglein and/or a protocadherin) or the nucleic acid encodes same. For example, the protein is selected from the group consisting of desmoglein 2, desmoglein 3, Protocadherin Fat 2, Protocadherin alpha-4, Protocadherin alpha-C1 and protocadherin beta 8 or the nucleic acid encodes same.

In one example, the protein is a lectin or the nucleic acid encodes same. For example, the protein is selected from the group consisting of sialic acid binding Ig-like lectin 10, sialic acid binding Ig-like lectin 6 or the nucleic acid encodes same.

In one example, the protein is an immunoglobulin, cell adhesion protein, such as an embigin homolog or sialic acid binding Ig-like lectin 10 or sialic acid binding Ig-like lectin 6 or ALCAM pr the nucleic acid encodes same. In this regard, an immunoglobulin, cell adhesion protein is a cell adhesion protein comprising an immunoglobulin domain. The skilled artisan will be aware that an immunoglobulin domain is an art recognized protein structure, which generally (however not necessarily) comprises a 2-layer sandwich of between 7 and 9 antiparallel β-strands arranged in two β-sheets. For example, the immunoglobulin, cell adhesion protein is a member of the immunoglobulin superfamily.

TABLE 3
Transport proteins and nucleic acids encoding same that are
upregulated in, on or secreted from EPCs.

| | | | | Exemplary | Exemplary |
|---|---|---|---|---|---|
| | | Reference | | Nucleotide | Amino Acid |
| Gene ID | Entrez Gene Name | Sequence(s) | Category | SEQ ID NO | SEQ ID NO |
| SLC39A8 | solute carrier family 39 | NM_022154; | transport | 3 | 4 |
| | (zinc transporter), member 8 | NM_001135148; | | | |
| | | NM_001135147; | | | |
| | | NM_001135146 | | | |
| PKD2L1 | polycystic kidney disease 2- | Q9P0L9; O75972; | transport | 227 | 228 |
| | like 1 protein | Q5W039; | | | |
| | | Q9UP35; | | | |
| | | Q9UPA2 | | | |
| SLC1A5 | Neutral amino acid | Q15758; | transport | 225 | 226 |
| | transporter B(0) | A8K9H5; | | | |
| | | D0EYG6; | | | |
| | | O95720; | | | |
| | | Q96RL9; | | | |
| | | Q9BWQ3; | | | |
| | | Q9UNP2 | | | |
| SORL1(LRP9) | sortilin-related receptor, | NM_003105 | transport | 45 | 46 |
| | L(DLR class) A repeats- | | | | |
| | containing | | | | |
| SLC15A2 | solute carrier family 15 | NM_021082; | transport | 19 | 20 |
| | (H+/peptide transporter), | NM_001145998 | | | |
| | member 2 | | | | |
| SLC22A16 | solute carrier family 22 | NM_033125 | transport | 49 | 50 |
| | (organic cation/carnitine | | | | |
| | transporter), member 16 | | | | |
| SLC24A3 | solute carrier family 24 | NM_020689 | transport | 51 | 52 |
| | (sodium/potassium/calcium | | | | |
| | exchanger), member 3 | | | | |
| SLC2A5 | solute carrier family 2 | NM_003039; | transport | 53 | 54 |
| | (facilitated glucose/fructose | NM_001135585 | | | |
| | transporter), member 5 | | | | |
| SLC1A3 | solute carrier family 1 (glial | NM_004172; | transport | 103 | 104 |
| | high affinity glutamate | NM_001166695; | | | |
| | transporter), member 3 | NM_001166696 | | | |
| ABCC4 | ATP-binding cassette, sub- | NM_005845\|NM_001105515 | transport | 43 | 44 |
| | family C (CFTR/MRP), | | | | |
| | member 4 | | | | |
| SLC16A6 | solute carrier family 16, | NM_004694; | transport | 21 | 22 |
| | member 6 (monocarboxylic | NM_001174166 | | | |
| | acid transporter 7) | | | | |
| SLC8A1 | solute carrier family 8 | NM_021097; NM_001112800; | transport | 47 | 48 |
| | (sodium/calcium exchanger), | NM_001112801; | | | |
| | member 1 | NM_001112802 | | | |
| KCNQ5 | potassium voltage-gated | NM_019842; | transport | 59 | 60 |
| | channel | NM_001160130; | | | |
| | | NM_001160132; | | | |
| | | NM_001160133; | | | |
| | | NM_001160134 | | | |
| KCNAB2 | potassium voltage-gated | NM_003636 | transport | 83 | 84 |
| | channel, shaker-related | NM_172130 | | | |
| | subfamily, beta member 2 | | | | |
| KCNE3 | potassium voltage-gated | NM_005472 | transport | 85 | 86 |
| | channel, Isk-related family, | | | | |
| | member 3 | | | | |
| MLC1 | megalencephalic | NM_015166 | transport | 89 | 90 |
| | leukoencephalopathy with | NM_139202 | | | |
| | subcortical cysts 1 | | | | |
| FLVCR1 | feline leukemia virus | NM_014053 | transport | 93 | 94 |
| | subgroup C cellular receptor 1 | | | | |
| SLC1A4 | solute carrier family 1 | NM_003038; | transport | 105 | 106 |
| | (glutamate/neutral amino | NM_001193493 | | | |
| | acid transporter), member 4 | | | | |
| LAPTM5 | lysosomal protein | NM_006762 | transport | 113 | 114 |
| | transmembrane 5 | | | | |
| VAMP8 | vesicle-associated | NM_003761 | transport | 115 | 116 |
| | membrane protein 8 | | | | |
| | (endobrevin) | | | | |
| AKAP7 | A kinase (PRKA) anchor | NM_016377; | transport | 117 | 118 |
| | protein 7 | NM_138633; | | | |
| | | NM_004842 | | | |
| SLC38A1 | solute carrier family 38, | NM_001077484; | transport | 121 | 122 |
| | member 1 | Q9H2H9; | | | |
| | | NM_030674; | | | |
| | | Q9H2H9 | | | |
| CD302 | CD302 molecule | NM_014880 | receptor | 123 | 124 |
| GBRA3_HUMAN | gamma-aminobutyric acid | P34903; Q8TAF9 | transport | 159 | 160 |
| | (GABA) A receptor, alpha 3 | | | | |
| CRCM1_HUMAN | ORAI calcium release- | Q96D31; | transport | 165 | 166 |
| | activated calcium modulator 1 | Q3MHV3; | | | |
| | | Q6DHX2; | | | |
| | | Q96BP7; Q96K71 | | | |
| ACCN4 | Amiloride-sensitive cation | Q96FT7; | transport | 187 | 188 |
| | channel 4 | Q53SB7; | | | |
| | | Q6GMS1; | | | |
| | | Q6PIN9; | | | |
| | | Q9NQA4 | | | |
| CACNA1D | Voltage-dependent L-type | Q01668; Q13916; | transport | 189 | 190 |
| | calcium channel subunit | Q13931; | | | |
| | alpha-1D | Q9UDC3 | | | |
| SCN5A | Sodium channel protein type | Q14524; | transport | 209 | 210 |
| | 5 subunit alpha | A5H1P8; | | | |
| | | A6N922; | | | |
| | | A6N923; | | | |
| | | B2RTU0; | | | |
| | | Q75RX9; | | | |
| | | Q75RY0; | | | |
| | | Q86UR3; | | | |
| | | Q8IZC9; Q96J69 | | | |
| SERINC5 | Serine incorporator 5 | Q86VE9; | transport | 211 | 212 |
| | | Q495A4; Q495A6 | | | |
| SLC12A1 | Solute carrier family 12 | Q13621; A8JYA2 | transport | 213 | 214 |
| | member 1 | | | | |
| SLC46A1 | Proton-coupled folate | Q96NT5; | transport | 215 | 216 |
| | transporter | Q1HE20; | | | |
| | | Q86T92; | | | |
| | | Q8TEG3; | | | |
| | | Q96FL0 | | | |
| SLCO1B1 | Solute carrier organic anion | Q9Y6L6; | transport | 217 | 218 |
| | transporter family member | B2R7G2; | | | |
| | 1B1 | Q9NQ37; | | | |
| | | Q9UBF3; | | | |
| | | Q9UH89 | | | |
| ANO2 | Anoctamin-2 | Q9NQ90; | transport | 219 | 220 |
| | | C4N787; Q9H847 | | | |
| ABCA12 | ATP-binding cassette sub- | Q86UK0; | transport | 221 | 222 |
| | family A member 12 | Q53QE2; | | | |
| | | Q53S55; | | | |
| | | Q8IZW6; | | | |
| | | Q96JT3; | | | |
| | | Q9Y4M5 | | | |
| ATP10A | Probable phospholipid | O60312; Q969I4 | transport | 229 | 230 |
| | transporting ATPase VA | | | | |
| CACNA1B | Voltage-dependent N-type | Q00975; | transport | 235 | 236 |
| | calcium channel subunit | B1AQK5 | | | |
| | alpha-1B | | | | |
| AMPA2 | glutamate receptor, | NM_000826; | transport | 247 | 248 |
| | ionotropic, AMPA2 | NM_001083619, | | | |
| | | NM_001083620 | | | |
| SYT15 | synaptotagmin XV | NM_031912, | transport | 249 | 250 |
| | | NM_181519 | | | |
| SLC30A10 | solute carrier family 30, | NM_001004433 | transport | 255 | 256 |
| | member 10 | | | | |
| CLCN4 | chloride channel 4 | NM_001830 | transport | 263 | 264 |
| SLC7A14 | solute carrier, family 7 | NM_020949 | transport | 269 | 270 |
| | member 14 | | | | |
| KCNIP1 | Kv channel interacting | NM_001034837 | transport | 279 | 280 |
| | protein 1 (variant 1) | | | | |
| SLC45A4 | solute carrier family 45, | BC033223 | transport | 281 | 282 |
| | member 4 | | | | |
| SLCO1B3 | solute carrier organic anion | NM_019844 | transport | 299 | 300 |
| | transporter family, member | | | | |
| | 1B3 | | | | |
| PDPN | podoplanin-differentiation | NM_001006624; | transport | 309 | 310 |
| | factor; O-glycosylated | NM_006474, | | | |
| | | NM_198389, | | | |
| | | NM_001006625 | | | |

In one example, the protein is a solute carrier family protein or the nucleic acid encodes same. For example, the protein is selected from the group consisting of solute carrier family 39 (zinc transporter), member 8, solute carrier family 15 (H+/peptide transporter), member 2, solute carrier family 16, member 6 (monocarboxylic acid transporter 7), solute carrier family 8 (sodium/calcium exchanger), member 1, solute carrier family 22 (organic cation/carnitine transporter), member 16, solute carrier family 24 (sodium/potassium/calcium exchanger), member 3, solute carrier family 2 (facilitated glucose/fructose transporter), member 5, solute carrier family 1 (glial high affinity glutamate transporter), member 3, solute carrier family 1 (glutamate/neutral amino acid transporter), member 4, solute carrier family 38, member 1, solute carrier family 12 member 1, solute carrier family 30, member 10, solute carrier, family 7 member 14, solute carrier family 45, member 4 and solute carrier organic anion transporter family, member 1B3 or the nucleic acid encodes same.

In one example, the protein is an ion channel protein (e.g., a potassium channel and/or a sodium channel and/or a calcium channel) or a subunit thereof. For example, the protein is potassium voltage-gated channel, potassium voltage-gated channel, shaker-related subfamily, beta member 2, potassium voltage-gated channel, Isk-related family, member 3, amiloride-sensitive cation channel 4, voltage-dependent L-type calcium channel subunit alpha-1D, sodium channel protein type 5 subunit alpha, voltage-dependent N-type calcium channel subunit alpha-1B, chloride channel 4 and gamma-aminobutyric acid (GABA) A receptor, alpha 3 or the nucleic acid encodes same.

TABLE 4
Growth factor proteins and nucleic acids encoding same
that are upregulated in, on or secreted from EPCs.

| | | | | Exemplary | Exemplary |
|---|---|---|---|---|---|
| | | Reference | | Nucleotide | Amino Acid |
| Gene ID | Entrez Gene Name | Sequence(s) | Category | SEQ ID NO | SEQ ID NO |
| AREG | amphiregulin | NM_001657 | growth factor | 27 | 28 |
| NRG4 | neuregulin 4 | NM_138573 | growth factor | 37 | 38 |
| EPGN | epithelial mitogen homolog | NM_001013442 | growth factor | 39 | 40 |
| | (mouse) | | | | |
| OGN | osteoglycin | NM_033014; | growth factor | 137 | 138 |
| | | NM_014057 | | | |
| NPTN_HUMAN | neuroplastin | Q9Y639; | cell adhesion | 155 | 156 |
| | | B7Z4D3; | | | |
| | | B7ZLL2; | | | |
| | | Q17R52; Q59EJ9; | | | |
| | | Q6NVX7; | | | |
| | | Q9Y640 | | | |

TABLE 5
Receptor proteins and nucleic acids encoding same
that are upregulated in, on or secreted from EPCs.

| | | | | Exemplary | Exemplary |
|---|---|---|---|---|---|
| | | Reference | | Nucleotide | Amino Acid |
| Gene ID | Entrez Gene Name | Sequence(s) | Category | SEQ ID NO | SEQ ID NO |
| EMR2 | egf-like module containing, | NM_013447; | receptor | 17 | 18 |
| | mucin-like, hormone | NM_152916; | | | |
| | receptor-like 2 | NM_152919; | | | |
| | | NM_152917; | | | |
| | | NM_152920; | | | |
| | | NM_152921; | | | |
| | | NM_152918 | | | |
| SIRPB1 | signal-regulatory protein | NM_006065 | receptor | 331 | 332 |
| | beta 1 | | | | |
| CSF2RA | GM-CSF receptor subunit | NM_006140 | receptor | 333 | 334 |
| | alpha precursor | NM_172245 | | | |
| | | NM_172246 | | | |
| | | NM_172247 | | | |
| | | NM_172249 | | | |
| | | NM_001161529 | | | |
| | | NM_001161530 | | | |
| | | NM_001161531 | | | |
| | | NR_027760 | | | |
| | | NM_001161532 | | | |
| CNR2 | cannabinoid receptor 2 | NM_001841 | receptor | 33 | 34 |
| | (macrophage) | | | | |
| OR52B6 | olfactory receptor, family | NM_001005162 | receptor | 11 | 12 |
| | 52, subfamily B, member 6 | | | | |
| P2RY14 | purinergic receptor P2Y, G- | NM_014879; | receptor | 61 | 62 |
| | protein coupled, 14 | Q15391; | | | |
| | | BC034989; | | | |
| | | Q15391; | | | |
| | | NM_001081455 | | | |
| HTR1F | 5-hydroxytryptamine | NM_000866; | receptor | 63 | 64 |
| | (serotonin) receptor 1F | Q4QRI9; | | | |
| | | BC069125; | | | |
| | | P30939; | | | |
| | | BC069125; | | | |
| | | Q4QRI9 | | | |
| GPR183 | G protein-coupled receptor | NM_004951 | receptor | 67 | 68 |
| | 183 | | | | |
| OR13D1 | olfactory receptor, family | NM_001004484 | receptor | 69 | 70 |
| | 13, subfamily D, member 1 | | | | |
| TAS2R4 | taste receptor, type 2, | NM_016944 | receptor | 73 | 74 |
| | member 4 | | | | |
| GPR18 | G protein-coupled receptor | NM_005292; | receptor | 75 | 76 |
| | 18 | NM_001098200 | | | |
| TAS2R3 | taste receptor, type 2, | NM_016943 | receptor | 77 | 78 |
| | member 3 | | | | |
| MR1 | major histocompatibility | NM_001531; | receptor | 79 | 80 |
| | complex, class I-related | Q95460; U22963; | | | |
| | | Q53GM1; | | | |
| | | NM_001531; | | | |
| | | Q53GM1; | | | |
| | | U22963; Q95460; | | | |
| | | NM_001194999, | | | |
| | | NM_001195000, | | | |
| | | NM_001195035 | | | |
| GPR34 | G protein-coupled receptor | NM_001097579 | receptor | 81 | 82 |
| | 34 | NM_005300 | | | |
| GPR65 | G protein-coupled receptor | NM_003608 | receptor | 95 | 96 |
| | 65 | | | | |
| OPN3 | opsin 3 | NM_014322; | receptor | 97 | 98 |
| | | NM_001821 | | | |
| TAS2R13 | taste receptor, type 2, | NM_023920 | receptor | 99 | 100 |
| | member 13 | | | | |
| SEMA3C | sema domain, | NM_006379 | receptor | 119 | 120 |
| | immunoglobulin domain | | | | |
| | (Ig), short basic domain, | | | | |
| | secreted, (semaphorin) 3C | | | | |
| CD302 | CD302 molecule | NM_014880 | receptor | 123 | 124 |
| CELR2_HUMAN | cadherin, EGF LAG seven- | Q9HCU4; | receptor | 153 | 154 |
| | pass G-type receptor 2 | Q5T2Y7; Q92566 | | | |
| | (flamingo homolog, | | | | |
| | Drosophila) | | | | |
| PLXB2_HUMAN | plexin B2 | O15031; | receptor | 163 | 164 |
| | | A6QRH0; | | | |
| | | Q7KZU3; | | | |
| | | Q9BSU7 | | | |
| DAG1 | Dystroglycan | Q14118; | receptor | 167 | 168 |
| | | A8K6M7; | | | |
| | | Q969J9 | | | |
| MPZL1 | Myelin protein zero-like | O95297; | receptor | 171 | 172 |
| | protein 1 | B2REB9; | | | |
| | | Q5R332; | | | |
| | | Q8IX11; | | | |
| | | Q9BWZ3; | | | |
| | | Q9NYK4; | | | |
| | | Q9UL20 | | | |
| GPR125 | Probable G-protein coupled | Q8IWK6; | receptor | 175 | 176 |
| | receptor 125 | Q6UXK9; | | | |
| | | Q86SQ5; | | | |
| | | Q8TC55 | | | |
| ODZ3 | Teneurin-3 | Q9P273; | receptor | 181 | 182 |
| | | Q5XUL9; | | | |
| | | Q96SY2; | | | |
| | | Q9NV77; | | | |
| | | Q9NVW1; | | | |
| | | Q9NZJ2 | | | |
| DCC | Netrin receptor DCC | P43146 | receptor | 183 | 184 |
| LRP12 | Low-density lipoprotein | Q9Y561; A8K137 | receptor | 197 | 198 |
| | receptor-related protein 12 | | | | |
| NPY2R | Neuropeptide Y receptor | P49146; Q13281; | receptor | 199 | 200 |
| | type 2 | Q13457; | | | |
| | | Q4W5G7; | | | |
| | | Q6AZZ6; | | | |
| | | Q9UE67 | | | |
| OR11H4 | Olfactory receptor 11H4 | Q8NGC9; | receptor | 201 | 202 |
| | | B2RNQ4; Q6IF07 | | | |
| CHRNG | Acetylcholine receptor | P07510; | receptor | 231 | 232 |
| | subunit gamma | B3KWM8; | | | |
| | | Q53RG2 | | | |
| ILDR1 | immunoglobulin-like | NM_175924 | receptor | 241 | 242 |
| | domain containing receptor 1 | | | | |
| OR1C1 | Olfactory receptor, family 1, | NM_012353 | receptor | 259 | 260 |
| | subfamily C, member 1 | | | | |
| OR12D3 | olfactory receptor, family | NM_030959 | receptor | 265 | 266 |
| | 12, subfamily D, member 3 | | | | |
| OR7D4 | olfactory receptor, family 7 | NM_001005191.1 | receptor | 271 | 272 |
| | subfamily D member 4 | | | | |
| TRGC2 | T-cell receptor gamma chain | BC039116 | receptor | 275 | 276 |
| | C region PT-gamma-1/2 | | | | |
| OR5M10 | olfactory receptor family 5, | NM_001004741 | receptor | 289 | 290 |
| | subfamily M, member 10 | | | | |
| OR4S1 | olfactory receptor family 4, | NM_001004725 | receptor | 291 | 292 |
| | subfamily S, member 1 | | | | |
| GPR83 | G protein-coupled receptor | NM_016540 | receptor | 293 | 294 |
| | 83 | | | | |
| | Gene and two pseudogenes | AL133267 | receptor | 301 | 302 |
| | for 7 transmembrane | | | | |
| | receptor (rhodopsin family) | | | | |
| | (olfactory receptor like) | | | | |
| | proteins and a 60S acidic | | | | |
| | ribosomal protein P2 | | | | |
| | (RPLP2) pseudogene | | | | |
| HLA_DQB1 | major histocompatability | M60028 | receptor | 303 | 304 |
| | complex, class II, DQ beta 1 | | | | |
| IL20RB | IL-20Rbeta - Interleukin 20 | NM_144717 | receptor | 307 | 308 |
| | receptor beta | | | | |
| CHRM3 | cholinergic receptor, | NM_000740 | receptor | 311 | 312 |
| | muscarinic 3 | | | | |
| ITGB1 | intergrin, beta 1 (fibronectin | NM_002211; | receptor | 313 | 314 |
| | receptor, beta polypeptide, | NM_133376, | | | |
| | antigen CD29 includes | NM_033668 | | | |
| | MDF2, MSK12) | | | | |
| SIGLEC8 | sialic acid binding Ig-like | NM_014442 | receptor | 315 | 316 |
| | lectin 8, CD329 | | | | |
| PLXNA2 | Plexin A2 | NM_025179 | receptor | 319 | 320 |
| KIR2DL3 | CD158b (KIR2DL3) killer | NM_014511 | receptor | 321 | 322 |
| | cell immunoglobulin-like | | | | |
| | receptor, 2 domains, ligand 3 | | | | |
| KLRK1 | CD314, killer cell lectin-like | NM_007360.2 | receptor | 323 | 324 |
| | receptor, subfamily K, | | | | |
| | member 1 | | | | |
| CX3CR1 | chemokine (C—X3—C) | NM_001337; | receptor | 325 | 326 |
| | receptor 1, CCRL1 | NM_001171171, | | | |
| | | NM_001171172, | | | |
| | | NM_001171174 | | | |
| GPR174 | G protein-coupled receptor | NM_032553 | receptor | 327 | 328 |
| | 174 | | | | |

In one example, the protein is an olfactory receptor, or the nucleic acid encodes same. For example, the protein is selected from the group consisting of olfactory receptor, family 52, subfamily B, member 6, olfactory receptor, family 13, subfamily D, member 1, Olfactory receptor 11H4, olfactory receptor, family 1, subfamily C, member 1, olfactory receptor, family 7 subfamily D member 4, olfactory receptor family 5, subfamily M, member 10 and olfactory receptor family 4, subfamily S, member 1 or the nucleic acid encodes same.

In another example, the protein is a taste receptor. For example, the protein is selected from the group consisting of taste receptor, type 2, member 4, taste receptor, type 2, member 3, taste receptor, type 2, member 13, and taste receptor, type 2, member 1 or the nucleic acid encodes same.

In a further example, the protein is a G protein coupled receptor. For example, the protein is selected from the group consisting of G protein-coupled receptor 183, G protein-coupled receptor 18, G protein-coupled receptor 34, Probable G-protein coupled receptor 125, G protein-coupled receptor 83, chemokine (C-X3-C) receptor 1 and G protein coupled receptor 174, CCRL1 or the nucleic acid encodes same.

TABLE 6
Enzyme proteins and nucleic acids encoding same
that are upregulated in, on or secreted from EPCs.

| | | | | Exemplary | Exemplary |
|---|---|---|---|---|---|
| | | Reference | | Nucleotide | Amino Acid |
| Gene ID | Entrez Gene Name | Sequence(s) | Category | SEQ ID NO | SEQ ID NO |
| LOXL4 | lysyl oxidase-like 4 | NM_032211 | enzyme | 337 | 338 |
| ADCY7 | adenylate cyclase 7 | NM_001114 | enzyme | 13 | 14 |
| NCSTN | Nicastrin | Q92542; Q5T207; | enzyme | 177 | 178 |
| | | Q86VV5 | | | |
| INSRR | insulin receptor-related | P14616; O60724; | enzyme | 233 | 234 |
| | protein | Q5VZS3 | | | |
| DPP6 | dipeptidyl aminopeptidase- | P42658 | enzyme | 193 | 194 |
| | like protein 6 | | | | |
| ADAM10 | Disintegrin and | NM_001110 | enzyme | 329 | 330 |
| | metalloproteinase domain- | | | | |
| | containing protein 10 | | | | |
| PRSS21 | protease, serine, 21 | NM_006799; | enzyme | 35 | 36 |
| | (testisin) | NM_144956; | | | |
| | | NM_144957 | | | |
| RHBDD1 | rhomboid domain | NM_032276; | enzyme | 41 | 42 |
| | containing 1 | NM_001167608 | | | |
| ENPP5 | ectonucleotide | NM_021572 | enzyme | 91 | 92 |
| | pyrophosphatase/phosphodie | | | | |
| | sterase 5 (putative function) | | | | |
| ADAMTS2 | ADAM metallopeptidase | NM_014244; | enzyme | 109 | 110 |
| | with thrombospondin type 1 | NM_021599 | | | |
| | motif, 2 | | | | |
| TBXAS1 | thromboxane A synthase 1 | NM_001061; | enzyme | 111 | 112 |
| | (platelet) | NM_030984; | | | |
| | | NM_001130966, | | | |
| | | NM_001166253, | | | |
| | | NM_001166254 | | | |
| PLBD1 | phospholipase B domain | NM_024829 | enzyme | 125 | 126 |
| | containing 1 | | | | |
| LOXL3 | lysyl oxidase-like 3 | NM_032603 | enzyme | 127 | 128 |
| CPM | Carboxypeptidase M | P14384; B2R800; | enzyme | 223 | 224 |
| | | Q9H2K9 | | | |
| NLGN1 | EST (IMAGE: 2110090) | AI401535 | enzyme | 253 | 254 |
| ENPP6 | ectonucleotide | NM_153343 | enzyme | 283 | 284 |
| | pyrophosphatase/ | | | | |
| | phosphodiesterase 6 | | | | |
| RAP1A | RAS-related protein RAP1A | NM_001010935; | enzyme | 317 | 318 |
| | | NM_002884 | | | |

In one example, the protein is a peptidase and/or a protease, or the nucleic acid encodes same. For example, the protein is selected from the group consisting of protease, serine, 21 (testisin), Disintegrin and metalloproteinase domain-containing protein 10, ADAM metallopeptidase with thrombospondin type 1 motif, 2, dipeptidyl aminopeptidase-like protein 6, and carboxypeptidase M or the nucleic acid encodes same.

In one example, a protein comprises an immunoglobulin domain or an immunoglobulin-like domain. Exemplary proteins falling within this class are embigin, Siglec6, Siglec8, Siglec10, VSIG4, SEMA3C, ILDR1, TRGC2, ALCAM, HLA-DQB1, NFASC, and KIR2DL3.

An exemplary protein or nucleic acid comprises a sequence at least about 75% nucleotide or amino acid sequence identity to the nucleotide or amino acid sequence set forth in any one of Tables 1 to 6, for example at least about 80% sequence identity, preferably at least about 85%, such as at least about 90%, such as at least about 91%, e.g., at least about 92%, e.g., at least about 93%, e.g., at least about 94%, for example at least about 95% e.g., at least about 96%, e.g., at least about 97%, e.g., at least about 98%, for example at least about 99% or 100%. The present disclosure is not to be restricted to the use of the exemplified Homo sapiens nucleic acids or proteins because, as will be known to those skilled in the art, it is possible to identify naturally-occurring variants and/or mutants of said nucleic acids and/or proteins using standard techniques, including in silico analysis, e.g., using BLAST.

The % identity of a nucleic acid or polypeptide is determined by GAP (Needleman and Wunsch, 1970) analysis (GCG program) with a gap creation penalty=5, and a gap extension penalty=0.3. The query sequence is at least 50 residues in length, and the GAP analysis aligns the two sequences over a region of at least 50 residues. For example, the query sequence is at least 100 residues in length and the GAP analysis aligns the two sequences over a region of at least 100 residues. For example, the two sequences are aligned over their entire length.

In one example, reference to a protein or nucleic acid shall be taken to be reference to an embigin homolog protein or nucleic acid encoding same.

In one example, reference to a protein or nucleic acid shall be taken to be reference to a solute carrier family 39 (zinc transporter), member 8 protein or nucleic acid encoding same.

In one example, reference to a protein or nucleic acid shall be taken to be reference to a transmembrane 7 superfamily member 3 protein or nucleic acid encoding same.

In one example, reference to a protein or nucleic acid shall be taken to be reference to a plexin C1 protein or nucleic acid encoding same.

In one example, reference to a protein or nucleic acid shall be taken to be reference to a natural killer cell group 7 sequence protein or nucleic acid encoding same.

In one example, reference to a protein or nucleic acid shall be taken to be reference to an olfactory receptor, family 52, subfamily B, member 6 protein or nucleic acid encoding same.

In one example, reference to a protein or nucleic acid shall be taken to be reference to an adenylate cyclase 7 protein or nucleic acid encoding same.

In one example, reference to a protein or nucleic acid shall be taken to be reference to a desmoglein 2 protein or nucleic acid encoding same.

In one example, reference to a protein or nucleic acid shall be taken to be reference to an egf-like module containing, mucin-like, hormone receptor-like 2 protein or nucleic acid encoding same.

In one example, reference to a protein or nucleic acid shall be taken to be reference to a solute carrier family 15 (H+/peptide transporter), member 2 protein or nucleic acid encoding same.

In one example, reference to a protein or nucleic acid shall be taken to be reference to a solute carrier family 16, member 6 (monocarboxylic acid transporter 7) protein or nucleic acid encoding same.

In one example, reference to a protein or nucleic acid shall be taken to be reference to a sialic acid binding Ig-like lectin 10 protein or nucleic acid encoding same.

In one example, reference to a protein or nucleic acid shall be taken to be reference to a sialic acid binding Ig-like lectin 6 protein or nucleic acid encoding same.

In one example, reference to a protein or nucleic acid shall be taken to be reference to an amphiregulin protein or nucleic acid encoding same.

In one example, reference to a protein or nucleic acid shall be taken to be reference to an integral membrane protein 2A protein or nucleic acid encoding same.

In one example, reference to a protein or nucleic acid shall be taken to be reference to a glycoprotein M6B protein or nucleic acid encoding same.

In one example, reference to a protein or nucleic acid shall be taken to be reference to a cannabinoid receptor 2 (macrophage) protein or nucleic acid encoding same.

In one example, reference to a protein or nucleic acid shall be taken to be reference to a protease, serine, 21 (testisin) protein or nucleic acid encoding same.

In one example, reference to a protein or nucleic acid shall be taken to be reference to a neuregulin 4 protein or nucleic acid encoding same.

In one example, reference to a protein or nucleic acid shall be taken to be reference to an epithelial mitogen homolog (mouse) protein or nucleic acid encoding same.

In one example, reference to a protein or nucleic acid shall be taken to be reference to a rhomboid domain containing 1 protein or nucleic acid encoding same.

In one example, reference to a protein or nucleic acid shall be taken to be reference to an ATP-binding cassette, sub-family C (CFTR/MRP), member 4 protein or nucleic acid encoding same.

In one example, reference to a protein or nucleic acid shall be taken to be reference to a sortilin-related receptor, L(DLR class) A repeats-containing protein or nucleic acid encoding same.

In one example, reference to a protein or nucleic acid shall be taken to be reference to a solute carrier family 8 (sodium/calcium exchanger), member 1 protein or nucleic acid encoding same.

In one example, reference to a protein or nucleic acid shall be taken to be reference to a solute carrier family 22 (organic cation/carnitine transporter), member 16 protein or nucleic acid encoding same.

In one example, reference to a protein or nucleic acid shall be taken to be reference to a solute carrier family 24 (sodium/potassium/calcium exchanger), member 3 protein or nucleic acid encoding same.

In one example, reference to a protein or nucleic acid shall be taken to be reference to a solute carrier family 2 (facilitated glucose/fructose transporter), member 5 protein or nucleic acid encoding same.

In one example, reference to a protein or nucleic acid shall be taken to be reference to a NCK-associated protein 1-like protein or nucleic acid encoding same.

In one example, reference to a protein or nucleic acid shall be taken to be reference to an ecotropic viral integration site 2B protein or nucleic acid encoding same.

In one example, reference to a protein or nucleic acid shall be taken to be reference to a potassium voltage-gated channel protein or nucleic acid encoding same.

In one example, reference to a protein or nucleic acid shall be taken to be reference to a purinergic receptor P2Y, G-protein coupled, 14 protein or nucleic acid encoding same.

In one example, reference to a protein or nucleic acid shall be taken to be reference to a 5-hydroxytryptamine (serotonin) receptor 1F protein or nucleic acid encoding same.

In one example, reference to a protein or nucleic acid shall be taken to be reference to a T cell receptor associated transmembrane adaptor 1 protein or nucleic acid encoding same.

In one example, reference to a protein or nucleic acid shall be taken to be reference to a G protein-coupled receptor 183 protein or nucleic acid encoding same.

In one example, reference to a protein or nucleic acid shall be taken to be reference to an olfactory receptor, family 13, subfamily D, member 1 protein or nucleic acid encoding same.

In one example, reference to a protein or nucleic acid shall be taken to be reference to a V-set and immunoglobulin domain containing 4 protein or nucleic acid encoding same.

In one example, reference to a protein or nucleic acid shall be taken to be reference to a taste receptor, type 2, member 4 protein or nucleic acid encoding same.

In one example, reference to a protein or nucleic acid shall be taken to be reference to a G protein-coupled receptor 18 protein or nucleic acid encoding same.

In one example, reference to a protein or nucleic acid shall be taken to be reference to a taste receptor, type 2, member 3 protein or nucleic acid encoding same.

In one example, reference to a protein or nucleic acid shall be taken to be reference to a major histocompatibility complex, class I-related protein or nucleic acid encoding same.

In one example, reference to a protein or nucleic acid shall be taken to be reference to a G protein-coupled receptor 34 protein or nucleic acid encoding same.

In one example, reference to a protein or nucleic acid shall be taken to be reference to a potassium voltage-gated channel, shaker-related subfamily, beta member 2 protein or nucleic acid encoding same.

In one example, reference to a protein or nucleic acid shall be taken to be reference to a potassium voltage-gated channel, Isk-related family, member 3 protein or nucleic acid encoding same.

In one example, reference to a protein or nucleic acid shall be taken to be reference to a linker for activation of T cells family, member 2 protein or nucleic acid encoding same.

In one example, reference to a protein or nucleic acid shall be taken to be reference to a megalencephalic leukoencephalopathy with subcortical cysts 1 protein or nucleic acid encoding same.

In one example, reference to a protein or nucleic acid shall be taken to be reference to an ectonucleotide pyrophosphatase/phosphodiesterase 5 (putative function) protein or nucleic acid encoding same.

In one example, reference to a protein or nucleic acid shall be taken to be reference to a feline leukemia virus subgroup C cellular receptor 1 protein or nucleic acid encoding same.

In one example, reference to a protein or nucleic acid shall be taken to be reference to a G protein-coupled receptor 65 protein or nucleic acid encoding same.

In one example, reference to a protein or nucleic acid shall be taken to be reference to an opsin 3 protein or nucleic acid encoding same.

In one example, reference to a protein or nucleic acid shall be taken to be reference to a taste receptor, type 2, member 13 protein or nucleic acid encoding same.

In one example, reference to a protein or nucleic acid shall be taken to be reference to a claudin 20 protein or nucleic acid encoding same.

In one example, reference to a protein or nucleic acid shall be taken to be reference to a solute carrier family 1 (glial high affinity glutamate transporter), member 3 protein or nucleic acid encoding same.

In one example, reference to a protein or nucleic acid shall be taken to be reference to a solute carrier family 1 (glutamate/neutral amino acid transporter), member 4 protein or nucleic acid encoding same.

In one example, reference to a protein or nucleic acid shall be taken to be reference to a claudin 10 protein or nucleic acid encoding same.

In one example, reference to a protein or nucleic acid shall be taken to be reference to an ADAM metallopeptidase with thrombospondin type 1 motif, 2 protein or nucleic acid encoding same.

In one example, reference to a protein or nucleic acid shall be taken to be reference to a thromboxane A synthase 1 (platelet) protein or nucleic acid encoding same.

In one example, reference to a protein or nucleic acid shall be taken to be reference to a lysosomal protein transmembrane 5 protein or nucleic acid encoding same.

In one example, reference to a protein or nucleic acid shall be taken to be reference to a vesicle-associated membrane protein 8 (endobrevin) protein or nucleic acid encoding same.

In one example, reference to a protein or nucleic acid shall be taken to be reference to an A kinase (PRKA) anchor protein 7 protein or nucleic acid encoding same.

In one example, reference to a protein or nucleic acid shall be taken to be reference to a sema domain, immunoglobulin domain (Ig), short basic domain, secreted, (semaphorin) 3C protein or nucleic acid encoding same.

In one example, reference to a protein or nucleic acid shall be taken to be reference to a solute carrier family 38, member 1 protein or nucleic acid encoding same.

In one example, reference to a protein or nucleic acid shall be taken to be reference to a CD302 protein or nucleic acid encoding same.

In one example, reference to a protein or nucleic acid shall be taken to be reference to a phospholipase B domain containing 1 protein or nucleic acid encoding same.

In one example, reference to a protein or nucleic acid shall be taken to be reference to a lysyl oxidase-like 3 protein or nucleic acid encoding same.

In one example, reference to a protein or nucleic acid shall be taken to be reference to a family with sequence similarity 46, member C protein or nucleic acid encoding same.

In one example, reference to a protein or nucleic acid shall be taken to be reference to a microfibrillar-associated protein 4 protein or nucleic acid encoding same.

In one example, reference to a protein or nucleic acid shall be taken to be reference to an IQ motif containing B1 protein or nucleic acid encoding same.

In one example, reference to a protein or nucleic acid shall be taken to be reference to a fibrillin 2 protein or nucleic acid encoding same.

In one example, reference to a protein or nucleic acid shall be taken to be reference to an osteoglycin protein or nucleic acid encoding same.

In one example, reference to a protein or nucleic acid shall be taken to be reference to an osteomodulin protein or nucleic acid encoding same.

In one example, reference to a protein or nucleic acid shall be taken to be reference to an asporin protein or nucleic acid encoding same.

In one example, reference to a protein or nucleic acid shall be taken to be reference to a pregnancy-zone protein or nucleic acid encoding same.

In one example, reference to a protein or nucleic acid shall be taken to be reference to a hereditary sensory neuropathy, type II (WNK1) protein or nucleic acid encoding same.

In one example, reference to a protein or nucleic acid shall be taken to be reference to a serpin peptidase inhibitor, clade I (pancpin), member 2 protein or nucleic acid encoding same.

In one example, reference to a protein or nucleic acid shall be taken to be reference to an extracellular matrix protein 2, female organ and adipocyte specific protein or nucleic acid encoding same.

In one example, reference to a protein or nucleic acid shall be taken to be reference to an ER lipid raft associated 1 protein or nucleic acid encoding same.

In one example, reference to a protein or nucleic acid shall be taken to be reference to a cadherin, EGF LAG seven-pass G-type receptor 2 (flamingo homolog, Drosophila) protein or nucleic acid encoding same.

In one example, reference to a protein or nucleic acid shall be taken to be reference to a neuroplastin protein or nucleic acid encoding same.

In one example, reference to a protein or nucleic acid shall be taken to be reference to a protein encoded by chromosome 20 open reading frame 3 or nucleic acid encoding same.

In one example, reference to a protein or nucleic acid shall be taken to be reference to a gamma-aminobutyric acid (GABA) A receptor, alpha 3 protein or nucleic acid encoding same.

In one example, reference to a protein or nucleic acid shall be taken to be reference to a desmoglein 3 (pemphigus vulgaris antigen) protein or nucleic acid encoding same.

In one example, reference to a protein or nucleic acid shall be taken to be reference to a plexin B2 protein or nucleic acid encoding same.

In one example, reference to a protein or nucleic acid shall be taken to be reference to an ORAI calcium release-activated calcium modulator 1 protein or nucleic acid encoding same.

In one example, reference to a protein or nucleic acid shall be taken to be reference to a dystroglycan protein or nucleic acid encoding same.

In one example, reference to a protein or nucleic acid shall be taken to be reference to a transmembrane protein C14orf176 protein or nucleic acid encoding same.

In one example, reference to a protein or nucleic acid shall be taken to be reference to a myelin protein zero-like protein 1 protein or nucleic acid encoding same.

In one example, reference to a protein or nucleic acid shall be taken to be reference to a claudin-17 protein or nucleic acid encoding same.

In one example, reference to a protein or nucleic acid shall be taken to be reference to a probable G-protein coupled receptor 125 protein or nucleic acid encoding same.

In one example, reference to a protein or nucleic acid shall be taken to be reference to a nicastrin protein or nucleic acid encoding same.

In one example, reference to a protein or nucleic acid shall be taken to be reference to an uroplakin-1a protein or nucleic acid encoding same.

In one example, reference to a protein or nucleic acid shall be taken to be reference to a teneurin-3 protein or nucleic acid encoding same.

In one example, reference to a protein or nucleic acid shall be taken to be reference to a netrin receptor DCC protein or nucleic acid encoding same.

In one example, reference to a protein or nucleic acid shall be taken to be reference to an uncharacterized protein KIAA0090 protein or nucleic acid encoding same.

In one example, reference to a protein or nucleic acid shall be taken to be reference to an amiloride-sensitive cation channel 4 protein or nucleic acid encoding same.

In one example, reference to a protein or nucleic acid shall be taken to be reference to a voltage-dependent L-type calcium channel subunit alpha-1D protein or nucleic acid encoding same.

In one example, reference to a protein or nucleic acid shall be taken to be reference to a chondroitin sulfate proteoglycan 4 protein or nucleic acid encoding same.

In one example, reference to a protein or nucleic acid shall be taken to be reference to a dipeptidyl aminopeptidase-like protein 6 protein or nucleic acid encoding same.

In one example, reference to a protein or nucleic acid shall be taken to be reference to a protocadherin Fat 2 protein or nucleic acid encoding same.

In one example, reference to a protein or nucleic acid shall be taken to be reference to a low-density lipoprotein receptor-related protein 12 protein or nucleic acid encoding same.

In one example, reference to a protein or nucleic acid shall be taken to be reference to a neuropeptide Y receptor type 2 protein or nucleic acid encoding same.

In one example, reference to a protein or nucleic acid shall be taken to be reference to an olfactory receptor 11H4 protein or nucleic acid encoding same.

In one example, reference to a protein or nucleic acid shall be taken to be reference to a protocadherin alpha-4 protein or nucleic acid encoding same.

In one example, reference to a protein or nucleic acid shall be taken to be reference to a protocadherin alpha-C1 protein or nucleic acid encoding same.

In one example, reference to a protein or nucleic acid shall be taken to be reference to a rhomboid domain-containing protein 2 protein or nucleic acid encoding same.

In one example, reference to a protein or nucleic acid shall be taken to be reference to a sodium channel protein type 5 subunit alpha protein or nucleic acid encoding same.

In one example, reference to a protein or nucleic acid shall be taken to be reference to a serine incorporator 5 protein or nucleic acid encoding same.

In one example, reference to a protein or nucleic acid shall be taken to be reference to a solute carrier family 12 member 1 protein or nucleic acid encoding same.

In one example, reference to a protein or nucleic acid shall be taken to be reference to a proton-coupled folate transporter protein or nucleic acid encoding same.

In one example, reference to a protein or nucleic acid shall be taken to be reference to a solute carrier organic anion transporter family member 1B1 protein or nucleic acid encoding same.

In one example, reference to a protein or nucleic acid shall be taken to be reference to an anoctamin-2 protein or nucleic acid encoding same.

In one example, reference to a protein or nucleic acid shall be taken to be reference to an ATP-binding cassette sub-family A member 12 protein or nucleic acid encoding same.

In one example, reference to a protein or nucleic acid shall be taken to be reference to a carboxypeptidase M protein or nucleic acid encoding same.

In one example, reference to a protein or nucleic acid shall be taken to be reference to a neutral amino acid transporter B(0) protein or nucleic acid encoding same.

In one example, reference to a protein or nucleic acid shall be taken to be reference to a polycystic kidney disease 2-like 1 protein or nucleic acid encoding same.

In one example, reference to a protein or nucleic acid shall be taken to be reference to a probable phospholipid-transporting ATPase VA protein or nucleic acid encoding same.

In one example, reference to a protein or nucleic acid shall be taken to be reference to an acetylcholine receptor subunit gamma protein or nucleic acid encoding same.

In one example, reference to a protein or nucleic acid shall be taken to be reference to an insulin receptor-related protein or nucleic acid encoding same.

In one example, reference to a protein or nucleic acid shall be taken to be reference to a voltage-dependent N-type calcium channel subunit alpha-1B protein or nucleic acid encoding same.

In one example, reference to a protein or nucleic acid shall be taken to be reference to a sperm associated antigen 11B protein or nucleic acid encoding same.

In one example, reference to a protein or nucleic acid shall be taken to be reference to a Fraser Syndrome 1 protein or nucleic acid encoding same.

In one example, reference to a protein or nucleic acid shall be taken to be reference to an immunoglobulin-like domain containing receptor 1 protein or nucleic acid encoding same.

In one example, reference to a protein or nucleic acid shall be taken to be reference to an EPB41L1—erythrocyte membrane protein band 4.1 like 1 protein or nucleic acid encoding same.

In one example, reference to a protein or nucleic acid shall be taken to be reference to a B melanoma antigen protein or nucleic acid encoding same.

In one example, reference to a protein or nucleic acid shall be taken to be reference to a glutamate receptor, ionotropic, AMPA2 protein or nucleic acid encoding same.

In one example, reference to a protein or nucleic acid shall be taken to be reference to a synaptotagmin XV protein or nucleic acid encoding same.

In one example, reference to a protein or nucleic acid shall be taken to be reference to a NFASC—neurofascin homolog (chicken) protein or nucleic acid encoding same.

In one example, reference to a protein or nucleic acid shall be taken to be reference to a protein comprising a sequence encoded by EST IMAGE:2110090 or nucleic acid encoding same.

In one example, reference to a protein or nucleic acid shall be taken to be reference to a solute carrier family 30, member 10 protein or nucleic acid encoding same.

In one example, reference to a protein or nucleic acid shall be taken to be reference to an UNC-93 homologue A protein or nucleic acid encoding same.

In one example, reference to a protein or nucleic acid shall be taken to be reference to an olfactory receptor, family 1, subfamily C, member 1 protein or nucleic acid encoding same.

In one example, reference to a protein or nucleic acid shall be taken to be reference to a transmembrane and tetratricopeptide repeat containing 4 protein or nucleic acid encoding same.

In one example, reference to a protein or nucleic acid shall be taken to be reference to a chloride channel 4 protein or nucleic acid encoding same.

In one example, reference to a protein or nucleic acid shall be taken to be reference to a olfactory receptor, family 12, subfamily D, member 3 protein or nucleic acid encoding same.

In one example, reference to a protein or nucleic acid shall be taken to be reference to a butyrophilin-like protein 8 precursor protein or nucleic acid encoding same.

In one example, reference to a protein or nucleic acid shall be taken to be reference to a solute carrier, family 7 member 14 protein or nucleic acid encoding same.

In one example, reference to a protein or nucleic acid shall be taken to be reference to an olfactory receptor, family 7 subfamily D member 4 protein or nucleic acid encoding same.

In one example, reference to a protein or nucleic acid shall be taken to be reference to a mucin 12, cell surface associated protein or nucleic acid encoding same.

In one example, reference to a protein or nucleic acid shall be taken to be reference to a T-cell receptor gamma chain C region PT-gamma-1/2 protein or nucleic acid encoding same.

In one example, reference to a protein or nucleic acid shall be taken to be reference to a Defensin beta 109 protein or nucleic acid encoding same.

In one example, reference to a protein or nucleic acid shall be taken to be reference to a Kv channel interacting protein 1 (variant 1) protein or nucleic acid encoding same.

In one example, reference to a protein or nucleic acid shall be taken to be reference to a solute carrier family 45, member 4 protein or nucleic acid encoding same.

In one example, reference to a protein or nucleic acid shall be taken to be reference to an ectonucleotide pyrophosphatase/phosphodiesterase 6 protein or nucleic acid encoding same.

In one example, reference to a protein or nucleic acid shall be taken to be reference to a protocadherin beta 8 protein or nucleic acid encoding same.

In one example, reference to a protein or nucleic acid shall be taken to be reference to an olfactory receptor, family 2, sub family T, member 3 protein or nucleic acid encoding same.

In one example, reference to a protein or nucleic acid shall be taken to be reference to an olfactory receptor family 5, subfamily M, member 10 protein or nucleic acid encoding same.

In one example, reference to a protein or nucleic acid shall be taken to be reference to an olfactory receptor family 4, subfamily S, member 1 protein or nucleic acid encoding same.

In one example, reference to a protein or nucleic acid shall be taken to be reference to a G protein-coupled receptor 83 protein or nucleic acid encoding same.

In one example, reference to a protein or nucleic acid shall be taken to be reference to a taste receptor, type 2, member 19 protein or nucleic acid encoding same.

In one example, reference to a protein or nucleic acid shall be taken to be reference to a Kallmann syndrome 1 protein or nucleic acid encoding same.

In one example, reference to a protein or nucleic acid shall be taken to be reference to a solute carrier organic anion transporter family, member 1B3 protein or nucleic acid encoding same.

In one example, reference to a protein or nucleic acid shall be taken to be reference to a 7 transmembrane receptor (rhodopsin family) olfactory receptor like protein or a 60S acidic ribosomal protein P2 (RPLP2) or nucleic acid encoding same.

In one example, reference to a protein or nucleic acid shall be taken to be reference to a major histocompatability complex, class II, DQ beta 1 protein or nucleic acid encoding same.

In one example, reference to a protein or nucleic acid shall be taken to be reference to a CD166 (ALCAM) activated leukocyte cell adhesion molecule protein or nucleic acid encoding same.

In one example, reference to a protein or nucleic acid shall be taken to be reference to an IL-20Rbeta—Interleukin 20 receptor beta protein or nucleic acid encoding same.

In one example, reference to a protein or nucleic acid shall be taken to be reference to a podoplanin-differentiation factor; O-glycosylated protein or nucleic acid encoding same.

In one example, reference to a protein or nucleic acid shall be taken to be reference to a cholinergic receptor, muscarinic 3 protein or nucleic acid encoding same.

In one example, reference to a protein or nucleic acid shall be taken to be reference to an intergrin, beta 1 (fibronectin receptor, beta polypeptide, antigen CD29 includes MDF2, MSK12) protein or nucleic acid encoding same.

In one example, reference to a protein or nucleic acid shall be taken to be reference to a sialic acid binding Ig-like lectin 8, CD329 protein or nucleic acid encoding same.

In one example, reference to a protein or nucleic acid shall be taken to be reference to a RAS-related protein RAP1A protein or nucleic acid encoding same.

In one example, reference to a protein or nucleic acid shall be taken to be reference to a Plexin A2 protein or nucleic acid encoding same.

In one example, reference to a protein or nucleic acid shall be taken to be reference to a CD158b (KIR2DL3) killer cell immunoglobulin-like receptor, 2 domains, ligand 3 protein or nucleic acid encoding same.

In one example, reference to a protein or nucleic acid shall be taken to be reference to a CD314, killer cell lectin-like receptor, subfamily K, member 1 protein or nucleic acid encoding same.

In one example, reference to a protein or nucleic acid shall be taken to be reference to a chemokine (C-X3-C) receptor 1, CCRL1 protein or nucleic acid encoding same.

In one example, reference to a protein or nucleic acid shall be taken to be reference to a G protein-coupled receptor 174 protein or nucleic acid encoding same.

In one example, reference to a protein or nucleic acid shall be taken to be reference to a disintegrin and metalloproteinase domain-containing protein 10 (ADAM10) protein or nucleic acid encoding same.

In one example, reference to a protein or nucleic acid shall be taken to be reference to a signal-regulatory protein beta 1 (SIRPB1) protein or nucleic acid encoding same.

In one example, reference to a protein or nucleic acid shall be taken to be reference to a GM-CSF receptor subunit alpha precursor (CSF2RA) protein or nucleic acid encoding same.

In one example, reference to a protein or nucleic acid shall be taken to be reference to a Ecotropic viral integration 5 (EVI5) protein or nucleic acid encoding same.

In one example, reference to a protein or nucleic acid shall be taken to be reference to a lysyl oxidase-like 4 (LOXL4) protein or nucleic acid encoding same.

In one example, reference to a protein or nucleic acid shall be taken to be reference to a Leucine rich containing 33 (LRRC33) protein or nucleic acid encoding same.

In one example of the present disclosure, a marker set forth in any one or more of Tables 1-6 is expressed on an EPC (i.e., are positive for expression) or is expressed at a high or “hi” level on an EPC.

As used herein, the term “positive expression” or “+” shall be taken to mean expression above the level of background, e.g., as detected using an isotype control compound, e.g., antibody.

As used herein, the term “isotype control compound” shall be taken to mean a compound, e.g., an antibody of the same isotype as the compound (such as an antibody) used to detect expression of a protein, however having no relevant specificity to a protein and conjugated to the same detectable moiety as the compound used to detect expression of the protein. Such a control aids in distinguishing non-specific “background” binding from specific binding.

Reference to a “high” or “hi” level of expression the 50% of cells, such as 40%, 30% or for example 20%, such as 10% of cells expressing the highest level of the recited marker in a population of cells, e.g., as determined using FACS analysis.

The present disclosure also encompasses any combination of nucleic acids or proteins set forth in any one or more of Tables 1-6. For example, any example of the disclosure described herein shall be taken to apply mutatis mutandis to any two or more nucleic acids and/or proteins individually or collectively set forth in any one or more of Tables 1-6. Similarly, the present disclosure shall be taken to encompass detection of any combination of protein and nucleic acid markers individually or collectively set forth in any one or more of Tables 1-6.

Any example of the disclosure or example herein shall be taken to also apply to any nucleic acid or protein recited in the exemplified subject matter.

By “individually” is meant that the disclosure encompasses the recited nucleic acids or proteins or groups nucleic acids and/or proteins separately, and that, notwithstanding that individual nucleic acid(s) and/or protein(s) or groups of nucleic acids and/or proteins may not be separately listed herein, the accompanying claims may define such nucleic acid(s) and/or protein(s) or groups of nucleic acids and/or proteins separately and divisibly from each other.

By “collectively” is meant that the disclosure encompasses any number or combination of the recited nucleic acids and/or proteins or groups of nucleic acids and/or proteins, and that, notwithstanding that such numbers or combinations of nucleic acid(s) and/or proteins(s) or groups of nucleic acids and/or proteins may not be specifically listed herein, the accompanying claims may define such combinations or sub-combinations separately and divisibly from any other combination of nucleic acid(s) and/or protein(s) or groups of nucleic acids and/or proteins.

The present disclosure also contemplates detection of any individual or collection of proteins or nucleic acids described herein according to any example of the disclosure together with any other marker, e.g., of an EPC. Exemplary additional proteins or nucleic acids are described herein.

In another example, a method for detecting or isolating EPCs additionally comprises detecting a low or undetectable level of expression of a nucleic acid or protein expressed by a non-EPC. Exemplary nucleic acids and/or proteins include CD144, vWF, eNOS and/or Tie2.

### Detection/Isolation/Diagnostic/Therapeutic Compounds

The present disclosure encompasses a variety of reagents useful in detecting/isolating EPCs and/or diagnosing/prognosing/treating/preventing EPC-associated conditions. Compounds include antibodies, polypeptides comprising an antigen binding domain of an antibody, peptides, nucleic acid-based reagents, and small molecules. Any compound for treating a subject can be tested in vitro and/or in vivo using models of EPC activity and/or EPC-associated disease, e.g., as described herein.

### Protein Compounds

### Antibodies

For example, a method as described herein according to any example of the disclosure detects a protein and/or isolates a population enriched for EPCs using an antibody and/or polypeptide comprising an antigen binding domain of an antibody and/or involves administering an antibody or polypeptide comprising an antigen binding domain thereof.

As used herein, the term “antibody” refers to an immunoglobulin molecule capable of binding to a target protein and/or an epitope thereof and/or an immunogenic fragment thereof and/or a modified form thereof (e.g., glycosylated, etc.) through at least one antigen binding site, located in the variable region of the immunoglobulin molecule. This term encompasses not only intact polyclonal or monoclonal antibodies, but also variants, fusion polypeptides comprising an antibody, humanized antibodies, human antibodies and chimeric antibodies. This term also encompasses derivatives comprising the antibodies, e.g., conjugates comprising an additional component, e.g., a toxin and/or a compound that increases the stability of an antibody.

As used herein, the term “polypeptide comprising an antigen binding domain” shall be taken to mean any fragment or domain or polypeptide comprising same of an antibody that retains the ability to bind to the target protein specifically or selectively. This term also includes a polypeptide comprising a plurality of antigen binding domains from one or more antibody(ies). The polypeptide may form a component of a multimeric protein (e.g., in the case of Fab fragment or a diabody or higher order multimer). This term includes a Fab fragment, a Fab′ fragment, a F(ab′) fragment, a single chain antibody (SCA or SCAB or scFv), a diabody or higher order multimer amongst others. An “Fab fragment” consists of a monovalent antigen-binding fragment of an antibody molecule, and can be produced by digestion of a whole antibody molecule with the enzyme papain, to yield a fragment consisting of an intact light chain and a portion of a heavy chain. Such fragments can also be produced using recombinant means. An “Fab′ fragment” of an antibody molecule can be obtained by treating a whole antibody molecule with pepsin, followed by reduction, to yield a molecule consisting of an intact light chain and a portion of a heavy chain. Two Fab′ fragments are obtained per antibody molecule treated in this manner. Such fragments can also be produced using recombinant means. An “F(ab′)2 fragment” of an antibody consists of a dimer of two Fab′ fragments held together by two disulfide bonds, and is obtained by treating a whole antibody molecule with the enzyme pepsin, without subsequent reduction. Such fragments can also be produced using recombinant means. A “single chain antibody” (SCA) or “scFv” (single chain Fv, or single chain fragment variable) is a genetically engineered single chain molecule containing the variable region of a light chain and the variable region of a heavy chain, linked by a suitable, flexible polypeptide linker. The term “polypeptide comprising an antigen binding domain of an antibody” encompasses domain antibodies (dAbs) comprising a single variable domain, a heavy chain only antibody (e.g., from camelid or cartilaginous fish) or a minibody or a flex minibody or a diabody or a triabody or a tetrabody or a higher order multimer or any protein discussed above fused to a constant region of an antibody or a Fc region of an antibody or a C_{H}2 and/or C_{H}3 region of an antibody.

For some proteins described herein antibodies can be obtained from commercial sources, as will be apparent to the skilled artisan. For example, antibodies against ALCAM are commercially available from Abcam Ltd; antibodies against SPAG11B are commercially available from Santa Cruz Biotechnology, Inc; antibodies against FRAS1 are commercially available from Santa Cruz Biotechnology, Inc; antibodies against IL20RB are commercially available from Santa Cruz Biotechnology, Inc; antibodies against ILDR1 are commercially available from Abnova; antibodies against EPB41L1 are commercially available from Abcam Ltd; antibodies against BAGE are commercially available from Santa Cruz Biotechnology, Inc; antibodies against CHRM3 are commercially available from Santa Cruz Biotechnology, Inc; antibodies against GRIA2 are commercially available from Abcam Ltd; antibodies against SYT15 are commercially available from Santa Cruz Biotechnology, Inc; antibodies against NLGN1 are commercially available from Abnova; antibodies against ITGB1 are commercially available from Becton Dickinson Inc; antibodies against SIGLEC8 are commercially available from Abnova; antibodies against UNC93A are commercially available from Santa Cruz Biotechnology, Inc; antibodies against OR1C1 are commercially available from Santa Cruz Biotechnology, Inc; antibodies against RAP1A are commercially available from Abnova; antibodies against PLXNA2 are commercially available from Abnova; antibodies against TMTC4 are commercially available from Santa Cruz Biotechnology, Inc; antibodies against CLCN4 are commercially available from Abnova; antibodies against OR12D3 are commercially available from Santa Cruz Biotechnology, Inc; antibodies against BTNL8 are commercially available from Santa Cruz Biotechnology, Inc; antibodies against KIR2DL3 are commercially available from Becton Dickinson Inc; antibodies against SLC7A14 are commercially available from Abcam; antibodies against GPR18 are commercially available from Santa Cruz Biotechnology, Inc; antibodies against OR7D4 are commercially available from Santa Cruz Biotechnology, Inc; antibodies against KLRK1 are commercially available from Becton Dickinson Inc; antibodies against MUC12 are commercially available from Santa Cruz Biotechnology, Inc; antibodies against CX3CR1 are commercially available from Abnova; antibodies against DEFB109 are commercially available from Santa Cruz Biotechnology, Inc; antibodies against KCNIP1 are commercially available from Abnova; antibodies against SLC45A4 are commercially available from Santa Cruz Biotechnology, Inc; antibodies against ENPP6 are commercially available from Abcam; antibodies against PCDHB8 are commercially available from Santa Cruz Biotechnology, Inc; antibodies against EMR2 are commercially available from Abcam; antibodies against SLCO1B3 are commercially available from Abcam; antibodies against HLA_DQB1 are commercially available from Abnova; antibodies against GPR83 are commercially available from Santa Cruz Biotechnology, Inc; antibodies against TAS2R19 are commercially available from Abcam; antibodies against KAL1 are commercially available from Abcam; anti-GPR174 antibodies are available from Genway Biotech Inc; antibodies against EPGN are available from Sigma Aldrich or Abcam; antibodies against ALC15A2 are available from Abcam; EMB antibodies are available from Abcam; and AREG antibodies are available from Abnova; ITM2A antibodies are available from Sigma Aldrich; NRG4 antibodies are available from Abcam; antibodies against SLC16A6 are available from Sigma Aldrich; antibodies; antibodies against SLC39A8 are available from Santa Cruz Biotechnology; antibodies against GPM6B are commercially available from Sigma Aldrich; antibodies against SIGLEC10 are commercially available from Abcam; antibodies against CNR2 are commercially available from Genway Biotech Inc; antibodies against RHBDD1 are commercially available from Sigma Aldrich; antibodies against PRSS21 are commercially available from Abcam; antibodies against SIGLEC6 are commercially available from Abgent; antibodies against SORL1 are commercially available from Prosci Incorporated; antibodies against NCKAP1L are commercially available from Abcam; antibodies against EVI2B are commercially available from Novus Biologicals; antibodies against KCNQ5 are commercially available from KCNQ5; antibodies against PLXNC1 are commercially available from Santa Cruz Biotechnology; antibodies against P2RY14 are available from Novus Biologicals; antibodies against SLC8A1 are commercially available from Abnova; antibodies against HTR1F are commercially available from Sigma Aldrich; antibodies against TRAT1 are commercially available from Lifespan Biosciences; antibodies against GPR183 are commercially available from Abnova; antibodies against OR13D1 are commercially available from Abcam; antibodies against VSIG4 are commercially available from Sino Biologicals; antibodies against TAS2R4 are commercially available from Abcam; antibodies against GPR18 are commercially available from Genway Biologicals Inc; antibodies against EMR2 are commercially available from Novus Biologicals; antibodies against TAS2R3 are commercially available from Abcam; antibodies against TAS2R13 are commercially available from Abcam; antibodies against MR1 are commercially available from Abcam; antibodies against SLC22A16 are commercially available from Abcam; antibodies against GPR34 are commercially available from Novus Biologicals; antibodies against NKG7 are commercially available from Santa Cruz Biotechnology; antibodies against SLC24 are commercially available from Lifespan Biosciences; antibodies against GPR65 are commercially available from Abcam; antibodies against SLC2A5 are commercially available from Sigma Aldrich; antibodies against KCNAB2 are commercially available from Antibodies Online; antibodies against OPN3 are commercially available from Abcam; antibodies against KCNE3 are commercially available from Santa Cruz Biotechnology; antibodies against LAT2 are commercially available from Abcam; antibodies against ABCC4 are commercially available from Sigma Aldrich; antibodies against OR52B6 are commercially available from Santa Cruz Biotechnology; antibodies against ADCY7 are commercially available from Abcam; antibodies against MLC1 are commercially available from Genway Biologicals; antibodies against ENPP5 are commercially available from Abcam; antibodies against SLC38A1 are commercially available from Genway Biologicals; antibodies against DSG2 are commercially available from R&D Systems; antibodies against CD302 are commercially available from Santa Cruz Biotechnology; antibodies against SLC1A3 are commercially available from Novus Biologicals; antibodies against TBXAS1 are commercially available from Acris Antibodies; antibodies against SEMA3C are commercially available from Santa Cruz Biotechnology; antibodies against LAPTM5 are commercially available from Novus Biologicals; antibodies against VAMP8 are commercially available from Abcam; antibodies against SLC1A4 are commercially available from Novus Biologicals; antibodies against AKAP7 are commercially available from Abcam; antibodies against CLDN20 are commercially available from Sigma Aldrich; antibodies against CLDN10 are commercially available from Acris Antibodies; antibodies against ADAMTS2 are commercially available from Abcam; antibodies against PLBD1 are commercially available from Acris Antibodies; antibodies against IQCB 1 are commercially available from Novus Biologicals; antibodies against MFAP4 are commercially available from Abcam; antibodies against FBN2 are commercially available from Sigma Aldrich; antibodies against OGN are commercially available from Santa Cruz Biotechnology; antibodies against OMD are commercially available from R&D Systems; antibodies against ASPN are commercially available from Everest Biotech; antibodies against PZP are commercially available from Santa Cruz Biotechnology; antibodies against HSN2 are commercially available from Novus Biologicals; antibodies against FAM46C are commercially available from Abcam; antibodies against SERPINI2 are commercially available from Novus Biologicals; antibodies against Erlin-1 are commercially available from Abcam; and antibodies against LOXL3 are commercially available from Santa Cruz Biotechnology.

To generate antibodies, a protein or immunogenic fragment or epitope thereof or a cell expressing and displaying same, optionally formulated with any suitable or desired carrier, adjuvant, or pharmaceutically acceptable excipient, is conveniently administered in the form of an injectable composition. Injection may be intranasal, intramuscular, sub-cutaneous, intravenous, intradermal, intraperitoneal, or by other known route. For treatment of eye conditions, administration can be intraocular, or intravitreal. For intravenous injection, it is desirable to include one or more fluid and nutrient replenishers. Means for preparing and characterizing antibodies are known in the art. (See, e.g., ANTIBODIES: A LABORATORY MANUAL, Cold Spring Harbor Laboratory, 1988, incorporated herein by reference).

Immunogenic peptides for generating polyclonal or monoclonal antibodies can be covalently coupled to an immunogenic carrier protein, such as diphtheria toxoid (DT), Keyhole Limpet Hemocyanin (KLH), tetanus toxoid (TT) or the nuclear protein of influenza virus (NP), using one of several conjugation chemistries known in the art. This enhances the immunogenicity of peptides that are otherwise not highly immunogenic in animals e.g., mice, rats, rabbits, chickens etc. Methods of preparing and/or carrier proteins are known in the art and described, for example, in U.S. Pat. Nos. 4,709,017, 5,843,711, 5,601,827, and 5,917,017).

The conjugate molecules so produced may be purified and employed in immunogenic compositions to elicit, upon administration to a host, an immune response to the protein and/or peptide which is potentiated in comparison to the protein or peptide alone.

The efficacy of the protein or immunogenic fragment or epitope thereof or cell expressing same in producing an antibody is established by injecting an animal, for example, a mouse, chicken, rat, rabbit, guinea pig, dog, horse, cow, goat or pig, with a formulation comprising the protein or immunogenic fragment or epitope thereof, and then monitoring the immune response to the protein, epitope or fragment. Both primary and secondary immune responses are monitored. The antibody titer is determined using any conventional immunoassay, such as, for example, ELISA, or radio-immunoassay.

The production of polyclonal antibodies may be monitored by sampling blood of the immunized animal at various points following immunization. A second, booster injection, may be given, if required to achieve a desired antibody titer. The process of boosting and titering is repeated until a suitable titer is achieved. When a desired level of immunogenicity is obtained, the immunized animal is bled and the serum isolated and stored, and/or the animal is used to generate monoclonal antibodies (Mabs).

Monoclonal antibodies (mAbs) are exemplary antibodies useful in performance of the invention. The term “monoclonal antibody” or “mAb” refers to a homogeneous antibody population capable of binding to the same antigen(s) such as, to the same epitopic determinant within the antigen(s). This term is not intended to be limited as regards to the source of the antibody or the manner in which it is made.

For the production of mAbs any one of a number of known techniques may be used, such as, for example, the procedure exemplified in U.S. Pat. No. 4,196,265 or ANTIBODIES: A LABORATORY MANUAL, Cold Spring Harbor Laboratory, 1988, incorporated herein by reference.

For example, a suitable animal is immunized with an effective amount of the protein or immunogenic fragment or epitope thereof or cell expressing same under conditions sufficient to stimulate antibody producing cells. Rodents such as rabbits, mice and rats are exemplary animals, however, the use of sheep or frog cells is also possible. The use of rats may provide certain advantages, but mice or rabbits are useful, with the BALB/c or C57BL/6 mouse being a routinely used animal and one that generally gives a higher percentage of stable fusions. Alternatively, a mouse genetically-engineered to express human immunoglobulin proteins, and, for example, not express murine immunoglobulin proteins, is immunized to produce an antibody of the present disclosure. Such mice are known in the art and commercially available. For example, Regeneron, Inc. have produced the VelocImmune™ mouse in which human variable regions have been homologously recombined or knocked-in to the mouse genome to replace endogenous mouse variable region encoding genes. Such mice are described, for example, in WO2002/066630. Abgenix/Amgen, Inc. and Kirin Brewery/Medarex, Inc. have produced strains of mice in which the endogenous mouse immunoglobulin loci are inactivated or “knocked-out” and human immunoglobulin loci introduced using yeast artificial chromosomes. Examples of these mice are described or reviewed in Lonberg et al. (1994); Lonberg, (1994); Tomizuka et al. (2000) and Jakobovits et al. (2007).

Following immunization, somatic cells with the potential for producing antibodies, specifically B lymphocytes (B cells), are selected for use in the mAb generating protocol. These cells may be obtained from biopsies of spleens, tonsils or lymph nodes, or from a peripheral blood sample. Spleen cells and peripheral blood cells are exemplary, the former because they are a rich source of antibody-producing cells that are in the dividing plasmablast stage, and the latter because peripheral blood is easily accessible. Spleen lymphocytes are obtained by homogenizing the spleen with a syringe. The B cells from the immunized animal are then fused with cells of an immortal myeloma cell, generally derived from the same species as the animal that was immunized with the immunogen. Any one of a number of myeloma cells may be used and these are known to those of skill in the art (e.g. murine P3-X63/Ag8, X63-Ag8.653, NS1/1.Ag 4 1, Sp2/0-Ag14, FO, NSO/U, MPC-I 1, MPC11-X45-GTG 1.7 and S194/5XX0).

To generate hybrids of antibody-producing spleen or lymph node cells and myeloma cells, somatic cells are mixed with myeloma cells in the presence of an agent or agents (chemical or electrical) that promote the fusion of cell membranes. Fusion methods using Sendai virus have been described by Kohler and Milstein, (1975); and Kohler and Milstein, (1976). Methods using polyethylene glycol (PEG), such as 37% (v/v) PEG, are described in detail by Gefter et al, (1977). The use of electrically induced fusion methods is also appropriate.

Hybrids are amplified by culture in a selective medium comprising an agent that blocks the de novo synthesis of nucleotides in the tissue culture media. Exemplary agents are aminopterin, methotrexate and azaserine.

The amplified hybridomas are subjected to a functional selection for antibody specificity and/or titer, such as, for example, by immunoassay (e.g. radioimmunoassay, enzyme immunoassay, cytotoxicity assay, plaque assay, dot immunoassay, and the like).

The selected hybridomas are serially diluted and cloned into individual antibody-producing cell lines, which clones can then be propagated for an extended period to provide mAbs. The cell lines may be exploited for mAb production in at least two basic ways. A sample of the hybridoma is injected, usually in the peritoneal cavity, into a histocompatible animal of the type that was used to provide the somatic and myeloma cells for the original fusion. The injected animal develops tumors secreting the specific monoclonal antibody produced by the fused cell hybrid. The body fluids of the animal, such as serum or ascites fluid, can then be tapped to provide mAbs in high concentration. The individual cell lines could also be cultured in vitro, where the mAbs are naturally secreted into the culture medium from which they are readily obtained in high concentrations. MAbs produced by either means may be further purified, if desired, using filtration, centrifugation and various chromatographic methods such as HPLC or affinity chromatography.

Alternatively, ABL-MYC technology (NeoClone, Madison Wis. 53713, USA) is used to produce cell lines secreting monoclonal antibodies (mAbs) against a protein as described herein according to any example of the disclosure or an epitope or immunogenic fragment thereof. This technology comprises infecting splenocytes from immunized mice with replication-incompetent retrovirus comprising the oncogenes v-abl and c-myc. Splenocytes are transplanted into naive mice which then develop ascites fluid containing cell lines producing monoclonal antibodies (mAbs) against a protein as described herein according to any example of the disclosure or an epitope or immunogenic fragment thereof. The mAbs are purified from ascites using protein G or protein A, e.g., bound to a solid matrix, depending on the isotype of the mAb. The ABL-MYC technology is described generically in detail in Largaespada (1990); and Largaespada et al, (1996).

Antibodies can also be produced or isolated by screening a display library, e.g., a phage display library where, for example the phage express scFv fragments on the surface of their coat with a large variety of CDRs. For example, U.S. Pat. No. 6,521,404, U.S. Pat. No. 5,969,108 and U.S. Pat. No. 7,049,135 describe the isolation of murine and/or human antibodies, using phage libraries. Subsequent publications describe the production of high affinity (nM range) human antibodies by chain shuffling (Marks et al, 1992), as well as combinatorial infection and in vivo recombination as a strategy for constructing very large phage libraries (Waterhouse et al, 1993).

### Recombinant Antibody Production

The antibodies or proteins of the present disclosure can also be produced recombinantly, using techniques and materials readily obtainable.

For example, DNA encoding an antibody of the disclosure or a polypeptide comprising an antigen binding domain of an antibody, e.g., a Fab fragment is readily isolated and sequenced using conventional procedures (e.g., by using oligonucleotide probes that are capable of binding specifically to genes encoding the heavy and light chains of murine antibodies). A hybridoma cell serves as a source of such DNA. Once isolated, the DNA may be placed into expression vectors, which are then transfected into host cells such as E. coli cells, simian COS cells, Chinese Hamster Ovary (CHO) cells, or myeloma cells that do not otherwise produce antibody protein, to obtain the synthesis of monoclonal antibodies in the recombinant host cells. Review articles on recombinant expression in bacteria of DNA encoding the antibody include Skerra et al, (1993) and Pluckthun, (1992). Molecular cloning techniques to achieve these ends are known in the art and described, for example in Ausubel et al. (1988) or Sambrook et al. (1989). A wide variety of cloning and in vitro amplification methods are suitable for the construction of recombinant nucleic acids. Examples of these techniques and instructions sufficient to direct persons of skill through many cloning exercises are found in Berger and Kimmel; Sambrook et al., (1989); and Ausubel et al., eds., (1988). Methods of producing recombinant immunoglobulins are also known in the art. See U.S. Pat. No. 6,331,415; and U.S. Pat. No. 5,585,089.

For recombinant production of an antibody or protein, the nucleic acid encoding it is isolated and inserted into a replicable vector for further cloning (amplification of the DNA) or for expression. DNA encoding the antibody is readily isolated or synthesized using conventional procedures (e.g., by using oligonucleotide probes that are capable of binding specifically to DNAs encoding the heavy and light chains of the antibody). Many vectors are available. Exemplary vectors are described herein. The vector components generally include, but are not limited to, one or more of the following: a signal sequence, a sequence encoding an antibody or protein of the present disclosure (e.g., derived from the information provided herein), an enhancer element, a promoter, and a transcription termination sequence. The skilled artisan will be aware of suitable sequences for expression of an antibody. For example, exemplary signal sequences include prokaryotic secretion signals (e.g., alkaline phosphatase, penicillinase, Ipp, or heat-stable enterotoxin II), yeast secretion signals (e.g., invertase leader, a factor leader, or acid phosphatase leader) or mammalian secretion signals (e.g., herpes simplex gD signal or an immunoglobulin signal). Exemplary promoters include those active in prokaryotes (e.g., phoA promoter, β-lactamase and lactose promoter systems, alkaline phosphatase, a tryptophan (trp) promoter system, and hybrid promoters such as the tac promoter), and those active in mammalian cells (e.g., cytomegalovirus immediate early promoter (CMV), the human elongation factor 1-α promoter (EF1), the small nuclear RNA promoters (U1a and U1b), α-myosin heavy chain promoter, Simian virus 40 promoter (SV40), Rous sarcoma virus promoter (RSV), Adenovirus major late promoter, β-actin promoter; hybrid regulatory element comprising a CMV enhancer/β-actin promoter or an immunoglobulin promoter or active fragment thereof).

Suitable host cells for cloning or expressing the DNA in the vectors herein are the prokaryotic, yeast, or higher eukaryotic cells described above. Suitable prokaryotes for this purpose include eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis, Pseudomonas such as P. aeruginosa, and Streptomyces. One E. coli cloning host is E. coli 294 (ATCC 31,446), although other strains such as E. coli B, E. coli X 1776 (ATCC 31,537), and E. coli W3110 (ATCC 27,325) are suitable. These examples are illustrative rather than limiting.

In addition to prokaryotes, eukaryotic microbes such as filamentous fungi or yeast are suitable cloning or expression hosts for antibody-encoding vectors. Saccharomyces cerevisiae, or common baker's yeast, is the most commonly used among lower eukaryotic host microorganisms. However, a number of other genera, species, and strains are commonly available and useful herein, such as Schizosaccharomyces pombe; Pichia pastoris (EP 183,070); and filamentous fungi such as, e.g., Neurospora, Penicillium, Tolypocladium, and Aspergillus hosts such as A. nidulans and A. niger.

Suitable host cells for the expression of glycosylated antibody are derived from multicellular organisms. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruitfly), and Bombyx mori have been identified.

Examples of useful mammalian host cell lines are monkey kidney CV1 line transformed by SV40 (COS-7, ATCC CRL 1651); human embryonic kidney line (293 or 293 cells subcloned for growth in suspension culture, Graham et al. (1977); baby hamster kidney cells (BHK, ATCC CCL 10); Chinese hamster ovary cells (CHO); mouse Sertoli cells (TM4); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al. (1982); MRC 5 cells; FS4 cells; and PER.C6™ (Crucell NV).

The host cells used to produce the antibody of this disclosure may be cultured in a variety of media. Commercially available media such as Ham's F10 (Sigma), Minimal Essential Medium ((MEM), (Sigma), RPM1-1640 (Sigma), and Dulbecco's Modified Eagle's Medium ((DMEM), Sigma) are suitable for culturing the host cells. In addition, any of the media described in Ham et al. (1979), Barnes et al. (1980), U.S. Pat. No. 4,767,704; U.S. Pat. No. 4,657,866; U.S. Pat. No. 4,927,762; U.S. Pat. No. 4,560,655; U.S. Pat. No. 5,122,469; WO90/03430; WO87/00195; may be used as culture media for the host cells.

### Chimeric Antibodies

In one example an antibody of the disclosure is a chimeric antibody. The term “chimeric antibody” refers to antibodies in which a portion of the heavy and/or light chain is identical with or homologous to corresponding sequences in antibodies derived from a particular species (e.g., murine, such as mouse) or belonging to a particular antibody class or subclass, while the remainder of the chain(s) is identical with or homologous to corresponding sequences in antibodies derived from another species (e.g., primate, such as human) or belonging to another antibody class or subclass, as well as fragments of such antibodies, so long as they exhibit the desired biological activity (U.S. Pat. No. 4,816,397, U.S. Pat. No. 4,816,567; and U.S. Pat. No. 5,807,715).

Typically chimeric antibodies utilize rodent or rabbit variable regions and human constant regions, in order to produce an antibody with predominantly human domains. For example, a chimeric antibody comprises a variable region from a mouse antibody as described herein according to any example of the disclosure fused to a human constant region. The production of such chimeric antibodies is known in the art, and may be achieved by standard means (as described, e.g., U.S. Pat. No. 4,816,397, U.S. Pat. No. 4,816,567; and U.S. Pat. No. 5,807,715).

As used herein, “antibody variable domain” refers to the portions of the light and heavy chains of antibody molecules that include amino acid sequences of complementarity determining regions (CDRs; i.e., CDR1, CDR2, and CDR3), and Framework Regions (FRs). V_{H} refers to the variable domain of the heavy chain. V_{L} refers to the variable domain of the light chain. CDRs and FRs may be defined according to Kabat (1987 and 1991)) or Chothia and Lesk (1987) or any other known technique or combination thereof.

The term constant region (CR) as used herein, refers to the portion of the antibody molecule which confers effector functions. The heavy chain constant region can be selected from any of the five isotypes: alpha, delta, epsilon, gamma or mu. Further, heavy chains of various subclasses (such as the IgG subclasses of heavy chains) are responsible for different effector functions and thus, by choosing the desired heavy chain constant region, antibodies with desired effector function can be produced. Exemplary heavy chain constant regions are gamma 1 (IgG1), gamma 2 (IgG2), gamma 3 (IgG3) and gamma 4 (IgG4). Light chain constant regions can be of the kappa or lambda type, such as of the kappa type.

As used herein, the term “complementarity determining regions” (syn. CDRs; i.e., CDR1, CDR2, and CDR3) refers to the amino acid residues of an antibody variable domain the presence of which are necessary for antigen binding. Each variable domain typically has three CDR regions identified as CDR1, CDR2 and CDR3. Each complementarity determining region may comprise amino acid residues from a “complementarity determining region” as defined by Kabat et al. (1987 and 1991) and/or those residues from a “hypervariable loop” Chothia and Lesk (1987) or any other known technique or combination thereof.

“Framework regions” (hereinafter FR) are those variable domain residues other than the CDR residues.

### Humanized and Human Antibodies

The antibodies of the present disclosure may be humanized antibodies or human antibodies.

The term “humanized antibody” shall be understood to refer to a chimeric molecule, generally prepared using recombinant techniques, having an epitope binding site derived from an antibody from a non-human species and the remaining antibody structure of the molecule based upon the structure and/or sequence of a human antibody. The antigen-binding site comprises the complementarity determining regions (CDRs) from the non-human antibody grafted onto appropriate framework regions in the variable domains of a human antibody and the remaining regions from a human antibody. Antigen binding sites may be wild type or modified by one or more amino acid substitutions. Humanized forms of non-human (e.g., murine) antibodies are chimeric antibodies, antibody chains or polypeptides comprising antigen binding domains thereof (such as Fv, Fab, Fab′, F(ab′)₂ or other antigen-binding subsequences of antibodies) which contain minimal sequence derived from non-human antibody. In some instances, Fv framework residues of the human antibody are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human antibody and all or substantially all of the FR regions are those of a human antibody consensus sequence. The humanized antibody optimally also will comprise at least a portion of an antibody constant region (Fc), typically that of a human antibody.

Methods for humanizing non-human antibodies are known in the art. Humanization can be essentially performed following the method of U.S. Pat. No. 6,548,640, U.S. Pat. No. 5,585,089, U.S. Pat. No. 6,054,297 or U.S. Pat. No. 5,859,205. Other methods for humanizing an antibody are not excluded.

The term “human antibody” as used herein in connection with antibody molecules and binding proteins refers to antibodies having variable (e.g. V_{H}, V_{L}, CDR and FR regions) and constant antibody regions derived from or corresponding to sequences found in humans, e.g. in the human germline or somatic cells. The “human” antibodies can include amino acid residues not encoded by human sequences, e.g. mutations introduced by random or site directed mutations in vitro (in particular mutations which involve conservative substitutions or mutations in a small number of residues of the antibody, e.g. in 1, 2, 3, 4 or 5 of the residues of the antibody, e.g. in 1, 2, 3, 4 or 5 of the residues making up one or more of the CDRs of the antibody). These “human antibodies” do not actually need to be produced by a human, rather, they can be produced using recombinant means and/or isolated from a transgenic animal (e.g., mouse) comprising nucleic acid encoding human antibody constant and/or variable regions (e.g., as described above).

Human antibodies can also be produced using various techniques known in the art, including phage display libraries (e.g., as described in U.S. Pat. No. 5,885,793).

Completely human antibodies which recognize a selected epitope can also be generated using a technique referred to as “guided selection.” In this approach a selected non-human monoclonal antibody, e.g., a mouse antibody, is used to guide the selection of a completely human antibody recognizing the same epitope (U.S. Pat. No. 5,565,332).

### Multi-Specific Antibodies

Bispecific antibodies are antibodies that have binding specificities for at least two different epitopes. Exemplary bispecific antibodies may bind to two different epitopes of the target protein. Other such antibodies may combine a binding site for a protein described herein with a binding site for another protein. Alternatively, a region that binds a protein described herein may be combined with a region which binds to a triggering molecule on a leukocyte such as a T-cell receptor molecule (e.g., CD3), or Fc receptors for IgG (FcγR), such as FcγRI (CD64), FcγRII (CD32) and/or FcγRIII (CD16), so as to focus and localize cellular defense mechanisms to an EPC. Bispecific antibodies may also be used to localize cytotoxic agents to EPCs. These antibodies possess a target protein-binding region and a region which binds the cytotoxic agent (e.g., saporin, anti-interferon-α, vinca alkaloid, ricin A chain, methotrexate or radioactive isotope hapten). Bispecific antibodies can be prepared as full length antibodies or proteins comprising antigen binding domains of antibodies (e.g., F(ab′)2 bispecific antibodies). Exemplary bispecific antibodies and their method for production are described in WO96/16673, WO98/02463 and U.S. Pat. No. 5,821,337.

Methods for making bispecific antibodies are known in the art. Traditional production of full length bispecific antibodies is based on the co-expression of two immunoglobulin heavy chain-light chain pairs, where the two chains have different specificities (Millstein et al, 1983). Because of the random assortment of immunoglobulin heavy and light chains, these hybridomas (quadromas) produce a potential mixture of 10 different antibody molecules, of which only one has the correct bispecific structure. Purification of the correct molecule is usually done by affinity chromatography steps. Similar procedures are disclosed in WO93/08829, and in Traunecker et al. (1991). Other approaches for producing bispecific antibodies are known in the art and described for example, in WO94/04690; U.S. Pat. No. 5,731,168; Suresh et al, (1986).

Bispecific antibodies include cross-linked or “heteroconjugate” antibodies. For example, one of the antibodies in the heteroconjugate can be coupled to avidin, the other to biotin. Such antibodies are known in the art and described, for example, in U.S. Pat. No. 4,676,980; WO91/00360; and WO92/200373.

Bispecific antibodies can also be prepared using chemical linkage (Brennan et al, 1985) or using Fab′-SH fragments from E. coli, which can be chemically coupled to form bispecific antibodies (Shalaby et al, 1992). Other techniques make use of leucine zippers (Kostelny et al, 1992) or the “diabody” technology described by Hollinger et al, (1993).

Antibodies with more than two valencies are also contemplated by the present disclosure. For example, trispecific antibodies can be prepared (Tutt et al, (1991).

The antibodies of the present disclosure can be multivalent antibodies (which are other than of the IgM class) with three or more antigen binding sites (e.g., tetravalent antibodies), which can be readily produced by recombinant expression of nucleic acid encoding the polypeptide chains of the antibody. The multivalent antibody can comprise a dimerization domain and three or more antigen binding sites. The dimerization domain comprises (or consists of) an Fc region or a hinge region of an antibody. In this scenario, the antibody can comprise an Fc region and three or more antigen binding sites amino-terminal to the Fc region.

### Mutations to Antibodies

Amino acid sequence modification(s) of the antibodies described herein are encompassed by the present disclosure. For example, it may be desirable to improve the binding affinity and/or other biological properties of the antibody. Amino acid sequence variants of the antibody are prepared by introducing appropriate nucleotide changes into the encoding nucleic acid, or by peptide synthesis. Such modifications include, for example, deletions from, and/or insertions into and/or substitutions of, residues within the amino acid sequences of the antibody. Any combination of deletion, insertion, and substitution is made to arrive at the final construct, provided that the final construct possesses the desired characteristics. The amino acid changes also may alter post-translational processes of the antibody, such as changing the number or position of glycosylation sites.

Amino acid sequence insertions include amino- and/or carboxyl-terminal fusions ranging in length from one residue to polypeptides containing one hundred or more residues, as well as intrasequence insertions of single or multiple amino acid residues. Examples of terminal insertions include an antibody with an N-terminal methionyl residue or the antibody fused to a cytotoxic polypeptide. Other insertional variants of the antibody include the fusion to the N- or C-terminus of the antibody to an enzyme or a polypeptide which increases the serum half-life of the antibody.

Another type of variant is an amino acid substitution variant. These variants have at least one amino acid residue in the antibody replaced by a different residue. The sites of interest for substitutional mutagenesis include the CDRs, however FR alterations are also contemplated. Exemplary substitutions are conservative substitutions.

Any cysteine residue not involved in maintaining the proper conformation of the antibody also may be substituted, generally with serine, to improve the oxidative stability of the molecule and prevent aberrant crosslinking. Conversely, cysteine bond(s) may be added to the antibody to improve its stability (particularly where a polypeptide comprising an antigen binding domain is used, e.g., a protein comprising a Fv).

An exemplary type of substitutional variant involves substituting one or more CDR residues of a parent antibody (e.g., a humanized or human antibody). Generally, the resulting variant(s) selected for further development will have improved biological properties relative to the parent antibody from which they are generated. A convenient way for generating such substitutional variants involves affinity maturation using phage display e.g., as described in U.S. Pat. No. 5,223,409.

Another type of amino acid variant of the antibody alters the original glycosylation pattern of the antibody. By altering is meant deleting one or more carbohydrate moieties found in the antibody, and/or adding one or more glycosylation sites that are not present in the antibody. Modified glycoforms of antibodies may be useful for a variety of purposes, including but not limited to enhancing or reducing effector function and/or modifying half life of the antibody (see, for example, WO2007/010401). Such alterations may result in a decrease or increase of CIq binding and CDC or of FcγR binding and/or ADCC. Substitutions can, for example, be made in one or more of the amino acid residues of the heavy chain constant region thereby causing an alteration in an effector function while retaining the ability to bind to the antigen as compared with the modified antibody, e.g., as described in U.S. Pat. No. 5,624,821 and U.S. Pat. No. 5,648,260. Engineered glycoforms may be generated by any method known to one skilled in the art, for example by using engineered or variant expression strains, by co-expression with one or more enzymes, for example β(1,4)-N-acetylglucosaminyltransferase III (GnTIII), by expressing an antibody or protein in various organisms or cell lines from various organisms, or by modifying carbohydrate(s) after the antibody or protein has been expressed. Methods for generating engineered glycoforms are known in the art, and include but are not limited to those described in U.S. Pat. No. 6,602,684; U.S. Ser. No. 10/277,370; or U.S. Ser. No. 10/113,929.

Alternatively, or in addition, the antibodies or proteins can be expressed in a transfectoma which does not add the fucose unit normally attached to Asn at position 297 of the Fc region of an IgG (e.g., IgG1) in order to enhance the affinity of the Fc region for Fc-Receptors which, in turn, will result in an increased ADCC of the antibodies in the presence of NK cells, e.g., Shield et al. 2002.

To increase the serum half life of the antibody, one may incorporate a salvage receptor binding epitope into the antibody or polypeptide comprising an antigen binding domain of an antibody as described in U.S. Pat. No. 5,739,277, for example. As used herein, the term “salvage receptor binding epitope” refers to an epitope of the Fc region of an IgG molecule (e.g., IgG1, IgG2, IgG3, or IgG4) that is responsible for increasing the in vivo serum half-life of the IgG molecule, e.g., by binding to a neonatal Fc receptor (FcRn).

### Purification of Antibodies

When using recombinant techniques, the antibody can be produced intracellularly, in the periplasmic space, or directly secreted into the medium. If the antibody is produced intracellularly, as a first step, the particulate debris, either host cells or lysed fragments, is removed, for example, by centrifugation or ultrafiltration. Carter et al. (1992) describe a procedure for isolating antibodies which are secreted to the periplasmic space of E. coli. Briefly, cell paste is thawed in the presence of sodium acetate (pH 3.5), EDTA, and phenylmethylsulfonylfluoride (PMSF) over about 30 min. Cell debris can be removed by centrifugation. Where the antibody is secreted into the medium, supernatants from such expression systems are generally first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

The antibody prepared from the cells can be purified using, for example, hydroxyl apatite chromatography, gel electrophoresis, dialysis, and affinity chromatography, with affinity chromatography being an exemplary purification technique. The suitability of protein A as an affinity ligand depends on the species and isotype of any immunoglobulin Fc domain that is present in the antibody. Protein A can be used to purify antibodies that are based on human γ1, γ2, or γ4 heavy chains (Lindmark et al., 1983). Protein G is recommended for all mouse isotypes and for human γ3 (Guss et al., 1986). The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Other techniques for protein purification such as fractionation on an ion-exchange column, ethanol precipitation, Reverse Phase HPLC, chromatography on silica, chromatography on heparin SEPHAROSE™ chromatography on an anion or cation exchange resin (such as a polyaspartic acid column), chromatofocusing, SDS-PAGE, and ammonium sulfate precipitation are also available depending on the antibody to be recovered.

Following any preliminary purification step(s), the mixture comprising the antibody of interest and contaminants may be subjected to low pH hydrophobic interaction chromatography.

Antibody Derivatives The present disclosure also provides derivatives of an antibody or protein as described herein according to any example of the disclosure, e.g., a conjugate (immunoconjugate) comprising an antibody or protein of the present disclosure conjugated to a distinct moiety, e.g., a therapeutic agent which is directly or indirectly bound to the antibody. Examples of other moieties include, but are not limited to, an enzyme, a fluorophophore, a cytotoxin, a radioisotope (e.g., iodine-131, yttrium-90 or indium-111), an immunomodulatory agent, an anti-angiogenic agent, an anti-neovascularization and/or other vascularization agent, a toxin, an anti-proliferative agent, a pro-apoptotic agent, a chemotherapeutic agent and a therapeutic nucleic acid.

A cytotoxin includes any agent that is detrimental to (e.g., kills) cells. For a description of these classes of drugs which are known in the art, and their mechanisms of action, see Goodman et al. (1990). Additional techniques relevant to the preparation of antibody immunotoxins are provided in for instance U.S. Pat. No. 5,194,594. Exemplary toxins include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin and the tricothecenes. See, for example, WO93/21232.

Suitable therapeutic agents for forming immunoconjugates of the present disclosure include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin, antimetabolites (such as methotrexate, 6-mercaptopurine, 6-thioguanine, cytarabine, fludarabin, 5-fluorouracil, decarbazine, hydroxyurea, asparaginase, gemcitabine, cladribine), alkylating agents (such as mechlorethamine, thioepa, chlorambucil, melphalan, carmustine (BSNU), lomustine (CCNU), cyclophosphamide, busulfan, dibromomannitol, streptozotocin, dacarbazine (DTIC), procarbazine, mitomycin C, cisplatin and other platinum derivatives, such as carboplatin), antibiotics (such as dactinomycin (formerly actinomycin), bleomycin, daunorubicin (formerly daunomycin), doxorubicin, idarubicin, mithramycin, mitomycin, mitoxantrone, plicamycin, anthramycin (AMC)).

A variety of radionuclides are available for the production of radioconjugated antibodies. Examples include, but are not limited to, ²¹²Bi, ¹³¹I, ⁹⁰Y, and ¹⁸⁶Re.

In another example of the disclosure, the antibody may be conjugated to a “receptor” (such as streptavidin) for utilization in EPC pretargeting wherein the antibody-receptor conjugate is administered to the patient, followed by removal of unbound conjugate from the circulation using a clearing agent and then administration of a “ligand” (e.g., avidin) that is conjugated to a therapeutic agent (e.g., a radionucleotide).

The antibodies of the present disclosure can be further modified to contain additional nonproteinaceous moieties that are known in the art and readily available. For example, the moieties suitable for derivatization of the antibody are water soluble polymers. Non-limiting examples of water soluble polymers include, but are not limited to, polyethylene glycol (PEG), copolymers of ethylene glycol/propylene glycol, carboxymethylcellulose, dextran or polyvinyl alcohol.

### Peptides and Polypeptides

In another example of the disclosure, the compound that binds to a protein as described herein according to any example of the disclosure is a peptide. For example, the peptide is derived from a ligand of a cell surface marker or protein as described herein according to any example of the disclosure (e.g., from a ligand binding region of the protein or marker).

Alternatively, a ligand is a peptide isolated from a random peptide library. To identify a suitable ligand, a random peptide library is generated and screened as described in U.S. Pat. No. 5,733,731, U.S. Pat. No. 5,591,646 and U.S. Pat. No. 5,834,318. Generally, such libraries are generated from short random oligonucleotides that are expressed either in vitro or in vivo and displayed in such a way to facilitate screening of the library to identify a peptide that. is capable of specifically binding to a protein or peptide of interest. Methods of display include, phage display, retroviral display, bacterial surface display, bacterial flagellar display, bacterial spore display, yeast surface display, mammalian surface display, and methods of in vitro display including, mRNA display, ribosome display and covalent display.

A peptide that is capable of binding a protein or peptide of interest is identified by a number of methods known in the art, such as, for example, standard affinity purification methods as described, for example in Scopes, 1994) purification using FACS analysis as described in U.S. Pat. No. 645,563, or purification using biosensor technology as described in Gilligan et al, 2002.

Another polypeptide that reduces the activity of a protein set forth in any one or more of Tables 1-6 is a soluble form of the protein. For example, one or more extracellular domains of the protein is(are) fused to a Fc region of an antibody. Such a polypeptide binds to a ligand of a protein set forth in any one or more of Tables 1-6 and reduces or prevents the ligand's ability to bind to induce activity of the protein. Methods for producing Fc fusion proteins are known in the art and described, for example, in WO92/12994 and U.S. Pat. No. 6,710,169.

### Small Molecules

A chemical small molecule library is also clearly contemplated for the identification of ligands that specifically bind to a protein or cell surface marker as described herein according to any example of the disclosure. Chemical small molecule libraries are available commercially or alternatively may be generated using methods known in the art, such as, for example, those described in U.S. Pat. No. 5,463,564.

### Nucleic Acid Detection/Therapeutic Reagents

### Probe/Primer Design and Production

As will be apparent to the skilled artisan, the specific probe or primer used in an assay of the present disclosure will depend upon the assay format used. Clearly, a probe or primer that is capable of specifically hybridizing to or detecting the marker of interest is useful. Methods for designing probes and/or primers for, for example, PCR or hybridization are known in the art and described, for example, in Dieffenbach and Dveksler (1995). Furthermore, several software packages are publicly available that design optimal probes and/or primers for a variety of assays, e.g. Primer 3 available from the Center for Genome Research, Cambridge, Mass., USA. Probes and/or primers useful for detection of a marker associated with EPCs are assessed to determine those that do not form hairpins, self-prime or form primer dimers (e.g. with another probe or primer used in a detection assay).

Furthermore, a probe or primer (or the sequence thereof) is assessed to determine the temperature at which it denatures from a target nucleic acid (i.e. the melting temperature of the probe or primer, or Tm). Methods of determining Tm are known in the art and described, for example, in Santa Lucia (1995) or Bresslauer et al. (1986).

Methods for producing/synthesizing a probe or primer of the present disclosure are known in the art. For example, oligonucleotide synthesis is described, in Gait (1984). For example, a probe or primer may be obtained by biological synthesis (e.g. by digestion of a nucleic acid with a restriction endonuclease) or by chemical synthesis. For short sequences (up to about 100 nucleotides) chemical synthesis is desirable.

For longer sequences standard replication methods employed in molecular biology are useful, such as, for example, the use of M13 for single stranded DNA as described by Messing (1983).

Other methods for oligonucleotide synthesis include, for example, phosphotriester and phosphodiester methods (Narang, et al., 1979) and synthesis on a support (Beaucage, et al, 1981) as well as phosphoramidate technique, Caruthers, et al. (1988), and others described in Narang (1987), and the references contained therein.

LNA synthesis is described, for example, in Nielsen et al, (1997); Singh and Wengel, (1998). PNA synthesis is described, for example, in Egholm et al. (1992); Egholm et al. (1993); and Orum et al. (1993).

In one example of the disclosure, a probe or primer useful for performance of the method of the disclosure comprises a nucleotide sequence comprising at least about 20 consecutive nucleotides of a nucleic set forth in any one of Tables 1-6.

The present disclosure additionally contemplates the use of a probe or primer produced according to the methods described herein in the manufacture of a diagnostic or prognostic reagent for diagnosing or determining a predisposition to or diagnosing or prognosing an EPC-associated condition.

### Inhibition of Nucleic Acid Transcription/Translation

In one example of the disclosure, therapeutic and/or prophylactic methods as described herein according to any example of the disclosure involve reducing expression of any one or more nucleic acids set forth in any one or more of Tables 1-6. For example, such a method involves administering a compound that reduces transcription and/or translation of any one or more nucleic acids set forth in any one or more of Tables 1-6. In one example, the compound is a nucleic acid, e.g., an antisense polynucleotide, a ribozyme, a PNA, an interfering RNA, a siRNA, a microRNA

### Antisense Nucleic Acids

The term “antisense nucleic acid” shall be taken to mean a DNA or RNA or derivative thereof (e.g., LNA or PNA), or combination thereof that is complementary to at least a portion of a specific mRNA molecule encoding a polypeptide as described herein in any example of the disclosure and capable of interfering with a post-transcriptional event such as mRNA translation. The use of antisense methods is known in the art (see for example, Hartmann and Endres, 1999).

An antisense nucleic acid of the disclosure will hybridize to a target nucleic acid under physiological conditions. Antisense nucleic acids include sequences that correspond to structural genes or coding regions or to sequences that effect control over gene expression or splicing. For example, the antisense nucleic acid may correspond to the targeted coding region of a nucleic acid set forth in any one or more of Tables 1-6, or the 5′-untranslated region (UTR) or the 3′-UTR or combination of these. It may be complementary in part to intron sequences, which may be spliced out during or after transcription, for example only to exon sequences of the target gene. The length of the antisense sequence should be at least 19 contiguous nucleotides, for example, at least 50 nucleotides, such as at least 100, 200, 500 or 1000 nucleotides of a nucleic acid set forth in any one or more of Tables 1-6 or a structural gene encoding same. The full-length sequence complementary to the entire gene transcript may be used. The length can be 100-2000 nucleotides. The degree of identity of the antisense sequence to the targeted transcript should be at least 90%, for example 95-100%.

### Catalytic Nucleic Acid

The term “catalytic nucleic acid” refers to a DNA molecule or DNA-containing molecule (also known in the art as a “deoxyribozyme” or “DNAzyme”) or a RNA or RNA-containing molecule (also known as a “ribozyme” or “RNAzyme”) which specifically recognizes a distinct substrate and catalyses the chemical modification of this substrate. The nucleic acid bases in the catalytic nucleic acid can be bases A, C, G, T (and U for RNA).

Typically, the catalytic nucleic acid contains an antisense sequence for specific recognition of a target nucleic acid, and a nucleic acid cleaving enzymatic activity (also referred to herein as the “catalytic domain”). The types of ribozymes that are particularly useful in this disclosure are a hammerhead ribozyme (Haseloff and Gerlach, 1988; Perriman et al. 1992) and a hairpin ribozyme (Zolotukiin et al., 1996; Klein et al., 1998; Shippy et al., 1999).

### RNA Interference

RNA interference (RNAi) is useful for specifically inhibiting the production of a particular protein. Although not wishing to be limited by theory, Waterhouse et al. (1998) have provided a model for the mechanism by which dsRNA (duplex RNA) can be used to reduce protein production. This technology relies on the presence of dsRNA molecules that contain a sequence that is essentially identical to the mRNA of the gene of interest or part thereof, in this case an mRNA encoding a protein set forth in any one or more of Tables 1-6. Conveniently, the dsRNA can be produced from a single promoter in a recombinant vector host cell, where the sense and anti-sense sequences are flanked by an unrelated sequence which enables the sense and anti-sense sequences to hybridize to form the dsRNA molecule with the unrelated sequence forming a loop structure. The design and production of suitable dsRNA molecules for the present disclosure is well within the capacity of a person skilled in the art, particularly considering WO99/32619, WO99/53050, WO99/49029, and WO01/34815.

The length of the sense and antisense sequences that hybridize should each be at least 19 contiguous nucleotides, such as at least 30 or 50 nucleotides, for example at least 100, 200, 500 or 1000 nucleotides. The full-length sequence corresponding to the entire gene transcript may be used. The lengths can be 100-2000 nucleotides. The degree of identity of the sense and antisense sequences to the targeted transcript should be at least 85%, for example, at least 90% such as, 95-100%.

Exemplary small interfering RNA (“siRNA”) molecules comprise a nucleotide sequence that is identical to about 19-21 contiguous nucleotides of the target mRNA. For example, the siRNA sequence commences with the dinucleotide AA, comprises a GC-content of about 30-70% (for example, 30-60%, such as 40-60% for example about 45%-55%), and does not have a high percentage identity to any nucleotide sequence other than the target in the genome of the mammal in which it is to be introduced, for example as determined by standard BLAST search.

### Detectably Labeled Compounds

In one example, a compound as described herein according to any example of the disclosure comprises one or more detectable markers to facilitate detection and/or isolation. For example, the compound comprises a fluorescent label such as, for example, fluorescein (FITC), 5,6-carboxymethyl fluorescein, Texas red, nitrobenz-2-oxa-1,3-diazol-4-yl (NBD), coumarin, dansyl chloride, rhodamine, 4′-6-diamidino-2-phenylinodole (DAPI), and the cyanine dyes Cy3, Cy3.5, Cy5, Cy5.5 and Cy7, fluorescein (5-carboxyfluorescein-N-hydroxysuccinimide ester), rhodamine (5,6-tetramethyl rhodamine). The absorption and emission maxima, respectively, for some of these fluorescent compounds are: FITC (490 nm; 520 nm), Cy3 (554 nm; 568 nm), Cy3.5 (581 nm; 588 nm), Cy5 (652 nm: 672 nm), Cy5.5 (682 nm; 703 nm) and Cy7 (755 nm; 778 nm).

Alternatively, or in addition, the compound that binds to a protein or cell surface marker as described herein according to any example of the disclosure is labeled with, for example, a fluorescent semiconductor nanocrystal (as described, for example, in U.S. Pat. No. 6,306,610).

Alternatively, or in addition, the compound is labeled with, for example, a magnetic or paramagnetic compound, such as, iron, steel, nickel, cobalt, rare earth materials, neodymium-iron-boron, ferrous-chromium-cobalt, nickel-ferrous, cobalt-platinum, or strontium ferrite.

### Pharmaceutical Compositions

Compounds of the present disclosure suitable for treating or preventing an EPC-associated condition (syn. active ingredients) are useful for parenteral, topical, oral, or local administration, aerosol administration, or transdermal administration, for prophylactic or for therapeutic treatment. Accordingly, in some examples, the compositions comprise an effective amount of the compound or a therapeutically effective amount of the compound or a prophylactically effective amount of the compound.

As used herein, the term “effective amount” shall be taken to mean a sufficient quantity of a compound to bind to the target protein in vivo and to reduce or inhibit or prevent EPC activity in vivo, compared to the same level in a subject or cell, tissue or organ thereof prior to administration and/or compared to a subject or cell, tissue or organ thereof from a subject of the same species to which the compound has not been administered. For example, the term “effective amount” means a sufficient quantity of the compound to reduce, prevent, or ameliorate an EPC-associated condition and/or to kill EPCs in a subject. The skilled artisan will be aware that such an amount will vary depending on, for example, the specific compounds administered and/or the particular subject and/or the type or severity or level of disease. Accordingly, this term is not to be construed to limit the disclosure to a specific quantity, e.g., weight or amount of compound(s); rather the present disclosure encompasses any amount of the compound(s) sufficient to achieve the stated result in a subject.

As used herein, the term “therapeutically effective amount” shall be taken to mean a sufficient quantity of a compound to reduce or inhibit one or more symptoms of an EPC-associated condition to a level that is below that observed and accepted as clinically diagnostic or clinically characteristic of that disease. The skilled artisan will be aware that such an amount will vary depending on, for example, the specific compound(s) administered and/or the particular subject and/or the type or severity or level of disease. Accordingly, this term is not to be construed to limit the disclosure to a specific quantity, e.g., weight or amount of compound(s), rather the present disclosure encompasses any amount of the compound(s) sufficient to achieve the stated result in a subject.

As used herein, the term “prophylactically effective amount” shall be taken to mean a sufficient quantity of a compound to prevent or inhibit or delay the onset of one or more detectable symptoms of an EPC-associated condition. The skilled artisan will be aware that such an amount will vary depending on, for example, the specific compound(s) administered and/or the particular subject and/or the type or severity or level of disease and/or predisposition (genetic or otherwise) to the disease. Accordingly, this term is not to be construed to limit the disclosure to a specific quantity, e.g., weight or amount of compound(s), rather the present disclosure encompasses any amount of the compound(s) sufficient to achieve the stated result in a subject.

The pharmaceutical compositions can be administered in a variety of unit dosage forms depending upon the method of administration. For example, unit dosage forms suitable for oral administration include powder, tablets, pills, capsules and lozenges. It is recognized that the pharmaceutical compositions of this disclosure, when administered orally, must be protected from digestion. This is typically accomplished either by complexing the compound with a composition to render it resistant to acidic and enzymatic hydrolysis or by packaging the compound in an appropriately resistant carrier such as a liposome. Means of protecting proteins from digestion are known in the art.

The pharmaceutical compositions of this disclosure are particularly useful for parenteral administration, such as intravenous administration or administration into a body cavity or lumen of an organ or joint. The compositions for administration will commonly comprise a solution of the compound of the present disclosure dissolved in a pharmaceutically acceptable carrier, for example an aqueous carrier. A variety of aqueous carriers can be used, e.g., buffered saline and the like. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of compounds of the present disclosure in these formulations can vary widely, and will be selected primarily based on fluid volumes, viscosities, body weight and the like in accordance with the particular mode of administration selected and the patient's needs. Exemplary carriers include water, saline, Ringer's solution, dextrose solution, and 5% human serum albumin. Nonaqueous vehicles such as mixed oils and ethyl oleate may also be used. Liposomes may also be used as carriers. The vehicles may contain minor amounts of additives that enhance isotonicity and chemical stability, e.g., buffers and preservatives.

The compounds of the present disclosure can be formulated for parenteral administration, e.g., formulated for injection via the intravenous, intramuscular, sub-cutaneous, transdermal, or other such routes, including peristaltic administration and direct instillation into a tumor disease site (intracavity administration). The preparation of an aqueous composition that contains the compounds of the present disclosure as an active ingredient will be known to those of skill in the art.

Suitable pharmaceutical compositions in accordance with the disclosure will generally include an amount of the compounds of the present disclosure admixed with an acceptable pharmaceutical diluent or excipient, such as a sterile aqueous solution, to give a range of final concentrations, depending on the intended use. The techniques of preparation are generally known in the art as exemplified by Remington's Pharmaceutical Sciences, 16th Ed. Mack Publishing Company, 1980, incorporated herein by reference.

Upon formulation, compounds of the present disclosure will be administered in a manner compatible with the dosage formulation and in such amount as is therapeutically/prophylactically effective. Formulations are easily administered in a variety of dosage forms, such as the type of injectable solutions described above, but other pharmaceutically acceptable forms are also contemplated, e.g., tablets, pills, capsules or other solids for oral administration, suppositories, pessaries, nasal solutions or sprays, aerosols, inhalants, liposomal forms and the like. Pharmaceutical “slow release” capsules or compositions may also be used. Slow release formulations are generally designed to give a constant drug level over an extended period and may be used to deliver compounds of the present disclosure.

WO2002/080967 describes compositions and methods for administering aerosolized compositions comprising antibodies for the treatment of, e.g., asthma, which are also suitable for administration of an antibody of the present disclosure.

Suitable dosages of compounds of the present disclosure will vary depending on the specific compound, the condition to be treated and/or the subject being treated. It is within the ability of a skilled physician to determine a suitable dosage, e.g., by commencing with a sub-optimal dosage and incrementally modifying the dosage to determine an optimal or useful dosage. Alternatively, to determine an appropriate dosage for treatment/prophylaxis, data from the cell culture assays or animal studies are used, wherein a suitable dose is within a range of circulating concentrations that include the ED50 of the active compound with little or no toxicity. The dosage may vary within this range depending upon the dosage form employed and the route of administration utilized. A therapeutically/prophylactically effective dose can be estimated initially from cell culture assays. A dose may be formulated in animal models to achieve a circulating plasma concentration range that includes the IC50 (i.e., the concentration of the compound which achieves a half-maximal inhibition of symptoms) as determined in cell culture. Such information can be used to more accurately determine useful doses in humans. Levels in plasma may be measured, for example, by high performance liquid chromatography.

In one example, a composition of the present disclosure comprising a compound that inhibits or kills EPCs additionally comprises a chemotherapeutic agent. Such a composition is useful for treating a cancer, e.g., by inhibiting neovascularization and by killing or preventing proliferation of cancer cells. Exemplary chemotherapeutic agents are described, for example, in W02006/0334488 and include alkylating agents such as thiotepa; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including the synthetic analogue topotecan); bryostatin; callystatin; dolastatin; duocarmycin; eleutherobin; pancratistatin; a sarcodictyin; spongistatin; nitrogen mustards such as chlorambucil; nitrosureas such as carmustine; antibiotics such as the enediyne antibiotics; dynemicin, dactinomycin, daunorubicin, detorubicin, doxorubicin, epirubicin, mitomycins such as mitomycin C, peplomycin, potfiromycin, puromycin, streptozocin, zinostatin, zorubicin; anti-metabolites such as methotrexate and 5-fluorouracil (5-FU); folic acid analogues such as denopterin; purine analogs such as fludarabine; pyrimidine analogs such as ancitabine, azacitidine; androgens such as calusterone; hydroxyurea; maytansinoids such as maytansine; vindesine; and pharmaceutically acceptable salts, acids or derivatives of any of the above.

In another example, a composition of the disclosure additionally comprises an anti-inflammatory compound or is administered with an anti-inflammatory compound, e.g., celecoxib, diclofenac potassium, diclofenac sodium, etodolac, fenoprofen calcium, flurbiprofen, ibuprofen, indomethacin, indomethacin sodium trihydrate, ketoprofen, ketorolac tromethamine, nabumetone, naproxen, naproxen sodium, oxaprozin, piroxicam, rofecoxib, or sulindac.

In another example, a composition of the disclosure additionally comprises a methotrexate or is administered with methotrexate.

### Cellular Compositions

In one example of the present disclosure EPCs and/or progeny cells thereof are administered in the form of a composition. For example, such a composition comprises a pharmaceutically acceptable carrier and/or excipient.

Suitable carriers for this disclosure include those conventionally used, e.g., water, saline, aqueous dextrose, lactose, Ringer's solution, a buffered solution, hyaluronan and glycols are exemplary liquid carriers, particularly (when isotonic) for solutions. Suitable pharmaceutical carriers and excipients include starch, cellulose, glucose, lactose, sucrose, gelatin, malt, rice, flour, chalk, silica gel, magnesium stearate, sodium stearate, glycerol monostearate, sodium chloride, glycerol, propylene glycol, water, ethanol, and the like.

In another example, a carrier is a media composition, e.g., in which a cell is grown or suspended. For example, such a media composition does not induce any adverse effects in a subject to whom it is administered.

Exemplary carriers and excipients do not adversely affect the viability of a cell and/or the ability of a cell to reduce, prevent or delay an EPC-associated condition.

In one example, the carrier or excipient provides a buffering activity to maintain the cells at a suitable pH to thereby exert a biological activity, e.g., the carrier or excipient is phosphate buffered saline (PBS). PBS represents an attractive carrier or excipient because it interacts with cells minimally and permits rapid release of the cells, in such a case, the composition of the disclosure may be produced as a liquid for direct application to the blood stream or into a tissue or a region surrounding or adjacent to a tissue, e.g., by injection.

EPCs and/or progeny cells thereof can also be incorporated or embedded within scaffolds that are recipient-compatible and which degrade into products that are not harmful to the recipient. These scaffolds provide support and protection for cells that are to be transplanted into the recipient subjects. Natural and/or synthetic biodegradable scaffolds are examples of such scaffolds. Other suitable scaffolds include polyglycolic acid scaffolds, e.g., as described by Vacanti, et al. (1988); Cima, et al. (1991); Vacanti, et al. (1991); or synthetic polymers such as polyanhydrides, polyorthoesters, and polylactic acid.

For example, the composition comprises an effective amount or a therapeutically or prophylactically effective amount of cells. For example, the composition comprises about 1×10⁵ EPCs/kg to about 1×10⁹ EPCs/kg or about 1×10⁶ EPCs/kg to about 1×10⁸ EPCs/kg or about 1×10⁶ EPCs/kg to about 1×10⁷ EPCs/kg. The exact amount of cells to be administered is dependent upon a variety of factors, including the age, weight, and sex of the patient, and the extent and severity of the EPC-associated condition.

The cellular compositions of this disclosure can be administered to the subject by any recognized methods, either systemically or at a localized site. In one example, the most convenient time to administer the cells to improve grafting is during the time of surgery. To treat an autoimmune disease, the composition can be administered at the onset of symptoms and/or following onset of symptoms or even prior to the onset of symptoms (e.g., following detection of an autoimmune response). To keep the cells at the site until completion of the surgical procedure, it is convenient to administer the cells in a pharmaceutically compatible artificial gel, or in clotted plasma, by utilizing any other known controlled release mechanism (see above), or immobilized on a solid or semi-solid support. When less invasive procedures are desired, the composition can be injected at a desired location through a needle. For deeper sites, the needle can be positioned using endoscopic ultrasound techniques, radioscintigraphy, or some other imaging technique, alone or in combination with the use of an appropriate scope or cannula. For such applications, the cell population is conveniently administered when suspended in isotonic saline or a neutral buffer.

In one example, a cellular composition of the present disclosure is administered together with an agent that enhances endothelialization, such as, VEGF. The cells and the agent can be administered in the same composition and/or can be administered separately.

As discussed herein, EPCs and/or compositions that bind to EPCs can be immobilized on a solid or semi-solid matrix prior to administration to a subject. Such matrices are useful for, for example, forming vascular grafts that are endothelialized, thereby reducing the risk of thrombosis. Exemplary matrices will be apparent to the skilled artisan and include hydrogel materials, blends of hydrophilic and hydrophobic polymers such as polyethylene glycol (PEG) and d,l-polylactic acid (d,l-PLA), polyester and polytetrafluoroethyle.

### Isolation or Enrichment of Cells

One exemplary approach to enrich for the desired cells is magnetic bead cell sorting (MACS) or any other cell sorting method making use of magnetism, e.g., Dynabeads®. A conventional MACS procedure is described by Miltenyi et al. (1990). In this procedure, cells are labeled with magnetic beads bound to an antibody or other compound that binds to a cell surface marker or protein and the cells are passed through a paramagnetic separation column or exposed to another form of magnetic field. Cells that are magnetically labeled are trapped in the column; cells that are not pass through. The trapped cells are then eluted from the column.

The MACS technique is equally applicable to negative selection, e.g., removal of cells expressing an undesirable marker, i.e., undesirable cells. Such a method involves contacting a population of cells with a magnetic particle labeled with a compound that binds to a cell surface marker expressed at detectable levels on the undesirable cell type(s). Following incubation, samples are washed and resuspended and passed through a magnetic field to remove cells bound to the immunomagnetic beads. The remaining cells depleted of the undesirable cell type(s) are then collected.

In another example, a compound that binds to a protein or cell surface marker is immobilized on a solid surface and a population of cells is contacted thereto. Following washing to remove unbound cells, cells bound to the compound can be recovered, e.g., eluted, thereby isolating or enriching for cells expressing the protein to which the compound binds. Alternatively, cells that do not bind to the compound can be recovered if desired.

In a further example, cells are isolated or enriched using fluorescence activated cell sorting (FACS). FACS is a known method for separating particles, including cells, based on the fluorescent properties of the particles and described, for example, in Kamarch (1987). Generally, this method involves contacting a population of cells with compounds capable of binding to one or more proteins or cell surface markers, wherein compounds that bind to distinct markers are labeled with different fluorescent moieties, e.g., fluorophores. The cells are entrained in the center of a narrow, rapidly flowing stream of liquid. The flow is arranged so that there is a separation between cells relative to their diameter. A vibrating mechanism causes the stream of cells to break into individual droplets. The system is adjusted so that there is a low probability of more than one cell being in a droplet. Just before the stream breaks into droplets the flow passes through a fluorescence measuring station where the fluorescent character of interest of each cell is measured, e.g., whether or not a labeled compound is bound thereto. An electrical charging ring is placed at the point where the stream breaks into droplets. A charge is placed on the ring based on the immediately prior fluorescence intensity measurement and the opposite charge is trapped on the droplet as it breaks from the stream. The charged droplets then fall through an electrostatic deflection system that diverts droplets into containers based upon their charge, e.g., into one container if a labeled compound is bound to the cell and another container if not. In some systems the charge is applied directly to the stream and the droplet breaking off retains charge of the same sign as the stream. The stream is then returned to neutral after the droplet separates.

### Cell Culture

Following isolation, cells of the disclosure can be maintained under standard cell culture conditions. For example, the cells can be maintained in Dulbecco's Minimal Essential Medium (DMEM) or any other appropriate cell culture medium known in the art, e.g., as described above. Other appropriate media include, for example, MCDB, Minimal Essential Medium (MEM), IMDM, and RPMI. Additional suitable media for culturing EPCs include endothelial growth media, such as EGM-2 plus Bullet kit (available from Lonza Group Ltd).

Cell cultures can be incubated at about 37° C. in a humidified incubator. Cell culture conditions can vary considerably for the cells of the present disclosure. For example, the cells are maintained in an environment suitable for cell growth, e.g., comprising 5% O₂, 10% CO₂, 85% N₂ or comprising 10% CO₂ in air.

In some examples, cells are cultured on an extracellular matrix, e.g., fibronectin, laminin or EGM-2 and/or type IV collagen.

In some examples, cells are cultured in the presence of one or more growth factors, e.g., VEGF, insulin-like growth factor-1 and/or basic fibroblast growth factor. The cells may also be cultured in the presence of one or more vitamins and/or antioxidants, e.g., ascorbic acid.

In another example, the cells are cultured in suspension, i.e., without adhering to tissue culture plastic-ware or an extracellular matrix or components thereof. In this regard, the inventors have clearly exemplified culturing of EPCs in suspension culture.

### Detection Assays

### Protein Detection Assays

In one example, the method of the disclosure detects the presence of a protein. The amount, level or presence of a protein is determined using any of a variety of techniques known to the skilled artisan such as, for example, a technique selected from the group consisting of, immunohistochemistry, immunofluorescence, an immunoblot, a Western blot, a dot blot, an enzyme-linked immunosorbent assay (ELISA), radioimmunoassay (RIA), enzyme immunoassay, fluorescence resonance energy transfer (FRET), matrix-assisted laser desorption/ionization time-of-flight mass spectrometry (MALDI-tof-MS), electrospray ionization (ESI-MS) (including tandem mass spectrometry, e.g. LC-ESI-MS/MS and MALDI-tof/tof-MS), biosensor technology, evanescent fiber-optics technology or protein chip technology.

In one example the assay used to determine the amount or level of a protein is a semi-quantitative method.

In another example the assay used to determine the amount or level of a protein is a quantitative method.

For example, the protein is detected with an immunoassay, e.g., using an assay selected from the group consisting of, immunohistochemistry, immunofluorescence, ELISA, fluorescence-linked immunosorbent assay (FLISA) Western blotting, RIA, a biosensor assay, a protein chip assay and an immunostaining assay (e.g. immunofluorescence).

Standard solid-phase ELISA or FLISA formats are particularly useful in determining the concentration of a protein from a variety of samples.

In one form such an assay involves immobilizing a biological sample onto a solid matrix, such as, for example a polystyrene or polycarbonate microwell or dipstick, a membrane, or a glass support (e.g. a glass slide). A compound (e.g., an antibody) that specifically binds to a protein set out in any one of Tables 1-6 is brought into direct contact with the immobilized biological sample, and forms a direct bond with any of its target protein present in said sample. This antibody is generally labeled with a detectable reporter molecule, such as, for example, a fluorescent label (e.g. FITC or Texas Red) or a fluorescent semiconductor nanocrystal (as described in U.S. Pat. No. 6,306,610) in the case of a FLISA or an enzyme (e.g. horseradish peroxidase (HRP), alkaline phosphatase (AP) or β-galactosidase) in the case of an ELISA, or alternatively a second labeled antibody can be used that binds to the first antibody. Following washing to remove any unbound antibody the label is detected either directly, in the case of a fluorescent label, or through the addition of a substrate, such as for example hydrogen peroxide, TMB, or toluidine, or 5-bromo-4-chloro-3-indol-beta-D-galactopyranoside (x-gal) in the case of an enzymatic label. Such ELISA- or FLISA-based systems are particularly suitable for quantification of the amount of a protein in a sample, by calibrating the detection system against known amounts of a protein standard to which the antibody binds, such as for example, an isolated and/or recombinant polypeptide or immunogenic fragment thereof or epitope thereof.

In another form, an ELISA or FLISA comprises of immobilizing a compound (e.g., an antibody) on a solid matrix, such as, for example, a membrane, a polystyrene or polycarbonate microwell, a polystyrene or polycarbonate dipstick or a glass support. A sample is then brought into physical relation with the compound, and the protein to which the compound binds is bound or ‘captured’. The bound protein is then detected using a second labeled compound that binds to a different protein or a different site in the same protein. Alternatively, a third labeled antibody can be used that binds the second (detecting) antibody.

It will be apparent to the skilled person that the assay formats described herein are amenable to high throughput formats, such as, for example, automation of screening processes or a microarray format as described in Mendoza et al. (1999). Furthermore, variations of the above-described assay will be apparent to those skilled in the art, such as, for example, a competitive ELISA.

In an alternative example, a polypeptide is detected within or on a cell, using methods known in the art, such as, for example, immunohistochemistry or immunofluorescence. Methods using immunofluorescence are exemplary, as they are quantitative or at least semi-quantitative. Methods of quantitating the degree of fluorescence of a stained cell are known in the art and described, for example, in Cuello (1984).

Biosensor devices generally employ an electrode surface in combination with current or impedance measuring elements to be integrated into a device in combination with the assay substrate (such as that described in U.S. Pat. No. 5,567,301). A compound that specifically binds to a protein or is incorporated onto the surface of a biosensor device and a biological sample contacted to said device. A change in the detected current or impedance by the biosensor device indicates protein binding to said antibody. Some forms of biosensors known in the art also rely on surface plasmon resonance to detect protein interactions, whereby a change in the surface plasmon resonance surface of reflection is indicative of a protein binding to a ligand or antibody (U.S. Pat. No. 5,485,277 and U.S. Pat. No. 5,492,840).

Biosensors are of particular use in high throughput analysis due to the ease of adapting such systems to micro- or nano-scales. Furthermore, such systems are conveniently adapted to incorporate several detection reagents, allowing for multiplexing of diagnostic reagents in a single biosensor unit. This permits the simultaneous detection of several proteins or peptides in a small amount of body fluids.

Evanescent biosensors are also useful as they do not require the pretreatment of a biological sample prior to detection of a protein of interest. An evanescent biosensor generally relies upon light of a predetermined wavelength interacting with a fluorescent molecule, such as for example, a fluorescent antibody attached near the probe's surface, to emit fluorescence at a different wavelength upon binding of the target polypeptide to the compound.

Micro- or nano-cantilever biosensors are also useful as they do not require the use of a detectable label. A cantilever biosensor utilizes a compound capable of specifically detecting the analyte of interest that is bound to the surface of a deflectable arm of a micro- or nano-cantilever. Upon binding of the analyte of interest (e.g. a marker within a polypeptide) the deflectable arm of the cantilever is deflected in a vertical direction (i.e. upwards or downwards). The change in the deflection of the deflectable arm is then detected by any of a variety of methods, such as, for example, atomic force microscopy, a change in oscillation of the deflectable arm or a change in pizoresistivity. Exemplary micro-cantilever sensors are described in US20030010097.

To produce protein chips, the proteins, peptides, polypeptides, antibodies or ligands that are able to bind specific antibodies or proteins of interest are bound to a solid support such as for example glass, polycarbonate, polytetrafluoroethylene, polystyrene, silicon oxide, metal or silicon nitride. This immobilization is either direct (e.g. by covalent linkage, such as, for example, Schiff's base formation, disulfide linkage, or amide or urea bond formation) or indirect. Methods of generating a protein chip are known in the art and are described in for example US20020136821, US20020192654, US20020102617 and U.S. Pat. No. 6,391,625. To bind a protein to a solid support it is often necessary to treat the solid support so as to create chemically reactive groups on the surface, such as, for example, with an aldehyde-containing silane reagent. Alternatively, an antibody or ligand may be captured on a microfabricated polyacrylamide gel pad and accelerated into the gel using microelectrophoresis as described in, Arenkov et al. (2000). In this regard, the present disclosure also provides a protein chip comprising a plurality of compounds capable of binding to at least two proteins set forth in any one or more of Tables 1-6. In one example, the compounds are antibodies or polypeptides comprising antigen binding domains thereof.

### Nucleic Acid Detection Assays

In another example, an EPC is detected and/or an EPC-associated condition is diagnosed/prognosed by detecting the level of expression of a nucleic acid. Exemplary assays for such detection include quantitative RT-PCR, NASBA, TMA or ligase-chain reaction.

Methods of RT-PCR are known in the art and described, for example, in Dieffenbach (ed) and Dveksler (ed) (1995).

Methods of TMA or self-sustained sequence replication (3SR) use two or more oligonucleotides that flank a target sequence, a RNA polymerase, RNase H and a reverse transcriptase. One oligonucleotide (that also comprises an RNA polymerase binding site) hybridizes to an RNA molecule that comprises the target sequence and the reverse transcriptase produces cDNA copy of this region. RNase H is used to digest the RNA in the RNA-DNA complex, and the second oligonucleotide used to produce a copy of the cDNA. The RNA polymerase is then used to produce a RNA copy of the cDNA, and the process repeated.

NASBA systems relies on the simultaneous activity of three enzymes (a reverse transcriptase, RNase H and RNA polymerase) to selectively amplify target mRNA sequences. The mRNA template is transcribed to cDNA by reverse transcription using an oligonucleotide that hybridizes to the target sequence and comprises a RNA polymerase binding site at its 5′ end. The template RNA is digested with RNase H and double-stranded DNA is synthesized. The RNA polymerase then produces multiple RNA copies of the cDNA and the process is repeated.

Clearly, the hybridization to and/or amplification of a nucleic acid using any of these methods is detectable using, for example, electrophoresis and/or mass spectrometry. In this regard, one or more of the probes/primers and/or one or more of the nucleotides used in an amplification reaction may be labeled with a detectable marker to facilitate rapid detection of a cellular marker, for example, a fluorescent label (e.g. Cy5 or Cy3) or a radioisotope (e.g. ³²P). Alternatively, amplification of a nucleic acid may be continuously monitored using a melting curve analysis method, such as that described in, for example, U.S. Pat. No. 6,174,670.

As exemplified herein, the present disclosure additionally contemplates microarray-based methods for detecting levels of expression of nucleic acids. Generally such methods involve the use of solid substrates having immobilized thereon a plurality of different oligonucleotides that specifically hybridize to nucleic acids, e.g., cDNA/cRNA of transcripts. A nucleic acid sample, e.g., cDNA/cRNA is labeled with a detectable marker. For example, two samples are prepared (e.g., from a population of EPCs and a population of non-EPCs, such as HUVECs) and each sample is labeled with a detectable marker. The samples are then mixed and contacted with the solid support under conditions sufficient to permit nucleic acid hybridization. Following a sufficient time to permit nucleic acid hybridization, the solid support is washed to remove non-hybridized nucleic acid and the level of the detectable marker hybridized to the oligonucleotides is determined so as to determine the level of expression of the transcript giving rise to each cDNA/cRNA. When two samples are hybridized to a solid support, the level of each detectable marker can be detected to determine the difference in the level of expression of each transcript (e.g., fold change in expression).

### Imaging Methods

As will be apparent to the skilled artisan from the foregoing, the present disclosure also contemplates imaging methods using a compound that binds to a protein of the disclosure. For imaging, a compound is generally conjugated to a detectable label, which can be any molecule or agent that can emit a signal that is detectable by imaging. However, a secondary labeled compound that specifically binds to a compound that binds to a protein of the disclosure may also be used. Exemplary detectable labels include a protein, a radioisotope, a fluorophore, a visible light emitting fluorophore, infrared light emitting fluorophore, a metal, a ferromagnetic substance, an electromagnetic emitting substance a substance with a specific magnetic resonance (MR) spectroscopic signature, an X-ray absorbing or reflecting substance, or a sound altering substance.

The compound that binds to a protein set forth in any one or more of Tables 1-6 (and, if used the labeled secondary compound) can be administered either systemically or locally to the tumor, organ, or tissue to be imaged, prior to the imaging procedure. Generally, the compound is administered in one or more doses effective to achieve the desired optical image of a tumor, tissue, or organ. Such doses may vary widely, depending upon the particular compound employed, condition to be imaged, tissue, or organ subjected to the imaging procedure, the imaging equipment being used, and the like.

In some examples of the disclosure, the compound is used as an in vivo optical imaging agent of tissues and organs in various biomedical applications including, but not limited to, imaging of tumors, tomographic imaging of organs, monitoring of organ functions, coronary angiography, fluorescence endoscopy, laser guided surgery, photoacoustic and sonofluorescence methods, and the like. Exemplary diseases in which a compound is useful for imaging are described herein and shall be taken to apply mutatis mutandis to the present example of the disclosure. In one example, the compounds of the disclosure are useful for the detection of the presence of tumors and other abnormalities (e.g., retinopathy and/or nephropathy) by monitoring where a particular protein of the disclosure is concentrated in a subject. In another example, the compound is useful for laser-assisted guided surgery.

Examples of imaging methods include magnetic resonance imaging (MRI), MR spectroscopy, radiography, computerized tomography (CT), ultrasound, planar gamma camera imaging, single-photon emission computed tomography (SPECT), positron emission tomography (PET), other nuclear medicine-based imaging, optical imaging using visible light, optical imaging using luciferase, optical imaging using a fluorophore, other optical imaging, imaging using near infrared light, or imaging using infrared light.

Certain examples of the methods of the present disclosure further include imaging a tissue during a surgical procedure on a subject.

A variety of techniques for imaging are known to those of ordinary skill in the art. Any of these techniques can be applied in the context of the imaging methods of the present disclosure to measure a signal from the detectable label. For example, optical imaging is one imaging modality that has gained widespread acceptance in particular areas of medicine. Examples include optical labeling of cellular components, and angiography such as fluorescein angiography and indocyanine green angiography. Examples of optical imaging agents include, for example, fluorescein, a fluorescein derivative, indocyanine green, Oregon green, a derivative of Oregon green, rhodamine green, a derivative of rhodamine green, an eosin, an erytlirosin, Texas red, a derivative of Texas red, malachite green, nanogold sulfosuccinimidyl ester, cascade blue, a coumarin derivative, a naphthalene, a pyridyloxazole derivative, cascade yellow dye, dapoxyl dye.

Gamma camera imaging is contemplated as a method of imaging that can be utilized for measuring a signal derived from the detectable label. One of skill in the art will be familiar with techniques for application of gamma camera imaging. In one example, measuring a signal can involve use of gamma-camera imaging of an ¹¹¹In or ^{99m}Tc conjugate, in particular ¹¹¹In-octreotide or ^{99m}Tc-somatostatin analogue.

CT is contemplated as an imaging modality in the context of the present disclosure. By taking a series of X-rays from various angles and then combining them with a computer, CT makes it possible to build up a three-dimensional image of any part of the body. A computer is programmed to display two-dimensional slices from any angle and at any depth. The slices may be combined to build three-dimensional representations.

In CT, intravenous injection of a radiopaque contrast agent conjugated to a compound, which binds to a protein identified herein can assist in the identification and delineation of soft tissue masses when initial CT scans are not diagnostic. Similarly, contrast agents aid in assessing the vascularity of a soft tissue lesion. For example, the use of contrast agents may aid the delineation of the relationship of a tumor and adjacent vascular structures.

CT contrast agents include, for example, iodinated contrast media. Examples of these agents include iothalamate, iohexol, diatrizoate, iopamidol, ethiodol, and iopanoate. Gadolinium agents have also been reported to be of use as a CT contrast agent, for example, gadopentate.

MRI is an imaging modality that uses a high-strength magnet and radio-frequency signals to produce images. In MRI, the sample to be imaged is placed in a strong static magnetic field and excited with a pulse of radio frequency (RF) radiation to produce a net magnetization in the sample. Various magnetic field gradients and other RF pulses then act to code spatial information into the recorded signals. By collecting and analyzing these signals, it is possible to compute a three-dimensional image which, like a CT image, is normally displayed in two-dimensional slices. The slices may be combined to build three-dimensional representations.

Contrast agents used in MRI or MR spectroscopy imaging differ from those used in other imaging techniques. Examples of MRI contrast agents include gadolinium chelates, manganese chelates, chromium chelates, and iron particles. For example, a protein of the disclosure is conjugated to a compound comprising a chelate of a paramagnetic metal selected from the group consisting of scandium, titanium, vanadium, chromium, manganese, iron, cobalt, nickel, copper, molybdenum, ruthenium, cerium, indium, praseodymium, neodymium, promethium, samarium, europium, gadolinium, terbium, dysprosium, holmium, erbium, thulium, and ytterbium. A further example of imaging agents useful for the present disclosure is halocarbon-based nanoparticle such as PFOB or other fluorine-based MRI agents. Both CT and MRI provide anatomical information that aid in distinguishing tissue boundaries and vascular structure.

Imaging modalities that provide information pertaining to information at the cellular level, such as cellular viability, include PET and SPECT. In PET, a patient ingests or is injected with a radioactive substance that emits positrons, which can be monitored as the substance moves through the body.

SPECT is closely related to PET. The major difference between the two is that instead of a positron-emitting substance, SPECT uses a radioactive tracer that emits high-energy photons. SPECT is valuable for diagnosing multiple illnesses including coronary artery disease, and already some 2.5 million SPECT heart studies are done in the United States each year.

For PET, a protein of the disclosure is commonly labeled with positron-emitters such as ¹¹C, ¹³N, ¹⁵O, ¹⁸F, ⁸²Rb, ⁶²Cu, and ⁶⁸Ga. Compounds that bind to a protein set forth in any one or more of Tables 1-6 are labeled with positron emitters such as ^{99m}Tc, ²⁰¹Tl, and ⁶⁷Ga, ¹¹¹In for SPECT.

Non-invasive fluorescence imaging of animals and humans can also provide in vivo diagnostic information and be used in a wide variety of clinical specialties. For instance, techniques have been developed over the years including simple observations following UV excitation of fluorophores up to sophisticated spectroscopic imaging using advanced equipment (see, e.g., Andersson-Engels et al, 1997). Specific devices or methods known in the art for the in vivo detection of fluorescence, e.g., from fluorophores or fluorescent proteins, include, but are not limited to, in vivo near-infrared fluorescence (see, e.g., Frangioni, 2003), the Maestro™ in vivo fluorescence imaging system (Cambridge Research & Instrumentation, Inc.; Woburn, Mass.), in vivo fluorescence imaging using a flying-spot scanner (see, e.g., Ramanujam et al, 2001), and the like.

Other methods or devices for detecting an optical response include, without limitation, visual inspection, CCD cameras, video cameras, photographic film, laser-scanning devices, fluorometers, photodiodes, quantum counters, epifluorescence microscopes, scanning microscopes, flow cytometers, fluorescence microplate readers, or signal amplification using photomultiplier tubes.

In some examples, an imaging agent is tested using an in vitro or in vivo assay prior to use in humans, e.g., using a model described herein.

### Samples

To the extent that the method of the present disclosure is performed in vitro, on an isolated tissue sample, rather than as an in vivo based screen, reference to “sample” should be understood as a reference to any sample of biological material derived from an animal such as, but not limited to, a body fluid (e.g., blood or synovial fluid or cerebrospinal fluid or bone marrow), cellular material (e.g. tissue aspirate), tissue biopsy specimens or surgical specimens. The term “sample” includes extracts and/or derivatives and/or fractions of said sample, e.g., serum, plasma, peripheral blood mononuclear cells (PBMC), a buffy coat fraction. For example, the sample comprises EPCs or is likely to comprise EPCs.

The sample which is used according to the method of the present disclosure may be used directly or may require some form of treatment prior to use. For example, a biopsy or surgical sample may require homogenization or other form of cellular dispersion prior to use. Furthermore, to the extent that the sample is not in liquid form, (if such form is required or desirable) it may require the addition of a reagent, such as a buffer, to mobilize the sample.

As will be apparent from the description and/or claims herein, such an assay may require the use of a suitable control, e.g. a normal or healthy individual or a typical population, e.g., for quantification.

As used herein, the term “normal individual” shall be taken to mean that the subject is selected on the basis that they do not have abnormal numbers of EPCs in a sample derived therefrom.

A “healthy subject” is one that has not been diagnosed as suffering from an EPC-associated condition and/or is not at risk of developing an EPC-associated condition.

Alternatively, or in addition, a suitable control sample is a control data set comprising measurements of the marker being assayed for a typical population of normal and/or healthy subjects, e.g., subjects known not to suffer from an EPC-associated condition.

In one example, a reference sample is not included in an assay. Instead, a suitable reference sample is derived from an established data set previously generated from a typical population. Data derived from processing, analyzing and/or assaying a test sample is then compared to data obtained for the sample population.

### Screening Assays

As discussed hereinabove, the present disclosure also provides methods for identifying or isolating a compound that binds to and/or modulate EPC activity. Suitable compounds for screening include, for example, antibodies, peptides or small molecules, e.g., as described herein according to any example.

In some examples, this method comprises determining an agent that binds to the recited protein. Such assays will be apparent to the skilled artisan. For example, the protein or a cell expressing same is immobilized on a solid surface and contacted with a labeled compound. Following washing to remove unbound compound the level of label is detected, which is indicative of the amount of bound compound.

In some examples, the method additionally comprises determining the effect of a compound on expression of a nucleic acid or protein. Suitable methods for determining expression levels are known in the art and/or described herein.

Assays for determining EPC function are also described herein and are to be taken to apply mutatis mutandis to the present example of the disclosure.

This disclosure also encompasses for the provision of information concerning the identified or isolated compound. Accordingly, the screening methods are further modified by:

(i) optionally, determining the structure of the compound; and
(ii) providing the compound or the name or structure of the compound such as, for example, in a paper form, machine-readable form, or computer-readable form.

Naturally, for compounds that are known, albeit not previously tested, for their function using a screen provided by the present disclosure, determination of the name and/or structure of the compound is implicit. This is because the skilled artisan will be aware of the name and/or structure of the compound at the time of performing the screen.

As used herein, the term “providing the compound” shall be taken to include any chemical or recombinant synthetic means for producing the compound or alternatively, the provision of a compound that has been previously synthesized by any person or means. This clearly includes isolating the compound.

In an example, the compound or the name or structure of the compound is provided with an indication as to its use e.g., as determined by a screen described herein.

The screening assays can be further modified by:

(i) optionally, determining the structure of the compound;
(ii) optionally, providing the name or structure of the compound such as, for example, in a paper form, machine-readable form, or computer-readable form; and
(iii) providing the compound.

In an example, the synthesized compound or the name or structure of the compound is provided with an indication as to its use e.g., as determined by a screen described herein.

In one example, the compound is provided in a library of compounds, each of which or a subset of which may be separated from other members (i.e., physically isolated). In such cases, a compound is isolated from the library by its identification, which then permits a skilled person to produce that compound in isolation, e.g., in the absence of other members of the library.

In some examples, the screening methods described herein comprise determining the effect of an isolated and/or identified compound on EPC activity and/or cell numbers (e.g., cell death. Such an assay may be performed in vitro and/or in vivo.

### In Vitro Assays of EPC Activity

An exemplary in vitro method for determining EPC activity is, for example, a CFU assay in which cells are cultured on an extracellular matrix and the ability to form clonal colonies is determined. For example, EPCs are cultured for several days, e.g., at least 2 or 3 or 4 or 5 or 6 or 7 days in a suitable culture medium and the number of cell colonies adhering to the chamber in which the cells are cultured are counted. Optionally, the chamber is coated with extracellular matrix or a component thereof. Functional EPCs will be capable of forming colonies, with each colony representing a CFU. When assessing the effect of a reduction in the amount of colonies (i.e., CFUs) in the presence of the compound compared to the number of colonies (CFUs) in the absence of the compound indicates that the compound inhibits or reduces EPC activity.

Another assays include, for example, migration assays, in which the ability of an EPC to migrate in vitro to an angiogenic compound, such as, VEGF. For example, a chamber comprising a porous membrane is coated with an extracellular matrix or component thereof and EPCs cultured in the chamber. The chamber is inserted into another chamber comprising an angiogenic factor, e.g., VEGF and the cells maintained for a time sufficient for the EPCs to migrate through the pores (e.g., 4-6 hours or 1-2 days). Cells having EPC activity migrate towards the angiogenic factor and are detectable in the chamber comprising the angiogenic factor. As will be apparent to the skilled person, a compound that reduces the number of cells detectable in the chamber comprising the angiogenic factor is considered to reduce EPC activity.

Other assays include those involving culturing cells and determining those capable of uptake of acetylated-LDL and/or that bind to Ulex europaeus I lectin. In such assays, cells are cultured in the presence of labeled acetylated LDL (e.g., 1,1′-dioctadecyl-3,3,3′,3-tetramethyl-indocarbocyanine perchlorate (Dil)-Ac-LDL) and/or Ulex europaeus lectin (e.g., labeled with a detectable compound). Cells that take up acetylated LDL and/or bind to Ulex europaeus lectin are considered to have EPC activity. For example, EPCs take up acetylated LDL and bind to Ulex europaeus lectin. A compound that inhibits or reduces EPC activity reduces uptake of acetylated LDL and/or binding of Ulex europaeus lectin.

A further method for assessing EPC function is a tube formation method. In such a method, cells are cultured in a tissue culture chamber, e.g., coated with extracellular matrix or a component thereof. Cells are cultured for a sufficient period to form tubes (e.g., 1-6 days) and the tissue culture chambers observed, using microscopy. Tubes are observed between two discrete cells or clusters thereof. Tube formation is indicative of EPC activity, and a compound that reduces tube formation is considered to inhibit or reduce EPC activity.

Alternatively, or in addition, EPCs function is assessed by detecting secretion of an angiogenic factor, e.g., VEGF, hepatocyte growth factor, granulocyte-colony stimulating factor, Macrophage migration inhibitory factor interleukin 8. For example, cells are cultured for a suitable period of time (e.g., 1-6 days) and the level of angiogenic factors in culture medium determined using, for example, an ELISA or a FLISA. Secretion of higher levels of angiogenic factors than a non-EPC endothelial cell indicates EPC activity. Compounds that reduce secretion of angiogenic factors are considered to be inhibitors of EPC activity.

As will be apparent to the skilled artisan, methods of screening may involve detecting levels of cell death, cell proliferation and/or cell survival. Such methods are known in the art.

In one example, death of isolated EPCs in the presence or absence of a compound is assayed (e.g., to isolate a compound that kills EPCs), e.g., using a method for the detection of cellular components associated with cell death, such as, for example, apoptosis. Methods for detecting cell death in a cell are known in the art. For example, APOPTEST (available from Immunotech) stains cells early in apoptosis, and does not require fixation of the cell sample (Martin et al., 1994). This method utilizes an annexin V antibody to detect cell membrane re-configuration that is characteristic of cells undergoing apoptosis. Apoptotic cells stained in this manner can then be sorted either by FACS, ELISA or by adhesion and panning using immobilized annexin V antibodies. Alternatively, a terminal deoxynucleotidyl transferase-mediated biotinylated UTP nick end-labeling (TUNEL) assay is used to determine the level of cell death. The TUNEL assay uses the enzyme terminal deoxynucleotidyl transferase to label 3′-OH DNA ends, generated during apoptosis, with biotinylated nucleotides. The biotinylated nucleotides are then detected by using streptavidin conjugated to a detectable marker. Kits for TUNEL staining are available from, for example, Intergen Company, Purchase, N.Y. Alternatively, or in addition, an activated caspase, such as, for example, Caspase 3 is detected. Several caspases are effectors of apoptosis and, as a consequence, are only activated to significant levels in a cell undergoing programmed cell death. Kits for detection of an activated caspase are available from, for example, Promega Corporation, Madison Wis., USA. Such assays are useful for both immunocytochemical or flow cytometric analysis of cell death. Such assays can be performed with other cells, e.g. mature endothelial cells to identify and/or isolate compounds that selectively kill EPCs.

In one example, the phenotype being assayed is cell survival. Cell survival may simply be detected by maintaining the cells for a sufficient time for a visible colony of cells to form. Clearly, this provides a simple method for high-throughput screening of compounds as compounds capable of inducing cell survival are easily recovered from the colony of cells.

Alternatively, cell viability or cell metabolism may be detected and/or assayed. By way of example, non-fluorescent resazurin is added to cells cultured in the presence of a peptide of the present disclosure. Viable cells reduce resazurin to red-fluorescent resorufin, easily detectable using, for example, microscopy or a fluorescent plate reader. This marker of cell viability is useful for a variety of different cell types, from bacteria to higher eukaryotes. Kits for analysis of cell viability are available, for example, from Molecular Probes, Eugene, Oreg., USA. Other assays for cell viability include, for example, assays that detect Water-Soluble Tetrazolium GLT008 (WST-8) reduction to formazan salt in live cells (Alexis Biochemicals), staining of live cells with cell-permeable calcein acetoxymethyl (calcein AM) which is converted to fluorescent calcein by intracellular esterases, detection of reduction of 3′-{1-[(phenylamino)carbonyl]-3,4-tetrazolium}-bis(4-methoxy-6-nitro)benzenesulfonic acid hydrate] (XTT) to formazan salt (Intergen), or (4,5-dimethylthiazol-2-yl)-5-(3-carboxymethoxyphenyl)-2-(4-sulfophenyl)-2H-tetrazolium) PES: phenazine ethosulfate (MTS) reduction to formazan salt (Promega Corporation).

In yet another example, the phenotype of interest is cellular proliferation. Methods for determining cellular proliferation are known in the art. For example, incorporation of ³H-thymidine or ¹⁴C-thymidine into DNA as it is synthesized is an assay for DNA synthesis associated with cell division. In such an assay, a cell is incubated in the presence of labeled thymidine for a time sufficient for cell division to occur. Following washing to remove any unincorporated thymidine, the label (e.g. the radioactive label) is detected, e.g., using a scintilation counter. Assays for the detection of thymidine incorporation into a live cell are available from, for example, Amersham Pharmacia Biotech. In another example, cellular proliferation is measured using a 3-(4, 5-dimethylthiazolyl-2)-2,5-diphenyltetrazolium bromide (MTT) assay. The yellow tetrazolium MTT is reduced by metabolically active cells, in part by the action of dehydrogenase enzymes, to generate reducing equivalents such as nicotinamide adenine dinucleotide (NADH) and nicotinamide adenine dinucleotide phosphate (NADPH). The resulting intracellular purple formazan is then solubilized and quantified by spectrophotometric means. Assay kits for MTT assays are available from, for example, American Type Culture Collection (ATCC; Rockville, Md.).

Alternative assays for determining cellular proliferation, include, for example, measurement of DNA synthesis by 5-bromo-2-deoxyuridine (BrdU) incorporation (by ELISA or immunohistochemistry, kits available from Amersham Pharmacia Biotech), expression of proliferating cell nuclear antigen (PCNA) (by ELISA, FACS or immunohistochemistry, kits available from Oncogen Research Products) or a Hoechst cell proliferation assay that detects DNA synthesis (available from Trevigen Inc.).

In the case of a compound that is an antibody, an assay to determine a compound that inhibits or reduces EPC activity can assess the ability of the compound to induce ADCC or CDC or antibody-dependent cell-mediated phagocytosis (ADCP) and kill (including lyse) an EPC. Methods for assessing ADCC, CDC and ADCP are known in the art.

For example, the ability of an antibody to induce CDC involves culturing the antibody and EPCs in the presence of complement factors (commercially available from, e.g., Sigma Aldrich) and a compound that is taken up by viable cells. Following washing, the amount of compound taken up by cells is detected. A reduction in the amount of compound taken up in the presence of an antibody compared to in the absence of the antibody indicates that the antibody induces CDC. Other methods for assessing CDC are known in the art and encompassed by the present disclosure, e.g., as described by Gazzano-Santoro et al., (1996).

A method for assessing ADCC activity involves incubating EPCs in the presence of an antibody and immune effector cells, e.g., PBMCs. The amount of lactate dehydrogenase activity in the supernatant of cell cultures is indicative of the amount of ADCC activity. Lactate dehydrogenase activity is assessed sing a commercially available kit (e.g., from Roche). Increased lactate dehydrogenase levels in the presence of antibody compared to in the absence of antibody indicates that the antibody induces ADCC. Alternatively, or in addition, a ⁵¹Cr release assay is performed to assess EPC cell death mediated by ADCC. Additional methods for assessing ADCC are described, for example, in U.S. Pat. No. 5,500,362 or U.S. Pat. No. 5,821,337.

ADCP is assessed, for example, by labeling EPCs with a fluorescent label, e.g., PKH2 green fluorescence dye. The labeled EPCs are then incubated with mononuclear cells (e.g., PBMCs) in the presence or absence of antibody. Following a sufficient time, cells are incubated with a labeled antibody against, for example, CD14 or CD11b. Cells staining for both the EPC label and CD14 or CD11b are considered to be mononuclear cells that have phagocytosed an EPC. An antibody that increases the number of double labeled cells (compared to the number present in the absence of antibody) is considered to induce ADCP.

### In Vivo Assays of EPC Function

In another example, a population of cells isolated by a method as described herein according to any example is determined by administering the cells to an animal model of a condition associated with EPCs. For example, the cells are administered to an animal lacking EPCs e.g., as a result of myeloablation or mice having defects in angiogenesis (e.g., Id1-deficient mice; Lyden et al., 2001). Cells that facilitate or contribute to neovascularization are considered to have EPC function. Alternatively, or in addition, cells are administered to an animal model of ischemia, such as, hind-limb ischemia and/or cardiovascular ischemia and/or stroke and the effect of the cells on neovascularization is determined Exemplary models are described, for example, in Couffinhal et al. (1998) or Carmeliet et al. (2000).

In another example, EPC activity is assessed by mixing EPCs with matrigel to form a plug and administering the plug subcutaneously to a non-human mammal, e.g., a mouse. After a sufficient period, e.g., about 7 days, the plug is removed and analyzed microscopically for evidence of formation of blood vessels, i.e., neovascularization. An exemplary method is described in Bagley et al., (2003).

Compounds to be tested for their ability to suppress EPC activity and/or numbers can be administered to a test subject and the number of EPCs detected/isolated using standard methods or methods described herein. A reduction in the number of EPCs compared to the number of EPCs from an untreated subject indicates that the compound reduces EPC numbers.

Alternatively, or in addition, a compound is administered to an animal model of angiogenesis and the level of blood vessel formation determined. For example, a compound is administered to a test subject at the time of, prior to or following administration of tumor cells or induction of angiogenesis. The presence/absence and/or size of any resulting tumor are then assessed and compared to subjects to which the cells but not the compound has been administered. For example, the amount of vascularization is determined in the tumor test tissue to determine a compound that suppresses neovascularization. Models of excessive angiogenesis include Iris Pharma Inc's models of ocular angiogenesis, or an alginate encapsulated tumor cell model, e.g., as described in Hoffmann et al., (1997).

### Kits

The present disclosure also provides therapeutic/prophylactic/diagnostic kits comprising compounds of the present disclosure for use in the present detection/isolation/diagnostic/prognostic/treatment/prophylactic methods. Such kits will generally contain, in suitable container means, a compound of the present disclosure. The kits may also contain other compounds, e.g., for detection/isolation/diagnosis/imaging or combined therapy. For example, such kits may contain any one or more of a range of anti-inflammatory drugs and/or chemotherapeutic or radiotherapeutic drugs; anti-angiogenic agents; anti-tumor cell antibodies; and/or anti-tumor vasculature or anti-tumor stroma antibodies or coaguligands or vaccines.

Exemplary kits comprise a compound that binds to a protein set forth in any one or more of Tables 1-6, e.g., an antibody of the disclosure.

In one example, the kit is for detecting a protein set forth in any one or more of Tables 1-6 and additionally comprises a reagent to facilitate detection (a detectable label and/or a substrate of a detectable label. Such kits may additionally comprise a positive control.

In another example, the kit is for isolating an EPC. In such kits the compound may be labeled with a detectable label to facilitate FACS. The compound may also be labeled with a magnetic or paramagnetic particle to facilitate MACS. The compound may also be immobilized on a solid or semi-solid substrate to facilitate isolation.

In a further example, the kit is for treatment or prevention of an EPC-associated condition. In such kits the molecule may be provided in solution or in a lyophilized form, optionally with a solution for resuspension. The compound may be conjugated to a therapeutic compound or the kit may include a therapeutic compound for conjugation thereto. As discussed above, the kit may also comprise additional therapeutic or prophylactic compounds.

Alternatively or in addition, a kit for therapy or prophylaxis comprises one or more compounds that bind(s) to a protein set forth is any one of Tables 1-6 immobilized on a solid support suitable for administering to a subject in the form of a vascular graft.

The present disclosure includes the following non-limiting examples.

### Example 1

### Identification of Markers of EPCs Using Recombinant Cells

### 1.1 Materials and Methods

### Cell Treatment and Harvesting

Human umbilical vein endothelial cells (HUVECs) were isolated from human umbilical cords using collagenase type 1 and then grown on gelatin-coated T flasks. Cells at passage 2 at 60% confluence were transduced with adenovirus containing sphingosine kinase 1 cDNA (Ad-SK-1) or empty vector adenovirus (Ad-EV) (Limaye et al., 2005; and Bonder et al., 2009) and harvested four days later. Cells were sorted based on CD34 expression using CD34 microbeads and miniMACS columns (Miltenyi Biotec). CD34 surface expression was detected by staining an aliquot of the sorted cells with anti-human CD34-PE antibodies followed by flow cytometry. Cell number and viability was determined by staining an aliquot with trypan blue and then counting with a hemocytometer.

### RNA Isolation and Purification

CD34 sorted cells from untreated, Ad-SK-1 and Ad-EV treated HUVEC were lysed in RLT buffer (RNeasy Micro kit, Qiagen) supplemented with 0.1% beta mercapto-ethanol and stored at −70° C. Lysates were thawed on ice, triturated 10× using a 26G needle/1 ml syringe; and RNA was purified using the RNeasy Micro kit, which included an on-column DNase step, and eluted in RNase-free water and then stored at −70° C. RNA quantity and integrity were determined using an Agilent Bioanalyzer. RNA samples obtained from cell lines which showed increased SK-1 activity of ˜5-10 fold (³²P-based kinase assay), an increase in CD34 surface expression, and good RNA yield and quality were chosen for microarray analysis.

### Microarray Analysis

RNA expression was analyzed using two different microarray platforms, one at the Adelaide Microarray Centre (AMC) and the other at Amgen, Inc. USA. For microarray analysis performed at AMC, generation and labeling of complementary RNA was achieved using the Whole Transcript (WT) Sense Target Labeling Assay. Labeled complementary RNA was hybridized to GeneChip® Human Gene 1.0 ST Arrays (Affymetrix, Inc). For the analysis performed at Amgen, Inc, labeling was achieved using the Nugen Ovation kit, followed by hybridization to the Affymetrix U133 Plus 2.0 arrays (i.e. 3′ arrays).

### Data Analysis

For the AMC microarray data, RNA expression differences were initially analyzed using the Partek Genomics Suite, including normalization using robust multiarray averaging (RMA) with GC probe content correction. A list of genes was generated for all 4 cell line comparisons as well as 3-way comparisons with standard p-values calculated. More in-depth analysis was performed using software obtained through the Bioconductor project (Gentleman et al., 2004) using mainly affy (Gautier, et al., 2004) and limma (Smyth, 2005) packages. P-values from the more in-depth analysis were adjusted for multiple testing by controlling the false discovery rate, the expected proportion of false discoveries amongst the rejected hypotheses (Benjamini, et al., 1995). Using this more in-depth analysis, the top 100 potentially regulated genes were selected from comparisons of SK-1 over-expressing cells to either untreated controls or cells transduced with an empty vector adenovirus. The analysis was performed using all 4 cell lines as well as with all combinations of 3-way comparisons. A resulting list of 319 genes was generated and combined with the list of genes from the preliminary Partek data analysis.

Data analysis of the microarray data generated at Amgen, Inc. was performed in Rosetta Resolver. Intensities were generated in the Affymetrix Rosetta Intensity Profile Builder pipeline, followed by normalisation using the Affymetrix Rosetta Intensity Experiment Builder. Differential expression was obtained using the Affymetrix Ratio Builder (no error weighting). Standard p-values were calculated.

### 1.2 Results

A list of transcripts derived from both microarray data sets (see Table 7) was generated for the most highly over-expressed genes (lower limit of 1.3 fold increase) which can code for cell surface proteins.

TABLE 7
Genes selected from microarray analysis

| | | | | | | | Nucleo- | Amino |
|---|---|---|---|---|---|---|---|---|
| | | | | | Comparison | | tide | Acid |
| | | Fold | | Common Gene | Groups | | SEQ | SEQ |
| Accession | Gene Name | Change | P-value | Name | (Cell | Location | ID NO; | ID NO: |
| NM_016512 | SPAG11B | 3.17 | 0.04 | sperm associated | all 4 | Extracellular Space | 237 | 238 |
| | transcript variant A | | | antigen 11B, | | | | |
| | | | | transcript variant A | | | | |
| NM_025074 | FRAS1 | 2.98 | 0.02 | Fraser syndrome 1 | all 4 | Extracellular Space | 239 | 240 |
| NM_175924 | ILDR1 | 2.74 | 0.01 | immunoglobulin- | all 4 | Plasma Membrane | 241 | 242 |
| | | | | like domain | | | | |
| | | | | containing | | | | |
| | | | | receptor 1 | | | | |
| NM_012156 | EPB41L1, | 2.3 | 0.02 | erythrocyte | all 4 | Plasma Membrane | 243 | 244 |
| | transcript variant 1 | | | membrane protein | | | | |
| | | | | band 4.1-like 1, | | | | |
| | | | | transcript variant 1 | | | | |
| NM_001187 | BAGE | 2.22 | 0.02 | B melanoma | all 4 | unknown | 245 | 246 |
| | | | | antigen | | | | |
| NM_000826 | AMPA2, | 2.17 | 0.03 | glutamate | all 4 | Plasma Membrane | 247 | 248 |
| | transcript variant 1 | | | receptor, | | | | |
| | | | | ionotropic, | | | | |
| | | | | AMPA2 | | | | |
| NM_031912 | SYT15, transcript | 2.16 | 0.05 | synaptotagmin | all 4 | Cytoplasm | 249 | 250 |
| | variant a | | | XV, transcript | | | | |
| | | | | variant a | | | | |
| NM_015090 | NFASC, | 2.03 | 0.04 | neurofascin | all 4 | Plasma Membrane | 251 | 252 |
| | transcript variant 4 | | | homolog | | | | |
| | | | | (chicken), | | | | |
| | | | | transcript variant 4 | | | | |
| AI401535 | EST (Clone | 1.89 | 0.03 | expressed | all 4 | Plasma Membrane | 253 | 254 |
| | IMAGE: 2110090) | | | sequence tag; low | | | | |
| | | | | quality annotation - | | | | |
| | | | | neuroligin 1 | | | | |
| NM_001004433 | SLC30A10, | 1.72 | 0.04 | solute carrier | all 4 | unknown | 255 | 256 |
| | transcript variant 2 | | | family 30, | | | | |
| | | | | member 10, | | | | |
| | | | | transcript variant 2 | | | | |
| NM_018974 | UNC93A, | 1.71 | 0.01 | unc-93 homologue | all 4 | Plasma Membrane | 257 | 258 |
| | transcript variant 1 | | | A (C. elegans), | | | | |
| | | | | transcript variant 1 | | | | |
| NM_012353 | OR1C1 | 1.69 | 0.04 | olfactory receptor, | all 4 | Plasma Membrane | 259 | 260 |
| | | | | family 1, | | | | |
| | | | | subfamily C, | | | | |
| | | | | member 1 | | | | |
| NM_001079669 | TMTC4, | 1.45 | 0.03 | transmembrane | all 4 | unknown | 261 | 262 |
| | transcript variant 2 | | | and | | | | |
| | | | | tetratricopeptide | | | | |
| | | | | repeat containing | | | | |
| | | | | 4, transcript | | | | |
| | | | | variant 2 | | | | |
| NM_001830 | CLCN4 | 1.39 | 0.02 | chloride channel 4 | all 4 | Plasma Membrane | 263 | 264 |
| NM_030959 | OR12D3 | 2.46 | | olfactory receptor, | 2, 3, 5 | Plasma Membrane | 265 | 266 |
| | | | | family 12, | | | | |
| | | | | subfamily D, | | | | |
| | | | | member 3 | | | | |
| NM_024850 | BTNL8, transcript | 1.96 | | butyrophilin-like | 1, 3, 5 | unknown | 267 | 268 |
| | variant 1 | | | protein 8 | | | | |
| | | | | precursor, | | | | |
| | | | | transcript variant 1 | | | | |
| NM_020949 | SLC7A14 | 1.66 | | solute carrier | all 4 | unknown | 269 | 270 |
| | | | | family 7, member | | | | |
| | | | | 14 | | | | |
| NM_001005191.1 | OR7D4 | 1.63 | | olfactory receptor, | 2, 3, 5 | Plasma Membrane | 271 | 272 |
| | | | | family 7, | | | | |
| | | | | subfamily D, | | | | |
| | | | | member 4 | | | | |
| AF147791 | MUC12 | 1.574 | | mucin 12, cell | 2, 3, 5 | Extracellular Space | 273 | 274 |
| | | | | surface associated | | | | |
| BC039116 | TRGC2 | 1.554 | | T cell receptor | 2, 3, 5 | Plasma Membrane | 275 | 276 |
| | | | | gamma constant 2 | | | | |
| | | | | (cDNA clone | | | | |
| | | | | IMAGE: 4829750) | | | | |
| NR_003668 | DEF109P1B | 1.55 | 0.03 | defensin, beta 109, | all 4 | unknown | 277 | 278 |
| | | | | pseudogene 1B, | | | | |
| | | | | noncoding RNA | | | | |
| NM_001034837 | KCNIP1, | 1.52 | | Kv channel | 1, 3, 5 | Plasma Membrane | 279 | 280 |
| | transcript variant 1 | | | interacting protein | | | | |
| | | | | 1, transcript | | | | |
| | | | | variant 1 | | | | |
| BC033223 | SLC45A4 | 1.5 | | solute carrier | 1, 3, 5 | unknown | 281 | 282 |
| | | | | family 45, | | | | |
| | | | | member 4 (cDNA | | | | |
| | | | | clone | | | | |
| | | | | IMAGE: 5019517) | | | | |
| NM_153343 | ENPP6 | 1.49 | | ectonucleotide | 1, 2, 5 | Cytoplasm | 283 | 284 |
| | | | | pyrophosphatase/ | | | | |
| | | | | phosphodiesterase 6 | | | | |
| NM_019120 | PCDHB8 | 1.49 | | protocadherin beta 8 | 1, 2, 3 | Plasma Membrane | 285 | 286 |
| NM_001005495 | OR2T3 | 1.45 | | olfactory receptor, | all 4 | unknown | 287 | 288 |
| | | | | family 2, | | | | |
| | | | | subfamily T, | | | | |
| | | | | member 3 | | | | |
| NM_001004741 | OR5M10 | 1.34 | 0.053 | olfactory receptor, | all 4 | Plasma Membrane | 289 | 290 |
| | | | | family 5, | | | | |
| | | | | subfamily M, | | | | |
| | | | | member 10 | | | | |
| NM_001004725 | OR4S1 | 1.32 | 0.048 | olfactory receptor, | all 4 | unknown | 291 | 292 |
| | | | | family 4, | | | | |
| | | | | subfamily S, | | | | |
| | | | | member 1 | | | | |
| NM_016540 | GPR83 | 1.31 | 0.017 | G protein-coupled | all 4 | Plasma Membrane | 293 | 294 |
| | | | | receptor 83 | | | | |
| NM_176888 | TAS2R19 | 1.3 | 0.048 | taste receptor, type | all 4 | unknown | 295 | 296 |
| | | | | 2, member 19 | | | | |
| NM_000216 | KAL1 | 1.3 | 0.011 | Kallmann | all 4 | Extracellular Space | 297 | 298 |
| | | | | syndrome 1 | | | | |
| | | | | sequence | | | | |
| NM_019844 | SLCO1B3 | 1.37 | | solute carrier | all 4 | Plasma Membrane | 299 | 300 |
| | | | | organic anion | | | | |
| | | | | transporter family, | | | | |
| | | | | member 1B3 | | | | |
| AL133267 | multiple (1 gene; | 1.42 | 0.022 | Human DNA | all 4 | Plasma Membrane | 301 | 302 |
| | 3 pseudogenes) | | | sequence from | | | | |
| | | | | clone RP3- | | | | |
| | | | | 408B20 on | | | | |
| | | | | chromosome 6. | | | | |
| | | | | Contains a gene | | | | |
| | | | | and two | | | | |
| | | | | pseudogenes for 7 | | | | |
| | | | | transmembrane | | | | |
| | | | | receptor | | | | |
| | | | | (rhodopsin family) | | | | |
| | | | | (olfactory receptor | | | | |
| | | | | like) proteins and | | | | |
| | | | | a 60S acidic | | | | |
| | | | | ribosomal protein | | | | |
| | | | | P2 (RPLP2) | | | | |
| | | | | pseudogene, | | | | |
| | | | | complete | | | | |
| | | | | sequence. 7 | | | | |
| | | | | transmembrane | | | | |
| | | | | receptor | | | | |
| | | | | (rhodopsin family) | | | | |
| | | | | (olfactory receptor | | | | |
| | | | | like) pseudogene | | | | |
| | | | | (hs6M1-33P) | | | | |
| M60028 | HLA_DQB1 | 1.31 | 0.04 | major | all 4 | Plasma Membrane | 303 | 304 |
| | | | | histocompatability | | | | |
| | | | | complex, class II, | | | | |
| | | | | DQ beta 1 | | | | |
| NM_001627 | ALCAM | 3.3 | 0.02 | activated | all 4 | Plasma Membrane | 305 | 306 |
| | | | | leukocyte cell | | | | |
| | | | | adhesion molecule | | | | |
| | | | | (CD166) | | | | |
| NM_013447 | EMR2, transcript | 1.457 | 0.0184 | egf-like module | all 4 | Plasma Membrane | 17 | 18 |
| | variant 1 | | | containing, mucin- | | | | |
| | | | | like, hormone | | | | |
| | | | | receptor-like 2, | | | | |
| | | | | transcript variant 1 | | | | |
| | | | | (CD312) | | | | |
| NM_144717 | IL20RB | 2.77 | 0.03 | Interleukin 20 | all 4 | Plasma Membrane | 307 | 308 |
| | | | | receptor beta | | | | |
| NM_001006624 | PDPN, transcript | 2.22 | 0.04 | podoplanin | all 4 | Plasma Membrane | 309 | 310 |
| | variant 3 | | | transcript variant 3 | | | | |
| NM_000740 | CHRM3 | 2.2 | 0.02 | cholinergic | all 4 | Plasma Membrane | 311 | 312 |
| | | | | receptor, | | | | |
| | | | | muscarinic 3 | | | | |
| NM_002211 | ITGB1, transcript | 1.74 | 0.04 | integrin, beta 1 | all 4 | Plasma Membrane | 313 | 314 |
| | variant 1A | | | (fibronectin | | | | |
| | | | | receptor, beta | | | | |
| | | | | polypeptide, | | | | |
| | | | | antigen CD29 | | | | |
| | | | | includes MDF2, | | | | |
| | | | | MSK12), | | | | |
| | | | | transcript variant | | | | |
| | | | | 1A | | | | |
| NM_014442 | SIGLEC8 | 1.72 | 0.04 | sialic acid binding | all 4 | Plasma Membrane | 315 | 316 |
| | | | | Ig-like lectin 8 | | | | |
| | | | | (CD329) | | | | |
| NM_001010935 | RAP1A, | 1.55 | 0.008 | member of RAS | all 4 | Cytoplasm | 317 | 318 |
| | transcript variant 1 | | | oncogene gamily, | | | | |
| | | | | transcript variant 1 | | | | |
| NM_025179 | PLXNA2 | 1.52 | 0.03 | plain A2 | all 4 | Plasma Membrane | 319 | 320 |
| NM_014511 | KIR2DL3, | 1.9 | 0.01 | killer cell | all 4 | Plasma Membrane | 321 | 322 |
| | transcript variant 1 | | | immunoglobulin- | | | | |
| | | | | like receptor, two | | | | |
| | | | | domains, long | | | | |
| | | | | cytoplasmic tail, | | | | |
| | | | | 3, transcript | | | | |
| | | | | variant 1 | | | | |
| | | | | (CD158b) | | | | |
| NM_005292 | GPR18, transcript | 1.65 | | G protein-coupled | 2, 3, 5 | Plasma Membrane | 75 | 76 |
| | variant 1 | | | receptor 18, | | | | |
| | | | | transcript variant 1 | | | | |
| NP_031386.1 | KLRK1 | 1.59 | | CD314, killer cell | all 4 | Plasma Membrane | 323 | 324 |
| | | | | lectin-like | | | | |
| | | | | receptor, | | | | |
| | | | | subfamily K, | | | | |
| | | | | member 1 | | | | |
| NM_001337 | CX3CR1 | 1.562 | | chemocine | 2, 3, 5 | Plasma Membrane | 325 | 326 |
| | | | | (C-X3-C) | | | | |
| | | | | receptor 1, | | | | |
| | | | | CCRL1 | | | | |

### Example 2

### Identification of Markers of EPCs Using Non-Adherent CD133⁺ EPCs

### 2.1 Materials and Methods

### Isolation of Target Cells

Donor blood (20-170 ml) was diluted in 1:1 ratio with sterile phosphate buffered saline (PBS) and layered on 15 mL of Lymphoprep™ (Axis-Shield, Oslo, Norway) in falcon tubes. Cells were then centrifuged at 400 g for 30 minutes at room temperature. Mononuclear cells (MNCs) were isolated and washed thrice with HUVE media (Media 199 (Sigma); supplemented with 20% FCS, 1.5% sodium bicarbonate, 2% HEPES buffer solution, penicillin-streptomycin, non-essential amino acids and sodium pyruvate (GIBCO)).

Mononuclear cells (MNCs) were incubated with 100 μl of human FcR blocking reagent (Miltenyi Biotec, Auburn, Calif., USA) and 100 μl of CD133⁺ antibody microbeads (MACS, Miltenyi Biotec) for 30 min at 4° C. as per manufacturer's instructions prior to re-suspension in MACS buffer (2 mM ethylenediaminetetraacetic acid (EDTA)/PBS and 0.5% BSA/PBS). The CD133⁺ cells were isolated using an AutoMacsPro (Miltenyi Biotec). Isolated cells were then centrifuged at 4° C. and resuspended at a concentration of 0.5-1×10⁶ cells/ml in endothelial growth media (EGM-2) complete with bullet kit (Lonza) and supplemented with 10% FCS, vascular endothelial growth factor (VEGF; 5 ng/mL, Sigma, St Louis, Mo., USA), insulin-like growth factor-1 (IGF-1; 1 pg/mL, Gibco Invitrogen, Gaithersburg, Md., USA), basic fibroblast growth factor (bFGF; ing/mL, 1/25000, R&D) and ascorbic acid (1 mM, Sigma). Cells were seeded in a 24-well plate pre-coated with fibronectin and incubated at 37° C. and 5% CO₂. During culture, non-adherent cells were transferred to a new pre-coated fibronectin well and cultured for 48-72 hours in fresh EGM-2 media. These cells were cultured for 2, 4, 7, or 10 days prior to harvesting for further analysis.

### Preparation of Human Umbilical Vein Endothelial Cells (HUVECs)

Primary HUVECs were extracted from human umbilical veins by collagenase digestion, as described previously (Litwen et al., 1998). HUVECs were used no later than two passages.

### Gene Array

Total RNA was isolated from natural EPCs and donor matched mature endothelial cells from human umbilical cords (HUVECs) from 4 biological replicates using an RNEasy micro plus kit (QIAGEN, Hilden, Germany). RNA integrity and quantity was determined using an Experion analysis kit prior to conducting microarray experiments (BioRad). 150 ng of RNA was amplified and labelled using Ovation system by NuGen. The labelled and amplified RNA was hybridized to Affymetrix Human Exon 1.0ST arrays as per the manufacturer's protocol (Affymetrix) in the microarray facility at Mater Adult Hospital, Brisbane.

Human affymetrix exon arrays were scanned with GeneScanner 3000, 7G. The raw CEL and CHP data was acquired and imported into GeneSpring GX version 11 (Agilent) for data analysis. Robust multi-array analysis (RMA) was used for normalizing and summarizing probe level intensity measurements from Affymetrix gene chips. Hybridization quality for each array was assessed using box plots and principal component analysis (PCA) of probe-level data.

Expression profiling was performed on the following group of experiments created using Genespring GX11 to identify differentially expressed genes.

1. Day 4 natural EPCs vs matched day 4 HUVECs.

2. Day 7 natural EPCs vs matched day 4 HUVECs

3. Day 4 natural EPCs vs day 7 natural EPCs.

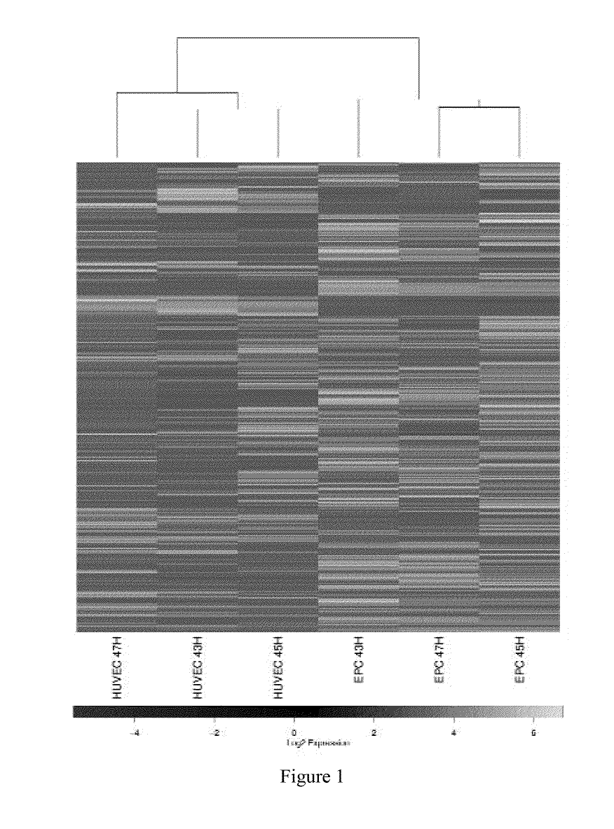

A parametric Welch's t-test (where variances were not assumed equal) was performed on 19524 probes independently for both day 4 and day 7 EPCs with a p-value cut off of 0.05 and a fold change cut off of 1.5. Multiple testing correction (Benjamini and Hochberg False Discovery Rate) was then applied to genes that had passed the parametric Welch's t-test based on the total detected probe-set of 14246 probes to reduce false positives. Following this statistical filtering, a total of 977 genes in experiment condition 1 (day 4 EPCs vs HUVECs) and 1650 genes in experiment condition 2 (day 7 EPCs vs HUVECs) were significantly upregulated in EPCs. There was no change observed in the gene expression in the third experimental condition (day 4 EPCs vs day 7 EPCs). A heatmap representing gene expression changes between EPCs cultured for 4 days and HUVECs cultured for less than 2 passages are shown in FIG. 1. This figure indicates there are considerable gene expression differences between the EPC and HUVEC cell populations.

The significantly upregulated genes were grouped according to their potential relevant functions in EPCs. Functional categorization of genes was performed using a combination of Agilent technologies gene ontology classifications and Ingenuity Pathway Analysis (IPA, http://www.ingenuity.com). The up-regulated genes included those which are known surface markers for EPCs including CD133 and c-KIT. The uniqueness of EPCs was illustrated by the differences in the expression level of well established endothelial markers (e.g., CD31, CD144 and CD62E) compared to HUVECS.

Functionally categorized genes revealed a total of 137 membrane proteins in experiment condition 1. The gene list was further screened using Gene card, IPA, pubmed, BioGPS and genes that had been previously described in EPCs, endothelial cells and/or hematopoietic stem cells were excluded.

Multiple significant probes for the same gene were removed from final data tables with the probe with highest fold change being chosen.

### 2.2 Results

Significantly differentially expressed genes for the first and second experiment group were selected and categorised as follows:
a) Category A list: Significantly upregulated with high fold change value in Day 4 EPCs vs HUVECs
b) Category B: High fold change value with a close to significant p-value in day 4 EPCs vs HUVECs.
c) Category C: Significantly upregulated with high fold change in Day 7 EPCs vs HUVECs.

The three lists were then combined to create a list of biomarkers.

The biomarkers were then analysed to identify those likely to be expressed on the cell surface using ‘Gene Card’ ‘Phobius’ and ‘IPA’.

Results of these analyses are set out below in Table 8.

### Example 3

### Validation of Biomarkers by Low Density Array

Total RNA was isolated from CD133⁺ sorted 4 day cultured EPCs (prepared essentially as described in Example 2) and from donor matched HUVEC from 4 biological replicates using an RNEasy micro plus and RNEasy mini kit, respectively (QIAGEN, Germany) Total EPC RNA (300-700 ng) is converted to cDNA using a High Capacity cDNA Transcription Kit (Applied Biosysytems) with an equivalent amount of HUVEC RNA isolated using the same protocol. Each cDNA synthesis reaction was combined with TaqMan® Universal PCR master mix and loaded equally into 4 sample fill-reservoirs of a Custom TaqMan® Low Density Array (Format 96b). Amplification and data acquisition was carried out on a 7900HT Real-Time PCR System (Applied Biosystems). Donor matched EPCs and HUVEC were loaded on the same array. Relative quantitation (RQ) of targets is performed using the comparative Ct (ΔΔCT) method using RQ manager (SDSv2.3 software, Applied Biosystems). The Custom TaqMan® Low Density Array was built using validated TaqMan® gene expression assays. Each target was validated in duplicates with 4 different biological donors.

Results of low density array analysis are set out in Table 8.

### Example 4

### Validation of Biomarkers by Flow Cytometry

### 4.1 Materials and Methods

Antibodies were obtained from commercial sources. For each antibody the appropriate isotype-control (species, Ig isotype and company) is used.

Analyses of the reactivity of the target antibodies was performed using a three-step “high sensitivity” staining protocol on HUVEC, natural EPCs (prepared essentially as described in Example 2), peripheral blood (collected using lithium-heparin anticoagulant) or umbilical cord blood. Cells were sedimented using centrifugation and resuspended in HUVE wash (Media 199 (Sigma), 2% fetal calf serum, 1% 10 mM HEPES and 1% penicillin streptomycin solution (Gibco)) at a concentration of about 5×10⁴-10⁶ cells per assay. For peripheral blood samples, 100 μl was used per assay.

Cells (EPCs, HUVECs and peripheral blood cells) were sedimented using centrifugation, resuspended and treated with 10 μl Human FcR block (Miltenyi Biotec) diluted in 30 μHUVE wash. Samples were then incubated on ice for 10 minutes prior to addition of primary antibodies. Cells were incubated in 100 μl of diluted primary antibody for 30 minutes followed by a wash. Cells were sedimented by centrifugation, resuspended and incubated for 30 minutes on ice with appropriate secondary antibody diluted in cold HUVE wash. Cells were washed with 1 ml of FACS wash, sedimented by centrifugation and resuspended. Cells were then blocked with 5 μl of normal mouse serum at 4° C. for 10 minutes. Conjugated streptavidin (PE, APC or PE-Cy7 conjugated) (BD Biosciences Pharmingen) was added at 0.2 μg per test along with panels of mouse anti-human conjugated antibodies; anti-CD34-Pe-Cy7 for progenitor cells, CD144-FITC for HUVEC, anti-VEGFR2, anti-CD117-APC and anti-CD133-PE for EPC, anti-CD31 for vascular cells and antiCD45, anti-CD11b-PE-Cy7 and anti-CD14-APC for PB, (all BD Biosciences) were used according to the manufacturer's instructions for flow cytometry. Cells were then washed with 1 ml FACS wash. Blood samples were incubated with 1.5 ml 1× BD Pharmingen Lyse™ diluted in water at room temperature. Cells were again sedimented by centrifugation and resuspended. Cells were resuspended in FACS fix (1% formaldehyde, 20 g/L glucose, 5 mM sodium azide, made up in PBS) prior to analysis using a FACS Aria II (BD Biosciences) with FACS DIVA (BD Biosciences). Further analysis was performed using FCS Express V3.0 (De Novo Software, LA, Calif., USA).

Biomarkers were screened for surface expression on HUVEC (test antibody/CD34 or CD144) and PBMCs (test/forward scatter/side scatter settings). If the biomarker was not detectable at levels significantly above isotype control on HUVECs and PBMCs, targets were screened for EPC staining (test antibody/CD133⁺/CD117⁺). For EMR2 studies, expression was also studied on U937 cells and Jurkat T cells.

### 4.2 Results

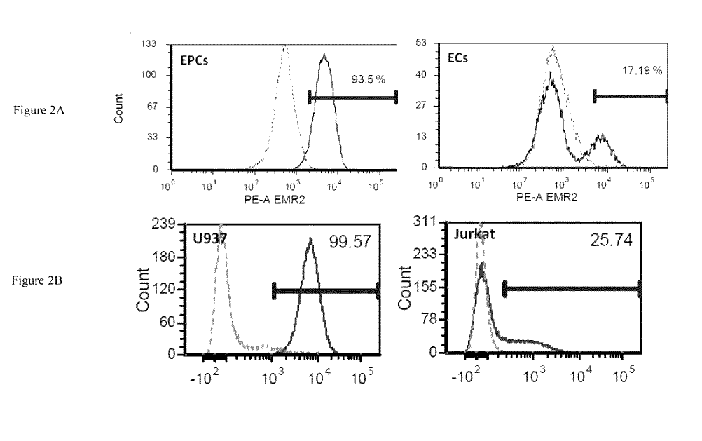

Results of analyses of expression of DSG2 and EMR2 are shown in FIGS. 2-5 and Table 8.

Results presented in FIG. 2 show that EMR2 is expressed on a large percentage of EPCs analyzed and to a much lesser degree on HUVECs. EMR2 was also expressed on U937 myeloid cells, but not on Jurkat T cells.

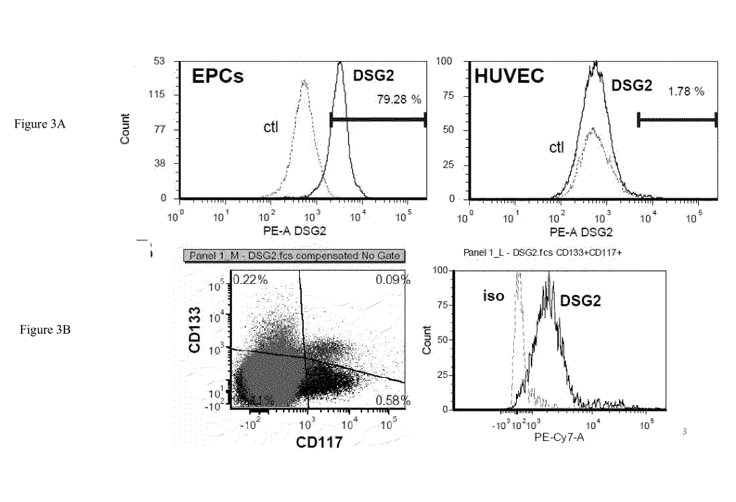

FIG. 3 shows that DSG2 is expressed on a significant proportion of EPCs analysed and on very few HUVECs. Panel B of FIG. 3 also demonstrates that DSG2 is expressed on CD133⁺CD117⁺ progenitor cells in PBMNCs. The data presented in FIG. 3 suggest that DSG2 can be used to isolate EPCs from peripheral blood samples.

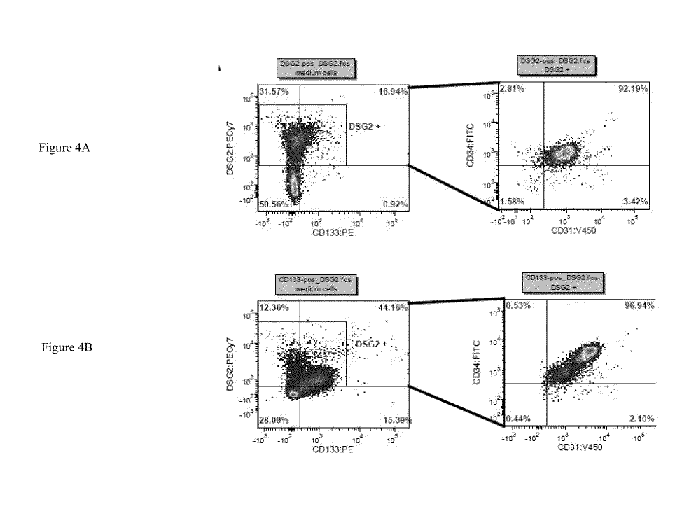

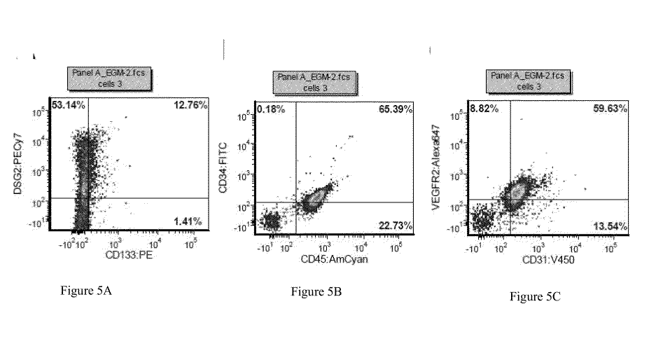

An anti-DSG2 antibody was used to isolate cells from freshly isolated umbilical cord blood and those cells analysed for cell surface marker expression. As shown in FIG. 4, cells isolated using anti-DSG2 antibody express the progenitor cell marker CD34 and the vascular marker CD31. Cells expressing CD34 and CD31 could also be isolated using anti-CD133 antibody. However, the populations isolated using anti-CD133 antibody or anti-DSG2 antibody as the capture reagent do not appear to be identical.

Further characterization of DSG2 expressing cells isolated from freshly isolated human umbilical cord blood showed that after culturing in EC supportive medium for four days the cells express vascular markers VEGFR2 and CD31, the progenitor marker CD34 and express low levels of progenitor markers CD133 and CD45. These cultured cells retain DSG2 expression.

TABLE 8
Biomarkers of EPCs.

| | | micro- | | | | | | | Nucleo- | Amino |
|---|---|---|---|---|---|---|---|---|---|---|
| | | array | LDA | | | | | | tide | Acid |
| | Entrez Gene | Fold | Fold | EPC | EC | Reference | TM | | SEQ | SEQ |
| Gene ID | Name | Change | change | flow | flow | Sequence | domains | Location | ID NO | ID NO: |
| EMB | embigin | 6.0 | 85 | | | NM_198449; | 1 | Plasma | 1 | 2 |
| | homolog | | | | | NR_003955 | | Membrane | | |
| | (mouse) | | | | | | | | | |
| SLC15A2 | solute carrier | 7.4 | 60 | | | NM_021082 | 9 | Plasma | 19 | 20 |
| | family 15 | | | | | | | Membrane | | |
| | (H+/peptide | | | | | | | | | |
| | transporter), | | | | | | | | | |
| | member 2 | | | | | | | | | |
| SLC16A6 | solute carrier | 4.0 | 9 | | | NM_004694 | 12 | Plasma | 21 | 22 |
| | family 16, | | | | | | | Membrane | | |
| | member 6 | | | | | | | | | |
| | (monocarboxylic | | | | | | | | | |
| | acid | | | | | | | | | |
| | transporter 7) | | | | | | | | | |
| SLC39A8 | solute carrier | 3.6 | 4 | | | NM_022154 | 6 | Extracellular | 3 | 4 |
| | family 39 | | | | | | | Space | | |
| | (zinc | | | | | | | | | |
| | transporter), | | | | | | | | | |
| | member 8 | | | | | | | | | |
| SIGLEC10 | sialic acid | 3.2 | 44 | | negative | NM_033130 | 1 | Plasma | 23 | 24 |
| | binding Ig-like | | | | | | | Membrane | | |
| | lectin 10 | | | | | | | | | |
| SIGLEC6 | sialic acid | 1.6 | 34 | | | NM_001245; | 1 | Extracellular | 25 | 26 |
| | binding Ig-like | | | | | NM_198845; | | Space | | |
| | lectin 6 | | | | | NM_198846 | | | | |
| AREG | amphiregulin | 5.7 | 30 | | negative | NM_001657 | 1 | Extracellular | 27 | 28 |
| | | | | | | | | Space | | |
| ITM2A | integral | 5.6 | 19 | | | NM_004867 | none | Plasma | 29 | 30 |
| | membrane | | | | | | | Membrane | | |
| | protein 2A | | | | | | | | | |
| GPM6B | glycoprotein | 3.2 | 41 | | | NM_001001995; | 4 | Plasma | 31 | 32 |
| | M6B | | | | | NM_001001996; | | Membrane | | |
| | | | | | | NM_005278; | | | | |
| | | | | | | NM_001001994 | | | | |
| CNR2 | cannabinoid | 3.1 | 128 | | negative | NM_001841 | 7 | Plasma | 33 | 34 |
| | receptor 2 | | | | | | | Membrane | | |
| | (macrophage) | | | | | | | | | |
| TM7SF3 | transmembrane 7 | 2.0 | 2 | | | NM_016551 | 7 | Plsma | 5 | 6 |
| | superfamily | | | | | | | Membrane | | |
| | member 3 | | | | | | | | | |
| PRSS21 | protease, | 1.7 | 112 | | | NM_006799; | none | Extracellular | 35 | 36 |
| | serine, 21 | | | | | NM_144956; | | Space | | |
| | (testisin) | | | | | NM_144957 | | | | |
| GPR174 | G protein- | 20.6 | 67 | | | NM_032553 | 7 | Plasma | 327 | 328 |
| | coupled | | | | | | | membrane | | |
| | receptor 174 | | | | | | | | | |
| NRG4 | neuregulin 4 | 4.5 | Not done | | | NM_138573 | 1 | Extracellular | 37 | 38 |
| | | | | | | | | Space | | |
| EPGN | epithelial | 9.1 | 0.5 | | | NM_001013442 | 1 | Extracellular | 39 | 40 |
| | mitogen | | | | | | | Space | | |
| | homolog | | | | | | | | | |
| | (mouse) | | | | | | | | | |
| RHBDD1 | rhomboid | 1.8 | 0.9 | | | NM_032276 | 4 | Extracellular | 41 | 42 |
| | domain | | | | | | | Space | | |
| | containing 1 | | | | | | | | | |
| PLXNC1 | plexin C1 | 8.6 | 51 | | negative | NM_005761 | 2 | Plasma | 7 | 8 |
| (CD232) | | | | | | | | Membrane | | |
| ABCC4 | ATP-binding | 2.1 | 2 | | negative | NM_005845; | 8 | Plasma | 43 | 44 |
| | cassette, sub- | | | | | NM_001105515 | | Membrane | | |
| | family C | | | | | | | | | |
| | (CFTR/MRP), | | | | | | | | | |
| | member 4 | | | | | | | | | |
| SORL1(LRP9) | sortilin-related | 18.7 | 145 | | negative | NM_003105 | 1 | Plasma | 45 | 46 |
| | receptor, | | | | | | | Membrane | | |
| | L(DLR class) | | | | | | | | | |
| | A repeats- | | | | | | | | | |
| | containing | | | | | | | | | |
| SLC8A1 | solute carrier | 6.4 | 1 | | | NM_021097; | 10 | Plasma | 47 | 48 |
| | family 8 | | | | | NM_001112800; | | Membrane | | |
| | (sodium/calcium | | | | | NM_001112801; | | | | |
| | exchanger), | | | | | NM_001112802 | | | | |
| | member 1 | | | | | | | | | |
| SLC22A16 | solute carrier | 3.8 | 594 | | | NM_033125 | 12 | Plasma | 49 | 50 |
| | family 22 | | | | | | | Membrane | | |
| | (organic | | | | | | | | | |
| | cation/carnitine | | | | | | | | | |
| | transporter), | | | | | | | | | |
| | member 16 | | | | | | | | | |
| SLC24A3 | solute carrier | 3.0 | 306 | | | NM_020689 | 11 | Plasma | 51 | 52 |
| | family 24 | | | | | | | Membrane | | |
| | (sodium/potassium/ | | | | | | | | | |
| | calcium | | | | | | | | | |
| | exchanger), | | | | | | | | | |
| | member 3 | | | | | | | | | |
| SLC2A5 | solute carrier | 2.8 | 1323 | | | NM_003039 | 5 | Plasma | 53 | 54 |
| | family 2 | | | | | | | Membrane | | |
| | (facilitated | | | | | | | | | |
| | glucose/fructose | | | | | | | | | |
| | transporter), | | | | | | | | | |
| | member 5 | | | | | | | | | |
| NCKAP1L | NCK- | 14.9 | 59 | | | NM_005337 | 1 | Plasma | 55 | 56 |
| | associated | | | | | | | Membrane | | |
| | protein 1-like | | | | | | | | | |
| EVI2B | ecotropic viral | 9.2 | 47 | | negative | NM_001003927 | 1 | Plasma | 57 | 58 |
| | integration site | | | | | | | Membrane | | |
| | 2B | | | | | | | | | |
| KCNQ5 | potassium | 8.8 | 164 | | negative | NM_019842 | | Plasma | 59 | 60 |
| | voltage-gated | | | | | | | Membrane | | |
| | channel | | | | | | | | | |
| P2RY14 | purinergic | 7.5 | 38 | | | NM_014879; | 7 | Plasma | 61 | 62 |
| | receptor P2Y, | | | | | Q15391; | | Membrane | | |
| | G-protein | | | | | BC034989; | | | | |
| | coupled, 14 | | | | | Q15391 | | | | |
| HTR1F | 5- | 6.3 | 1393 | | | NM_000866; | 7 | Plasma | 63 | 64 |
| | hydroxytryptamine | | | | | Q4QRI9; | | Membrane | | |
| | (serotonin) | | | | | BC069125; | | | | |
| | receptor 1F | | | | | P30939; | | | | |
| | | | | | | BC069125; | | | | |
| | | | | | | Q4QRI9 | | | | |
| TRAT1 | T cell receptor | 5.9 | 49 | | negative | NM_016388; | 1 | Plasma | 65 | 66 |
| | associated | | | | | Q6PIZ9; | | Membrane | | |
| | transmembrane | | | | | BC025713; | | | | |
| | adaptor 1 | | | | | Q6PIZ9 | | | | |
| GPR183 | G protein- | 5.5 | 5 | | | NM_004951 | 7 | Plasma | 67 | 68 |
| | coupled | | | | | | | Membrane | | |
| | receptor 183 | | | | | | | | | |
| OR13D1 | olfactory | 4.9 | 95 | | | NM_001004484 | 8 | Plasma | 69 | 70 |
| | receptor, | | | | | | | Membrane | | |
| | family 13, | | | | | | | | | |
| | subfamily D, | | | | | | | | | |
| | member 1 | | | | | | | | | |
| VSIG4 | V-set and | 4.9 | 29 | | negative | NM_007268; | 1 | Plasma | 71 | 72 |
| | immunoglobulin | | | | | NM_001100431 | | Membrane | | |
| | domain | | | | | | | | | |
| | containing 4 | | | | | | | | | |
| TAS2R4 | taste receptor, | 4.7 | 4 | | | NM_016944 | 7 | Plasma | 73 | 74 |
| | type 2, | | | | | | | Membrane | | |
| | member 4 | | | | | | | | | |
| GPR18 | G protein- | 4.6 | 8 | | | NM_005292; | 7 | Plasma | 75 | 76 |
| | coupled | | | | | NM_001098200 | | Membrane | | |
| | receptor 18 | | | | | | | | | |
| TAS2R3 | taste receptor, | 4.1 | 5 | | | NM_016943 | 7 | Plasma | 77 | 78 |
| | type 2, | | | | | | | Membrane | | |
| | member 3 | | | | | | | | | |
| MR1 | major | 4.0 | 5 | | | NM_001531; | 1 | Plasma | 79 | 80 |
| | histocompatibility | | | | | Q95460; | | Membrane | | |
| | complex, | | | | | U22963; | | | | |
| | class I-related | | | | | Q53GM1; | | | | |
| | | | | | | NM_001531; | | | | |
| | | | | | | Q53GM1; | | | | |
| | | | | | | U22963; | | | | |
| | | | | | | Q95460 | | | | |
| GPR34 | G protein- | 3.8 | 15 | | | NM_001097579; | 7 | Plasma | 81 | 82 |
| | coupled | | | | | NM_005300 | | Membrane | | |
| | receptor 34 | | | | | | | | | |
| NKG7 | natural killer | 3.6 | 12 | | | NM_005601 | 3 | Plasma | 9 | 10 |
| | cell group 7 | | | | | | | Membrane | | |
| | sequence | | | | | | | | | |
| KCNAB2 | potassium | 2.8 | 29 | | | NM_003636; | none | Plasma | 83 | 84 |
| | voltage-gated | | | | | NM_172130 | | Membrane | | |
| | channel, | | | | | | | | | |
| | shaker-related | | | | | | | | | |
| | subfamily, | | | | | | | | | |
| | beta member 2 | | | | | | | | | |
| KCNE3 | potassium | 2.4 | 28 | | | NM_005472 | 1 | Plasma | 85 | 86 |
| | voltage-gated | | | | | | | Membrane | | |
| | channel, Isk- | | | | | | | | | |
| | related family, | | | | | | | | | |
| | member 3 | | | | | | | | | |
| LAT2 | linker for | 2.3 | 33 | | | NM_032464; | none | Plasma | 87 | 88 |
| | activation of T | | | | | NM_032463; | | Membrane | | |
| | cells family, | | | | | NM_014146 | | | | |
| | member 2 | | | | | | | | | |
| OR52B6 | olfactory | 1.9 | 5 | | | NM_001005162 | 7 | Plasma | 11 | 12 |
| | receptor, | | | | | | | Membrane | | |
| | family 52, | | | | | | | | | |
| | subfamily B, | | | | | | | | | |
| | member 6 | | | | | | | | | |
| ADCY7 | adenylate | 1.7 | 5 | | | NM_001114 | 12 | Plasma | 13 | 14 |
| | cyclase 7 | | | | | | | Membrane | | |
| MLC1 | megalencephalic | 1.7 | 1169 | | | NM_015166; | 8 | Plasma | 89 | 90 |
| | leukoencephalopathy | | | | | NM_139202 | | Membrane | | |
| | with | | | | | | | | | |
| | subcortical | | | | | | | | | |
| | cysts 1 | | | | | | | | | |
| ENPP5 | ectonucleotide | 1.6 | 19 | | | NM_021572 | 1 | Extracellular | 91 | 92 |
| | pyrophosphatase/ | | | | | | | Space | | |
| | phosphodiesterase 5 | | | | | | | | | |
| | (putative | | | | | | | | | |
| | function) | | | | | | | | | |
| EMR2 | egf-like | 4.2 | 2 | 65.5% | 5.7% | NM_013447; | 7 | Plasma | 17 | 18 |
| | module | | | | | NM_152916; | | Membrane | | |
| | containing, | | | | | NM_152919; | | | | |
| | mucin-like, | | | | | NM_152917; | | | | |
| | hormone | | | | | NM_152920; | | | | |
| | receptor-like 2 | | | | | NM_152921; | | | | |
| | | | | | | NM_152918 | | | | |
| FLVCR1 | feline | 2.4 | 1.4 | | | NM_014053 | 12 | Plasma | 93 | 94 |
| | leukemia virus | | | | | | | Membrane | | |
| | subgroup C | | | | | | | | | |
| | cellular | | | | | | | | | |
| | receptor 1 | | | | | | | | | |
| GPR65 | G protein- | 2.8 | 18 | | | NM_003608 | 6 | Plasma | 95 | 96 |
| | coupled | | | | | | | Membrane | | |
| | receptor 65 | | | | | | | | | |
| OPN3 | opsin 3 | 2.7 | 2 | | | NM_014322; | 7 | Plasma | 97 | 98 |
| | | | | | | NM_001821 | | Membrane | | |
| TAS2R13 | taste receptor, | 4.0 | 1 | | | NM_023920 | 7 | Plasma | 99 | 100 |
| | type 2, | | | | | | | Membrane | | |
| | member 13 | | | | | | | | | |
| CLDN20 | claudin 20 | 2.0 | 1 | | | NM_001001346 | 3 | Plasma | 101 | 102 |
| | | | | | | | | Membrane | | |
| DSG2 | desmoglein 2 | 7.4 | 183 | ~50% | <1% | NM_001943 | 1 | Plasma | 15 | 16 |
| | | | | | | | | Membrane | | |
| SLC1A3 | solute carrier | 5.0 | 100 | | | NM_004172 | 7 | Plasma | 103 | 104 |
| | family 1 (glial | | | | | | | Membrane | | |
| | high affinity | | | | | | | | | |
| | glutamate | | | | | | | | | |
| | transporter), | | | | | | | | | |
| | member 3 | | | | | | | | | |
| SLC1A4 | solute carrier | 2.2 | Not | | | NM_003038 | 9 | Plasma | 105 | 106 |
| | family 1 | | done | | | | | Membrane | | |
| | (glutamate/neutral | | | | | | | | | |
| | amino acid | | | | | | | | | |
| | transporter), | | | | | | | | | |
| | member 4 | | | | | | | | | |
| CLDN10 | claudin 10 | 1.9 | 26 | | | NM_182848; | 3 | Plasma | 107 | 108 |
| | | | | | | NM_006984 | | Membrane | | |
| ADAMTS2 | ADAM | 1.7 | 75 | | | NM_014244; | none | Extracellular | 109 | 110 |
| | metallopeptidase | | | | | NM_021599 | | Space | | |
| | with | | | | | | | | | |
| | thrombospondin | | | | | | | | | |
| | type 1 | | | | | | | | | |
| | motif, 2 | | | | | | | | | |
| TBXAS1 | thromboxane | 4.7 | 33 | | | NM_001061; | 2 | Plasma | 111 | 112 |
| | A synthase 1 | | | | | NM_030984 | | Membrane | | |
| | (platelet) | | | | | | | | | |
| LAPTM5 | lysosomal | 3.5 | 5 | | | NM_006762 | | Plasma | 113 | 114 |
| | protein | | | | | | | Membrane | | |
| | transmembrane 5 | | | | | | | | | |
| VAMP8 | vesicle- | 2.7 | 3 | | negative | NM_003761 | 1 | Plasma | 115 | 116 |
| | associated | | | | | | | Membrane | | |
| | membrane | | | | | | | | | |
| | protein 8 | | | | | | | | | |
| | (endobrevin) | | | | | | | | | |
| AKAP7 | A kinase | 2.1 | 3 | | negative | NM_016377; | none | Plasma | 117 | 118 |
| | (PRKA) | | | | | NM_138633; | | Membrane | | |
| | anchor protein 7 | | | | | NM_004842 | | | | |
| SEMA3C | sema domain, | 3.6 | 1 | | | NM_006379 | none | Extracellular | 119 | 120 |
| | immunoglobulin | | | | | | | Space | | |
| | domain | | | | | | | | | |
| | (Ig), short | | | | | | | | | |
| | basic domain, | | | | | | | | | |
| | secreted, | | | | | | | | | |
| | (semaphorin) | | | | | | | | | |
| | 3C | | | | | | | | | |
| SLC38A1 | solute carrier | 8.6 | 1 | | | NM_001077484; | 11 | Plasma | 121 | 122 |
| | family 38, | | | | | Q9H2H9; | | Membrane | | |
| | member 1 | | | | | NM_030674; | | | | |
| | | | | | | Q9H2H9 | | | | |
| CD302 | CD302 | 6.8 | 3 | | | NM_002349 | 1 | Plasma | 123 | 124 |
| | molecule | | | | | | | Membrane | | |
| PLBD1 | phospholipase | 9.6 | 9 | | | NM_024829 | | Extracellular | 125 | 126 |
| | B domain | | | | | | | Space | | |
| | containing 1 | | | | | | | | | |
| LOXL3 | lysyl oxidase- | 1.5 | 4 | | | NM_032603 | | Extracellular | 127 | 128 |
| | like 3 | | | | | | | Space | | |
| FAM46C | family with | 1.9 | 8 | | | NM_017709 | | Extracellular | 129 | 130 |
| (includes | sequence | | | | | | | Space | | |
| EG: 54855) | similarity 46, | | | | | | | | | |
| | member C | | | | | | | | | |
| MFAP4 | microfibrillar- | 5.9 | 105 | | | NM_002404 | | Extracellular | 131 | 132 |
| | associated | | | | | | | Space | | |
| | protein 4 | | | | | | | | | |
| IQCB1 | IQ motif | 8.6 | 1 | | | NM_001023570; | | Extracellular | 133 | 134 |
| | containing B1 | | | | | NM_001023571 | | Space | | |
| FBN2 | fibrillin 2 | 4.0 | 2 | | | NM_001999 | | Extracellular | 135 | 136 |
| (includes | | | | | | | | Space | | |
| EG: 2201) | | | | | | | | | | |
| OGN | osteoglycin | 3.9 | 5 | | | NM_033014; | | Extracellular | 137 | 138 |
| | | | | | | NM_014057 | | Space | | |
| OMD | osteomodulin | 3.3 | 1 | | | NM_005014 | | Extracellular | 139 | 140 |
| | | | | | | | | Space | | |
| ASPN | asporin | 2.8 | 3 | | | NM_017680 | | Extracellular | 141 | 142 |
| | | | | | | | | Space | | |
| PZP | pregnancy- | 2.1 | 1 | | | NM_002864 | | Extracellular | 143 | 144 |
| | zone protein | | | | | | | Space | | |
| HSN2 | hereditary | 2.1 | NOT | | | NM_213655 | | Cytoplasm | 145 | 146 |
| | sensory | | DONE | | | | | | | |
| | neuropathy, | | | | | | | | | |
| | type II | | | | | | | | | |
| | (WNK1) | | | | | | | | | |
| SERPINI2 | serpin | 1.9 | 1 | | | NM_006217 | | Extracellular | 147 | 148 |
| | peptidase | | | | | | | Space | | |
| | inhibitor, | | | | | | | | | |
| | clade I | | | | | | | | | |
| | (pancpin), | | | | | | | | | |
| | member 2 | | | | | | | | | |
| ECM2 | extracellular | 1.8 | 1 | | | NM_001393 | | Extracellular | 149 | 150 |
| | matrix protein | | | | | | | Space | | |
| | 2, female | | | | | | | | | |
| | organ and | | | | | | | | | |
| | adipocyte | | | | | | | | | |
| | specific | | | | | | | | | |
| ERLIN1 | ER lipid raft | 1.5 | 1 | | | NM_006459; | | Plasma | 151 | 152 |
| | associated 1 | | | | | NM_001100626 | | Membrane | | |

LDA - results of low density microarray analysis, wherein a ✓ indicates significantly increased expression in EPCs.
EPC flow - results of flow cytometry showing percentage of EPCs in a population expressing the biomarker.
EC flow - results of flow cytometry showing percentage of endothelial cells in a population expressing the biomarker.
PBMC flow - results of flow cytometry showing percentage of peripheral blood monocytes (PBMCs) in a population expressing the biomarker.

### Example 5

### Detection of Protein Biomarkers of EPCs

Non-adherent CD133⁺ EPCs were isolated from umbilical cord blood using Miltenyi AutoMacsPro essentially as described in Example 2. These cells were cultured essentially as described in Example 2. Non-adherent natural EPCs were harvested at days 4 and 7. HUVECs were also prepared essentially as described in Example 2. Cells were then gently washed to remove any extraneous material while ensuring cell integrity. The carbohydrate moieties of the outer membrane protein was oxidized using 10 mM sodium periodate. Following removal of excess periodate the cells were lysed for 15 minutes in 100 mM Na Acetate pH 5.5 and 0.5% Triton-X/1% octyl-glucoside/150 mM NaCl. After removal of cell debris by centrifugation the oxidised glycoproteins were bound to beads via hydrazone coupling. The beads were extensively washed to remove any non-covalently bound cell related material. Proteins bound to the beads were reduced for 1 hour at 60° C. with 10 mM DTT followed by alkylation with 5 times molar excess of iodoacetamide. Following further washing proteins attached to the beads were digested with trypsin for 1.5 hour at 45° C. in 25 mM Tris pH 8.0. The tryptic peptides were then removed and the glycopeptides remaining attached to the beads released by cleavage of the asparagine linked carbohydrate using PNGase F enzyme over night at 37° C. Solution containing the released peptides was dried in an injection vial for mass spectrometric analysis.

The dried sample was then injected onto a HPLC (Ultimate 3000, Dionex) and fractionated by a pepmap 150 mm×150 μm column (C18 5μ) using 0.1% formic acid (aq) as A-buffer and 98% acetonitrile in 2% A-buffer as B-buffer. The peptides were eluted onto a 384 spot MALDI-MS target plate using a spotter (FC-proteineer, Bruker Daltonics, Germany) After drying the target was washed once with 10 mM ammonium phosphate buffer.

Molecular ion spectra of the 384 spots were automatically acquired and from the result a list of approximately 4000 major peptides was generated. Each of these peptides was collated into one datafile by the program WarpLC 1.2 and the most significant peak from each peptide was fragmented and analysed by MALDI-tof/tof-MS (Bruker-Daltonics, Germany). The resulting spectra were annotated and imported into Biotools 3.2 which controls the search parameters for a search using the Mascot search engine (Matrix Science, UK). The mass tolerance was set to 50 ppm for molecular ions and 0.5 Da for fragment ions.

The protein list resulting from the Mascot search was manually curated for the presence of a glycosylation site(s) and obvious miss assigned spectra.

Results of this analysis are summarised in Table 9.

TABLE 9
Summary of cell proteins identified in, on or secreted from EPCs.

| | | | Reference | | | | | | | |
|---|---|---|---|---|---|---|---|---|---|---|
| | | | Sequence | | | | | | Nucleo- | Amino |
| | | | (SwissProt | | | | | Location | tide | Acid |
| | Gene | | Accession | | Fold | EPC | EC | TM | SEQ | SEQ |
| Protein ID | name | Protein | Number) | P-Value | Change | flow | flow | domains | ID NO | ID NO |
| EMB_HUMAN | EMB | Embigin | NM_198449; | 0.009503 | 6.0 | | | Plasma | 1 | 2 |
| | | precursor - | NR_003955 | | | | | Membrane 1 | | |
| | | Homo sapiens | (Q6PCB8; | | | | | | | |
| | | (Human) | B7Z6S3) | | | | | | | |
| CELR2_HUMAN | CELSR2 | Cadherin | NM_014246 | 0.028395 | 1.73 | | | Plasma | 153 | 154 |
| | | EGF LAG | (Q9HCU4; | | | | | Membrane 7 | | |
| | | seven-pass | Q5T2Y7; | | | | | | | |
| | | G-type | Q92566) | | | | | | | |
| | | receptor 2 | | | | | | | | |
| | | precursor - | | | | | | | | |
| | | Homo sapiens | | | | | | | | |
| | | (Human) | | | | | | | | |
| NPTN_HUMAN | NPTN | Neuroplastin | NM_012428; | 0.0233 | 2.17 | | | Plasma | 155 | 156 |
| | | OS = | NM_017455 | | | | | Membrane 1 | | |
| | | Homo sapiens | (Q9Y639; | | | | | | | |
| | | GN = NPTN | B7Z4D3; | | | | | | | |
| | | PE = 1 SV = 2 | B7ZLL2; | | | | | | | |
| | | | Q17R52; | | | | | | | |
| | | | Q59EJ9; | | | | | | | |
| | | | Q6NVX7; | | | | | | | |
| | | | Q9Y640) | | | | | | | |
| APMAP_HUMAN | C20ORF3 | Adipocyte | NM_020531 | 0.090422 | 1.53 | | | Plasma | 157 | 158 |
| | | plasma | (Q9HDC9; | | | | | Membrane 1 | | |
| | | membrane- | A8K514; | | | | | | | |
| | | associated | B4DXG1; | | | | | | | |
| | | protein - | Q6UVZ8; | | | | | | | |
| | | Homo sapiens | Q9GZS8; | | | | | | | |
| | | (Human) | Q9NUB2) | | | | | | | |
| CLD20_HUMAN | CLDN20 | Claudin-20 | NM_001001346 | 0.023075 | 2.0 | | | Plasma | 101 | 102 |
| | | | (P56880) | | | | | Membrane 3 | | |
| GBRA3_HUMAN | GABRA3 | Gamma- | NM_000814; | 0.062762 | 2.81 | | | Plasma | 159 | 160 |
| | | aminobutyric | NM_021912 | | | | | Membrane 4 | | |
| | | acid receptor | (P34903; | | | | | | | |
| | | subunit | Q8TAF9) | | | | | | | |
| | | alpha-3 | | | | | | | | |
| DSG3_HUMAN | DSG3 | Desmoglein- | NM_001943 | 0.028143 | 7.4 | | | Plasma | 161 | 162 |
| | | 3 OS = | (P32926; | | | | | Membrane 1 | | |
| | | Homo sapiens | A8K2V2) | | | | | | | |
| | | GN = DSG3 | | | | | | | | |
| | | PE = 1 SV = 2 | | | | | | | | |
| PLXB2_HUMAN | PLXNB2 | Plexin-B2 | NM_005761 | 0.015987 | 8.6 | | | Plasma | 163 | 164 |
| | | precursor - | (O15031; | | | | | Membrane 1 | | |
| | | Homo sapiens | A6QRH0; | | | | | | | |
| | | (Human) | Q7KZU3; | | | | | | | |
| | | | Q9BSU7) | | | | | | | |
| CRCM1_HUMAN | ORAI1 | Calcium | NM_152288 | 0.070157 | 1.30 | | | Plasma | 165 | 166 |
| | | release- | (Q96D31; | | | | | Membrane 4 | | |
| | | activated | Q3MHV3; | | | | | | | |
| | | calcium | Q6DHX2; | | | | | | | |
| | | channel | Q96BP7; | | | | | | | |
| | | protein 1 | Q96K71) | | | | | | | |
| | | OS = | | | | | | | | |
| | | Homo sapiens | | | | | | | | |
| | | GN = ORAI1 | | | | | | | | |
| | | PE = 1 SV = 2 | | | | | | | | |
| DAG1_HUMAN | DAG1 | Dystroglycan | (Q14118; | | | | | Plasma | 167 | 168 |
| | | OS = | A8K6M7; | | | | | Membrane 1 | | |
| | | Homo sapiens | Q969J9) | | | | | | | |
| | | GN = DAG1 | | | | | | | | |
| | | PE = 1 SV = 2 | | | | | | | | |
| CN176_HUMAN | C14ORF176 | Transmembrane | (P0C7T8) | | | | | unknown | 169 | 170 |
| | | protein | | | | | | | | |
| | | C14orf176 | | | | | | | | |
| MPZL1_HUMAN | MPZL1 | Myelin | (O95297; | | | | | Plasma | 171 | 172 |
| | | protein zero- | B2REB9; | | | | | Membrane 1 | | |
| | | like protein 1 | Q5R332; | | | | | | | |
| | | precursor - | Q8IX11; | | | | | | | |
| | | Homo sapiens | Q9BWZ3; | | | | | | | |
| | | (Human) | Q9NYK4; | | | | | | | |
| | | | Q9UL20) | | | | | | | |
| CLD17_HUMAN | CLDN17 | Claudin-17 - | P56750; Q3MJB5; | | | | | Plasma | 173 | 174 |
| | | Homo sapiens | Q6UY37 | | | | | Membrane 3 | | |
| | | (Human) | | | | | | | | |
| GP125_HUMAN | GPR125 | Probable G- | (Q8IWK6; | | | | | Plasma | 175 | 176 |
| | | protein | Q6UXK9; | | | | | Membrane 7 | | |
| | | coupled | Q86SQ5; | | | | | | | |
| | | receptor 125 | Q8TC55) | | | | | | | |
| | | precursor - | | | | | | | | |
| | | Homo sapiens | | | | | | | | |
| | | (Human) | | | | | | | | |
| NICA_HUMAN | NCSTN | Nicastrin | (Q92542; | | | | negative | Plasma | 177 | 178 |
| | | precursor - | Q5T207; | | | | | Membrane 1 | | |
| | | Homo sapiens | Q86VV5) | | | | | | | |
| | | (Human) | | | | | | | | |
| UPK1A_HUMAN | UPK1A | Uroplakin-1a | (O00322; | | | | | Plasma | 179 | 180 |
| | | | Q3KNU5; | | | | | Membrane 4 | | |
| | | | Q3KNU6) | | | | | | | |
| TEN3_HUMAN | ODZ3 | Teneurin-3 - | (Q9P273; | | | | | Plasma | 181 | 182 |
| | | Homo sapiens | Q5XUL9; | | | | | Membrane 1 | | |
| | | (Human) | Q96SY2; | | | | | | | |
| | | | Q9NV77; | | | | | | | |
| | | | Q9NVW1; | | | | | | | |
| | | | Q9NZJ2) | | | | | | | |
| DCC_HUMAN | DCC | Netrin | (P43146) | | | | | Plasma | 183 | 184 |
| | | receptor | | | | | | Membrane 1 | | |
| | | DCC | | | | | | | | |
| | | OS = | | | | | | | | |
| | | Homo sapiens | | | | | | | | |
| | | GN = DCC | | | | | | | | |
| | | PE = 1 SV = 1 | | | | | | | | |
| K0090_HUMAN | KIAA0090 | Uncharacterized | (Q8N766; | | | | | Plasma | 185 | 186 |
| | | protein | A8K6F3; | | | | | Membrane 1 | | |
| | | KIAA0090 | Q14700; | | | | | | | |
| | | precursor - | Q5TG62; | | | | | | | |
| | | Homo sapiens | Q63HL0; | | | | | | | |
| | | (Human) | Q63HL3; | | | | | | | |
| | | | Q8NBH8) | | | | | | | |
| ACCN4_HUMAN | ACCN4 | Amiloride- | (Q96FT7; | | | | | Plasma | 187 | 188 |
| | | sensitive | Q53SB7; | | | | | Membrane 1 | | |
| | | cation | Q6GMS1; | | | | | | | |
| | | channel 4 | Q6PIN9; | | | | | | | |
| | | | Q9NQA4) | | | | | | | |
| CAC1D_HUMAN | CACNA1D | Voltage- | (Q01668; | | | | | Plasma | 189 | 190 |
| | | dependent L- | Q13916; | | | | | Membrane | | |
| | | type calcium | Q13931; | | | | | 19 | | |
| | | channel | Q9UDC3) | | | | | | | |
| | | subunit | | | | | | | | |
| | | alpha-1D - | | | | | | | | |
| | | Homo sapiens | | | | | | | | |
| | | (Human) | | | | | | | | |
| CSPG4_HUMAN | CSPG4 | Chondroitin | (Q6UVK1; | | | | | Plasma | 191 | 192 |
| | | sulfate | D3DW77; | | | | | Membrane 1 | | |
| | | proteoglycan | Q92675) | | | | | | | |
| | | 4 precursor - | | | | | | | | |
| | | Homo sapiens | | | | | | | | |
| | | (Human) | | | | | | | | |
| DPP6_HUMAN | DPP6 | Dipeptidyl | (P42658) | | | | negative | Plasma | 193 | 194 |
| | | aminopeptidase- | | | | | | Membrane 1 | | |
| | | like | | | | | | | | |
| | | protein 6 | | | | | | | | |
| | | OS = | | | | | | | | |
| | | Homo sapiens | | | | | | | | |
| | | GN = DPP6 | | | | | | | | |
| | | PE = 1 SV = 2 | | | | | | | | |
| FAT2_HUMAN | FAT2 | Protocadherin | (Q9NYQ8; | | | | | Plasma | 195 | 196 |
| | | Fat 2 | O75091; | | | | | Membrane 1 | | |
| | | precursor - | Q9NSR7) | | | | | | | |
| | | Homo sapiens | | | | | | | | |
| | | (Human) | | | | | | | | |
| LRP12_HUMAN | LRP12 | Low-density | (Q9Y561; | | | | | Plasma | 197 | 198 |
| | | lipoprotein | A8K137) | | | | | Membrane 1 | | |
| | | receptor- | | | | | | | | |
| | | related | | | | | | | | |
| | | protein 12 | | | | | | | | |
| | | precursor - | | | | | | | | |
| | | Homo sapiens | | | | | | | | |
| | | (Human) | | | | | | | | |
| NPY2R_HUMAN | NPY2R | Neuropeptide | (P49146; | | | | | Plasma | 199 | 200 |
| | | Y receptor | Q13281; | | | | | Membrane | | |
| | | type 2 - | Q13457; | | | | | GPCR = 7 | | |
| | | Homo sapiens | Q4W5G7; | | | | | | | |
| | | (Human) | Q6AZZ6; | | | | | | | |
| | | | Q9UE67) | | | | | | | |
| O11H4_HUMAN | OR11H4 | Olfactory | (Q8NGC9; | | | | | Plasma | 201 | 202 |
| | | receptor | B2RNQ4; | | | | | Membrane | | |
| | | 11H4 - | Q6IF07) | | | | | GPCR = 7 | | |
| | | Homo sapiens | | | | | | | | |
| | | (Human) | | | | | | | | |
| PCDA4_HUMAN | PCDHA4 | Protocadherin | (Q9UN74; | | | | | Plasma | 203 | 204 |
| | | alpha-4 | O75285; | | | | | Membrane 1 | | |
| | | precursor - | Q2M253) | | | | | | | |
| | | Homo sapiens | | | | | | | | |
| | | (Human) | | | | | | | | |
| PCDC1_HUMAN | PCDHAC1 | Protocadherin | (Q9H158; | | | | | Plasma | 205 | 206 |
| | | alpha-C1 | Q9Y5F5; | | | | | Membrane 1 | | |
| | | precursor - | Q9Y5I5) | | | | | | | |
| | | Homo sapiens | | | | | | | | |
| | | (Human) | | | | | | | | |
| RHBD2_HUMAN | RHBDD2 | Rhomboid | (Q6NTF9; | | | | | Plasma | 207 | 208 |
| | | domain- | Q7L534; | | | | | Membrane 5 | | |
| | | containing | Q9H5W6; | | | | | | | |
| | | protein 2 - | Q9UDT2) | | | | | | | |
| | | Homo sapiens | | | | | | | | |
| | | (Human) | | | | | | | | |
| SCN5A_HUMAN | SCN5A | Sodium | (Q14524; | | | | | Plasma | 209 | 210 |
| | | channel | A5H1P8 | | | | | Membrane | | |
| | | protein type | A6N922; | | | | | 22 | | |
| | | 5 subunit | A6N923; | | | | | | | |
| | | alpha | B2RTU0; | | | | | | | |
| | | OS = | Q75RX9; | | | | | | | |
| | | Homo sapiens | Q75RY0; | | | | | | | |
| | | GN = SCN5A | Q86UR3; | | | | | | | |
| | | PE = 1 SV = 2 | Q8IZC9; | | | | | | | |
| | | | Q96J69) | | | | | | | |
| SERC5_HUMAN | SERINC5 | Serine | (Q86VE9; | | | | | Plasma | 211 | 212 |
| | | incorporator | Q495A4; | | | | | Membrane 8 | | |
| | | 5 OS = | Q495A6) | | | | | | | |
| | | Homo sapiens | | | | | | | | |
| | | GN = SERINC5 | | | | | | | | |
| | | PE = 2 | | | | | | | | |
| | | SV = 1 | | | | | | | | |
| S12A1_HUMAN | SLC12A1 | Solute | (Q13621; | | | | | Plasma | 213 | 214 |
| | | carrier | A8JYA2) | | | | | Membrane | | |
| | | family 12 | | | | | | 11 | | |
| | | member 1 | | | | | | | | |
| | | OS = | | | | | | | | |
| | | Homo sapiens | | | | | | | | |
| | | GN = SLC12A1 | | | | | | | | |
| | | PE = 1 | | | | | | | | |
| | | SV = 2 | | | | | | | | |
| PCFT_HUMAN | SLC46A1 | Proton- | (Q96NT5; | | | | | Plasma | 215 | 216 |
| | | coupled | Q1HE20; | | | | | Membrane | | |
| | | folate | Q86T92; | | | | | 11 | | |
| | | transporter | Q8TEG3; | | | | | | | |
| | | | Q96FL0) | | | | | | | |
| SO1B1_HUMAN | SLCO1B1 | Solute | (Q9Y6L6; | | | | | Plasma | 217 | 218 |
| | | carrier | B2R7G2; | | | | | Membrane | | |
| | | organic | Q9NQ37; | | | | | 12 | | |
| | | anion | Q9UBF3; | | | | | | | |
| | | transporter | Q9UH89) | | | | | | | |
| | | family | | | | | | | | |
| | | member 1B1 | | | | | | | | |
| ANO2_HUMAN | ANO2 | Anoctamin-2 | (Q9NQ90; | | | | | Unknown 7 | 219 | 220 |
| | | OS = | C4N787; | | | | | | | |
| | | Homo sapiens | Q9H847) | | | | | | | |
| | | GN = ANO2 | | | | | | | | |
| | | PE = 1 SV = 2 | | | | | | | | |
| ABCAC_HUMAN | ABCA12 | ATP-binding | (Q86UK0; | | | | | Plasma | 221 | 222 |
| | | cassette sub- | Q53QE2; | | | | | Membrane | | |
| | | family A | Q53S55; | | | | | 12 | | |
| | | member 12 | Q8IZW6; | | | | | | | |
| | | OS = | Q96JT3; | | | | | | | |
| | | Homo sapiens | Q9Y4M5) | | | | | | | |
| | | GN = ABCA12 | | | | | | | | |
| | | PE = 1 | | | | | | | | |
| | | SV = 3 | | | | | | | | |
| CBPM_HUMAN | CPM | Carboxypeptidase M | (P14384; | | | | | Plasma | 223 | 224 |
| | | precursor - | B2R800; | | | | | Membrane 1 | | |
| | | Homo sapiens | Q9H2K9) | | | | | | | |
| | | (Human) | | | | | | | | |
| AAAT_HUMAN | SLC1A5 | Neutral | (Q15758; | | | | | Plasma | 225 | 226 |
| | | amino acid | A8K9H5; | | | | | Membrane 9 | | |
| | | transporter | D0EYG6; | | | | | | | |
| | | B(0) - | O95720; | | | | | | | |
| | | Homo sapiens | Q96RL9; | | | | | | | |
| | | (Human) | Q9BWQ3; | | | | | | | |
| | | | Q9UNP2) | | | | | | | |
| PK2L1_HUMAN | PKD2L1 | Polycystic | (Q9P0L9; | | | | negative | Plasma | 227 | 228 |
| | | kidney | O75972; | | | | | Membrane 5 | | |
| | | disease 2- | Q5W039; | | | | | | | |
| | | like 1 protein - | Q9UP35; | | | | | | | |
| | | Homo sapiens | Q9UPA2) | | | | | | | |
| | | (Human) | | | | | | | | |
| AT10A_HUMAN | ATP10A | Probable | (O60312; | | | | | Plasma | 229 | 230 |
| | | phospholipid- | Q969I4) | | | | | Membrane | | |
| | | transporting | | | | | | 10 | | |
| | | ATPase VA - | | | | | | | | |
| | | Homo sapiens | | | | | | | | |
| | | (Human) | | | | | | | | |
| ACHG_HUMAN | CHRNG | Acetylcholine | (P07510; | | | | | Plasma | 231 | 232 |
| | | receptor | B3KWM8; | | | | | Membrane 4 | | |
| | | subunit | Q53RG2) | | | | | | | |
| | | gamma | | | | | | | | |
| | | precursor - | | | | | | | | |
| | | Homo sapiens | | | | | | | | |
| | | (Human) | | | | | | | | |
| INSRR_HUMAN | INSRR | Insulin | (P14616; | | | | negative | Plasma | 233 | 234 |
| | | receptor- | O60724; | | | | | Membrane 1 | | |
| | | related | Q5VZS3) | | | | | | | |
| | | protein | | | | | | | | |
| | | precursor - | | | | | | | | |
| | | Homo sapiens | | | | | | | | |
| | | (Human) | | | | | | | | |
| SIRPBL_HUMAN | SIRPB1 | signal- | NM_006065 | | | | negative | Plasma | 331 | 332 |
| | | regulatory | | | | | | membrane 1 | | |
| | | protein beta 1 | | | | | | | | |
| LOXL4_HUMAN | LOXL4 | lysyl | NM_032211 | | | | negative | Extracellular | 337 | 338 |
| | | oxidase-like 4 | | | | | | space | | |
| CAC1B_HUMAN | CACNA1B | Voltage- | (Q00975; | | | | | Plasma | 235 | 236 |
| | | dependent | B1AQK5) | | | | | Membrane | | |
| | | N-type | | | | | | 22 | | |
| | | calcium | | | | | | | | |
| | | channel | | | | | | | | |
| | | subunit | | | | | | | | |
| | | alpha-1B - | | | | | | | | |
| | | Homo sapiens | | | | | | | | |
| | | (Human) | | | | | | | | |
| ADAM0_HUMAN | ADAM10 | Disintegrin | NM_001110 | | | | | | 329 | 330 |
| | | and | | | | | | | | |
| | | metalloproteinase | | | | | | | | |
| | | domain- | | | | | | | | |
| | | containing | | | | | | | | |
| | | protein 10 | | | | | | | | |
| CSF2RA_HUMAN | CSF2RA | GM-CSF | NM_006140, | | | | | | 333 | 334 |
| | | receptor | NM_172245, | | | | | | | |
| | | subunit alpha | NM_172246, | | | | | | | |
| | | precursor | NM_172247, | | | | | | | |
| | | | NM_172249, | | | | | | | |
| | | | NM_001161529, | | | | | | | |
| | | | NM_001161530, | | | | | | | |
| | | | NM_001161531, | | | | | | | |
| | | | NR_027760, | | | | | | | |
| | | | NM_001161532 | | | | | | | |
| EVI5_HUMAN | EVI5 | Ecotropic | NM_005665, | | | | | | 335 | 336 |
| | | viral | Q59FE7 | | | | | | | |
| | | integration 5 | | | | | | | | |
| LRRC33_HUMAN | LRRC33 | sapiens | NM_198565, | | | | | | 339 | 340 |
| | | Leucine rich | Q86YC3 | | | | | | | |
| | | containing | | | | | | | | |
| | | 33 | | | | | | | | |

### Example 6

### Role for DSG2 in Tube Formation and Angiogenesis

### 6.1 Materials and Methods

### Matrigel Assays

In vitro tube formation of HUVEC and DSG2 positive (C32) or DSG2 negative (MM200) melanoma cells (used as a model for EPCs) was assessed using a Matrigel matrix. HUVEC were stained with 10 μg/ml DiI-Ac-LDL (Biomedical Technologies, Stoughton, Mass.) for 4 hours at 37° C., 5% CO₂, washed once and incubated overnight at 37° C., 5% CO₂, after the addition of fresh media. C32 or MM200 were stained with 0.5 μM CFDA-SE (Invitrogen) in 0.1% FCS in PBS for 10 minutes. Labelled cells were incubated in fresh media for 30 minutes and then washed to ensure the residual CFDA-SE was completely removed. Fresh media was then added and the labelled cells were incubated overnight at 37° C., 5% CO₂. The next day, 12 μl Matrigel (BD Biosciences) was added to wells in a pre-warmed ibiTreat Angiogenesis μ-slide (Ibidi, Munich, Germany) and incubated at 37° C. for ≧30 minutes. Labelled cells were seeded together in Matrigel at a cell density of 1×10⁴ HUVEC or 0.7×10⁴ HUVEC and 0.5×10⁴ C32 or MM200 per well, in duplicate. Tube formation was monitored regularly and fluorescent and phase contrast images were captured using an IX81 microscope (Olympus) with 10×/0.4NA obj and a Hamamatsu Orca-ER camera after 6 hours. Fluorescence images were acquired using CellR software (Olympus Soft Imaging System).

### Small Interfering RNA Transfection

Using the manufacturer's protocol, DSG2 siRNA or scrambled control siRNA (1 nM, OriGene, Rockville, Md., USA) were transfected into DSG2 expressing cells (eg C32 cells) using Lipofectamine RNAiMAX (Invitrogen, Carlsbad, Calif., USA) in Opti-MEM medium (Invitrogen) when cells were at 30-40% confluency. The cells were then incubate for 24-48 hrs (for assessment of gene expression by qPCR) or 72 hrs (for assessment of protein expression by flow cytometry or function).

### Tissue Staining

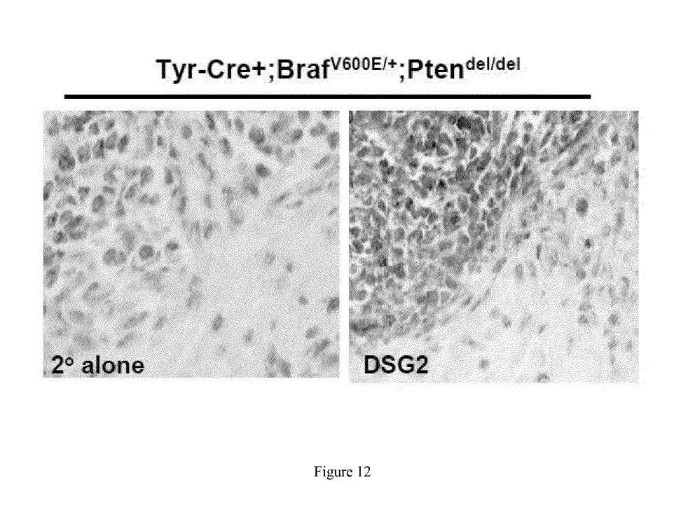

Human Tissue Array (T8234700-2) was purchased from Biochain (Hayward, Calif., USA) and following epitope retrieval the cores were stained with the mouse anti-human DSG2 mAb (1/50, clone 3G132, Abcam, Cambridge, Mass., USA) overnight at 4° C. prior to washing, peroxidise block, washing, incubation with anti-mouse-HRP (Vector Labs Impress), 30 min at room temperature, washing, incubation with DAB chromogen, washing and haematoxylin counter-stain. An adapted method, using an alkaline phosphatase/red chromagen system for detection of DSG2 in mouse melanoma (FIG. 12) was used as the natural pigmentation of melanocytes can interfere with detection using a brown chromagen.

### 6.2 Results

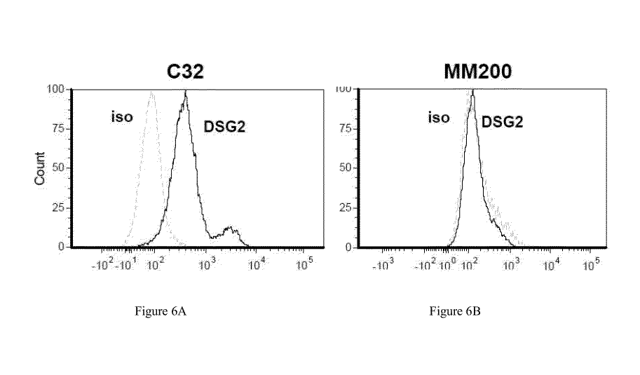

FIG. 6 shows that some melanoma cells express DSG2 on their cell surface. For example, melanoma cell line C32 expresses DSG2, whereas MM200 cells do not. Based on these data, C32 cells and MM200 were used for further experiments analyzing the role for DSG2 in tube formation. In some experiments, C32 cells were co-cultured with HUVECs in Matrigel®. Within about 7 hours after seeding cells formed tube-like structures that comprised both C32 melanoma cells and HUVECs, suggesting that these cells may contribute to tube formation in vitro and in vivo.

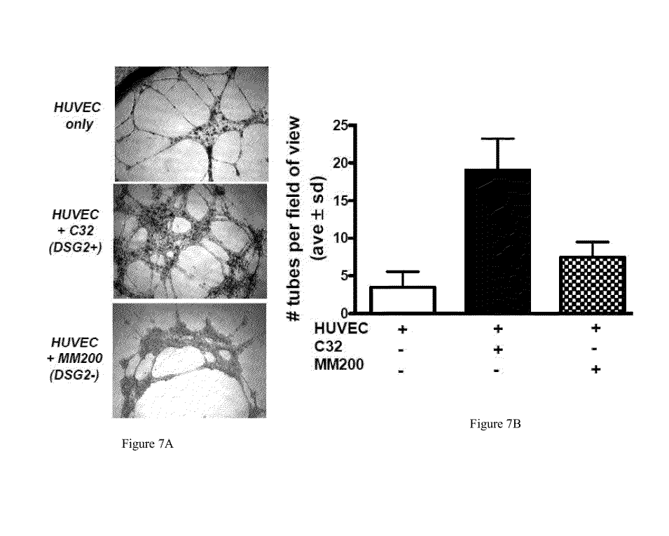

FIG. 7 shows that results of co-culturing HUVECs with C32 cells or MM200 cells in Matrigel®. As shown, culturing C32 (DSG2⁺) cells with HUVECs results in an increased number of tubes compared to HUVECs cultured alone or in the presence of MM200 cells. In contrast, culturing MM200 (DSG⁻) cells with HUVECs did not enhance tube formation in vitro.

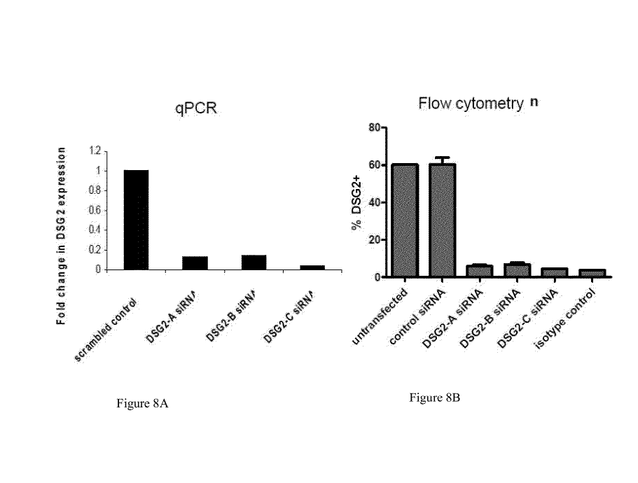

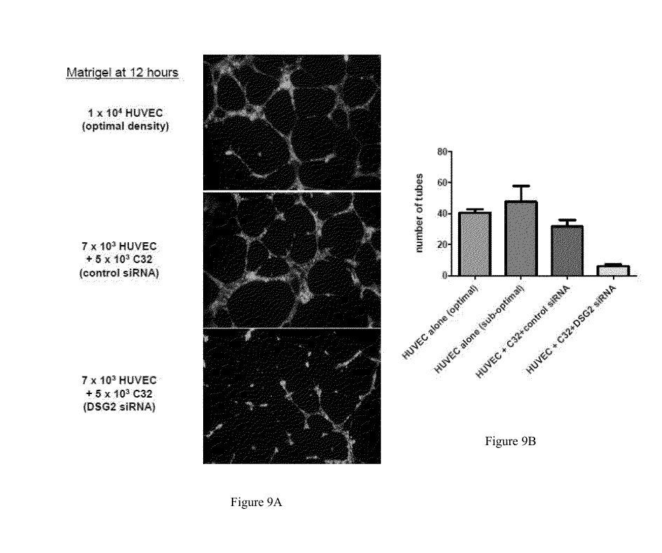

To further study the effect of DSG2 in tube formation, experiments were conducted in which DSG2 expression was knocked down suing siRNA. As shown in FIG. 8, siRNA targeting DSG2 can reduce DSG2 expression at the mRNA and protein level. FIG. 9 shows that when DSG2 expression is knocked-down in C32 cells, the amount of tubes formed when the cells were cultured in the presence of HUVECs in Matrigel® was dramatically reduced.

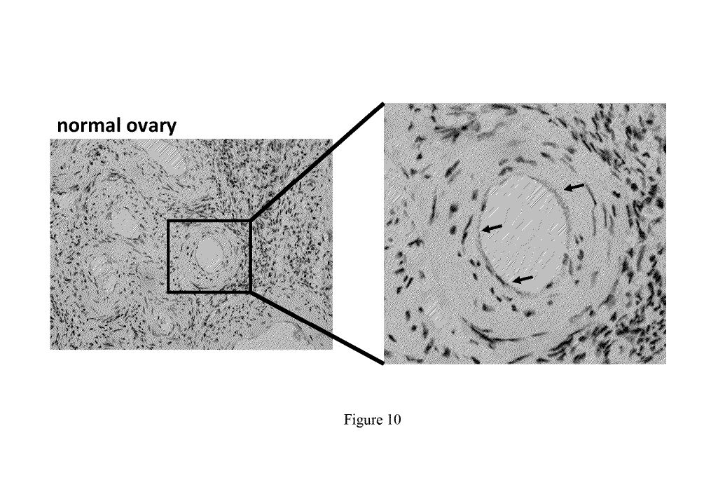

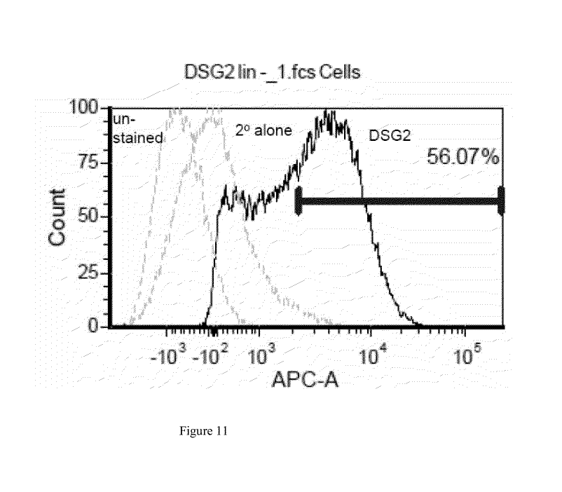

DSG2 expression was also assessed in vivo, and FIG. 10 shows that this protein is expressed on the vasculature of human tissue (ovary in this case). DSG2 was also shown to be expressed by melanocytes in melanomas.

FIG. 11 also shows that DSG2 is expressed on freshly isolated bone marrow cells from mouse, indicating a potential source and method for isolating EPCs based on DSG2 expression.

DSG2 was also identified on melanoma cells in a spontaneous model of this condition (Tyr-Cre+:Braf^{V600E/+};Pten^{del/del}).

### Example 7

### Expansion of EPCs

CD133⁺ cells were isolated from human umbilical cord blood as previously described prior to culturing at ˜7.5×10⁵ cells/ml in StemSpan media (Stem Cell Technologies, Vancouver, BC, Canada) in BD tissue culture plates (BD BioSciences, San Francisco, Calif., USA) for up to 7 days.

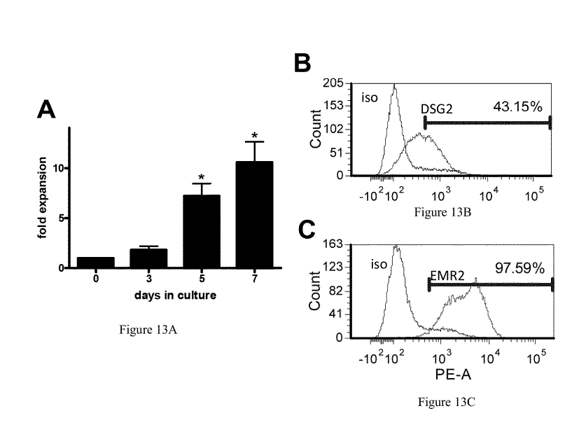

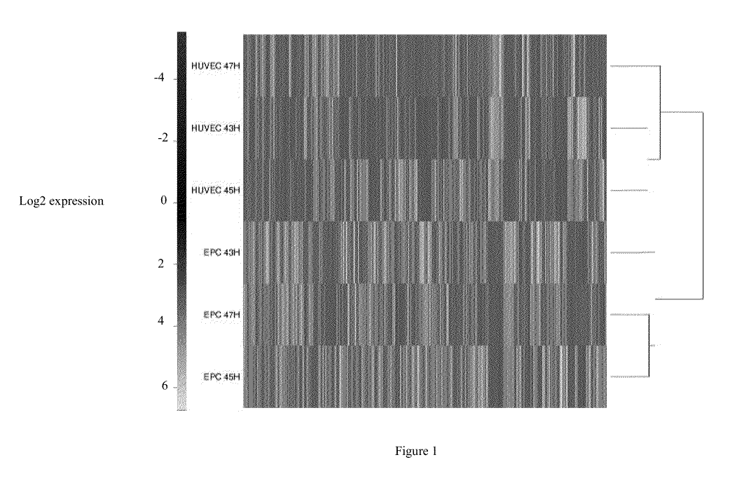

As shown in FIG. 13A, CD133⁺ EPCs could be isolated and cultured to expand the population. Even after seven days expansion the EPCs in the cultures expressed DSG2 or EMR2.

### Example 8

### Production of Monoclonal Antibodies

A monoclonal antibody that specifically binds to a protein set forth in one or more of Tables 1-6 is produced using methods known in the art. Briefly, a recombinant protein or a cell expressing said protein is administered to female Balb/C mice. Initially mice are sensitized by intraperitoneal injection of an adjuvant. Three boosts of the polypeptide or cells are administered at about 2, 5.5 and 6.5 months post initial sensitization. The first of these boosts is a subcutaneous injection while the remaining are administered by intraperitoneal injection. The final boost is administered 3 days prior to fusion.

The splenocytes of one of the immunized mice is fused to suitable myeloma cells, e.g., X63-Ag8.653 mouse myeloma cells, e.g., using PEG 1500. Following fusion, cells are incubated at 37° C. for 1 hour in heat inactivated fetal bovine serum. Fused cells are then transferred to normal medium and incubated overnight at 37° C. with 10% CO₂. The following day cells are plated using medium that has been supplemented with macrophage culture supernatants.

Two weeks after fusion, hybridoma cells are screened for antibody production by solid phase ELISA assay. Standard microtiter plates are coated with recombinant protein. Plates are then blocked, washed and then the test samples (i.e. supernatant from the fused cells) are added, in addition to control samples, (i.e. supernatant from an unfused cell). Antigen-antibody binding is detected by incubating the plates with anti-mouse or anti-human HRP conjugate (Jackson ImmunoResearch Laboratories) and 2,2′-azino-bis(3-ethylbenzthiazoline-6-sulphonic acid) (ABTS) peroxidase substrate system (Vector Laboratories, Burlingame, Calif. 94010, USA). Absorbance is read on an automatic plate reader at a wavelength of 405 nm.

Any colonies that are identified as positive by these screens continue to be grown and screened for several further weeks. Stable colonies are then isolated and stored at −80° C.

Positive stable hybridomas are then cloned by growing in culture for a short period of time and diluting the cells to a final concentration of 0.1 cells/well of a 96 well tissue culture plate. These clones are then screened using the previously described assay. This procedure is then repeated in order to ensure the purity of the clone.

Four different dilutions, 5 cells/well, 2 cells/well, 1 cell/well, 0.5 cells/well of the primary clone are prepared in 96-wells microtiter plates to start the secondary cloning. Cells are diluted in tissue culture media. To determine clones that antibodies that bind the antigen, supernatants from individual wells of the 0.5 or 1 cells/well microtiter plate are withdrawn after two weeks of growth and tested for the presence of antibody by ELISA assay as described above.

All positive clones are then adapted and expanded. A specific antibody is purified by Protein A affinity chromatography from the cell culture supernatant of cell culture.

The titer of the antibodies produced using this method are determined, e.g., using the Easy Titer kit available from Pierce (Rockford, Ill., USA). This kit utilizes beads that specifically bind mouse antibodies, and following binding these beads aggregate and no longer absorb light to the same degree as unassociated beads. Accordingly, the amount of an antibody in the supernatant of a hybridoma is assessed by comparing the OD measurement obtained from this sample to the amount detected in a standard, such as for example mouse IgG.

The specificity of the monoclonal antibody is then determined using Western blot analysis.

### Example 9

### Determining the Level of EPCs in a Biological Sample

Monoclonal antibodies essentially as described in Example 8 and/or commercially available antibodies, e.g., for sources described herein are used in the production of a two-site ELISA to determine the level of a protein expressed on EPCs in a biological sample.

Generally this method comprises capturing EPCs with a monoclonal antibody against one protein described herein and detecting those cells with an antibody against a different protein or lysing cells and capturing with an antibody against one epitope in a protein and detecting with an antibody against a different epitope against the same protein.

A capture antibody absorbed to a microtiter plate at about 20° C. for about 16 hours. Plates are then washed and blocked.

A test sample or a control sample comprising a known amount of EPCs or protein is contacted to the immobilized protein. A further control is cord blood derived sorted EPC (e.g., isolated based on expression of CD34 and/or VEGFR2)

The detection monoclonal antibody is conjugated to, e.g., HRP using an HRP conjugation kit (e.g., Alpha Diagnostics International, Inc., San Antonio, Tex., USA).

Following washing of the microtiter plates, the HRP conjugated monoclonal antibody is added to each well of the plate and incubated. Plates are then washed and ABTS (Sigma Aldrich, Sydney, Australia) is added to each well. Reactions are stopped after an appropriate time, e.g., approximately 20 minutes. Absorbance values are measured at 415 nm.

The amount of absorbance detected in negative control wells (cells or protein) is subtracted from the absorbance of each other well to determine the amount of detection antibody bound.

The amount of EPCs or protein is also assessed in normal and/or healthy subjects and/or subjects known to suffer from, e.g., rheumatoid arthritis. Samples use include, for example, buffy coat fraction. In this manner, an ELISA is produced to diagnose/prognose an EPC-associated condition, e.g., rheumatoid arthritis.

### Example 10

### Enumeration of EPCs

Monoclonal antibodies as described in Example 8 are labeled with a fluorophore using standard techniques.

Peripheral blood mononuclear cells, umbilical cord or bone marrow are resuspended in PBS in an optimally pre-titered cocktail of antibodies and incubated for about 20 minutes on ice. Labeled cells are washed in excess PBS and resuspended at about 5-10×10⁶ cells/mL and held on ice for flow cytometric analysis and sorting. Propidium iodide (PI; about 1 μg/mL) or Trypan Blue (about 0.2%), is used as a viability dye for exclusion of non-viable cells. FACS is performed using standard methods.

### Example 11

### EPC Transplantation to Models of Ischemia

Athymic nude mice or rats age 8-10 wk are anesthetized with 160 mg/kg pentobarbital (or equivalent anaesthetic) intraperitoneally for operative resection of one femoral artery or coronary artery, and subsequently for perfusion imaging Immediately before sacrifice, rodents are injected with an overdose of pentobarbital (or equivalent anaesthetic).

The impact of administration of EPCs isolated as described in Example 10 on therapeutic neovascularization is investigated in a murine model of hindlimb ischemia or a rat model of acute myocardial infarction. One day after operative excision of one femoral or coronary artery, athymic nude mice or rats, respectively, in which angiogenesis is characteristically impaired, receive an intracardiac injection of about 5×10⁵ culture-expanded EPCs. Two control groups are identically injected with either human vascular ECs (HVECs), harvested at 80-90% confluence, or media from the culture plates used for human (h)EPC ex vivo expansion.

For the study of EPC tracking, cells are marked with a fluorescent dye, e.g., carbocyanine DiI dye (Molecular Probes). Before cellular transplantation, cells in suspension are washed with PBS and incubated with the dye for 5 min at 37° C. and 15 min at 4° C. After two washing steps in PBS, the cells are resuspended in medium. Rodents receive dye-labeled EPCs at a total concentration of about 5×10⁵ to 10⁷ cells. Before sacrifice, a subgroup of rodents receive an intracardiac injection of either 50 μg of Bandeiraea simplicifolia lectin I (BS I; Vector Laboratories) or UEA-1 (Sigma).

Laser Doppler perfusion imaging (Moor Instrument, Wilmington, Del.) is used to record serial blood flow measurements over the course of 4 weeks postoperatively. For myocardial infarct model, magnetic resonance imaging (MRI) is used to record blood flow measurements over the course of 4 weeks postoperatively

Tissue sections from the lower calf muscles of ischemic and healthy limbs or hearts are harvested on days 3, 7, 14, and 28. Tissue from other organs and the healthy hindlimb are also examined for incorporation of EPCs. For immunohistochemistry, tissues are embedded in OCT compound (Miles Scientific, Elkhardt, Ind.) and snap frozen in liquid nitrogen. Frozen sections of 6-nm thickness are mounted on glass slides, air-dried for 1 h, and counterstained with biotinylated antibodies to UEA-1, mouse and human CD31 (platelet/endothelial cell adhesion molecule-1 (PECAM-1); Dako). Sections from other organs, including liver and spleen, are also examined for incorporation of hEPCs. The extent of neovascularization is assessed by measuring capillary density in light microscopic sections of muscles retrieved from ischemic mouse hindlimbs or the heart. The entire infrapatellar segment of each limb or the heart is examined. Sections were stained for alkaline phosphatase with indoxyl-tetrazolium and counterstained with eosin to detect capillary ECs.

### Example 12

### Inhibition of Angiogenesis

A mouse model of angiogenesis is produced essentially as described in Hoffmann et al. (1997). Briefly, sodium alginate of low viscosity and FITC-dextran with an average Mr of 150,000 is purchased from Sigma. FITC-dextran is dissolved in saline to a final concentration of 1%. Fluorescent microspheres with a size of 1 μm are obtained from Molecular Probes Europe (Leiden, Netherland). Cancer cells lines are also obtained, e.g., the murine Lewis lung carcinoma cell line LL2, the murine lymphoma line EL4, the murine myeloma line, B16, the human renal carcinoma cell line Caki-1, and the human renal carcinoma cell line Caki-2 are available from ATCC.

Sodium alginate of low viscosity is dissolved in sterile saline to a final concentration of 1.5%. Tumor cells are harvested from cell culture at 60-80% confluence. After centrifugation, the tumor cell pellet is directly resuspended with the alginate solution to the desired cell number and thereafter placed into a reservoir. Droplets containing tumor cells are produced by extrusion of the alginate solution through a 12-gauge cannula. The tumor cell alginate solution is dropped into a swirling bath of 80 mM CaCl₂. The calcium ions cause immediate gelling of each droplet by an exchange of sodium from the alginate. The size of the beads is minimized by a laminar air flow along the cannula. After incubation in the CaCl₂ bath for an additional 30 min, the beads are washed twice with buffer, centrifuged, and prepared for injection.

C57B16 mice or nude mice are injected subcutaneously with 0.1 ml of alginate beads containing tumor cells into the upper third of the back. Control mice are implanted with 0.1 ml of alginate beads without tumor cells. At the end of the experiment, 0.2 ml of 1% FITC-dextran solution (100 mg/kg) is injected intravenously (i.v.) into the lateral tail vein of mice.

Alginate implants are rapidly removed 20 mm after FITC-dextran injection and weighed, and after disection of the implant capsula, alginate beads are transferred to tubes containing 2 ml of saline. The tubes are mixed by vortexing for 20 s and centrifuged (3 min; 1000×g). After dilution (1:1), the fluorescence of the supernatant is measured.

Microspheres labeled with a fluorescent yellow-green dye at a size of 1 μm are used as indicated by the manufacturer. An aliquot of microspheres is injected into the lateral tail vein of mice (7×10⁹ microspheres/0.2 ml). Alginate implants are removed from the animals 20 min after injection of the microsphere solution and incubated with 2 ml of 2-ethoxyethylacetate for at least 24 hr to release the fluorescent dye from the disintegrated polystyrene latex membrane. The fluorescence of the samples is measured by excitation at 490 nm and emission at 506 nm.

Mice are injected i.v. with 0.2 ml of 1% FITC-dextran solution (100 mg/kg), and blood samples are taken at 10, 20, and 40 min post-injection. Heparinized blood samples are vortexed and centrifuged, and the fluorescence of the plasma is measured with a fluorescence spectrophotometer by excitation at 492 nm and emission at 515 nm. The amount of FITC-dextran within alginate implants is determined from the incubation supernatant. The corresponding blood volume of alginate implant is calculated using the following formula: blood volume (μl/alginate implant)=(FITC-dextran/alginate implant)/(FITC-dextran/μl blood).

C57BL/6 mice bearing implants encapsulating cancer cells are also treated with test antibodies from day 2 until day 10 after alginate implantation.

The assays described above permit both histological and quantitative assessment of angiogensis induced by tumors in the presence of antibodies of the disclosure.

### Example 13

### Treatment of Myocardial Infarction

Subjects diagnosed with acute ST segment myocardial infarction (STEMI) and a left ventricular ejection fraction (n LVEF)≦50% as determined by echocardiography are enrolled. Subjects are enrolled randomly as controls (n=15), receiving the standard of care, or assigned to the open-label cell therapy group (n=15) from whom 100 ml blood is collected. A monoclonal antibody that binds to a protein described in Table 1, such as a monoclonal antibody to DSG2, is used to isolate EPCs prior to expansion for 72 h. Nuclear-tracer cell labeling can be employed for high-sensitivity in vivo imaging of the transplanted cells. For example, cells can be labelled with 99mTc-extametazime (110 MBq), a lipophilic compound that turns hydrophilic after crossing the cell membrane and remains intracellular during cell tracking. The cells are then reinfused proximal to the infarct-related artery. Whole-body planar (static) scan and cardiac tomographic (SPECT) images are acquired 60 min after cell transfer to validate cell delivery. All subjects have serial electrocardiograms for 24 hours, measurements of cardiac biomarkers once a month for 3 months, twice-daily temperature measurements for 1 month echocardiograms and MRI at discharge and once a month for 3 months.

### Example 14

### Treatment of Melanoma

Subjects diagnosed with solid melanoma tumours confirmed histologically to be refractory to standard therapy or for which standard or curative therapy does not exist life expectancy of 3 months or longer, absence of known progressing or unstable brain metastases, and adequate hematologic, hepatic, and renal function are enrolled. Subjects are enrolled randomly as controls (n=32), receiving the standard of care, or assigned to antibody treatment group (n=32) from whom 10 ml blood is collected. A monoclonal antibody that binds to a protein described in Table 1, such as a monoclonal antibody to DSG2, is used to enumerate the circulating EPCs prior to treatment. The antibody will be administered biweekly for 12 months. Safety evaluations are conducted at baseline, day 8, day 15, day 29, and every 4 weeks thereafter. These evaluations included a physical examination, electrocardiography, laboratory studies that included a complete blood count, clinical chemical testing, and urinalysis. Patients undergo dermatologic evaluations at baseline and every 2 months during the study; and computed tomographic (CT) scans of the chest are analyzed for the appearance of new lesions suggestive of a primary cancer. CT studies are performed at 8-week intervals during therapy in all patients. The findings are judged according to the Response Evaluation Criteria in Solid Tumours (RECIST).

### Example 15

### Treatment of Diabetes

Subjects diagnosed with type 1 diabetes and a history of severe hypoglycemia and metabolic instability are enrolled. Subjects are enrolled randomly as controls (n=7), receiving the standard of care of islet transplantation alone, or assigned to the cell therapy group (n=7) undergoing islet transplantation in conjunction with EPC co-transplantation. 100 ml blood is collected and a monoclonal antibody that binds to a protein described in Table 1, such as a monoclonal antibody to DSG2, is used to isolate the EPCs prior to expansion for 72 h. Nuclear-tracer cell labeling may be employed for high-sensitivity in vivo imaging of the transplanted cells, e.g., as described in Example 11).

Islet preparations with more than 4000 islet equivalents per kilogram of the recipient's body weight in a packed-tissue volume of less than 10 ml are injected into the portal vein under fluoroscopic guidance. Portal venous pressure is measured at base line and after islet infusion. The final islet/EPC preparation (ratio of about 2:1) is suspended in 120 ml of medium that contains 500 U of heparin and 20 percent human albumin and is infused over a period of five minutes. Doppler ultrasonography of the portal vein and liver function tests are performed within 24 hours after transplantation. MRI is also performed within 24 hours after transplantation and once a month for 12 months to identify labelled EPCs.

### REFERENCES

Air, Proc. Natl. Acad. Sci USA 78: 7639-7643, 1981;
Andersson-Engels et al., Phys. Med. Biol, 42:815-824, 1997;
Arenkov et al., Anal. Biochem. 278:123-131, 2000;
Asahara et al., Science, 275: 964-967, 1997;
Audibert et al., Nature 289: 543, 1981;
F. M. Ausubel et al., (editors), Current Protocols in Molecular Biology, Greene Pub. Associates and Wiley-Interscience (1988, including all updates until present);
Avouac et al., Joint Bone Spine, 75: 131-137, 2008;
Bagley et al., Cancer Res 63: 5866, 2003;
Barnes et al., Anal. Biochem., 102: 255, 1980;
Beachey, Adv. Exp. Med. Biol., 185: 193, 1985;
Becchi et al., Int. J. Immunopharmacol., 21: 697-705, 2008;
Bendele J. Musculoskel. Neuron. Interact., 1(4): 377-385, 2001;
Bengtsson and Bengtsson BMC Bioinformatics, 7: 96, 2006;
Bengtsson and Hossjer BMC Bioinformatics, 7: 100, 2006;
Benjamini, et al., Journal of the Royal Statistical Society Series B, 57: 289-300, 1995;
Beaucage, et al., Tetrahedron Letters, 22: 1859-1862, 1981;
Berger and Kimmel, Guide to Molecular Cloning Techniques, Methods in Enzymology volume 152 Academic Press, Inc., San Diego, Calif.;
Berkner et al., BioTechniques, 6: 616, 1988;
Bolstad et al., Bioinformatics (Oxford, England), 19: 185-193, 2003;
Bresatz et al., Immunol. Methods, 327: 53-62, 2007;
Bernhard et al., Science, 245: 301-304, 1989;
Bhattacharya-Chatterjee et al., J of Immunospecificity. 141: 1398-1403, 1986;
Boerner et al., J Immunol, 147:86-95, 1991;
Bonder et al., Blood, 113(9):2108-17, 2009;
Bradl and Linington Brain Pathol., 6:303-311, 1996;
Brennan et al., Science, 229: 81 1985;
Bresslauer et al., Proc. Natl. Acad. Sci. USA, 83: 3746-3750, 1986;
Brown (editor), Essential Molecular Biology: A Practical Approach, Volumes 1 and 2, IRL Press (1991);
Bzik et al., Virology, 155: 322-333, 1986;
Carmeliet et al., J. Pathol., 190: 387-405, 2000;
Caron et al., J. Exp Med., 176: 191-1 195 1992;
Carter et al., Bio/Technology, 10: 163-167, 1992;
Caruthers, M. H., et al., “Methods in Enzymology,” Vol. 154, pp. 287-314 (1988);
Chen and Okayama, Mol. Cell Biol., 7: 2745-2752, 1987;
Chen et al., Nature, 446: 203-207, 2007;
Chothia and Lesk, J. Mol Biol., 196: 901-917, 1987;
J. E. Coligan et al. (editors) Current Protocols in Immunology, John Wiley &
Sons (including all updates until present);
Cima, et al., Biotechnol. Bioeng., 38:145 1991;
Cohen et al., Hum. Gene Ther., 10: 2701-2707, 1999;
Cole et al., Monoclonal Antibodies and Cancer Therapy, Alan R. Liss, p. 77 (1985);
Collins et al., Proc. Natl. Acad. Sci USA, 11: 7683, 1984;
Couffinhal et al., Am. J. Pathol., 152: 1667-1679, 1998;
Cunningham et al., Science, 244: 1081-1085 1989;
Cuello (editor), Immunohistochemistry, John Wiley and Sons, ASIN 0471900524 (1984);
Dai et al., J. Bioinform. Comput. Biol. 1: 627-645, 2004;
Denny et al., Blood, 110: 2907-2915, 2007;
Devereaux et al., Nucl. Acids Res., 12, 387-395, 1984;
Dieffenbach and Dveksler (editors) PCR Primer: A Laboratory Manual, Cold Spring Harbour Laboratories, NY, (1995);
Ding and Triggle, Vascular and Risk Management, 1: 55-71, 2005;
Edelson, The Cancer Journal, 4: 62, 1998;
Egan et al., Rheumatol., 47: 1484-1488, 2008;
Egholm et al., Am. Chem. Soc., 114: 1895, 1992;
Egholm et al., Nature, 365: 566, 1993;
Emini et al., Nature, 304: 699, 1983;
Estin et al., J Natl. Cancer Inst., 81: 445-446, 1989;
Fecheimer et al., Proc. Natl Acad. Sci. USA, 84: 8463-8467, 1987;
Feizi, Nature, 314: 53-57: 1985;
Flotte et al., J. Biol. Chem., 268: 3781-3790, 1993;
Folkman et al., N. Engl., J. Med., 285: 1182-1186, 1971;
Foon et al., Proc. Am. Soc. Clin. Oncol., 13: 294, 1994;
Fraley et al., Proc. Natl Acad. Sci. USA, 76: 3348-3352, 1979;
Frangioni, Curr. Opin. Chem. Biol, 7: 626-634, 2003;
Gait (editor) Oligonucleotide Synthesis: A Practical Approach, IRL Press, Oxford (1984);
Gautier, et al., Bioinformatics., 20: 307-315, 2004;
Gazzano-Santoro et al., J Immunol. Methods 202: 163 (1996);
Gefter et al., Somatic Cell Genet., 3: 231-236, 1977;
Ghetie et al., Blood, 83: 1329-1336, 1994;
Gentleman et al., Genome Biol. 5: R80, 2004;
Gilligan et al., Anal Chem., 74: 2041-2047, 2002;
Glover and Hames (editors), DNA Cloning: A Practical Approach, Volumes 1-4, IRL Press (1995 and 1996);
Gonzales-Scarano et al., Virology, 120: 42, 1982;
Goodman et al., (editors) Goodman and Gilman's The Pharmacological Basis of Therapeutics, 8th Ed., Macmillan Publishing Co. (1990);
Gopal, Mol. Cell Biol., 5: 1188-1190, 1985;
Graham et al., Gen Virol., 36: 59, 1977;
Graham and Van Der Eb, Virology, 52: 456-467, 1973;
Grisar et al., Circulation, 111: 204-211, 2005;
Gruber et al., J. Immunol., 152: 5368, 1994;
Gupta and Zhang Postgrad. Med. J., 81: 236-242, 2005;
Guss et al., EMBO J., 5: 1567-1575, 1986;
Hahn et al., Virology, 162: 167-180, 1988;
Ham et al., Meth. Enz., 58:44, 1979;
Harlow and Lane (editors) Antibodies: A Laboratory Manual, Cold Spring Harbour Laboratory, (1988);
Hartmann and Endres (editors), Manual of Antisense Methodology, Kluwer (1999);
Hellstrom et al., Cancer. Res., 45: 2210-2188, 1985;
Hellstrom et al., Cancer Res., 46: 3917-3923, 1986;
Henttu and Vihko, Biochem. Biophys. Res. Comm., 160: 903-910, 1989;
Herbrig et al., Ann. Rheum. Dis., 65: 157-163, 2006;
Herlyn et al., J Clin. Immunol 2: 135, 1982;
Hermonat et al., Proc. Natl. Acad. Sci. USA, 81: 6466-6470, 1984;
Hilkens et al., Trends in Bio. Chem. Sci., 17: 359, 1992;
Hochberg and Benjamin, Stat. Med., 9: 811-818, 1990;
Hoffmann et al., Cancer Res., 57: 3847-3851, 1997;
Hollinger et al., Proc. Natl. Acad. Sci. USA, 90: 6444-6448 1993;
Hoon et al., Cancer Res., 53: 5244-5250, 1993;
Irizarry et al., Biostatistics, 4: 249-264, 2003;
Itoh et al., Nature, 308: 19, 1986;
Jakobovits et al., Nature Biotechnology, 25, 1134-1143 2007;
Jung and Roh, J. Clin. Neurol, 4: 139-147, 2008;
Kabat et al., Sequences of Proteins of Immunological Interest, 5th Ed. Public Health Service, National Institutes of Health, Bethesda, Md., (1987 and 1991);
Kam et al., Proc. Natl. Acad. Sci. USA, 102: 11600-11605, 2005;
Kamarch, Methods Enzymol., 151:150-165, 1987;
Kang et al., Clin. Exp. Pharmacol. Physiol., Published online 22 Jun. 2009;
Kawamoto and Losordo, Trends Cardiovasc. Med., 18: 33-37, 2008;
Klein et al., Proc. Natl. Acad. Sci USA, 100: 8886-8891, 2003;
Kohler and Milstein, Nature 256: 495-497, 1975;
Kohler and Milstein, Eur. J. Immunol., 6: 511-519, 1976;
Kostelny et al., J Immunol., 148:1547-1553 1992;
Krenning et al., Trends in Mol. Med., 15: 180-189, 2009;
Largaespada et al., Curr. Top. Microbiol. Immunol., 166: 91-96, 1990;
Largaespada et al., J Immunol. Methods., 197: 85-95, 1996;
Levine et al., J. Immunol., 159: 5921-5930, 1997;
Limaye et al., Blood, 105: 3169-77, 2005;
Lindmark et al., J Immunol Meth., 62:1-13, 1983;
Litwin, et al., J. Cell Biol. 139, 219-228, 1997;
Liu et al., Antioxid. Redox Signal., 10: 1869-1882, 2008;
Livingston et al., J Clin. Oncol., 12: 1036-1044, 1994;
Lonberg et al., Nature, 368: 856-859, 1994;
Lonberg, In: “Transgenic Approaches to Human Monoclonal Antibodies.” Handbook of Experimental Pharmacology, 113: 49-101 (1994);
Lukacs et al., J. Exp. Med., 194: 551-555, 2001;
Lyden et al., Nature Med., 7: 1194-1201, 2001;
Marks et al., Bio/Technology, 10:779-783, 1992;
Mather et al., Annals N.Y. Acad. Sci., 383: 44-68, 1982;
Mendoza et al., Biotechniques 27: 778-788, 1999;
Messing Methods Enzymol, 101: 20-78, 1983;
Millstein et al., Nature, 305: 537-539, 1983;
Miltenyi et al., Cytometry, 11: 231-238, 1990;
Narang, et al., Meth. Enzymol., 68: 90, 1979;
Narang, editor, “Synthesis and Applications of DNA and RNA” Academic Press, New York (1987);
Natali et al., Cancer, 59: 55-63, 1987;
Needleman and Wunsch, J. Mol. Biol., 48: 443-453, 1970;
Nelson et al., Electrophoresis, 21: 1155-1163, 2000;
Nicolau and Sene, Biochim. Biophys. Acta, 721: 185-190, 1982;
Nielsen et al., J. Chem. Soc. Perkin Trans., 1: 3423, 1997;
Novellino et al., Cancer Immunol. Immunother. 54:187-207, 2005;
Orum et al., Nucl. Acids Res., 21: 5332, 1993;
Perbal (editor), “A Practical Guide to Molecular Cloning”, John Wiley and Sons (1984);
Perez and Walker, J. Immunol., 142: 3662-3667, 1990;
Perikles, Bioinformatics, 19: 1439-1440, 2003;
Pluckthun, Immunol. Revs., 130:151-188, 1992;
Putney et al., Science, 234: 1392-1395, 1986;
Rafat, Crit. Care Med., 35: 1677-1684, 2007;
Ramanujam et al., IEEE Transactions on Biomedical Engineering, 48: 1034-1041, 2001;
Ragnhammar et al., Int. J. Cancer, 53: 751-758, 1993;
Reff et al., Blood, 83: 435-445, 1994;
Rehman et al., Circulation, 107: 1164-1169, 2007;
Rippe et al., Mol. Cell Biol., 10: 689-695, 1990;
Rosenfeld et al., Science, 252: 431-434, 1991;
Rosenfeld et al., Cell, 68: 143-155, 1992;
Rota et al., Virology, 188: 135-142, 1982;
Rotmans et al., Can. J. Cardiol., 22: 929-932, 2006;
Rusinova et al., Cancer Cell., 4: 277-289, 2003;
Saleh et al., J. Immunol., 151: 3390-3398, 1993;
Sambrook et al. (editors), “Molecular Cloning: A Laboratory Manual”, Cold Spring Harbour Laboratory Press (1989);
Santa Lucia, Proc. Natl. Acad. Sci. USA, 95: 1460-1465, 1995;
Schatterman et al., J. Clin. Invest., 106: 571-578, 2000;
Scopes (editor), “Protein purification: principles and practice”, Third Edition, Springer Verlag (1994);
Sgouros et al., J Nucl. Med., 34: 422-430, 1993;
Shaked et al., Cancer Cell., 7: 101-111, 2005;
Shalaby et al., J Exp. Med., 175: 217-225, 1992;
Shitara et al., Cancer Immunol. Immunother., 36: 373-380, 1993;
Shield et al., 2002, J. Biol. Chem. 277: 26733-26740;
Shinkawa et al., J. Biol. Chem. 278: 3466-3473, 2003;
Shopes, Immunol., 148: 2918-2922, 1992;
Singh and Wengel, “Universality of LNA-Mediated High-Affinity Nucleic Acid Recognition”, Chem. Commun., 1247-1248 (1998);
Skerra et al., Curr. Opinion in Immunol., 5: 256-262, 1993;
Smyth et al., Bioinformatics (Oxford, England) 21: 2067-2075, 2005;
Smyth, In: Bioinformatics and Computational Biology Solutions using R and Bioconductor, Gentleman, Carey, Dudoit, Irizarry, Huber (eds.), Springer, New York, pages 397-420, 2005;
Sobrino et al., Stroke, 38: 2759-2764, 2007;
Spandidos et al., BMC Genomics, 9: 633, 2008;
Suresh et al., Methods in Enzymology, 121: 210, 1986;
Stevenson et al., Anti-Cancer Drug Design, 3:219-230 1989;
Taguchi et al., J. Clin. Invest., 114: 330-338, 2004;
Tailor et al., Nucl. Acids Res. 1816: 4928, 1990;
Tepper et al., Circulation, 106: 2781-2786, 2002;
Thompson et al., Nucl. Acids Res., 22: 4673-4680, 1994;
Tomizuka et al., Proc. Natl. Acad. Sci., USA 97: 722-727, 2000;
Tratschin et al., Mol. Cell. Biol., 5: 3251-3260, 1985;
Tratschin et al., J Virol., 51: 611-619, 1984;
Traunecker et al., EMBO J., 10: 3655-3659, 1991;
Tur-Kaspa et al., Mol. Cell Biol., 6: 716-718, 1986;
Tutt et al., J Immunol., 147: 60, 1991;
Ukai et al., J. Neurotrauma, 24: 508-520, 2007;
Vacanti, et al., J. Ped. Surg., 23: 3-9 1988;
Vacanti, et al., Plast. Reconstr. Surg., 88: 753-9 1991;
Van der Sluis et al., Gastroenterology, 131: 117-129, 2006;
Vijayasardahl et al., J Exp. Med., 171: 1375-1380, 1990;
Wang and Seed Nucl. Acids Res. 31: e154, 2003;
Wang et al., J Clin Invest., 118: 2629-2639, 2008;
Westerweel et al., Rheum. Dis., 66: 865-870, 2007;
Westerweel and Verhaar, Nat. Rev. Rheumatol., 5: 332-340, 2009;
Wettenhall and Smyth, Bioinformatics (Oxford, England), 20: 3705-3706, 2004;
Wolff et al., Cancer Research, 53: 2560-2565, 1993;
Wondisford et al., Mol. Endocrinol., 2: 32-39, 1988;
Wu and Wu, J. Biol. Chem., 262: 4429-4432, 1987;
Yang et al., Proc. Natl Acad. Sci USA, 87: 9568-9572, 1990;
Yaniv, Nature, 297: 17-18, 1982;
Yip et al., Stroke, 63: 69-74, 2008;
Yokata et al., Cancer Res., 52: 3402-3408, 1982;
Young et al., Prog. Cardiovasc. Dis., 49: 421-429, 2007;
Yu et al., Cancer Res. 51: 468-475, 1991;
Zhang et al., Circ. Res., 90: 284-288, 2002;
Zhang et al., Blood, 105: 3286-3294, 2005;
Zhu et al., Circulation, 118: 2156-2165, 2008; and
Zondor et al., Ann. Pharmacal, 38: 1258-1264, 2004.

We claim:

1. A method for detecting an endothelial progenitor cell (EPC) comprising determining the level of expression of a nucleic acid or protein set forth in Table 1, in, on or secreted from a cell, wherein an increased level of expression of a nucleic acid or protein set forth in Table 1 compared to another cell type is indicative of an EPC.

2. The method according to claim 1, wherein the nucleic acid or protein is expressed in, on or secreted from EPCs at a level at least 1.5 fold greater or 2 fold greater or 3 fold greater or 4 fold greater or 5 fold greater than in, on or secreted by human umbilical cord vascular endothelial cells (HUVECs).

3. The method according to claim 2, wherein the nucleic acid or protein is expressed in, on or secreted by non-adherent CD133⁺ EPCs at a level at least 1.5 fold greater or 2 fold greater or 3 fold greater or 4 fold greater or 5 fold greater than in, on or secreted by HUVECs.

4. The method according to claim 1, wherein the protein is selected from the group consisting of DSG2, EMR2, EMB, ADCY7, SLC39A8, TM7SF3, NKG7, NCSTN, SIRBP1, EVI5, LOXL4, INSRR, PKD2L1, DPP6, LRRC33 and SCL1A5 or the nucleic acid encodes one of the foregoing proteins.

5. The method according to claim 1 additionally comprising isolating the cell detected by the method.

6. A method for isolating a population of cells enriched for endothelial progenitor cells (EPCs), the method comprising contacting a population of cells comprising EPCs with a compound that binds to a protein set forth in Table 1 for a time and under conditions sufficient for the compound to bind to a cell and isolating cells to which the compound is bound.

7. The method according to claim 5, additionally comprising culturing the isolated cells to increase the number of isolated EPCs and/or differentiating the isolated and/or cultured EPCs and/or additionally comprising determining the activity of the isolated and/or cultured EPCs and/or additionally comprising formulating the isolated EPCs and/or cells isolated therefrom with a pharmaceutically acceptable carrier or excipient to thereby produce a pharmaceutical composition and/or additionally comprising immobilizing the isolated EPCs and/or cells derived therefrom on a solid or semi-solid matrix.

8. A composition comprising a population of cells enriched for endothelial progenitor cells (EPCs) expressing one or more nucleic acids or proteins set forth in Table 1.

9. The composition according to claim 8, wherein the population of cells is enriched for EPCs expressing one or more proteins selected from the group consisting of DSG2, EMR2, EMB, ADCY7, SLC39A8, TM7SF3, NKG7, NCSTN, SIRBP1, EVI5, LOXL4, INSRR, PKD2L1, DPP6, LRRC33 and SCL1A5.

10. The composition according to claim 8, wherein the EPCs additionally express one or more proteins selected from the group consisting of CD133, CD117, CD34 CD144, VEGFR2 and CD31.

11. The method according to claim 1, wherein detection of EPC(s) or failure to detect EPCs is diagnostic or prognostic of the EPC-associated condition.

12. The method according to claim 1, which is for localising and/or detecting and/or diagnosing and/or prognosing an endothelial progenitor cell (EPC)-associated condition in a subject, the method comprising:
(i) administering to a subject a compound that binds specifically to a compound that binds to a protein set forth in Table 1 such that the compound binds to the protein, if present; and
(ii) detecting the compound bound to the protein in vivo, wherein detection of the bound compound detects an EPC and localises and/or detects and/or diagnoses and/or prognoses the EPC-associated condition.

13. A method of treating or preventing a condition associated with reduced endothelial progenitor cell (EPC) numbers or activity, treating or preventing a condition associated with insufficient neovascularization and/or improving grafting and/or improving wound healing in a subject, the method comprising administering the composition comprising a population of cells enriched for endothelial progenitor cells (EPCs) expressing one or more nucleic acids or proteins set forth in Table 1 or a solid support or a semi-solid support having immobilized thereon a compound that binds to a protein set forth in Table 1 for a time and under conditions for the compound to bind to EPCs from the subject or:
(i) performing the method according to claim 5; and
(ii) administering the cells at (i) to the subject.

14. A method of modulating neovascularization and/or EPC numbers or activity in a subject, the method comprising administering to a subject in need thereof a compound that modulates expression and/or activity of a protein or nucleic acid set forth in Table 1, and/or administering a compound that binds to a protein set forth in Table 1 and modulates EPC activity and/or induces EPC death and/or EPC proliferation.

15. The method of claim 11, wherein the condition is selected from the group consisting of cardiovascular disease, diabetes or melanoma.

16. The method of claim 13, wherein the condition is cardiovascular disease or diabetes.
